(19) United States
(12) Patent Application Publication
(10) Pub. No.: US 2026/0176347 A1
(43) Pub. Date: Jun. 25, 2026

(54) Anti-Activin E Antibodies

(71) Applicant: AstralBio, Inc., Boston, MA (US)

(72) Inventors: Patrick Crutcher, San Diego, CA (US); Justin DiMartino, Boston, MA (US); Cody Allen Moore, San Diego, CA (US); Hongyu Zhang, San Diego, CA (US); Tom Sih-Yuan Hsu, San Diego, CA (US); Tam Thi Thanh Phuong, San Diego, CA (US); Alexander Tomoaki Taguchi, San Diego, CA (US); Cory Schwartz, San Diego, CA (US); Martin Brenner, San Diego, CA (US)

(21) Appl. No.: 19/431,094

(22) Filed: Dec. 23, 2025

Related U.S. Application Data
(60) Provisional application No. 63/737,980, filed on Dec. 23, 2024.
(60) Provisional application No. 63/764,665, filed on Feb. 28, 2025.
(60) Provisional application No. 63/823,081, filed on Jun. 13, 2025.
(60) Provisional application No. 63/907,507, filed on Oct. 29, 2025.

(51) Int. Cl. C07K 16/22; A61K 31/138; A61K 31/155; A61K 31/165; A61K 31/167; A61K 31/17; A61K 31/197; A61K 31/401; A61K 31/4015; A61K 31/403; A61K 31/4035; A61K 31/4045; A61K 31/4184; A61K 31/426; A61K 31/4418; A61K 31/4439; A61K 31/451; A61K 31/4985; A61K 31/517; A61K 31/522; A61K 31/549; A61K 39/00; A61K 39/395; A61P 3/04

(52) U.S. Cl. CPC C07K 16/22; A61K 31/138; A61K 31/155; A61K 31/165; A61K 31/167; A61K 31/17; A61K 31/197; A61K 31/401; A61K 31/4015; A61K 31/403; A61K 31/4035; A61K 31/4045; A61K 31/4184; A61K 31/426; A61K 31/4418; A61K 31/4439; A61K 31/451; A61K 31/4985; A61K 31/517; A61K 31/522; A61K 31/549; A61K 39/3955; A61P 3/04; A61K 2039/505; C07K 2317/622

(57) ABSTRACT

Provided herein are anti-Activin E antibodies having specific complementarity determining regions (CDRs) for the heavy and light chains, heavy and light chains, antigen binding fragments thereof, polynucleotides that encode the same, vectors, and host cells, and methods for treating a metabolic disorder, type 2 diabetes, obesity, an elevated triglyceride level, lipodystrophy, liver inflammation, fatty liver disease, hypercholesterolemia, an elevated liver enzyme, nonalcoholic steatohepatitis (NASH), a cardiovascular disease, cardiomyopathy, high blood pressure, and/or heart failure with the anti-Activin E antibodies disclosed herein.

### CROSS-REFERENCE TO RELATED APPLICATIONS

This application claims priority to U.S. Provisional Application Ser. No. 63/737,980, filed Dec. 23, 2024, U.S. Provisional Application Ser. No. 63/764,665, filed Feb. 28, 2025, U.S. Provisional Application Ser. No. 63/823,081, filed Jun. 13, 2025 and U.S. Provisional Application Ser. No. 63/907,507, filed Oct. 29, 2025, the entire contents of which are incorporated herein by reference.

### TECHNICAL FIELD OF THE INVENTION

This document relates to materials and methods for treating conditions involving Activin E, and particularly to the use of anti-Activin E antibodies and binding domains thereof.

### REFERENCE TO A SEQUENCE LISTING

The application contains a Sequence Listing which has been submitted electronically in .XML format and is hereby incorporated by reference in its entirety. Said .XML copy, created on Dec. 23, 2025, is named “IBIO1046.xml” and is 581,356 bytes in size. The sequence listing contained in this .XML file is part of the specification and is hereby incorporated by reference herein in its entirety.

### BACKGROUND OF THE INVENTION

Without limiting the scope of the invention, its background is described in connection with treatments targeting Activin E.

Activin E is a peptide that's part of the transforming growth factor-β (TGF-β) superfamily. It's produced in the liver and plays a role in energy homeostasis and metabolic health. These include energy homeostasis in which Activin E activates thermogenesis in adipose tissue, which can improve insulin sensitivity and reduce body fat mass. In metabolic health Activin E promotes adipose SMAD2/3 signaling, which can reduce lipid mobilization and promote inflammation. Finally, in liver-adipose communication Activin E is a hepatokine that links the liver and adipose tissue. It is believed that disrupting Activin E signaling could be a potential therapeutic target for obesity and diabetes.

One such treatment is taught by Lotta, et al, in U.S. Patent Publication No. 20220184114, entitled, “Methods of Treating Metabolic Disorders and Cardiovascular Disease With Inhibin Subunit Beta E (INHBE) Inhibitors”. These applicants are said to teach methods of treating a subject having metabolic disorders and/or cardiovascular diseases, methods of identifying subjects having an increased risk of developing a metabolic disorder and/or a cardiovascular disease, and methods of detecting human Inhibin Subunit Beta E variant nucleic acid molecules and variant polypeptides.

Despite these advances, a need remains for novel agents, methods of making those agents, and the use of such inhibitors of Activin E activity in vitro and in vivo for treating a wide variety of diseases or conditions such as metabolic disorders and cardiovascular disease.

### SUMMARY OF THE INVENTION

As embodied and broadly described herein, an aspect of the present disclosure relates to an anti-Activin E antibody or antigen binding domain thereof comprising comprises: a heavy chain variable domain (VH) complementarity determining region 1 (CDR1), VH CDR2, and VH CDR3 comprising an amino acid sequence of any one of the following SEQ ID NOs: 1, 2, 3; 11, 12, 13; 21, 22, 23; 31, 32, 33; 41, 42, 43; 51, 52, 53; 61, 62, 63; 71, 72, 73; 81, 82, 83; 91, 92, 93; 101, 102, 103; 111, 112, 113; 121, 122, 123; 131, 132, 133; 141, 142, 143; 151, 152, 153; 161, 162, 163; 171, 172, 173; 181, 182, 183; 191, 192, 193; 201, 202, 203; 211, 212, 213; 221, 222, 223; 231, 232, 233; 241, 242, 243; 251, 252, 253; 261, 262, 263; 271, 272, 273; 281, 282, 283; 291, 292, 293; 301, 302, 303; 311, 312, 313; 321, 322, 323; 331, 332, 333; 341, 342, 343; 351, 352, 353; 361, 362, 363; 371, 372, 373; 381, 382, 383; 391, 392, 393; 401, 402, 403; 411, 412, 413; 421, 422, 423; 431, 432, 433; 441, 442, 443; 451, 452, 453; 461, 462, 463; 471, 472, 473; 481, 482, 483; or 491, 492, 493, respectively; and a light chain variable domain (VL) CDR1, VL CDR2, and VL CDR3 comprising the amino acid sequence of any one of the following SEQ ID NOs: 4, 5, 6; 14, 15, 16; 24, 25, 26; 34, 35, 36; 44, 45, 46; 54, 55, 56; 64, 65, 66; 74, 75, 76; 84, 85, 86; 94, 95, 96; 104, 105, 106; 114, 115, 116; 124, 125, 126; 134, 135, 136; 144, 145, 146; 154, 155, 156; 164, 165, 166; 174, 175, 176; 184, 185, 186; 194, 195, 196; 204, 205, 206; 214, 215, 216; 224, 225, 226; 234, 235, 236; 244, 245, 246; 254, 255, 256; 264, 265, 266; 274, 275, 276; 284, 285, 286; 294, 295, 296; 304, 305, 306; 314, 315, 316; 324, 325, 326; 334, 335, 336; 344, 345, 346; 354, 355, 356; 364, 365, 366; 374, 375, 376; 384, 385, 386; 394, 395, 396; 404, 405, 406; 414, 415, 416; 424, 425, 426; 434, 435, 436; 444, 445, 446; 454, 455, 456; 464, 465, 466; 474, 475, 476; 484, 485, 486; or 494, 495, 496, respectively. In one aspect, antibody comprises: a VH and VL pair comprising at least 95, 96, 97, 98, 99, or 100% sequence identify to SEQ ID NOS; 7, 8; 17, 18; 27, 28; 37, 38; 47, 48; 57, 58; 67, 68; 77, 78; 87, 88; 97, 98; 107, 108; 117, 118; 127, 128; 137, 138; 147, 148; 157, 158; 167, 168; 177, 178; 187, 188; 197, 198; 207, 208; 217, 218; 227, 228; 237, 238; 247, 248; 257, 258; 267, 268; 277, 278; 287, 288; 297, 298; 307, 308; 317, 318; 327, 328; 337, 338; 347, 348; 357, 358; 367, 368; 377, 378; 387, 388; 397, 398; 407, 408; 417, 418; 427, 428; 437, 438; 447, 448; 457, 458; 467, 468; 477, 478; 487, 488; or 497, 498. In another aspect, the antibody comprises: a heavy chain and light chain variable domains are encoded by a polynucleotide or polynucleotides comprising at least 95, 96, 97, 98, 99, or 100% sequence identify to SEQ ID NOS: 9, 10; 19, 20; 29, 30; 39, 40; 49, 50; 59, 60; 69, 70; 79, 80; 89, 90; 99, 100; 109, 110; 119, 120; 129, 130; 139, 140; 149, 150; 159, 160; 169, 170; 179, 180; 189, 190; 199, 200; 209, 210; 219, 220; 229, 230; 239, 240; 249, 250; 259, 260; 269, 270; 279, 280; 289, 290; 299, 300; 309, 310; 319, 320; 329, 330; 339, 340; 349, 350; 359, 360; 369, 370; 379, 380; 389, 390; 399, 400; 409, 410; 419, 420; 429, 430; 439, 440; 449, 450; 459, 460; 469, 470; 479, 480; 489, 490; 499, 500; or 601, 602. In another aspect, the antibody is a monoclonal antibody. In another aspect, the antibody is a full-length antibody. In another aspect, the antibody is an antibody fragment selected from F(ab′)₂, Fab, Fab′, Fv, or scFv. In another aspect, the antibody comprises an Fc domain selected from one of the following: human IgG1, human IgG2, human IgG3, and human IgG4.

As embodied and broadly described herein, an aspect of the present disclosure relates to a method of treating a disease in a subject in need thereof, comprising administering to the subject a therapeutically effective amount of the antibody described hereinabove.

As embodied and broadly described herein, an aspect of the present disclosure relates to a single chain fragment variable (scFv) Activin E antagonist comprising: a heavy chain variable domain (VH) complementarity determining region 1 (CDR1), VH CDR2, and VH CDR3 comprising an amino acid sequence of any one of the following SEQ ID NOs: 1, 2, 3; 11, 12, 13; 21, 22, 23; 31, 32, 33; 41, 42, 43; 51, 52, 53; 61, 62, 63; 71, 72, 73; 81, 82, 83; 91, 92, 93; 101, 102, 103; 111, 112, 113; 121, 122, 123; 131, 132, 133; 141, 142, 143; 151, 152, 153; 161, 162, 163; 171, 172, 173; 181, 182, 183; 191, 192, 193; 201, 202, 203; 211, 212, 213; 221, 222, 223; 231, 232, 233; 241, 242, 243; 251, 252, 253; 261, 262, 263; 271, 272, 273; 281, 282, 283; 291, 292, 293; 301, 302, 303; 311, 312, 313; 321, 322, 323; 331, 332, 333; 341, 342, 343; 351, 352, 353; 361, 362, 363; 371, 372, 373; 381, 382, 383; 391, 392, 393; 401, 402, 403; 411, 412, 413; 421, 422, 423; 431, 432, 433; 441, 442, 443; 451, 452, 453; 461, 462, 463; 471, 472, 473; 481, 482, 483; or 491, 492, 493; and a light chain variable domain (VL) CDR1, VL CDR2, and VL CDR3 comprising the amino acid sequence of any one of the following SEQ ID NOs: 4, 5, 6; 14, 15, 16; 24, 25, 26; 34, 35, 36; 44, 45, 46; 54, 55, 56; 64, 65, 66; 74, 75, 76; 84, 85, 86; 94, 95, 96; 104, 105, 106; 114, 115, 116; 124, 125, 126; 134, 135, 136; 144, 145, 146; 154, 155, 156; 164, 165, 166; 174, 175, 176; 184, 185, 186; 194, 195, 196; 204, 205, 206; 214, 215, 216; 224, 225, 226; 234, 235, 236; 244, 245, 246; 254, 255, 256; 264, 265, 266; 274, 275, 276; 284, 285, 286; 294, 295, 296; 304, 305, 306; 314, 315, 316; 324, 325, 326; 334, 335, 336; 344, 345, 346; 354, 355, 356; 364, 365, 366; 374, 375, 376; 384, 385, 386; 394, 395, 396; 404, 405, 406; 414, 415, 416; 424, 425, 426; 434, 435, 436; 444, 445, 446; 454, 455, 456; 464, 465, 466; 474, 475, 476; 484, 485, 486; or 494, 495, 496, respectively. In one aspect, the scFv comprises at least 95, 96, 97, 98, 99, or 100% amino acid sequence identify to: 501; 503; 505; 507; 509; 511; 513; 515; 517; 519; 521; 523; 525; 527; 529; 531; 533; 535; 537; 539; 541; 543; 545; 547; 549; 551; 553; 555; 557; 559; 561; 563; 565; 567; 569; 571; 573; 575; 577; 579; 581; 583; 585; 587; 589; 591; 593; 595; 597; or 599. In another aspect, the scFv comprises at least 95, 96, 97, 98, 99, or 100% nucleic sequence identify to: 502; 504; 506; 508; 510; 512; 514; 516; 518; 520; 522; 524; 526; 528; 530; 532; 534; 536; 538; 540; 542; 544; 546; 548; 550; 552; 554; 556; 558; 560; 562; 564; 566; 568; 570; 572; 574; 576; 578; 580; 582; 584; 586; 588; 590; 592; 594; 596; 598; or 600. In another aspect, the scFv is a tandem scFv.

As embodied and broadly described herein, an aspect of the present disclosure relates to a method of treating a disease in a subject in need thereof, comprising administering to the subject a therapeutically effective amount of the antibody described hereinabove. In one aspect, the disease is selected from at least one of: a metabolic disorder, type 2 diabetes, obesity, an elevated triglyceride level, lipodystrophy, liver inflammation, fatty liver disease, hypercholesterolemia, an elevated liver enzyme, nonalcoholic steatohepatitis (NASH), a cardiovascular disease, cardiomyopathy, high blood pressure, or heart failure. In another aspect, the subject is human.

As embodied and broadly described herein, an aspect of the present disclosure relates to a nucleic acid comprising an anti-Activin E antibody or antigen binding domain thereof, comprising: a heavy chain variable domain (VH) complementarity determining region 1 (CDR1), VH CDR2, and VH CDR3 comprising an amino acid sequence of any one of the following SEQ ID NOs: 1, 2, 3; 11, 12, 13; 21, 22, 23; 31, 32, 33; 41, 42, 43; 51, 52, 53; 61, 62, 63; 71, 72, 73; 81, 82, 83; 91, 92, 93; 101, 102, 103; 111, 112, 113; 121, 122, 123; 131, 132, 133; 141, 142, 143; 151, 152, 153; 161, 162, 163; 171, 172, 173; 181, 182, 183; 191, 192, 193; 201, 202, 203; 211, 212, 213; 221, 222, 223; 231, 232, 233; 241, 242, 243; 251, 252, 253; 261, 262, 263; 271, 272, 273; 281, 282, 283; 291, 292, 293; 301, 302, 303; 311, 312, 313; 321, 322, 323; 331, 332, 333; 341, 342, 343; 351, 352, 353; 361, 362, 363; 371, 372, 373; 381, 382, 383; 391, 392, 393; 401, 402, 403; 411, 412, 413; 421, 422, 423; 431, 432, 433; 441, 442, 443; 451, 452, 453; 461, 462, 463; 471, 472, 473; 481, 482, 483; or 491, 492, 493; and a light chain variable domain (VL) CDR1, VL CDR2, and VL CDR3 comprising the amino acid sequence of any one of the following SEQ ID NOs: 4, 5, 6; 14, 15, 16; 24, 25, 26; 34, 35, 36; 44, 45, 46; 54, 55, 56; 64, 65, 66; 74, 75, 76; 84, 85, 86; 94, 95, 96; 104, 105, 106; 114, 115, 116; 124, 125, 126; 134, 135, 136; 144, 145, 146; 154, 155, 156; 164, 165, 166; 174, 175, 176; 184, 185, 186; 194, 195, 196; 204, 205, 206; 214, 215, 216; 224, 225, 226; 234, 235, 236; 244, 245, 246; 254, 255, 256; 264, 265, 266; 274, 275, 276; 284, 285, 286; 294, 295, 296; 304, 305, 306; 314, 315, 316; 324, 325, 326; 334, 335, 336; 344, 345, 346; 354, 355, 356; 364, 365, 366; 374, 375, 376; 384, 385, 386; 394, 395, 396; 404, 405, 406; 414, 415, 416; 424, 425, 426; 434, 435, 436; 444, 445, 446; 454, 455, 456; 464, 465, 466; 474, 475, 476; 484, 485, 486; or 494, 495, 496, respectively. In one aspect, the antibody or antigen binding domain thereof comprises a heavy chain and light chain variable domain encoding polynucleotide having at least 95, 96, 97, 98, 99, or 100% sequence identify to SEQ ID NOS: 9, 10; 19, 20; 29, 30; 39, 40; 49, 50; 59, 60; 69, 70; 79, 80; 89, 90; 99, 100; 109, 110; 119, 120; 129, 130; 139, 140; 149, 150; 159, 160; 169, 170; 179, 180; 189, 190; 199, 200; 209, 210; 219, 220; 229, 230; 239, 240; 249, 250; 259, 260; 269, 270; 279, 280; 289, 290; 299, 300; 309, 310; 319, 320; 329, 330; 339, 340; 349, 350; 359, 360; 369, 370; 379, 380; 389, 390; 399, 400; 409, 410; 419, 420; 429, 430; 439, 440; 449, 450; 459, 460; 469, 470; 479, 480; 489, 490; and 499, 500. In another aspect, the e antibody is a monoclonal, bispecific, multivalent, multi-specific, diabody, chimeric, scFv antibody, or domain thereof. In another aspect, the scFv encodes a polypeptide that comprises at least 95, 96, 97, 98, 99, or 100% amino acid sequence identify to: 501; 503; 505; 507; 509; 511; 513; 515; 517; 519; 521; 523; 525; 527; 529; 531; 533; 535; 537; 539; 541; 543; 545; 547; 549; 551; 553; 555; 557; 559; 561; 563; 565; 567; 569; 571; 573; 575; 577; 579; 581; 583; 585; 587; 589; 591; 593; 595; 597; or 599. In another aspect, the scFv comprises at least 90, 91, 92, 93, 94, 95, 96, 97, 98, 99, or 100% nucleic sequence identify to: 502; 504; 506; 508; 510; 512; 514; 516; 518; 520; 522; 524; 526; 528; 530; 532; 534; 536; 538; 540; 542; 544; 546; 548; 550; 552; 554; 556; 558; 560; 562; 564; 566; 568; 570; 572; 574; 576; 578; 580; 582; 584; 586; 588; 590; 592; 594; 596; 598; or 600. In another aspect, the antibody binding domain is fused to an Fc domain of any one of the following: human IgG1, human IgG2, human IgG3, and human IgG4. In another aspect, the nucleic acid sequence is optimized for expression in a bacterial, fungal, mammalian, insect, or plant cell.

As embodied and broadly described herein, an aspect of the present disclosure relates to a vector comprising the nucleic acid described hereinabove.

As embodied and broadly described herein, an aspect of the present disclosure relates to a host cell comprising nucleic acid the vector described hereinabove.

As embodied and broadly described herein, an aspect of the present disclosure relates to a method of treating a subject having or at risk of developing a metabolic disorder, type 2 diabetes, obesity, an elevated triglyceride level, lipodystrophy, liver inflammation, fatty liver disease, hypercholesterolemia, an elevated liver enzyme, nonalcoholic steatohepatitis (NASH), a cardiovascular disease, cardiomyopathy, high blood pressure, and/or heart failure, the method comprising administering an anti-Inhibin E antibody administering an antibody, scFv, or antigen binding domain thereof described hereinabove or any the nucleic acid described hereinabove to the subject. In another aspect, the anti-Activin antibody or binding domain thereof comprises a variable heavy chain and light chain nucleic acid sequence having at least 95, 96, 97, 98, 99, or 100% sequence identify to SEQ ID NOS: 9, 10; 19, 20; 29, 30; 39, 40; 49, 50; 59, 60; 69, 70; 79, 80; 89, 90; 99, 100; 109, 110; 119, 120; 129, 130; 139, 140; 149, 150; 159, 160; 169, 170; 179, 180; 189, 190; 199, 200; 209, 210; 219, 220; 229, 230; 239, 240; 249, 250; 259, 260; 269, 270; 279, 280; 289, 290; 299, 300; 309, 310; 319, 320; 329, 330; 339, 340; 349, 350; 359, 360; 369, 370; 379, 380; 389, 390; 399, 400; 409, 410; 419, 420; 429, 430; 439, 440; 449, 450; 459, 460; 469, 470; 479, 480; 489, 490; 499, 500; or 601, 602. In another aspect, the anti-Activin antibody or binding domain thereof comprises an scFv comprising at least 95, 96, 97, 98, 99, or 100% amino acid sequence identify to: 501; 503; 505; 507; 509; 511; 513; 515; 517; 519; 521; 523; 525; 527; 529; 531; 533; 535; 537; 539; 541; 543; 545; 547; 549; 551; 553; 555; 557; 559; 561; 563; 565; 567; 569; 571; 573; 575; 577; 579; 581; 583; 585; 587; 589; 591; 593; 595; 597; or 599. In another aspect, the subject is administered a therapeutic agent that treats or inhibits metabolic disorders or cardiovascular diseases in a standard dosage amount. In another aspect, the subject is administered a therapeutic agent that treats or inhibits metabolic disorders or cardiovascular diseases that is the same as or lower than a standard dosage amount.

As embodied and broadly described herein, an aspect of the present disclosure relates to a method of treating a subject with a therapeutic agent that treats or inhibits a metabolic disorder, type 2 diabetes, obesity, an elevated triglyceride level, lipodystrophy, liver inflammation, fatty liver disease, hypercholesterolemia, an elevated liver enzyme, nonalcoholic steatohepatitis (NASH), a cardiovascular disease, cardiomyopathy, high blood pressure, or heart failure, the method comprising administering an antibody or antigen binding domain thereof described hereinabove, or any the nucleic acid described hereinabove to the subject. In one aspect, the subject is administered or continued to be administered a therapeutic agent that treats or inhibits the metabolic disorder, type 2 diabetes, obesity, an elevated triglyceride level, lipodystrophy, liver inflammation, fatty liver disease, hypercholesterolemia, an elevated liver enzyme, nonalcoholic steatohepatitis (NASH), a cardiovascular disease, cardiomyopathy, high blood pressure, or heart failure in a standard dosage amount; or the subject is administered or continued to be administered the therapeutic agent that treats or inhibits the metabolic disorder, type 2 diabetes, obesity, an elevated triglyceride level, lipodystrophy, liver inflammation, fatty liver disease, hypercholesterolemia, an elevated liver enzyme, nonalcoholic steatohepatitis (NASH), a cardiovascular disease, cardiomyopathy, high blood pressure, or heart failure in an amount that is the same as or lower than a standard dosage amount. In another aspect, the metabolic disorder is selected from at least one of: type 2 diabetes, and the therapeutic agent is chosen from metformin, insulin, glyburide, glipizide, glimepiride, repaglinide, nateglinide, thiazolidinediones, rosiglitazone, pioglitazone, sitagliptin, saxagliptin, linagliptin, exenatide, liraglutide, semaglutide, canagliflozin, dapagliflozin, and empagliflozin, or any combination thereof; obesity, and the therapeutic agent is chosen from orlistat, phentermine, topiramate, bupropion, naltrexone, and liraglutide, or any combination thereof: elevated triglyceride, and the therapeutic agent is chosen from rosuvastatin, simvastatin, atorvastatin, fenofibrate, gemfibrozil, fenofibric acid, niacin, and an omega-3 fatty acid, or any combination thereof, lipodystrophy, and the therapeutic agent is chosen from tesamorelin, metformin, poly-L-lactic acid, calcium hydroxyapatite, polymethylmethacrylate, bovine collagens, human collagens, silicone, and hyaluronic acid, or any combination thereof; liver inflammation, and the therapeutic agent is a hepatitis therapeutic or a hepatitis vaccine; fatty liver disease include, and the subject is administered bariatric surgery and/or dietary intervention; hypercholesterolemia, and the therapeutic agent is chosen from: atorvastatin, fluvastatin, lovastatin, pitavastatin, pravastatin, rosuvastatin calcium, simvastatin, cholestyramine, colesevelam, and colestipol, alirocumab, evolocumab, niaspan, niacor, fenofibrate, gemfibrozil, and bempedoic, or any combination thereof; an elevated liver enzyme, and the therapeutic agent is chosen from coffee, folic acid, potassium, vitamin B6, a statin, and fiber, or any combination thereof; or nonalcoholic steatohepatitis (NASH) and the therapeutic agent is obeticholic acid, Selonsertib, Elafibranor, Cenicriviroc, GR MD 02, MGL 3196, IMM124E, arachidyl amido cholanoic acid, GS0976, Emricasan, Volixibat, NGM282, GS9674, Tropifexor, MN 001, LMB763, BI 1467335, MSDC 0602, PF 05221304, DF102, Saroglitazar, BMS986036, Lanifibranor, Semaglutide, Nitazoxanide, GRI 0621, EYP001, VK2809, Nalmefene, LIK066, MT 3995, Elobixibat, Namodenoson, Foralumab, SAR425899, Sotagliflozin, EDP 305, Isosabutate, Gemcabene, TERN 101, KBP 042, PF 06865571, DUR928, PF 06835919, NGM313, BMS 986171, Namacizumab, CER 209, ND L02 s0201, RTU 1096, DRX 065, IONIS DGAT2Rx, INT 767, NC 001, Seladepar, PXL770, TERN 201, NV556, AZD2693, SP 1373, VK0214, Hepastem, TGFTX4, RLBN1127, GKT 137831, RYI 018, CB4209-CB4211, and JH 0920. In another aspect, the cardiovascular disease is selected from at least one of: high blood pressure, and the therapeutic agent is chosen from chlorthalidone, chlorothiazide, hydrochlorothiazide, indapamide, metolazone, acebutolol, atenolol, betaxolol, bisoprolol fumarate, carteolol hydrochloride, metoprolol tartrate, metoprolol succinate, nadolol, benazepril hydrochloride, captopril, enalapril maleate, fosinopril sodium, lisinopril, moexipril, perindopril, quinapril hydrochloride, ramipril, trandolapril, candesartan, eprosartan mesylate, irbesartan, losartan potassium, telmisartan, valsartan, amlodipine besylate, bepridil, diltiazem hydrochloride, felodipine, isradipine, nicardipine, nifedipine, nisoldipine, verapamil hydrochloride, doxazosin mesylate, prazosin hydrochloride, terazosin hydrochloride, methyldopa, carvedilol labetalol hydrochloride, alpha methyldopa, clonidine hydrochloride, guanabenz acetate, guanfacine hydrochloride, guanadrel, guanethidine monosulfate, reserpine, hydralazine hydrochloride, and minoxidil, or any combination thereof; cardiomyopathy, and the therapeutic agent is an ACE inhibitor, an angiotensin II receptor blocker, a beta blocker, a calcium channel blocker, digoxin, an antiarrhythmic, an aldosterone blocker, a diuretic, an anticoagulant, a blood thinner, and a corticosteroid; or heart failure, and the therapeutic agent is an ACE inhibitor, an angiotensin-2 receptor blocker, a beta blocker, a mineralocorticoid receptor antagonist, a diuretic, ivabradine, sacubitril valsartan, hydralazine with nitrate, and digoxin.

### BRIEF DESCRIPTION OF THE DRAWINGS

For a more complete understanding of the features and advantages of the present invention, reference is now made to the detailed description of the invention along with the accompanying figures and in which:

FIG. 1 is a graph that shows the antagonism of Activin E at 0.2 nM by the antibodies in reporter cell line.

FIG. 2 is a graph that shows the antagonism of Activin E at 0.02 nM by candidate antibodies in reporter cell line.

FIG. 3A is a graph that shows the establishment of Activin E signaling assay in differentiated human adipocytes, and FIG. 3B is a graph that shows the antagonism of Activin E signaling in human adipocytes by AVE-06-H08 at 100 nM Activin E using the antibodies disclosed herein.

FIG. 4A to 4G show a Diet Induced Obesity (DIO) mouse model study plan and results. FIG. 4A shows the DIO mouse model study plan. FIG. 4B is a graph that shows baseline and vehicle corrected change in body weight post dose. 2-way ANOVA used for statistical measures to compare mean body weight at each timepoint to the vehicle only group. FIG. 4C is a graph that shows body composition analysis of total fat mass. Statistics calculated using 2-way ANOVA, and comparing to baseline value. FIG. 4D is a graph that shows body composition analysis of total lean mass. Statistics calculated using 2-way ANOVA, and comparing to baseline value. FIG. 4E is a graph that shows weight of various fat depots at terminal end point. One-way ANOVA used for statistical analysis. FIG. 4F is a graph that shows a histological analysis of eWAT adipose tissue from terminal endpoint, measuring adipocyte minimum diameter and area. One-way ANOVA used for statistical analysis. FIG. 4G shows representative images of adipocyte histology. One-way ANOVA used for statistical analysis.

FIGS. 5A to 5F show the study design and results for weight regain prevention in DIO mice. Semaglutide dosed daily, mAbs dosed twice per week. FIG. 5B are graphs that show the baseline and vehicle-corrected body weights. Arrow indicates initiation of BIW mAb dosing. 2-way ANOVA used for statistical analysis. FIG. 5C is a graph that shows the food intake, reported on per-mouse basis. FIG. 5D is a graph that shows the terminal liver weight. FIG. 5E is a graph that shows the terminal adipose depot weights. FIG. 5F is a graph that shows the Terminal quadriceps weight.

FIG. 6A shows the study design for PK study in obese mature nonhuman primates (NHPs). FIG. 6B shows the non-human primate (NHP) selection criteria. FIG. 6C shows the serum concentration of AVE-06-H08_IgG4 at various timepoints. FIG. 6D shows the average serum concentration of AVE-06-H08_IgG4 with half-life calculated using linear elimination from days 14-56.

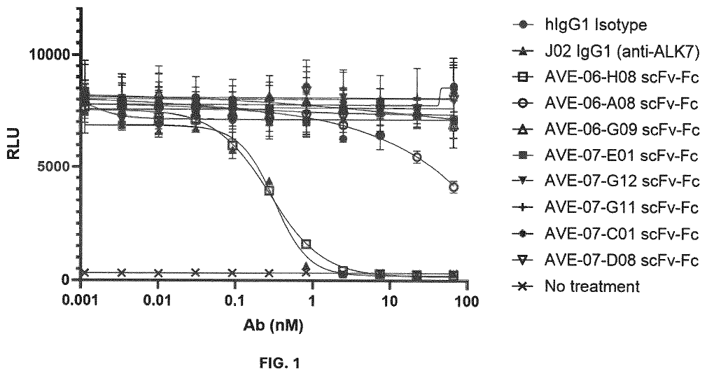

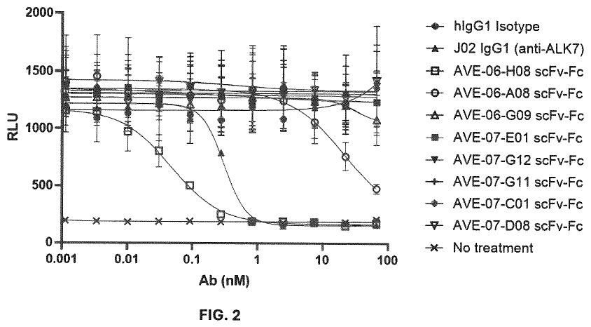

### DETAILED DESCRIPTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

It should be understood that, unless clearly indicated, in any method described or disclosed herein that includes more than one act, the order of the acts is not necessarily limited to the order in which the acts of the method are recited, but the disclosure encompasses exemplary embodiments in which the order of the acts is so limited.

All publications, patents, and patent applications mentioned in this specification are herein incorporated by reference to the same extent as if each individual publication, patent, or patent application was specifically and individually indicated to be incorporated by reference.

### Antibodies.

As used herein, the term “antibody” refers to an intact antibody or a binding fragment thereof that binds specifically to a target antigen—anti-Activin E. Antigen binding domains or fragments are produced by recombinant DNA techniques, or by enzymatic or chemical cleavage of intact antibodies. Antibody binding domain(s) or fragment(s) include, e.g., antigen-binding domain or fragment (Fab), F(ab′)₂ (mono-specific, bi-specific, or multivalent), fragment variable (Fv) containing VH and VL sequences, single chain variable domain or fragment (scFv) containing VH and VL sequences linked together in one chain (single, tandem, or multivalent), single chain antibody fragments (scAb) or other antibody variable region domain or fragment, such as retaining antigen binding specificity such as diabodies, minibodies, or combinations thereof. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay). The antigen binding domain or fragment of the disclosure retain anti-Activin E antigen binding specificity. Antibodies (Abs) and immunoglobulins (Igs) are glycoproteins having the same structural characteristics. The present invention includes monoclonal antibodies and binding fragments thereof that are completely recombinant, in other words, where the complementarity determining regions (CDRs) are genetically spliced into an antibody backbone, often referred to as veneering an antibody, e.g., a human antibody framework Thus, in certain aspects, the monoclonal antibody is a fully synthesized antibody. In certain embodiments, the monoclonal antibodies (and binding fragments thereof) can be made in bacterial or eukaryotic cells, including mammalian, non-mammalian animals, yeast, insect, or plant cells.

As used herein, a “subject” may be a mammalian subject. Mammalian subjects include, humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate, for example a cynomolgus monkey. In some embodiments, the subject is a companion animal (e.g., cats, dogs).

As used herein, the terms “antigen binding domain” or “antibody fragment” refers to a portion of a full-length antibody, generally the antigen-binding or variable region, and include Fab, Fab′, F(ab′)₂, Fv, and scFv fragments. Papain digestion of antibodies produces two identical antigen-binding fragments, called the Fab fragment, each with a single antigen-binding site, and a residual “Fc” fragment, so-called for its ability to crystallize readily. Pepsin treatment yields an F(ab′)₂ fragment that has two antigen-binding fragments which are capable of cross-linking antigen, and a residual other fragment (which is termed pFc′). As used herein, “functional fragment” with respect to antibodies, refers to Fv, F(ab) and F(ab′)₂ fragments.

As used herein, the “fragment variable” or “Fv” fragment is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment, also designated as F(ab), also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)₂ pepsin digestion product. Additional chemical couplings of antibody fragments are known to those of ordinary skill in the art.

Native antibodies and immunoglobulins are usually heterotetrameric glycoproteins of about 150,000 daltons, composed of two identical light (L) chains and two identical heavy (H) chains. Each light chain is linked to a heavy chain by at least one covalent disulfide bond, however, the number of disulfide linkages varies between the heavy chains of different immunoglobulin isotypes. Each heavy and light chain also has regularly spaced intrachain disulfide bridges. Each heavy chain has at one end a variable domain (V_{H}) followed by the constant domains. Each light chain has a variable domain at one end (V_{L}) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82 4592-4596 (1985), relevant portions incorporated herein by reference.

As used herein, an “isolated” antibody is one that has been identified and separated and/or recovered from a component of the environment in which it was produced. Contaminant components of its production environment are materials, which would interfere with diagnostic or therapeutic uses for the antibody, and may include enzymes, hormones, and other proteinaceous or nonproteinaceous solutes. In certain embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequenator, such as at least 15 residues of sequence; or 3) to homogeneity by SDS-PAGE under reducing or non-reducing conditions using, e.g., Coomasie blue or silver stain. Isolated antibody includes the antibody in situ within recombinant cells since at least one component of the antibody's natural environment will not be present. Ordinarily, however, isolated antibody will be prepared by at least one purification step.

As used herein, the terms “antibody mutant” or “antibody variant” refer to an amino acid sequence variant of an antibody wherein one or more of the amino acid residues have been modified. Such mutants or variants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90, 91, 92, 93, 94, 95, 96, 97, 98, 99, or 100% sequence identity to the amino acid and nucleic acid sequences disclosed herein. Generally, the “antibody mutant” or “antibody variant” will maintain the same CDR sequences (that is, CDR sequences having 100% sequence identity to the CDRs), but will have the variances of at least 80%, or at least 85%, or at least 90, 91, 92, 93, 94, 95, 96, 97, 98, or 99% sequence identity in the framework regions. However, the skilled artisan will recognize that 100% sequence identity of the CDRs is not required, as conservative amino acid changes can be made to the CDRs, which can then be tested for binding of Activin E as taught herein, without undue experimentation.

As used herein, the term “variable” in the context of the variable domain of antibodies, refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. et al. (1989), Nature 342: 877), or both, that is Chothia plus Kabat. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al.) The constant domains are not involved directly in binding an antibody to its cognate antigen but exhibit various effector function, such as participation of the antibody in antibody-dependent cellular toxicity.

The light chains of antibodies (immunoglobulin) from any vertebrate species can be assigned to one of two clearly distinct types, called kappa and lambda, based on the amino sequences of their constant domain. Depending on the amino acid sequences of the constant domain of their heavy chains, “immunoglobulins” can be assigned to different classes. There are at least five (5) major classes of immunoglobulins: IgA, IgD, IgE, IgG, and IgM, and several of these may be further divided into subclasses (isotypes), e.g., IgG-1, IgG-2, IgG-3, and IgG4; IgA-1 and IgA-2. The subunit structures and three-dimensional configurations of different classes of immunoglobulins are well known.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed and claimed invention may be made by the hybridoma method first described by Kohler and Milstein, Nature 256, 495 (1975), relevant portions incorporated herein by reference.

All monoclonal antibodies used in accordance with the presently disclosed and claimed invention will be either (1) the result of a deliberate immunization protocol, as described in more detail hereinbelow; or (2) the result of an immune response that results in the production of antibodies naturally in the course of a disease or cancer.

The uses of the monoclonal antibodies of the presently disclosed and claimed invention may require administration of such or similar monoclonal antibody to a subject, such as a human. However, when the monoclonal antibodies are produced in a non-human animal, such as a rodent or chicken, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the presently disclosed and claimed invention can be “humanized”, that is, the antibodies are engineered such that antigenic portions thereof are removed and like portions of a human antibody are substituted therefore, while the antibodies' affinity for Activin E is retained. This engineering may only involve a few amino acids, or may include entire framework regions of the antibody, leaving only the complementarity determining regions of the antibody intact. Several methods of humanizing antibodies are known in the art and are disclosed in U.S. Pat. No. 6,180,370, issued to Queen et al on Jan. 30, 2001; U.S. Pat. No. 6,054,927, issued to Brickell on Apr. 25, 2000; U.S. Pat. No. 5,869,619, issued to Studnicka on Feb. 9, 1999; U.S. Pat. No. 5,861,155, issued to Lin on Jan. 19, 1999; U.S. Pat. No. 5,712,120, issued to Rodriquez et al on Jan. 27, 1998; and U.S. Pat. No. 4,816,567, issued to Cabilly et al on Mar. 28, 1989, relevant portions incorporated herein by reference.

Humanized forms of antibodies are chimeric immunoglobulins, immunoglobulin chains or fragments thereof (such as Fab, Fab′, F(ab′)₂, Fv, scFv or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., 1986; Riechmann et al., 1988; Verhoeyen et al., 1988), by substituting nonhuman (i.e., rodent, chicken) CDRs or CDR sequences for the corresponding sequences of a human antibody, see, e.g., U.S. Pat. No. 5,225,539. In some instances, F, framework residues of the human immunoglobulin are replaced by corresponding non-human residues from the donor antibody. Humanized antibodies can also comprise residues that are found neither in the recipient antibody nor in the imported CDR or framework sequences. In general, the humanized antibody will comprise substantially all of, at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin.

The presently disclosed and claimed invention further includes the use of fully human monoclonal antibodies cross-reactive against Activin E. Fully human antibodies essentially relate to antibody molecules in which the entire sequence of both the light chain and the heavy chain, including the CDRs, arise from human genes. Such antibodies are termed “human antibodies” or “fully human antibodies” herein. Human monoclonal antibodies can be prepared by, e.g., the trioma technique; the human B-cell hybridoma technique (see Kozbor, et al., Hybridoma, 2:7 (1983)) and the EBV hybridoma technique to produce human monoclonal antibodies (see Cole, et al., PNAS 82:859 (1985)), or as taught herein. Human monoclonal antibodies may be utilized in the practice of the presently disclosed and claimed invention and may be produced by using human hybridomas (see Cote, et al., PNAS 80:2026 (1983)) or by transforming human B-cells with Epstein Barr Virus in vitro (see Cole, et al., 1985), relevant portions incorporated herein by reference.

In addition, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example but not by way of limitation, in U.S. Pat. Nos. 5,545,807; 5,545,806; 5,569,825; 5,625,126; 5,633,425; 5,661,016, and in Marks et al., J Biol. Chem. 267:16007, (1992); Lonberg et al., Nature, 368:856 (1994); Morrison, 1994; Fishwild et al., Nature Biotechnol. 14:845 (1996); Neuberger, Nat. Biotechnol. 14:826 (1996); and Lonberg and Huszar, Int Rev Immunol. 13:65 (1995), relevant portions incorporated herein by reference.

A method for producing an antibody of interest, such as a human antibody, is disclosed in U.S. Pat. No. 5,916,771, issued to Hori et al. on Jun. 29, 1999, and incorporated herein by reference. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95, 95, 97, 98, 99% or 100% purity. When used in this context, the term “isolated” does not exclude the presence of the same nucleic acid molecule or polypeptide in alternative physical forms, such as dimers or phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “treatment” refers to both therapeutic treatment and prophylactic or preventative measures. Those in need of treatment include those already with the disorder as well as those in which the disorder is to be prevented.

As used herein, the term “disorder” refers to any condition that would benefit from treatment with the polypeptide. This includes chronic and acute disorders or diseases including those infectious or pathological conditions that predispose the mammal to the disorder in question.

An antibody or antibody fragment can be generated with an engineered sequence or glycosylation state to confer preferred levels of activity in antibody-dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

Alternatively, or additionally, it may be useful to combine amino acid modifications with one or more further amino acid modifications that alter complement component Clq binding and/or the complement-dependent cytotoxicity (CDC) function of the Fc region of an IL-23p19 binding molecule. The binding polypeptide of particular interest may be one that binds to Clq and displays complement-dependent cytotoxicity. Polypeptides with pre-existing Clq binding activity, optionally further having the ability to mediate CDC may be modified such that one or both of these activities are enhanced. Amino acid modifications that alter Clq and/or modify its complement-dependent cytotoxicity function are described, for example, in WO/0042072, which is hereby incorporated by reference.

An Fc region of an antibody can be designed to alter the effector function, e.g., by modifying Clq binding and/or FcγR binding and thereby changing complement-dependent cytotoxicity (CDC) activity and/or antibody-dependent cell-mediated cytotoxicity (ADCC) activity. These “effector functions” are responsible for activating or diminishing a biological activity (e.g., in a subject). Examples of effector functions include, but are not limited to: Clq binding; CDC; Fc receptor binding; ADCC; phagocytosis; down-regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

As used herein, a single chain variable fragment (scFv) refers to a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen-binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine, and glycine. However, other residues can function as well. Phage display can be used to rapidly select tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) is displayed on filamentous phage and subjected to affinity selection with hapten. The population of selected variants exhibited significant increases in binding activity but retained considerable sequence diversity. Sequence analysis revealed a conserved proline in the linker two residues after the VH C terminus and an abundance of arginines and prolines at other positions as the only common features of the selected tethers. In certain embodiments, the antibody fragments are further modified to increase their serum half-life by using modified Fc regions or mutations to the various constant regions, as are known in the art.

In certain embodiments, the antibodies of the present invention are formulated for administration to humans. For example, the antibodies of the present invention can be included in a pharmaceutical composition formulated for an administration that is: intranasal, intrapulmonary, intrabronchial, intravenous, oral, intraadiposal, intraarterial, intraarticular, intracranial, intradermal, intralesional, intramuscular, intrapericardial, intraperitoneal, intrapleural, intravesicular, local, mucosal, parenteral, enteral, subcutaneous, sublingual, topical, transbuccal, transdermal, via inhalation, via injection, in creams, in lipid compositions, via a catheter, via a lavage, via continuous infusion, via infusion, via local delivery, or via localized perfusion, and wherein the composition is a serum, drop, gel, ointment, spray, reservoir, or mist.

As used herein, the term “antigen” refers to a molecule containing one or more epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a cytotoxic T lymphocyte (CTL) epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term includes polypeptides, which include modifications, such as deletions, additions and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts, which produce the antigens.

As used herein, the term “epitope” refers to a specific amino acid sequence or molecule (such as a carbohydrate, small molecule, lipid, etc.) that when present in the proper conformation, provides a reactive site for an antibody (e.g., B cell epitope) or in the case of a peptide to a T cell receptor (e.g., T cell epitope).

Portions of a given polypeptide that include a B-cell epitope can be identified using any number of epitope mapping techniques that are known in the art. (See, e.g., Epitope Mapping Protocols in Methods in Molecular Biology, Vol. 66, Glenn E. Morris, Ed., 1996, Humana Press, Totowa, N.J.). For example, linear epitopes can be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad Sci. USA 81:3998-4002; Geysen et al. (1986) Molec. Immunol. 23:709-715.

As used herein, the term “substantially purified” refers to isolation of a substance (compound, polynucleotide, protein, polypeptide, polypeptide composition) such that the substance comprises the majority percent of the sample in which it resides. Typically, in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

The practice of the present invention employs, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Fundamental Virology, Second Edition (Fields & Knipe eds., 1991, Raven Press, New York), relevant portion incorporated herein by reference.

Conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

The present disclosure provides methods of treating a subject having a metabolic disorder or at risk of developing a metabolic disorder, the methods comprising administering an anti-Activin E antibody, scFv, or binding fragment thereof to the subject that is antagonistic to the activity of Activin E.

The present disclosure also includes methods of treating a subject having type 2 diabetes or at risk of developing type 2 diabetes, the methods comprising administering an inhibitory anti-Activin E antibody, scFv, or binding fragment thereof to the subject.

The present disclosure also includes methods of treating a subject having obesity or at risk of developing obesity, the methods comprising administering an inhibitory anti-Activin E antibody, scFv, or binding fragment thereof to the subject.

The present disclosure also includes methods of treating a subject having elevated triglyceride level (hypertriglyceridemia) or at risk of developing elevated triglyceride level (hypertriglyceridemia), the methods comprising administering an inhibitory anti-Activin E antibody, scFv, or binding fragment thereof to the subject.

The present disclosure also includes methods of treating a subject having lipodystrophy or at risk of developing lipodystrophy, the methods comprising administering an inhibitory anti-Activin E antibody, scFv, or binding fragment thereof to the subject.

The present disclosure also includes methods of treating a subject having liver inflammation or at risk of developing liver inflammation, the methods comprising administering an inhibitory anti-Activin E antibody, scFv, or binding fragment thereof to the subject.

The present disclosure also includes methods of treating a subject having fatty liver disease or at risk of developing fatty liver disease, the methods comprising administering an inhibitory anti-Activin E antibody, scFv, or binding fragment thereof to the subject.

The present disclosure also includes methods of treating a subject having hypercholesterolemia or at risk of developing hypercholesterolemia, the methods comprising administering an inhibitory anti-Activin E antibody, scFv, or binding fragment thereof to the subject.

The present disclosure also includes methods of treating a subject having elevated liver enzymes (such as, for example, alanine transaminase (ALT) and/or aspartate transaminase (AST)) or at risk of developing elevated liver enzymes (such as, for example, ALT and/or AST), the methods comprising administering an inhibitory anti-Activin E antibody, scFv, or binding fragment thereof to the subject.

The present disclosure also includes methods of treating a subject having nonalcoholic steatohepatitis (NASH) or at risk of developing NASH, the methods comprising administering an inhibitory anti-Activin E antibody, scFv, or binding fragment thereof to the subject.

The present disclosure also includes methods of treating a subject having a cardiovascular disease or at risk of developing a cardiovascular disease, the methods comprising administering an inhibitory anti-Activin E antibody, scFv, or binding fragment thereof to the subject.

The present disclosure also includes methods of treating a subject having cardiomyopathy or at risk of developing cardiomyopathy, the methods comprising administering an inhibitory anti-Activin E antibody, scFv, or binding fragment thereof to the subject.

The present disclosure also includes methods of treating a subject having heart failure or at risk of developing heart failure, the methods comprising administering an inhibitory anti-Activin E antibody, scFv, or binding fragment thereof to the subject.

The present disclosure also includes methods of treating a subject having high blood pressure or at risk of developing high blood pressure, the methods comprising administering an inhibitory anti-Activin E antibody, scFv, or binding fragment thereof to the subject.

The present disclosure also includes methods of treating a subject with a therapeutic agent that treats or inhibits a metabolic disorder comprising administering or continuing to administer to the subject the therapeutic agent that treats or inhibits the metabolic disorder in a standard dosage amount, and administering to the subject an inhibitory anti-Activin E antibody, scFv, or binding fragment thereof.

The present disclosure also includes methods of treating a subject with a therapeutic agent that treats or inhibits a cardiovascular disease, wherein the subject is suffering from a cardiovascular disease, comprising administering or continuing to administer to the subject the therapeutic agent that treats or inhibits the cardiovascular disease in a standard dosage amount, and administering to the subject an inhibitory anti-Activin E antibody, scFv, or binding fragment thereof.

For subjects that have an increased risk of developing a metabolic disorder, such as type 2 diabetes, lipodystrophy, liver inflammation, fatty liver disease, hypercholesterolemia, elevated liver enzymes (such as, for example, ALT and/or AST), obesity, high blood pressure, and/or elevated triglyceride level (hypertriglyceridemia), and/or a cardiovascular disease, such as cardiomyopathy, heart failure, and high blood pressure. These subjects can be treated with an inhibitory anti-Activin E antibody, scFv, or binding fragment thereof of the present disclosure.

In certain embodiments of the disclosure, the metabolic disorder is type 2 diabetes, obesity, NASH, and/or elevated triglyceride level. In any of the embodiments disclosed herein, the metabolic disorder is type 2 diabetes. In any of the embodiments disclosed herein, the metabolic disorder is obesity. In any of the embodiments disclosed herein, the metabolic disorder is NASH. In any of the embodiments disclosed herein, the metabolic disorder is elevated triglyceride level. In any of the embodiments disclosed herein, the metabolic disorder is lipodystrophy. In any of the embodiments disclosed herein, the metabolic disorder is liver inflammation. In any of the embodiments disclosed herein, the metabolic disorder is fatty liver disease. In any of the embodiments disclosed herein, the metabolic disorder is hypercholesterolemia. In any of the embodiments disclosed herein, the metabolic disorder is elevated liver enzymes (such as, for example, ALT and/or AST).

In addition, other metabolic disorders/conditions associated with body fat distribution also include, but are not limited to: type 2 diabetes, hyperlipidemia or dyslipidemia (high or altered circulating levels of low-density lipoprotein cholesterol (LDL-C), triglycerides, very low-density lipoprotein cholesterol (VLDL-C), apolipoprotein B or other lipid fractions), obesity (particularly abdominal obesity), lipodystrophy (such as an inability to deposit fat in adipose depots regionally (partial lipodystrophy) or in the whole body (lipoatrophy)), insulin resistance or higher or altered insulin levels at fasting or during a glucose or insulin challenge, liver fat deposition or fatty liver disease and their complications (such as, for example, cirrhosis, fibrosis, or inflammation of the liver), higher or elevated or altered liver enzyme levels or other markers of liver damage, inflammation or fat deposition, higher blood pressure and/or hypertension, higher blood sugar or glucose or hyperglycemia, metabolic syndrome, coronary artery disease, and other atherosclerotic conditions, and the complications of each of the aforementioned conditions.

In any of the embodiments disclosed herein, the cardiovascular disease is cardiomyopathy, heart failure, or high blood pressure. In any of the embodiments disclosed herein, the cardiovascular disease is cardiomyopathy. In any of the embodiments disclosed herein, the cardiovascular disease is heart failure. In any of the embodiments disclosed herein, the cardiovascular disease is high blood pressure.

The present disclosure provides methods of treating a subject having or at risk of developing a metabolic disorder, the methods comprising administering an inhibitory anti-Activin E antibody, scFv, or binding fragment thereof to the subject.

The present disclosure also includes methods of treating a subject having or at risk of developing type 2 diabetes, the methods comprising administering an inhibitory anti-Activin E antibody, scFv, or binding fragment thereof to the subject. The present disclosure also includes methods of treating a subject having or at risk of developing obesity, the methods comprising administering an inhibitory anti-Activin E antibody, scFv, or binding fragment thereof to the subject.

The present disclosure also includes methods of treating a subject having or at risk of developing elevated triglyceride level, the methods comprising administering an inhibitory anti-Activin E antibody, scFv, or binding fragment thereof to the subject.

The present disclosure also includes methods of treating a subject having or at risk of developing NASH, the methods comprising administering an inhibitory anti-Activin E antibody, scFv, or binding fragment thereof to the subject.

The present disclosure also includes methods of treating a subject having or at risk of developing lipodystrophy, the methods comprising administering an inhibitory anti-Activin E antibody, scFv, or binding fragment thereof to the subject.

The present disclosure also includes methods of treating a subject having or at risk of developing liver inflammation, the methods comprising administering an inhibitory anti-Activin E antibody, scFv, or binding fragment thereof to the subject.

The present disclosure also includes methods of treating a subject having or at risk of developing fatty liver disease, the methods comprising administering an inhibitory anti-Activin E antibody, scFv, or binding fragment thereof to the subject.

The present disclosure also includes methods of treating a subject having or at risk of developing hypercholesterolemia, the methods comprising administering an inhibitory anti-Activin E antibody, scFv, or binding fragment thereof to the subject.

The present disclosure also includes methods of treating a subject having or at risk of developing elevated liver enzymes (such as, for example, ALT and/or AST), the methods comprising administering an inhibitory anti-Activin E antibody, scFv, or binding fragment thereof to the subject.

The present disclosure also includes methods of treating a subject having or at risk of developing a cardiovascular disease, the methods comprising administering an inhibitory anti-Activin E antibody, scFv, or binding fragment thereof to the subject.

The present disclosure also includes methods of treating a subject having or at risk of developing cardiomyopathy, the methods comprising administering an inhibitory anti-Activin E antibody, scFv, or binding fragment thereof to the subject.

The present disclosure also includes methods of treating a subject having or at risk of developing heart failure, the methods comprising administering an inhibitory anti-Activin E antibody, scFv, or binding fragment thereof to the subject.

The present disclosure also includes methods of treating a subject having or at risk of developing high blood pressure, the methods comprising administering an inhibitory anti-Activin E antibody, scFv, or binding fragment thereof to the subject.

Engineered Epitope Design. Engineered epitopes were built to embody solvent-accessible epitopes on the human Activin E protein.

Peptide scaffolds were computationally designed to support the native sequence and structure of target epitopes with the iBio Engineered Epitope machine learning engine. The design process starts with an initial set of blueprints indicating the positions of the scaffold residues to design and the positions of the epitope residues to hold fixed. The machine learning engine optimizes the scaffold residues in these initial blueprints for [1] structural match to the native epitope structure, [2] overall structural stability of the molecule, and [3] solubility of the peptide design. The number of scaffold residues in the blueprints are then iteratively removed until the machine learning engine can no longer satisfy all three of the loss functions described above. The final design is based on the blueprint that satisfies all loss functions using the minimal number of scaffold residues.

When the engineered epitopes are made by peptide synthesis the peptides are biotinylated for in-vitro selection and antibody screening assays.

In-Vitro Antibody Selections. Antibody in-vitro selections were carried out with two rounds of phage display using a previously constructed naïve library of fully human scfv sequences. 5×10\hat{ }12 virions were used as the input for each phage display round.

In the first round of selection, streptavidin-coated magnetic beads were incubated with biotinylated PSR, followed by incubation with the phage display library. Phages that bound to PSR were captured by the beads and removed using the KINGFISHER™ system, while the unbound phages were retained for subsequent positive selection. In the positive selection step, the remaining phage pool was incubated with soluble biotinylated engineered epitope or avi-tagged Activin E. After competitive incubation, streptavidin-coated magnetic beads coupled with an anti-avi-tag antibody were added to specifically capture the engineered epitope or Activin E and any bound phages. Phages were then separated using the KINGFISHER™ system, washed to remove non-specifically bound phages, eluted from the beads, and amplified in bacterial host cells for use in the second round of selection.

In the second round, another round of negative selection was performed against biotinylated PSR using the same method as in the first round. For positive selection, the amplified phage pool was incubated with soluble avi-tagged Activin E. Streptavidin-coated magnetic beads coupled to an anti-avi-tag antibody were added to specifically capture the Activin E and any bound phages. These beads were separated using the KINGFISHER™ system, washed to remove any non-specifically bound phages, and the Activin E-bound phages were eluted and amplified for subsequent characterization.

Phagemid from the round 2 selection was collected by midiprep of the transduced E. coli from the phage propagation step. ScFv inserts were transferred into a soluble mammalian expression vector to enable monoclonal screening by SPR kinetics.

Label-Free Activin E Binding Kinetics as Measured by SPR. The binding kinetics of antibodies to Activin E were measured using a CARTERRA® LSA. All steps were conducted in 1×HBST (HBS-T 0.01 M HEPES, 0.15 M NaCl, and 0.1% T20 at pH 7.4)+0.05% BSA) bovine serum albumin) kinetics buffer. Antibodies were captured on an anti-Human Fc-functionalized HC200M chip for 25 minutes, followed by six buffer injections to establish a baseline. This was followed by a series of injections of Activin E, arranged in order of increasing concentration. Each injection cycle consisted of a baseline, followed by an association phase with Activin E, and then a dissociation step in the assay buffer. This was followed by two 40-second injections of 0.1M glycine at pH 2.0 to regenerate the anti-Fc capture surface. Kinetic parameters were then calculated from the data using the LSA Kinetics software.

Antibody Expression and purification. Antibody expression plasmids were transiently introduced into an animal cell line using the EXPIFECTAMINE™ CHO Transfection Kit (ThermoFisher; Cat #A29129) to yield transfectants that produced antibody. For a host cell line, ExpiCHO-S (ThermoFisher; Cat #A29127) was used. After 6-12 days of growth post introduction of DNA, cell suspensions of ExpiCHO™ were harvested via centrifugation for 20 min at 4,000×g, and then filtered using 0.2 μm Disposable PES Filter units (FisherScientific, Cat #FB12566504). Antibody was recovered from filtrate using Protein A purification (HiTrap™ MabSelect SuRe™; Cytiva Cat #GE11-0034-93).

HEK293SBE/ALK7 SMAD Signaling Assay. HEK293 cells stably expressing a luciferase reporter gene linked to a SMAD binding element promoter (SBE) (BPS Bioscience, Cat #60653) were transfected with human ALK7 expression plasmid (Sino Biologicals, Cat #HG10869-UT) using Lipofectamine 3000 (ThermoFisher, Cat #L3000001) as indicated in manufacturer's instructions. The transfected cells were selected for ALK7 expression using 100 ug/mL hygromycin B (ThermoFisher, Cat #10687010) until the mock transfection control cells have no viable cells remaining. A monoclonal cell population was isolated from the polyclonal pool of ALK7 stable cells via limiting dilution then tested for ALK7 expression and activity. The resulting HEK293SBE/ALK7 reporter cell line was cultured in MEM media (Cytiva, Cat #SH30024.01) supplemented with 10% FBS (MilliporeSigma, Cat #F4135), 1% non-essential amino acids (Gibco, Cat #11140-050), 1 mM Na pyruvate (Gibco, Cat #11360-070), 1% Penicillin/Streptomycin (Corning, Cat #30-002-CI), 400 μg/mL of Geneticin (Gibco, Cat #10131035) and 100 ug/mL hygromycin B.

To determine SMAD 2/3 activation, the HEK293SBE/ALK7 cells were seeded in white, clear bottom 384-well plate (Greiner Bio-One, Cat #781098) at 3000 cells/well using Assay Media consisting of MEM media supplemented with 0.5% FBS (Gibco, Cat #26400044), 1% non-essential amino acids, 1 mM Na pyruvate, and 1% Penicillin/Streptomycin. 18-24 hours following plating, the cells were treated with the indicated antibodies at final dose range of 0.001-66.67 nM after an one hour, 37° C./5% CO2, antibody pre-incubation with either 0.02 nM Activin E (qKine, Cat #Qk067), 5 nM Myostatin (PeproTech, Cat #120-00), 5 nM GDF-11 (PeproTech, Cat #120-11), 1 nM Activin A (PeproTech, Cat #120-14E), or 1 nM Activin C (R&D Systems, Cat #1629-AC) final concentration for each ligand. Following 18 hours of antibody/ligand incubation, 1 volume of ONE-Step Luciferase reagent (BPS Bioscience, Cat #60690) was added to cells and plates were shaken for 15 minutes at room temperature to facilitate lysis according to manufacturer's instructions. After 15 minutes, luminescence was read on a SpectraMax ID5 (Molecular Devices) with 1000 ms integration time. GraphPad Prism 10.2.2 was used to calculate IC50 values, generate graphs, and perform statistical analysis.

pSmad2 assay. Primary human visceral preadipocyte cells were purchased from Lonza (Cat #PT-5005). The cells were cultured in PMG-2 preadipocyte growth media-2 bulletkit (Lonza, Cat #PT-8002), Cell passage 3rd-10^{th} were used for this experiment.

To evaluate the activins induced pSmads protein in differentiated adipocytes, preadipocyte cells were seeded at 10,000 cells per well in a 96-well plate (Corning, high throughput content imaging, cat #4680) and incubated overnight in a cell culture incubator. Cell samples were then treated with differentiation media (Lonza, Cat #PT-3004) to induced preadipocyte differentiate into adipocytes. Adipocytes post differentiation 4 days-6 days were used for this assay.

To induce Smad2 protein phosphorylation, Adipocytes post differentiation 4 days-6 days were treated with the indicated antibodies at a final dose range of 0.27-200 nM after a one-hour pre-incubation at room temperature with either Activin E, or Activin C, at a final concentration of 100 nM for each ligand. Cell samples were incubated in a cell culture incubator at 37° C. with 5% CO2 for 30 minutes. After incubation, the samples were washed twice with 1×PBS and then fixed with 4% paraformaldehyde (Thermo Scientific, Cat #J19943.K2) for 30 minutes at room temperature. The samples were then washed twice with 1×PBS and blocked with blocking buffer (2% FBS, 0.2% Triton X-100 in 1×PBS) for 1 hour at room temperature. Prepare the anti-pSmad2 antibody by diluting the stock antibody 1:100 and DAPI 1:10000 in antibody dilution buffer (2% FBS, 0.01% Triton X-100 in 1×PBS). Remove the blocking buffer and add 25 μL/well of the antibody solution. Incubate the samples for 1 hour at room temperature. Remove the antibody solution and wash the samples twice with 1×.

PBS. The samples are now ready for imaging. Acquire images of the samples using a 20× objective on the IMAGEEXPRESS® (Molecular Devices). Analyze the samples using custom analysis with IMAGEEXPRESS® software. GraphPad Prism 10.2.2 was used to calculate EC50 values and generate graphs.

### Exemplary Anti-Activin E Antibodies—CDR Sequences.

Provided herein are sequences for exemplary Anti-Activin E antibodies of the disclosure. Included are complementarity determining region (CDR) sequences and the variable heavy and light domain sequences (VH, VL) that constitute the Activin E antigen binding domains of the disclosure. The discovery of these antibodies is detailed in the Examples section.

As referred below, a light chain variable (VL) domain CDR1 region is referred to as CDR-L1; a VL CDR2 region is referred to as CDR-L2; a VL CDR3 region is referred to as CDR-L3; a heavy chain variable (VH) domain CDR1 region is referred to as CDR-H1; a VH CDR2 region is referred to as CDR-H2; and a VH CDR3 region is referred to as CDR-H3. Table 1 provides exemplary CDR combinations of antibodies of the disclosure.

TABLE 1
Exemplary Activin E antagonist antibody, scFv, or antigen binding domain
complementarity determining regions (CDR) combinations
(heavy chain CDR-hCDR1-3, light chain lCDR1-3).

| Clone ID | hCDR1 | hCDR2 | hCDR3 | lCDR1 | lCDR2 | lCDR3 |
|---|---|---|---|---|---|---|
| AVE- | GFAFNNY | ISGSGTS | AKPRPGSIFW | QSISSY | AAS | QQSYGS |
| 01-D07 | A | K | GAGPFDY | | | PT |
| SEQ ID | 1 | 2 | 3 | 4 | 5 | 6 |
| NO | | | | | | |
| AVE- | GFTFGAS | ISGLGRT | ARVAPGAYA | QNIGHY | DAS | QQSYST |
| 01-B08 | A | T | YAMDY | | | PPT |
| SEQ ID | 11 | 12 | 13 | 14 | 15 | 16 |
| NO | | | | | | |
| AVE- | GFNFRSY | ISDVGRR | AKAEILGDYA | ESIGNY | AAS | QQSYST |
| 02-A03 | V | T | YMDY | | | PT |
| SEQ ID | 21 | 22 | 23 | 24 | 25 | 26 |
| NO | | | | | | |
| AVE- | GFAFSAY | ITESGAA | ARGLLASYTG | QSISSY | ATS | QQRDNA |
| 02-B04 | A | T | DV | | | PWT |
| SEQ ID | 31 | 32 | 33 | 34 | 35 | 36 |
| NO | | | | | | |
| AVE- | GFDFSSFA | ITGSGGTI | ASASSYYEPG | QAIKNY | AGS | QQTYST |
| 06-B07 | | | DP | | | PLT |
| SEQ ID | 41 | 42 | 43 | 44 | 45 | 46 |
| NO | | | | | | |
| AVE- | GFPFSSHG | ITGSGRS | ASDYRDAPGT | QSISSY | DAS | QQSYST |
| 06-F07 | | T | FDV | | | PV |
| SEQ ID | 51 | 52 | 53 | 54 | 55 | 56 |
| NO | | | | | | |
| AVE- | GFPFASH | ITGSGRS | ASDYRDAPGT | QSISSY | DAS | QQSYST |
| 06-A08 | A | T | FDV | | | PV |
| SEQ ID | 61 | 62 | 63 | 64 | 65 | 66 |
| NO | | | | | | |
| AVE- | GFDFSKF | ISGSGGTI | ASASSYYEPG | QAIKNY | DAS | QQSYST |
| 06-D08 | A | | DP | | | PV |
| SEQ ID | 71 | 72 | 73 | 74 | 75 | 76 |
| NO | | | | | | |
| AVE- | GFDFSKF | ISGSGGTI | ASDSSYYEPG | QAIKNY | AGS | QQTYST |
| 06-G08 | A | | DP | | | PLT |
| SEQ ID | 81 | 82 | 83 | 84 | 85 | 86 |
| NO | | | | | | |
| AVE- | GFDFSKF | ITRGSET | ATLGLGYYY | QPISSY | SAS | QQSYNA |
| 06-H08 | A | T | YFDV | | | PPT |
| SEQ ID | 91 | 92 | 93 | 94 | 95 | 96 |
| NO | | | | | | |
| AVE- | GFPFASH | ITGSGRS | ASASSYYEPG | QTISSF | AAT | QQSYHT |
| 06-A09 | A | T | DP | | | RS |
| SEQ ID | 101 | 102 | 103 | 104 | 105 | 106 |
| NC | | | | | | |
| AVE- | GLTFSNFA | IRGSGAT | ARSREAYGFD | QSISSY | AAS | QQSYST |
| 06-B09 | | T | Y | | | PYT |
| SEQ ID | 111 | 112 | 113 | 114 | 115 | 116 |
| NO | | | | | | |
| AVE- | GFTFSHYS | ISGSGSA | ASDRYLTFDV | QSISTH | AAS | QQSHRT |
| 06-D09 | | T | | | | PLI |
| SEQ ID | 121 | 122 | 123 | 124 | 125 | 126 |
| NO | | | | | | |
| AVE- | GFDFSKF | ISGSGGTI | ASASSYYEPG | QAIKNY | AGS | QQTYST |
| 06-F09 | A | | DP | | | PLT |
| SEQ ID | 131 | 132 | 133 | 134 | 135 | 136 |
| NO | | | | | | |
| AVE- | GFSFDNY | ISSSGGS | ARGVVPGGF | QSINNY | AAS | QQSRTT |
| 06-G09 | G | A | DY | | | PWT |
| SEQ ID | 141 | 142 | 143 | 144 | 145 | 146 |
| NO | | | | | | |
| AVE- | GFDFSKF | ITGTSGA | ARDIRVRRSS | QTIGIY | SAS | QQSYST |
| 06-C10 | A | T | WAMDP | | | PHIT |
| SEQ ID | 151 | 152 | 153 | 154 | 155 | 156 |
| NO | | | | | | |
| AVE- | GFDFSKF | ISGSGGTI | ASASSYYEPG | QAIKNY | AGS | QQTYST |
| 06-G10 | A | | DP | | | PLT |
| SEQ ID | 161 | 162 | 163 | 164 | 165 | 166 |
| NO | | | | | | |
| AVE- | GFTFTNY | ISGSGGTI | ASASSYYEPG | QAIKNY | AGS | QQTYST |
| 06-H10 | A | | DP | | | PLT |
| SEQ ID | 171 | 172 | 173 | 174 | 175 | 176 |
| NO | | | | | | |
| AVE- | GFDFSKF | ISGSGGTI | ASASSYYEPG | QAIKNY | AGS | QQTYST |
| 06-B11 | A | | DP | | | PLT |
| SEQ ID | 181 | 182 | 183 | 184 | 185 | 186 |
| NO | | | | | | |
| AVE- | GIRFSSYA | ISGSGGTI | ASASSYYEPG | QAIKNY | AGS | QQTYST |
| 06-E11 | | | DP | | | PLT |
| SEQ ID | 191 | 192 | 193 | 194 | 195 | 196 |
| NO | | | | | | |
| AVE- | GFDFSKF | ISGSGGTI | ASASSYYEPG | QAIKNY | AGS | QQTYST |
| 06-H11 | V | | DP | | | PLT |
| SEQ ID | 201 | 202 | 203 | 204 | 205 | 206 |
| NO | | | | | | |
| AVE- | GFDFSKF | ISGSGGTI | ASASSYYEPG | QAIKNY | AGS | QQNYST |
| 06-A12 | A | | DP | | | PFA |
| SEQ ID | 21 | 212 | 213 | 214 | 215 | 216 |
| NO | | | | | | |
| AVE- | GFDFSKF | ISGSGGTI | ASASSYYEPG | QAIKNY | AGS | QQTYST |
| 06-B12 | A | | DP | | | PLT |
| SEQ ID | 221 | 222 | 223 | 224 | 225 | 226 |
| NO | | | | | | |
| AVE- | GFDFSKF | ISGSGGTI | ASASSYYEPG | QAIKNY | AGS | QQNYST |
| 06-C12 | A | | DP | | | PLT |
| SEQ ID | 231 | 232 | 233 | 234 | 235 | 236 |
| NO | | | | | | |
| AVE- | GFPFSSHG | ISGSGAT | ARGQPYYGF | HSIASY | RAS | QQTYSIP |
| 06-E12 | | T | DY | | | WT |
| SEQ ID | 241 | 242 | 243 | 244 | 245 | 246 |
| NO | | | | | | |
| AVE- | GFDFSKF | ISGSGGTI | ASASSYYEPG | QAIKNY | GAS | QQSEST |
| 06-G12 | A | | DP | | | PLT |
| SEQ ID | 251 | 252 | 253 | 254 | 255 | 256 |
| NO | | | | | | |
| AVE- | GFALTDF | ISVSGGV | ARSRSYYAGA | APVGRY | AAS | QQSYSA |
| 07-A01 | A | G | FDV | | | VT |
| SEQ ID | 261 | 262 | 263 | 264 | 265 | 266 |
| NO | | | | | | |
| AVE- | GFTFGSY | IGTTDRY | ARSRSYYAGA | QTLRNY | AAS | QQRFSP |
| 07-B01 | A | T | FDV | | | PWT |
| SEQ ID | 271 | 272 | 273 | 274 | 275 | 276 |
| NO | | | | | | |
| AVE- | GYTFSNF | ITGSGVR | ARSRSYYAGA | QSIGSS | AAS | QQSDNN |
| 07-C01 | A | T | FDV | | | PWT |
| SEQ ID | 281 | 282 | 283 | 284 | 285 | 286 |
| NO | | | | | | |
| AVE- | GFTFNNA | ISSSGGY | ARSRSYYAGA | APVGRY | AAS | QQAYSI |
| 07-D01 | W | T | FDV | | | PIT |
| SEQ ID | 291 | 292 | 293 | 294 | 295 | 296 |
| NO | | | | | | |
| AVE- | GFSVRDF | ITGTDRT | ARSRSYYAGA | QNIFSY | AAS | QQTHSIP |
| 07-E01 | A | P | FDV | | | QT |
| SEQ ID | 301 | 302 | 303 | 304 | 305 | 306 |
| NO | | | | | | |
| AVE- | GFTFSSHN | IGGGGRT | ARAAPGAYA | QRISTY | GAS | QQTYST |
| 07-F01 | | T | YALDY | | | PPT |
| SEQ ID | 311 | 312 | 313 | 314 | 315 | 316 |
| NO | | | | | | |
| AVE- | GFSVRDF | IRISGGG | ARAAPGAYA | QRISTY | SAS | QQNYRT |
| 07-C02 | A | T | YALDY | | | PPT |
| SEQ ID | 321 | 322 | 323 | 324 | 325 | 326 |
| NO | | | | | | |
| AVE- | GFTFSNY | ITSSASK | ARTRYLERFA | QSISSY | AAS | QQSFTA |
| 07-F02 | A | T | GGLDI | | | PLT |
| SEQ ID | 331 | 332 | 333 | 334 | 335 | 336 |
| NO | | | | | | |
| AVE- | GLTFSNY | ISGTGGS | ARSRSYYAGA | VSISTF | AAS | QQSLRT |
| 07-G02 | T | T | FDV | | | PIT |
| SEQ ID | 341 | 342 | 343 | 344 | 345 | 346 |
| NO | | | | | | |
| AVE- | GFPFSSFV | LSGSGDI | ARAAPGAYA | QSVSTY | AAS | QQSYRT |
| 07-H03 | | T | YALDY | | | PPT |
| SEQ ID | 351 | 352 | 353 | 354 | 355 | 356 |
| NO | | | | | | |
| AVE- | GLTFNAY | LTGSGAS | ARSRSYYAGA | RTVTSY | GAS | QQSYRT |
| 07-D04 | A | T | FDV | | | PPT |
| SEQ ID | 361 | 362 | 363 | 364 | 365 | 366 |
| NO | | | | | | |
| AVE- | GFSVRDF | IHRTGGG | ARSRSYYAGA | QNIGNY | AAS | QQSYST |
| 07-E06 | A | T | FDV | | | ST |
| SEQ ID | 371 | 372 | 373 | 374 | 375 | 376 |
| NO | | | | | | |
| AVE- | GFDFRSYP | ISGGGGS | ARSRSYYAGA | QKIARY | AAS | QQAYSI |
| 07-F06 | | T | FDV | | | PIT |
| SEQ ID | 381 | 382 | 383 | 384 | 385 | 386 |
| NO | | | | | | |
| AVE- | GFRFSNY | ISGGGGR | ARGYGAEYF | QPISRY | DAS | QQSHSIP |
| 07-A07 | A | T | DP | | | WT |
| SEQ ID | 391 | 392 | 393 | 394 | 395 | 396 |
| NO | | | | | | |
| AVE- | GFRFSNY | ISGGGGR | ARGYGAEYF | QPISRH | SAS | QQSYDR |
| 07-E07 | A | T | DP | | | TWT |
| SEQ ID | 401 | 402 | 403 | 404 | 405 | 406 |
| NO | | | | | | |
| AVE- | GFRFSNY | ISGGGGR | ARGYGAEYF | QRIATY | AAS | QQSYSIP |
| 07-D08 | A | T | DP | | | LT |
| SEQ ID | 411 | 412 | 413 | 414 | 415 | 416 |
| NO | | | | | | |
| AVE- | GFTVSRD | ISTGGGS | AKPRPYSIAW | QTISIY | AAS | QQNYSV |
| 07-E08 | Y | T | FADPFDY | | | PPT |
| SEQ ID | 421 | 422 | 423 | 424 | 425 | 426 |
| NO | | | | | | |
| AVE- | GFPFSNN | ISGSYGT | ARVADGAAA | QPISRY | DAS | QQSHSIP |
| 07-H08 | A | T | YAMDY | | | WT |
| SEQ ID | 431 | 432 | 433 | 434 | 435 | 436 |
| NO | | | | | | |
| AVE- | GFRFSNY | ISGGGGR | ARGYGAEYF | QPISRY | AAS | QQSYNP |
| 07-C10 | A | T | DP | | | PLT |
| SEQ ID | 441 | 442 | 443 | 444 | 445 | 446 |
| NO | | | | | | |
| AVE- | GFTFPHSA | IAGRGGS | ARVADGGAA | QNIITY | GAS | QQSFSTP |
| 07-D11 | | P | YAFDY | | | LT |
| SEQ ID | 451 | 452 | 453 | 454 | 455 | 456 |
| NO | | | | | | |
| AVE- | GFRFSNY | ISGGGGR | ARGYGAEYF | QPISRY | DAS | QQSHSIP |
| 07-F11 | A | T | DP | | | WT |
| SEQ ID | 461 | 462 | 463 | 464 | 465 | 466 |
| NO | | | | | | |
| AVE- | GFTFPHSA | VTGSGSP | ARVAGGAYG | QSISSY | DAS | QQSFSN |
| 07-G11 | | T | YAMDY | | | LYT |
| SEQ ID | 471 | 472 | 473 | 474 | 475 | 476 |
| NO | | | | | | |
| AVE- | GFPFSVY | FGGSGHS | ARVAAGSYA | QNIITY | GAS | QQSFSTP |
| 07-F12 | A | P | YAMDY | | | LT |
| SEQ ID | 481 | 482 | 483 | 484 | 485 | 486 |
| NO | | | | | | |
| AVE- | GFRFSNY | ISGGGGR | ARGYGAEYF | QPISRY | DAS | QQSFSTP |
| 07-G12 | A | T | DP | | | LT |
| SEQ ID | 491 | 492 | 493 | 494 | 495 | 496 |
| NO | | | | | | |

In some embodiments, provided herein is an anti-Activin E antibody, wherein the antibody comprises the amino acid sequences of the following three VH CDR1-3: SEQ ID NOS: 1, 2, 3; 11, 12, 13; 21, 22, 23; 31, 32, 33; 41, 42, 43; 51, 52, 53; 61, 62, 63; 71, 72, 73; 81, 82, 83; 91, 92, 93; 101, 102, 103; 111, 112, 113; 121, 122, 123; 131, 132, 133; 141, 142, 143; 151, 152, 153; 161, 162, 163; 171, 172, 173; 181, 182, 183; 191, 192, 193; 201, 202, 203; 211, 212, 213; 221, 222, 223; 231, 232, 233; 241, 242, 243; 251, 252, 253; 261, 262, 263; 271, 272, 273; 281, 282, 283; 291, 292, 293; 301, 302, 303; 311, 312, 313; 321, 322, 323; 331, 332, 333; 341, 342, 343; 351, 352, 353; 361, 362, 363; 371, 372, 373; 381, 382, 383; 391, 392, 393; 401, 402, 403; 411, 412, 413; 421, 422, 423; 431, 432, 433; 441, 442, 443; 451, 452, 453; 461, 462, 463; 471, 472, 473; 481, 482, 483; or 491, 492, 493; and/or comprises the amino acid sequences of the following three VL CDR1-3 SEQ ID NO: 4, 5, 6; 14, 15, 16; 24, 25, 26; 34, 35, 36; 44, 45, 46; 54, 55, 56; 64, 65, 66; 74, 75, 76; 84, 85, 86; 94, 95, 96; 104, 105, 106; 114, 115, 116; 124, 125, 126; 134, 135, 136; 144, 145, 146; 154, 155, 156; 164, 165, 166; 174, 175, 176; 184, 185, 186; 194, 195, 196; 204, 205, 206; 214, 215, 216; 224, 225, 226; 234, 235, 236; 244, 245, 246; 254, 255, 256; 264, 265, 266; 274, 275, 276; 284, 285, 286; 294, 295, 296; 304, 305, 306; 314, 315, 316; 324, 325, 326; 334, 335, 336; 344, 345, 346; 354, 355, 356; 364, 365, 366; 374, 375, 376; 384, 385, 386; 394, 395, 396; 404, 405, 406; 414, 415, 416; 424, 425, 426; 434, 435, 436; 444, 445, 446; 454, 455, 456; 464, 465, 466; 474, 475, 476; 484, 485, 486; or 494, 495, 496.

### Exemplary Anti-Activin E Antibodies—Variable Region Sequences.

The term variable domain and variable region are used interchangeably and refer to the portions of the light and heavy chains of an antibody that include the complementarity determining regions and framework regions (FRs).

In some embodiments, an anti-Activin E antibody of the disclosure comprises the combination of VH/VL variable chain amino acid sequences of any one of the combinations listed in Table 2.

TABLE 2
Exemplary Variable Heavy Chain and Variable Light Chain
Amino Acid Sequences of Anti-Activin E antibodies.

| CloneID | VH AA | VL AA |
|---|---|---|
| AVE-01-D07 | EVQLLESGGGLVQPGGSLRLSCAASGF | DIQMTQSPSSLSASVGDRVTI |
| | AFNNYAMTWVRQAPGKGLEWVSVISG | TCRASQSISSYLHWYQQKPG |
| | SGTSKYYADSVKGRFTISRDNSKNTLYL | KAPKLLIYAASSLQSGVPSRF |
| | QMNSLRAEDTAVYYCAKPRPGSIFWGA | SGSGSGTDFTLTISSLQPEDFA |
| | GPFDYWGQGTLVTVSS | TYYCQQSYGSPTFGGGTKVE |
| | | IK |
| | SEQ ID NO | |
| | 7 | 8 |
| AVE-01-B08 | EVQLLESGGGLVQPGGSLRLSCAASGFT | DIQMTQSPSSLSASVGDRVTI |
| | FGASAMTWVRQAPGKGLEWVSGISGL | TCRASQNIGHYLNWYQQKP |
| | GRTTDYADSVKGRFTISRDNSKNTLYL | GKAPKLLIYDASRLQVGVPS |
| | QMNSLRAEDTAVYYCARVAPGAYAYA | RFSGSGSGTDFTLTISSLQPED |
| | MDYWGQGTLVTVSS | FATYYCQQSYSTPPTFGGGT |
| | | KVEIK |
| | SEQ ID NO | |
| | 17 | 18 |
| AVE-02-A03 | EVQLLESGGGLVQPGGSLRLSCAASGF | DIQMTQSPSSLSASVGDRVTI |
| | NFRSYVMNWVRQAPGKGLEWVSAISD | TCRASESIGNYLSWYQQKPG |
| | VGRRTYYADSVKGRFTISRDNSKNTLY | KAPKLLIYAASRLQRGVPSRF |
| | LQMNSLRAEDTAVYYCAKAEILGDYA | SGSGSGTDFTLTISSLQPEDFA |
| | YMDYWGQGTLVTVSS | TYYCQQSYSTPTFGGGTKVEI |
| | | K |
| | SEQ ID NO | |
| | 27 | 28 |
| AVE-02-B04 | EVQLLESGGGLVQPGGSLRLSCAASGF | DIQMTQSPSSLSASVGDRVTI |
| | AFSAYAMNWVRQAPGKGLEWVSSITES | TCRASQSISSYLNWYQQKPG |
| | GAATYYADSVKGRFTISRDNSKNTLYL | KAPKLLIYATSTLQSGVPSRF |
| | QMNSLRAEDTAVYYCARGLLASYTGD | SGSGSGTDFTLTISSLQPEDFA |
| | VWGQGTLVTVSS | TYYCQQRDNAPWTFGGGTK |
| | | VEIK |
| | SEQ ID NO | |
| | 37 | 38 |
| AVE-06-B07 | EVQLLESGGGLVQPGGSLRLSCAASGF | DIQMTQSPSSLSASVGDRVTI |
| | DFSSFAMTWVRQAPGKGLEWVSHITGS | TCRASQAIKNYLNWYQQKP |
| | GGTIYYADSVKGRFTISRDNSKNTLYLQ | GKAPKLLIYAGSTLQSGVPSR |
| | MNSLRAEDTAVYYCASASSYYEPGDP | FSGSGSGTDFTLTISSLQPEDF |
| | WGQGTLVTVSS | ATYYCQQTYSTPLTFGGGTK |
| | | VEIK |
| | SEQ ID NO | |
| | 47 | 48 |
| AVE-06-F07 | EVQLLESGGGLVQPGGSLRLSCAASGFP | DIQMTQSPSSLSASVGDRVTI |
| | FSSHGMSWVRQAPGKGLEWVSTITGSG | TCRASQSISSYLNWYQQKPG |
| | RSTYYADSVKGRFTISRDNSKNTLYLQ | KAPKLLIYDASHLQSGVPSRF |
| | MNSLRAEDTAVYYCASDYRDAPGTFD | SGSGSGTDFTLTISSLQPEDFA |
| | VWGQGTLVTVSS | TYYCQQSYSTPVFGGGTKVE |
| | | IK |
| | SEQ ID NO | |
| | 57 | 58 |
| AVE-06-A08 | EVQLLESGGGLVQPGGSLRLSCAASGFP | DIQMTQSPSSLSASVGDRVTI |
| | FASHAMTWVRQAPGKGLEWVSTITGS | TCRASQSISSYLNWYQQKPG |
| | GRSTYYADSVKGRFTISRDNSKNTLYL | KAPKLLIYDASHLQSGVPSRF |
| | QMNSLRAEDTAVYYCASDYRDAPGTF | SGSGSGTDFTLTISSLQPEDFA |
| | DVWGQGTLVTVSS | TYYCQQSYSTPVFGGGTKVE |
| | | IK |
| | SEQ ID NO | |
| | 67 | 68 |
| AVE-06-D08 | EVQLLESGGGLVQPGGSLRLSCAASGF | DIQMTQSPSSLSASVGDRVTI |
| | DFSKFAMSWVRQAPGKGLEWVSHISGS | TCRASQAIKNYLNWYQQKP |
| | GGTIYYADSVKGRFTISRDNSKNTLYLQ | GKAPKLLIYDASHPQSGVPSR |
| | MNSLRAEDTAVYYCASASSYYEPGDP | FRGSGSGTDFPFTISSLQPEEF |
| | WGQGTLVTVSS | ATYYCQQSYSTPVFGGGTKV |
| | | EIK |
| | SEQ ID NO | |
| | 77 | 78 |
| AVE-06-G08 | EVQLLESGGGLVQPGGSLRLSCAASGF | DIQMTQSPSSLSASVGDRVTI |
| | DFSKFAMSWVRQAPGKGLEWVSHISGS | TCRAGQAIKNYLNWYQQKP |
| | GGTIYYADSVKGRFTISRDNSKNTLYLQ | GKAPKLLIYAGSPLQSGVPSR |
| | MNSLRAEDTA VYYCASDSSYYEPGDP | FSGSGSGTDFTLTISSLQPEDF |
| | WGQGTLVTVSS | ATYYCQQTYSTPLTFGGGTK |
| | | VEIK |
| | SEQ ID NO | |
| | 87 | 88 |
| AVE-06-H08 | EVQLLESGGGLVQPGGSLRLSCAASGF | DIQMTQSPSSLSASVGDRVTI |
| | DFSKFAMSWVRQAPGKGLEWVSSITRG | TCRASQPISSYVTWYQQKPG |
| | SETTYYADSVKGRFTISRDNSKNTLYLQ | KAPKLLIYSASHLRSGVPSRF |
| | MNSLRAEDTAVYYCATLGLGYYYYFD | SGSGSGTDFTLTISSLQPEDFA |
| | VWGQGTLVTVSS | TYYCQQSYNAPPTFGGGTKV |
| | | EIK |
| | SEQ ID NO | |
| | 97 | 98 |
| AVE-06-A09 | EVQLLESGGGLVQPGGSLRLSCAASGFP | DIQMTQSPSSLSASVGDRVTI |
| | FASHAMTWVRQAPGKGLEWVSTITGS | TCRASQTISSFVSWYQQKPG |
| | GRSTYYADSVKGRFTISRDNSKNTLYL | KAPKLLIYAATTLQKGVPSRF |
| | QMNSLRAEDTAVYYCASASSYYEPGDP | SGSGSGTDFTLTISSLQPEDFA |
| | WGQGTLVTVSS | TYYCQQSYHTRSFGGGTKVE |
| | | IK |
| | SEQ ID NO | |
| | 107 | 108 |
| AVE-06-B09 | EVQLLESGGGLVQPGGSLRLSCAASGL | DIQMTQSPSSLSASVGDRVTI |
| | TFSNFAMTWVRQAPGKGLEWVSSIRGS | TCRASQSISSYLNWYQQKPG |
| | GATTYYADSVKGRFTISRDNSKNTLYL | KAPKLLIYAASSLQSGVPSRF |
| | QMNSLRAEDTAVYYCARSREAYGFDY | SGSGSGTDFTLTISSLQPEDFA |
| | WGQGTLVTVSS | TYYCQQSYSTPYTFGGGTKV |
| | | EIK |
| | SEQ ID NO | |
| | 117 | 118 |
| AVE-06-D09 | EVHLLESGGGLVQPGGSLRLSCAASGFT | DIQMTQSPSSLSASVGDRVTI |
| | FSHYSMNWVRQAPGKGLEWVSGISGS | TCRASQSISTHLNWYQQKPG |
| | GSATYYADSVKGRFTISRDNSKNTLYL | KAPKLLIYAASSLQSGVPSRF |
| | QMNSLRAEDTAVYYCASDRYLTFDVW | SGSGSGTDFTLTISSLQPEDFA |
| | GQGTLVTVSS | TYYCQQSHRTPLIFGGGTKV |
| | | EIK |
| | SEQ ID NO | |
| | 127 | 128 |
| AVE-06-F09 | EVQLLESGGGLVQPGGSLRLSCAASGF | DIQMTQSPSSLSASVGDRVTI |
| | DFSKFAMSWVRQAPGKGLEWVSHISGS | TCRASQAIKNYLNWYQQKP |
| | GGTIYYADSVKGRFTISRDNSKNTLYLQ | GKAPKLLIYAGSTLQSGVPSR |
| | MNSLRAEDTAVYYCASASSYYEPGDP | FSGSGSGTDFTLTISSLQPEDF |
| | WGQGTLVTVSS | ATYYCQQTYSTPLTFGGGTK |
| | | GEIK |
| | SEQ ID NO | |
| | 137 | 138 |
| AVE-06-G09 | EVQFLESGGGLVQPGGSLRLSCAASGFS | DIQMTQSPSSLSASVGDRVTI |
| | FDNYGINWVRQAPGKGLEWVSSISSSG | TCRASQSINNYLNWYQQKPG |
| | GSAYYADSVKGRFTISRDNSKNTLYLQ | KAPKLLIYAASSLQSGVPSRF |
| | MNSLRAEDTAVYYCARGVVPGGFDYW | SGSGSGTDFTLTISSLQPEDFA |
| | GQGTLVTVSS | TYYCQQSRTTPWTFGGGTKV |
| | | EIK |
| | SEQ ID NO | |
| | 147 | 148 |
| AVE-06-C10 | EVQLLESGGGLVQPGGSLRLSCAASGF | DIQMTQSPSSLSASVGDRVTI |
| | DFSKFAMSWVRQAPGKGLEWVSSITGT | TCRASQTIGIYLNWYQQKPG |
| | SGATYYADSVKGRFTISRDNSKNTLYL | KGPKPAIYSASPLQRGVPSRF |
| | QMNSLRAEDTAVYYCARDIRVRRSSW | SGCGSGTDFTLTISSLQPEDF |
| | AMDPWGQGTLVTVSS | ATYYCQQSYSTPHITFGGGT |
| | | KVEIK |
| | SEQ ID NO | |
| | 157 | 158 |
| AVE-06-G10 | EVQLLESGGGLVQPGGSLRLSCAASGF | DIQMTQSPSSLSASVGDRVTI |
| | DFSKFAMSWVRQAPGKGLEWVSHISGS | TCRASQAIKNYLNWYQQKP |
| | GGTIYYADSVKGRFTISRDNSKNTLYLQ | GKAPKLLIYAGSTLQSGVPSR |
| | MNSLRAEDTAVYYCASASSYYEPGDP | FSGSGSGTDFTLTISSLQPEEF |
| | WGQGTLVTVSS | ATYYCQQTYSTPLTFGGGTK |
| | | GENQ |
| | SEQ ID NO | |
| | 167 | 168 |
| AVE-06-H10 | EVQLLESGGGLVQPGGSLRLSCAASGFT | DIQMTQSPSSLSASVGDRVTI |
| | FTNYALAWVRQAPGKGLEWVSHISGSG | TCRASQAIKNYLNWYQQKP |
| | GTIYYADSVKGRFTISRDNSKNTLYLQ | GKAPKLLIYAGSTLQSGVPSR |
| | MNSLRAEDTAVYYCASASSYYEPGDP | FSGSGSGTDFTLTISSLQPEDF |
| | WGQGTLVTVSS | ATYYCQQTYSTPLTFGGGTK |
| | | VEIK |
| | SEQ ID NO | |
| | 177 | 178 |
| AVE-06-B11 | EVQLLESGGGLVQPGGSLRLSCAASGF | DIQMTQSPSSLSASVGDRVTI |
| | DFSKFAMSWVRQAPGKGLEWVSHISGS | TCRASQAIKNYLNWYQQKP |
| | GGTIYYADSVKGRFTISRDNSKNTLYLQ | GKAPKLLIYAGSTLQSGVPSR |
| | MNSLRAEDTAVYYCASASSYYEPGDP | FSGSGSGTDFTLTISSLQPEDF |
| | WGQGTLVTVSS | ATYYCQQTYSTPLTFGGGTK |
| | | VEIK |
| | SEQ ID NO | |
| | 187 | 188 |
| AVE-06-E11 | EVQLLESGGGLVQPGGSLRLSCAASGIR | DIQMTQSPSSLSASVGDRVTI |
| | FSSYAMSWVRQAPGKGLEWVSHISGSG | TCRASQAIKNYLNWYQQKP |
| | GTIYYADSVKGRFTISRDNSKNTLYLQ | GKAPKLLIYAGSTLQSGVPSR |
| | MNSLRAEDTAVYYCASASSYYEPGDP | FSGSGSGTDFTLTISSLQPEDF |
| | WGQGTLVTVSS | ATYYCQQTYSTPLTFGGGTK |
| | | VEIK |
| | SEQ ID NO | |
| | 197 | 198 |
| AVE-06-H11 | EVQLLESGGGLVQPGGSLRLSCAVSGF | DIQMTQSPSSLSASVGDRVTI |
| | DFSKFVMSGVRQAPGKGLEWVSHISGS | TCRASQAIKNYLNWYQQKP |
| | GGTIYYADSVKGRFTISRDNSKNTLYLQ | GKAPKLLIYAGSTLQSGVPSR |
| | MNSLRAEDTAVYYCASASSYYEPGDP | FSGSGSGTDFTLTISSLQPEDF |
| | WGQGTLVTVSS | ATYYCQQTYSTPLTFGGGTK |
| | | VEIK |
| | SEQ ID NO | |
| | 207 | 208 |
| AVE-06-A12 | EVQLLESGGGLVQPGGSLRLSCAASGF | DIQMTQSPSSLSASVGDRVTI |
| | DFSKFAMSWVRQAPGKGLEWVSHISGS | TCRASQAIKNYLNWYQQKP |
| | GGTIYYADSVKGRFTISRDNSKNTLYLQ | GKAPKLLIYAGSTLQSGVPSR |
| | MNSLRAEDTAVYYCASASSYYEPGDP | FSGSGSGTDFTLTISSLQPEDF |
| | WGQGTLVTVSS | PTYYFQQNYSTPFAFGGGTK |
| | | VEIK |
| | SEQ ID NO | |
| | 217 | 218 |
| AVE-06-B12 | EVQLLESGGGLVQPGGSLRLSCAASGF | DIQMTQSPSSLSASVGDRVTI |
| | DFSKFAMSWVRPAPCKGLEWVSHISGS | TCRASQAIKNYLNWYQQKP |
| | GGTIYYADSVKGRFTISRDNSKNTLYLQ | GKAPKLLIYAGSTLQSGVPSR |
| | MNSLRAEDTAVYYCASASSYYEPGDP | FSGSGSGTDFTLTISSLQPEDF |
| | WGQGTLVTVSS | ATYYCQQTYSTPLTFGGGTK |
| | | VEIK |
| | SEQ ID NO | |
| | 227 | 228 |
| AVE-06-C12 | EVQLLESGGGLVQPGGSLRLSCAASGF | DIQMTQSPSSLSASVGDRVTI |
| | DFSKFAMSWVRQAPGKGLEWVSHISGS | TCRASQAIKNYLNWYQQKP |
| | GGTIYYADSVKGRFTISRDNSKNTLYLQ | GKAPKLLIYAGSTLQSGVPSR |
| | MNSLRAEDTAVYYCASASSYYEPGDP | FSGSGSGTDFTLTISSLQPEDF |
| | WGQGTLVTVSS | ATYYCQQNYSTPLTFGGGTK |
| | | VEIK |
| | SEQ ID NO | |
| | 237 | 238 |
| AVE-06-E12 | EVQFLKSGGGLVNPAGILELSCAASGFP | DIQMTQSPSSLSASVGDRVTI |
| | FSSHGMSWVRQAPGKGLEWVSVISGSG | TCRASHSIASYLHWYQQKPG |
| | ATTYYADSVKGRFTISRDNSKNTLYLQ | KAPKLLIYRASTLHTGVPSRF |
| | MNSLRAEDTAVYYCARGQPYYGFDYW | SGSGSGTDFTLTISSLQPEDFA |
| | GQGTLVTVSS | TYYCQQTYSIPWTFGGGTKV |
| | | EIK |
| | SEQ ID NO | |
| | 247 | 248 |
| AVE-06-G12 | EVQFLESGGGLVQPGGSLRLSCAASGF | DIQMTQSPSSLSASVGDRVTI |
| | DFSKFAMSWVRQAPGKGLEWVSHISGS | TCRASQAIKNYLNWYQQKP |
| | GGTIYYADSVKGRFTISRDNSKNTLYLQ | GKAPKLLIYGASNLQSGVPSR |
| | MNSLRAEDTAVYYCASASSYYEPGDP | FSGSGSGTDFTLTISSLQPEDF |
| | WGQGTLVTVSS | ATYYCQQSESTPLTFGGGTK |
| | | VEIK |
| | SEQ ID NO | |
| | 257 | 258 |
| AVE-07-A01 | EVQLLESGGGLVQPGGSLRLSCAASGF | DIQMTQSPSSLSASVGDRVTI |
| | ALTDFAMSWVRQAPGKGLEWVSQISVS | TCRASAPVGRYLNWYQQKP |
| | GGVGYYADSVKGRFTISRDNSKNTLYL | GKAPKLLIYAASSLQSGVPSR |
| | QMNSLRAEDTAVYYCARSRSYYAGAF | FSGSGSGTDFTLTISSLQPEDF |
| | DVWGQGTLVTVSS | ATYYCQQSYSAVTFGGGTKV |
| | | EIK |
| | SEQ ID NO | |
| | 267 | 268 |
| AVE-07-B01 | EVQLLESGGGLVQPGGSLRLSCAASGFT | DIQMTQSPSSLSASVGDRVTI |
| | FGSYAMTWVRQAPGKGLEWVSAIGTT | TCRASQTLRNYLNWYQQKP |
| | DRYTYYADSVKGRFTISRDNSKNTLYL | GKAPKLLIYAASNLQTGVPS |
| | QMNSLRAEDTAVYYCARSRSYYAGAF | RFSGSGSGTDFTLTISSLQPED |
| | DVWGQGTLVTVSS | FATYYCQQRFSPPWTFGGGT |
| | | KVEIK |
| | SEQ ID NO | |
| | 277 | 278 |
| AVE-07-C01 | EVQLLESGGGLVQPGGSLRLSCAASGY | DIQMTQSPSSLSASVGDRVTI |
| | TFSNFAISWVRQAPGKGLEWVSSITGSG | TCRASQSIGSSVNWYQQKPG |
| | VRTFYADSVKGRFTISRDNSKNTLYLQ | KAPKLLIYAASSLQSGVPSRF |
| | MNSLRAEDTAVYYCARSRSYYAGAFD | SGSGSGTDFTLTISSLQPEDFA |
| | VWGQGTLVTVSS | TYYCQQSDNNPWTFGGGTK |
| | | VEIK |
| | SEQ ID NO | |
| | 287 | 288 |
| AVE-07-D01 | EVQLLKSGSGLVQPGGSLRLSCAASGFT | DIQMTQSPSSLSASVGDRVTI |
| | FNNAWMHWVRQAPGKGLEWVSAISSS | TCRASAPVGRYLNWYQQKP |
| | GGYTYYADSVKGRFTISRDNSKNTLYL | GKAPKLLIYAASSLQSGVPSR |
| | QMNSLRAEDTAVYYCARSRSYYAGAF | FSGSGSGTDFTLTISSLQPEDF |
| | DVWGQGTLVTVSS | ATYYCQQAYSIPITFGGGTKV |
| | | EIK |
| | SEQ ID NO | |
| | 297 | 298 |
| AVE-07-E01 | EVQLLESGGGLVQPGGSLRLSCAASGFS | DIQMTQSPSSLSASVGDRVTI |
| | VRDFAMNWVRQAPGKGLEWVSTITGT | TCRAPQNIFSYINWYQQKPG |
| | DRTPYYADSVKGRFTISRDNSKNTLYL | KAPKLLIYAASSLQSGVPSRF |
| | QMNSLRAEDTAVYYCARSRSYYAGAF | SGSGSGTDFTLTISSLQPEDFA |
| | DVWGQGTLVTVSS | TYYCQQTHSIPQTFGGGTKG |
| | | EIK |
| | SEQ ID NO | |
| | 307 | 308 |
| AVE-07-F01 | EVQLLESGGGLVQPGGSLRLSCAASGFT | DIQMTQSPSSLSASVGDRVTI |
| | FSSHNMAWVRQAPGKGLEWVSSIGGG | TCRASQRISTYINWYQQKPG |
| | GRTTNYADSVKGRFTISRDNSKNTLYL | KAPKLLIYGASYLHSGVPSRF |
| | QMNSLRAEDTAVYYCARAAPGAYAYA | SGSGSGTDFTLTISSLQPEDFA |
| | LDYWGQGTLVTVSS | TYYCQQTYSTPPTFGGGTKV |
| | | EIK |
| | SEQ ID NO | |
| | 317 | 318 |
| AVE-07-C02 | EVQLLESGGGLVQPGGSLRLSCAASGFS | DIQMTQSPSSLSASVGDRVTI |
| | VRDFAMTWVRQAPGKGLEWVSAIRISG | TCRASQRISTYINWYQQKPG |
| | GGTFYADSVKGRFTISRDNSKNTLYLQ | KAPKLLIYSASVLENGVPSRF |
| | MNSLRAEDTAVYYCARAAPGAYAYAL | SGSGFGTDFTLTISSLQPEDFA |
| | DYWGQGTLVTVSS | TYYCQQNYRTPPTFGSGTKG |
| | | ENK |
| | SEQ ID NO | |
| | 327 | 328 |
| AVE-07-F02 | EVQLLESGGGLVQPGGSLRLSCAASGFT | DIQMTQSPSSLSASVGDRVTI |
| | FSNYAMTWVRQAPGKGLEWVSTITSSA | TCRASQSISSYLNWYQQKPG |
| | SKTNYADSVKGRFTISRDNSKNTLYLQ | KAPKLLIYAASSLQSGVPSRF |
| | MNSLRAEDTAVYYCARTRYLERFAGG | SGSGSGTDFTLTISSLQPEDFA |
| | LDIWGQGTLVTVSS | TYYCQQSFTAPLTFGGGTKV |
| | | EIK |
| | SEQ ID NO | |
| | 337 | 338 |
| AVE-07-G02 | EVQLLESGGGLVQPGGSLRLSCAASGL | DIQMTQSPSSLSASVGDRVTI |
| | TFSNYTMTWVRQAPGKGLEWVSGISGT | TCRASVSISTFLNWYQQKPG |
| | GGSTWYADSVKGRFTISRDNSKNTLYL | KAPKLLIYAASTLQSGVPSRF |
| | QMNSLRAEDTAVYYCARSRSYYAGAF | SGSGSGTDFTLTISSLQPEDFA |
| | DVWGQGTLVTVSS | TYYCQQSLRTPITFGGGTKVE |
| | | IK |
| | SEQ ID NO | |
| | 347 | 348 |
| AVE-07-H03 | EVQLLESGGGLVQPGGSLRLSCAASGFP | DIQMTQSPSSLSASVGDRVTI |
| | FSSFVMSWVRQAPGKGLEWVSSLSGSG | TCRTSQSVSTYFNWYQQKPG |
| | DITYYADSVKGRFTISRDNSKNTLYLQ | KAPKLLIYAASSLQSGVPSRF |
| | MNSLRAEDTAVYYCARAAPGAYAYAL | SGSGSGTDFTLTISSLQPEDFA |
| | DYWGQGTLVTVSS | TYYCQQSYRTPPTFGGGTKV |
| | | EIK |
| | SEQ ID NO | |
| | 357 | 358 |
| AVE-07-D04 | EVQLLESGGGLVQPGGSLRLSCAASGL | DIQMTQSPSSLSASVGDRVTI |
| | TFNAYAMSWVRQAPGKGLEWVSSLTG | TCRASRTVTSYLNWYQQKPG |
| | SGASTFYADSVKGRFTISRDNSKNTLYL | KAPKLLIYGASYLHSGVPSRF |
| | QMNSLRAEDTAVYYCARSRSYYAGAF | SGSGSGTDFTLTISSLQPEDFA |
| | DVWGQGTLVTVSS | TYYCQQSYRTPPTFGGGTKV |
| | | EIK |
| | SEQ ID NO | |
| | 367 | 368 |
| AVE-07-E06 | EVQLLESGGGLVQPGGSLRLSCAASGFS | DIQMTQSPSSLSASVGDRVTI |
| | VRDFAMNWVRQAPGKGLEWVSSIHRT | TCRANQNIGNYLNWYQQKP |
| | GGGTYYADSVKGRFTISRDNSKNTLYL | GKAPKLLIYAASSLQSGVPSR |
| | QMNSLRAEDTAVYYCARSRSYYAGAF | FSGSGSGTDFTLTISSLQPEDF |
| | DVWGQGTLVTVSS | ATYYCQQSYSTSTFGGGTKV |
| | | EIK |
| | SEQ ID NO | |
| | 377 | 378 |
| AVE-07-F06 | EVQLLESGGGLVQPGGSLRLSCAASGF | DIQMTQSPSSLSASVGDRVTI |
| | DFRSYPMAWVRQAPGKGLEWVSVISG | TCRASQKIARYVNWYQQKP |
| | GGGSTNYADSVKGRFTISRDNSKNTLY | GKAPKLLIYAASDLQSGVPSR |
| | LQMNSLRAEDTAVYYCARSRSYYAGA | FSGSGSGTDFTLTISSLQPEDF |
| | FDVWGQGTLVTVSS | ATYYCQQAYSIPITFGGGTKV |
| | | EIK |
| | SEQ ID NO | |
| | 387 | 388 |
| AVE-07-A07 | EVQLLESGGGLVQPGGSLRLSCAASGF | DIQMTQSPSSLSASVGDRVTI |
| | RFSNYAMTWVRQAPGKGLEWVSGISG | TCRASQPISRYLNWYQQKPG |
| | GGGRTFYADSVKGRFTISRDNSKNTLY | KAPKLLIYDASRLQVGVPSRF |
| | LQMNSLRAEDTAVYYCARGYGAEYFD | SGSGSGTDFTLTISSLQPEDFA |
| | PWGQGTLVTVSS | TYYCQQSHSIPWTFGGGTKV |
| | | EIK |
| | SEQ ID NO | |
| | 397 | 398 |
| AVE-07-E07 | EVQLLESGGGLVQPGGSLRLSCAASGF | DIQMTQSPSSLSASVGDRVTI |
| | RFSNYAMTWVRQAPGKGLEWVSGISG | TCRASQPISRHLNWYQQKPG |
| | GGGRTFYADSVKGRFTISRDNSKNTLY | KAPKLLIYSASSLQSGVPSRF |
| | LQMNSLRAEDTAVYYCARGYGAEYFD | SGSGSGTDFTLTISSLQPEDFA |
| | PWGQGTLVTVSS | TYYCQQSYDRTWTFGGGTK |
| | | GESK |
| | SEQ ID NO | |
| | 407 | 408 |
| AVE-07-D08 | EVQLLESGGGLVQPGGSLRLSCAASGF | DIQMTQSPSSLSASVGDRVTI |
| | RFSNYAMTWVRQAPGKGLEWVSGISG | TCRASQRIATYLNWYQQKPG |
| | GGGRTFYADSVKGRFTISRDNSKNTLY | KAPKLLIYAASHLHGGVPSRF |
| | LQMNSLRAEDTAVYYCARGYGAEYFD | SGSGSGTDFTLTISSLQPEDFA |
| | PWGQGTLVTVSS | TYYCQQSYSIPLTFGGGTKVE |
| | | IK |
| | SEQ ID NO | |
| | 417 | 418 |
| AVE-07-E08 | GGAALKSGGGLVQPGGSLRLSCAASGF | DIQMTQSPSSLSASVGDRVTI |
| | TVSRDYMSWVRQAPGKGLEWVSVIST | TCRTSQTISIYLNWYQQKPGK |
| | GGGSTYYADSVKGRFTISRDNSKNTLY | APKLLIYAASILHGGVPSRFS |
| | LQMNSLRAEDTAVYYCAKPRPYSIAWF | GSGSGTDFTLTISSLQPEDFAT |
| | ADPFDYWGQGTLVTVSS | YYCQQNYSVPPTFGGGTKVE |
| | | IK |
| | SEQ ID NO | |
| | 427 | 428 |
| AVE-07-H08 | ELQLLESGGGLVQPGGSLRLSCAASGFP | DIQMTQSPSSLSASVGDRVTI |
| | FSNNAMSWVRQAPGKGLEWVSVISGS | TCRASQPISRYLNWYQQKPG |
| | YGTTYYADSVKGRFTISRDNSKNTLYL | KAPKLLIYDASRLQVGVPSRF |
| | QMNSLRAEDTAVYYCARVADGAAAY | SGSGSGTDFTLTISSLQPEDFA |
| | AMDYWGQGTLVTVSS | TYYCQQSHSIPWTFGGGTKV |
| | | EIK |
| | SEQ ID NO | |
| | 437 | 438 |
| AVE-07-C10 | EVQLLESGGGLVQPGGSLRLSCAASGF | DIQMTQSPSSLSASVGDRVTI |
| | RFSNYAMTWVRQAPGKGLEWVSGISG | TCRASQPISRYLNWYQQKPG |
| | GGGRTFYADSVKGRFTISRDNSKNTLY | KAPKLLIYAASNLKKGVPSRF |
| | LQMNSLRAEDTAVYYCARGYGAEYFD | SGSGFGTDFTLTISSLQPEDFA |
| | PWGQGTLVTVSS | TYYCQQSYNPPLTFGGGTKV |
| | | EIK |
| | SEQ ID NO | |
| | 447 | 448 |
| AVE-07-D11 | EVQLLESGGGLVQPGGSLRLSCAASGFT | DIQMTQSPSSLSASVGDRVTI |
| | FPHSAMSWVRQAPGKGLEWVSSIAGRG | TCRSSQNIITYLNWYQQKPG |
| | GSPNYADSVKGRFTISRDNSKNTLYLQ | KAPKLAIYGASRVQSGVPSRF |
| | MNSLRAEDTAVYYCARVADGGAAYAF | SGSGSGADFTLTISSLQPEDFS |
| | DYWGQGTLVTVSS | TYYCQQSFSTPLTFGGGTKV |
| | | EIK |
| | SEQ ID NO | |
| | 457 | 458 |
| AVE-07-F11 | EVQLLESGGGLVQPGGSLRLSCAASGF | DIQMTQSPSSLSASVGDRVTI |
| | RFSNYAMTWVRQAPGKGLEWVSGISG | TCRASQPISRYLNWYQQKPG |
| | GGGRTFYADSVKGRFTISRDNSKNTLY | KAPKLLIYDASRLQVGVPSRF |
| | LQMNSLRAEDTAVYYCARGYGAEYFD | SGSGSGTDFTLTISSPQPEDFA |
| | PWGQGTLVTVSS | TYYCQQSHSIPWTFGGGTKG |
| | | EIK |
| | SEQ ID NO | |
| | 467 | 468 |
| AVE-07-G11 | EVQLLESGGGLVQPGGSLRLSCAASGFT | DIQMTQSPSSLSASVGDRVTI |
| | FPHSAMSWVRQAPGKGLEWVSTVTGS | TCRASQSISSYLNWYQQKPG |
| | GSPTYYADSVKGRFTISRDNSKNTLYLQ | KAPKLLIYDASNLQSGVPSRF |
| | MNSLRAEDTAVYYCARVAGGAYGYA | SGSGSGTDFTLTISSLQPEDFA |
| | MDYWGQGTLVTVSS | TYYCQQSFSNLYTFGGGTKV |
| | | EIK |
| | SEQ ID NO | |
| | 477 | 478 |
| AVE-07-F12 | EVQLLESGGGLVQPGGSLRLSCAASGFP | DIQMTQSPSSLSASVGDRVTI |
| | FSVYAMTWVRQAPGKGLEWVSSFGGS | TCRSSQNIITYLNWYQQKPG |
| | GHSPYYADSVKGRFTISRDNSKNTLYL | KAPKLLIYGASRLQSGVPSRF |
| | QMNSLRAEDTAVYYCARVAAGSYAYA | SGSGSGTDFTLTISSLQPEDFA |
| | MDYWGQGTLVTVSS | TYYCQQSFSTPLTFGGGTKE |
| | | VDK |
| | SEQ ID NO | |
| | 487 | 488 |
| AVE-07-G12 | EVQLLESGGGLVQPGGSLRLSCAASGF | DIQMTQSPSSLSASVGDRVTI |
| | RFSNYAMTWVRQAPGKGLEWVSGISG | TCRASQPISRYLNWYQQKPG |
| | GGGRTFYADSVKGRFTISRDNSKNTLY | KAPKLLIYDASRLQVGVPSRF |
| | LQMNSLRAEDTAVYYCARGYGAEYFD | SGSGSGTDFTLTISSLQPEDFA |
| | PWGQGTLVTVSS | TYYCQQSFSTPLTFGGGTKV |
| | | EIK |
| | SEQ ID NO | |
| | 497 | 498 |

In some embodiments, an anti-Activin E antibody of the disclosure comprises the combination of VH/VL variable chain nucleic acid sequences of any one of the combinations listed in Table 3.

TABLE 3
Exemplary Variable Heavy Chain and Variable Light Chain
Nucleic Acid Sequences of Anti-Activin E antibodies
(variable heavy nucleotides (VH NT) and variable
light nucleotides (VL NT).

| CloneID | VH NT | VL NT |
|---|---|---|
| AVE-01- | GAAGTGCAGCTCCTAGAATCCGGC | GACATCCAGATGACACAGTCTCC |
| D07 | GGTGGGCTGGTTCAGCCTGGAGGG | TTCTTCTCTGTCCGCTAGTGTGGG |
| | TCCCTTAGACTGTCTTGTGCCGCG | CGACAGAGTGACGATCACCTGTA |
| | AGTGGCTTCGCCTTTAACAATTAT | GGGCGAGTCAAAGCATTTCATCC |
| | GCAATGACCTGGGTGAGGCAGGC | TACTTGCACTGGTACCAGCAGAA |
| | ACCCGGCAAGGGACTGGAGTGGG | GCCCGGAAAAGCACCTAAACTGT |
| | TAAGCGTCATCAGCGGGAGCGGCA | TAATTTACGCCGCCAGTTCCCTC |
| | CTTCTAAGTACTACGCCGATTCTGT | CAAAGCGGTGTCCCATCTCGGTT |
| | TAAAGGTCGATTCACCATATCACG | TTCGGGGTCAGGGAGCGGCACTG |
| | GGACAACTCCAAGAACACTTTGTA | ATTTCACCCTTACCATCTCATCCC |
| | TCTGCAAATGAATAGTCTCCGCGC | TGCAGCCCGAAGATTTTGCTACA |
| | CGAGGATACGGCTGTCTACTACTG | TATTATTGCCAGCAGAGCTATGG |
| | CGCTAAACCGCGTCCAGGTTCAAT | GAGCCCAACATTCGGTGGAGGC |
| | TTTTTGGGGAGCTGGACCCTTCGA | ACTAAGGTTGAGATAAAG |
| | CTATTGGGGGCAGGGCACATTAGT | |
| | GACAGTGTCGAGC | |
| | SEQ ID NO | |
| | 9 | 10 |
| AVE-01- | GAAGTGCAATTGCTTGAGAGTGGC | GACATACAGATGACACAGTCTCC |
| B08 | GGCGGCCTGGTTCAGCCTGGGGGC | TTCCTCACTTAGCGCGTCGGTTG |
| | TCCCTGAGACTTAGCTGTGCTGCC | GCGACCGCGTGACCATCACATGT |
| | TCTGGCTTCACCTTCGGAGCTTCA | AGAGCATCACAGAACATTGGTCA |
| | GCAATGACCTGGGTGCGTCAGGCT | CTACCTGAATTGGTACCAGCAGA |
| | CCAGGTAAGGGCCTGGAGTGGGTG | AACCCGGAAAGGCCCCAAAATT |
| | TCAGGTATCAGCGGACTAGGGCGG | ATTGATCTATGACGCCTCCAGGC |
| | ACAACCGATTACGCTGATAGCGTC | TGCAAGTGGGCGTACCATCTCGG |
| | AAAGGGCGCTTTACGATTTCGAGG | TTCTCCGGTAGCGGGAGCGGCAC |
| | GACAACTCTAAGAACACTCTGTAT | CGATTTTACACTGACTATCAGTT |
| | CTCCAGATGAATAGTTTAAGGGCA | CTCTCCAGCCCGAAGATTTTGCT |
| | GAAGACACAGCCGTGTACTACTGC | ACGTATTATTGCCAACAGAGTTA |
| | GCCCGAGTAGCCCCCGGAGCGTAT | CAGCACCCCGCCTACCTTCGGGG |
| | GCCTATGCAATGGACTACTGGGGG | GAGGGACTAAGGTCGAGATTAA |
| | CAGGGAACTCTCGTCACAGTTTCC | G |
| | TCC | |
| | SEQ ID NO | |
| | 19 | 20 |
| AVE-02- | GAGGTCCAGCTGCTGGAGTCAGGC | GACATCCAGATGACCCAGAGTCC |
| A03 | GGTGGCCTTGTTCAGCCCGGCGGG | CTCATCTCTGAGTGCATCAGTGG |
| | TCTTTGCGGCTGTCCTGCGCCGCC | GGGACCGCGTGACTATTACCTGC |
| | AGCGGATTCAACTTCAGAAGCTAC | AGAGCTTCTGAGTCCATAGGTAA |
| | GTTATGAACTGGGTGCGCCAGGCC | CTATCTGTCCTGGTACCAGCAAA |
| | CCTGGCAAGGGCTTAGAATGGGTG | AACCTGGCAAGGCGCCCAAGTTG |
| | TCCGCTATCTCCGATGTCGGACGT | CTGATTTACGCCGCTTCGCGATT |
| | AGGACTTACTATGCAGACTCTGTG | ACAGAGGGGCGTCCCATCCCGGT |
| | AAAGGGAGATTTACCATTAGTCGA | TTAGCGGGTCAGGGTCCGGTACT |
| | GATAACTCAAAAAATACACTGTAT | GATTTCACACTTACAATCAGCAG |
| | CTCCAGATGAATAGTTTGAGGGCC | CCTCCAACCAGAAGATTTTGCCA |
| | GAGGACACCGCAGTATATTACTGT | CCTATTACTGTCAGCAGAGCTAT |
| | GCTAAGGCTGAAATACTCGGTGAC | TCTACACCTACCTTCGGAGGAGG |
| | TATGCGTACATGGATTACTGGGGG | CACGAAGGTTGAGATCAAA |
| | CAAGGAACACTAGTGACGGTGAG | |
| | CTCG | |
| | SEQ ID NO | |
| | 29 | 30 |
| AVE-02- | GAAGTGCAGCTCCTAGAGTCGGGC | GACATTCAGATGACACAAAGCCC |
| B04 | GGCGGGCTGGTGCAGCCCGGTGGC | CTCCAGCCTGAGCGCATCGGTGG |
| | TCCCTCCGGCTGAGCTGCGCCGCG | GGGACCGCGTCACCATAACTTGT |
| | AGCGGTTTCGCTTTCAGTGCCTAC | CGGGCCAGTCAGTCTATCAGCTC |
| | GCCATGAACTGGGTTCGTCAAGCC | ATATCTGAACTGGTACCAGCAGA |
| | CCTGGAAAAGGCCTGGAGTGGGTA | AGCCTGGCAAAGCTCCAAAGCTG |
| | TCAAGCATCACGGAGTCTGGGGCT | CTTATCTACGCTACTTCCACTTTA |
| | GCTACATATTATGCAGATTCTGTG | CAGTCCGGTGTTCCATCCAGGTT |
| | AAGGGAAGATTTACTATTTCCCGC | CTCAGGATCTGGCTCTGGTACAG |
| | GACAATAGCAAGAATACCCTGTAT | ATTTCACCTTGACCATTTCAAGT |
| | CTGCAGATGAACAGTTTGCGAGCA | CTCCAGCCTGAAGATTTTGCCAC |
| | GAAGACACCGCCGTCTACTACTGT | GTACTATTGCCAACAGAGAGACA |
| | GCAAGGGGGCTTCTTGCTTCATAC | ATGCGCCCTGGACATTTGGGGGC |
| | ACAGGGGATGTGTGGGGCCAGGG | GGAACCAAAGTGGAGATCAAG |
| | AACCTTAGTCACTGTTTCCTCT | |
| | SEQ ID NO | |
| | 39 | 40 |
| AVE-06- | GAAGTGCAGCTCCTAGAGTCGGGT | GACATCCAGATGACTCAGAGTCC |
| B07 | GGCGGGCTGGTGCAGCCGGGCGG | CAGCTCACTCTCCGCTTCAGTCG |
| | CTCCCTGCGCCTGTCTTGCGCGGCT | GTGATCGGGTTACTATAACATGT |
| | AGTGGATTTGACTTTTCTAGCTTCG | AGAGCCAGCCAAGCTATTAAGA |
| | CAATGACCTGGGTCAGACAGGCCC | ATTACCTGAACTGGTATCAGCAA |
| | CAGGGAAGGGATTGGAGTGGGTG | AAACCCGGGAAGGCACCTAAAC |
| | TCCCACATAACAGGTTCAGGAGGG | TGTTGATTTATGCGGGGTCCACA |
| | ACAATCTATTACGCCGATAGCGTT | CTCCAGAGCGGGGTGCCTAGTAG |
| | AAGGGACGGTTCACTATTAGCAGG | GTTCTCTGGCTCCGGATCTGGCA |
| | GATAATTCAAAAAACACGCTTTAC | CTGACTTTACCTTAACCATCTCTT |
| | TTACAAATGAACAGTCTGCGAGCC | CGCTTCAGCCAGAAGATTTTGCC |
| | GAGGACACCGCAGTATACTATTGT | ACATACTATTGCCAGCAGACATA |
| | GCTTCCGCCAGCTCCTATTACGAA | CAGCACGCCACTGACCTTCGGTG |
| | CCCGGGGACCCTTGGGGCCAGGGC | GCGGAACCAAGGTGGAGATCAA |
| | ACTCTCGTCACCGTGTCATCT | A |
| | SEQ ID NO | |
| | 49 | 50 |
| AVE-06-F07 | GAAGTTCAGCTGTTAGAAAGCGGG | GACATCCAAATGACCCAGTCTCC |
| | GGGGGCCTGGTGCAGCCTGGCGGC | CTCCTCGCTGAGCGCAAGCGTAG |
| | TCACTGAGATTGTCCTGCGCTGCA | GGGACCGGGTCACCATAACGTGT |
| | TCCGGATTCCCGTTTTCCTCACACG | AGAGCTTCTCAGTCAATCTCCTC |
| | GAATGAGCTGGGTTAGGCAGGCTC | ATACTTGAACTGGTATCAGCAGA |
| | CCGGGAAGGGCCTAGAGTGGGTC | AGCCCGGCAAAGCCCCTAAGCTC |
| | AGTACCATTACAGGAAGCGGTAGG | CTGATTTACGATGCCAGCCACCT |
| | AGCACGTACTACGCCGATTCAGTA | ACAAAGCGGAGTTCCATCCAGGT |
| | AAGGGCCGATTCACAATCTCCCGT | TTAGTGGCTCTGGGTCAGGAACA |
| | GACAACTCTAAAAATACTCTTTAT | GATTTCACACTGACAATCAGTTC |
| | CTCCAGATGAACAGTCTGCGGGCA | TCTTCAGCCTGAAGACTTCGCGA |
| | GAGGATACAGCCGTGTATTATTGT | CTTACTATTGCCAGCAGTCCTAT |
| | GCGTCGGACTACCGCGATGCCCCA | AGTACCCCAGTGTTTGGTGGGGG |
| | GGAACCTTTGACGTGTGGGGTCAA | CACTAAAGTGGAGATTAAG |
| | GGGACTCTCGTCACCGTGTCTTCT | |
| | SEQ ID NO | |
| | 59 | 60 |
| AVE-06- | GAGGTCCAGCTGTTGGAATCCGGG | GATATACAGATGACTCAATCTCC |
| A08 | GGAGGCTTAGTGCAACCGGGCGGC | CAGCAGTCTGTCCGCATCCGTCG |
| | TCCCTGCGACTGAGCTGTGCAGCC | GAGATAGGGTAACAATTACCTGC |
| | TCGGGATTCCCCTTTGCTTCTCACG | AGAGCGTCCCAATCGATCAGTTC |
| | CAATGACCTGGGTTCGTCAGGCCC | ATATCTTAACTGGTACCAGCAGA |
| | CAGGGAAAGGCCTGGAGTGGGTTT | AGCCTGGCAAAGCTCCAAAGCTG |
| | CTACTATCACCGGTTCAGGTCGGA | TTAATTTACGACGCCTCTCACCT |
| | GCACATATTACGCTGACAGCGTGA | CCAGAGTGGCGTTCCTTCTCGGT |
| | AGGGGAGATTTACCATTTCACGCG | TTTCTGGCAGCGGTTCCGGGACC |
| | ACAATAGCAAGAACACTCTCTATC | GACTTCACGTTGACAATCTCAAG |
| | TTCAGATGAACAGTCTCAGGGCCG | CCTGCAGCCAGAAGACTTTGCCA |
| | AAGATACAGCCGTCTATTACTGCG | CCTACTATTGTCAGCAGTCATAT |
| | CTAGTGACTACAGGGATGCGCCTG | AGCACTCCCGTGTTCGGGGGTGG |
| | GAACGTTCGATGTGTGGGGCCAGG | AACAAAAGTGGAGATCAAG |
| | GGACACTAGTGACCGTATCCTCT | |
| | SEQ ID NO | |
| | 69 | 70 |
| AVE-06- | GAAGTGCAACTGCTGGAATCGGGC | GATATACAGATGACTCAATCCCC |
| D08 | GGTGGACTGGTCCAGCCTGGAGGC | TTCTTCTCTGTCCGCGTCTGTTGG |
| | TCTTTGAGGCTTTCCTGTGCCGCCT | GGACAGGGTGACAATCACCTGTC |
| | CAGGGTTTGATTTCAGTAAATTCG | GGGCTTCGCAGGCTATTAAAAAT |
| | CTATGTCCTGGGTTCGCCAGGCTC | TATCTTAACTGGTATCAGCAGAA |
| | CCGGGAAAGGACTTGAGTGGGTGT | GCCCGGTAAGGCCCCAAAGTTGC |
| | CCCACATCAGCGGTAGCGGCGGAA | TCATCTATGATGCCAGCCACCCA |
| | CGATTTATTACGCAGACTCAGTAA | CAGTCAGGAGTACCCAGTCGCTT |
| | AGGGCAGATTTACTATATCTCGGG | CAGAGGCTCCGGAAGCGGCACC |
| | ACAATTCAAAGAACACACTCTACC | GACTTCCCTTTCACCATCTCAAG |
| | TGCAGATGAACAGTTTACGAGCAG | CCTGCAGCCGGAGGAATTTGCAA |
| | AGGATACCGCGGTCTATTACTGCG | CATACTACTGCCAGCAAAGCTAC |
| | CCAGTGCCAGCTCCTACTATGAGC | AGTACGCCCGTCTTTGGCGGTGG |
| | CAGGCGACCCGTGGGGGCAGGGG | GACTAAAGTGGAGATTAAA |
| | ACACTCGTGACCGTGAGCTCT | |
| | SEQ ID NO | |
| | 79 | 80 |
| AVE-06- | GAGGTACAACTCCTAGAGAGTGGC | GACATCCAGATGACACAGAGCC |
| G08 | GGAGGCCTTGTGCAGCCCGGAGGA | CGTCTTCACTCAGTGCAAGTGTT |
| | TCGCTGCGACTGTCATGCGCTGCA | GGGGACAGAGTGACCATTACAT |
| | TCCGGCTTTGATTTCTCTAAATTTG | GCAGGGCCGGACAGGCTATCAA |
| | CCATGAGCTGGGTGAGACAGGCCC | AAACTATCTGAATTGGTACCAAC |
| | CGGGTAAGGGCCTGGAATGGGTGT | AAAAACCTGGCAAGGCGCCCAA |
| | CTCACATATCAGGGAGCGGGGGA | ATTGCTCATCTATGCCGGTTCAC |
| | ACAATTTACTATGCCGACTCAGTT | CACTGCAGAGCGGTGTGCCATCG |
| | AAGGGGCGCTTCACCATCAGCAGG | CGGTTTAGCGGATCTGGCTCCGG |
| | GACAATTCCAAAAACACCCTGTAT | CACTGATTTCACCCTTACCATTTC |
| | TTACAGATGAACAGTTTGCGGGCG | CTCTTTACAGCCCGAGGATTTCG |
| | GAGGATACGGCAGTCTACTATTGT | CTACCTACTATTGTCAGCAGACT |
| | GCTTCCGACAGCAGTTACTACGAA | TACTCCACGCCTCTGACATTTGG |
| | CCAGGTGATCCTTGGGGCCAGGGG | AGGGGGGACTAAGGTCGAAATA |
| | ACTCTCGTGACAGTCTCCTCT | AAG |
| | SEQ ID NO | |
| | 89 | 90 |
| AVE-06- | GAGGTGCAGCTGCTGGAGAGCGG | GACATCCAGATGACTCAGTCGCC |
| H08 | CGGAGGGCTGGTGCAACCTGGGG | CTCCTCACTGTCTGCTTCAGTTGG |
| | GCTCACTGCGACTTTCCTGCGCTG | AGATCGGGTGACCATTACCTGCC |
| | CCTCAGGTTTCGATTTCTCTAAGTT | GCGCTAGTCAACCGATCAGCTCC |
| | CGCTATGAGCTGGGTCAGACAGGC | TATGTGACGTGGTATCAGCAGAA |
| | ACCCGGGAAAGGCTTAGAGTGGGT | GCCAGGCAAGGCCCCTAAATTGT |
| | GTCTTCTATTACGCGTGGCAGTGA | TAATCTATTCAGCGTCCCACCTC |
| | AACTACCTACTACGCCGACAGCGT | AGGAGCGGCGTCCCCAGCAGATT |
| | TAAAGGACGGTTTACAATCTCCAG | TTCTGGAAGCGGGAGTGGTACCG |
| | GGACAATTCCAAGAACACCCTCTA | ATTTCACTCTGACAATATCCTCTC |
| | TCTGCAGATGAACAGTCTCCGCGC | TTCAGCCCGAGGACTTTGCAACA |
| | GGAAGACACAGCCGTCTATTACTG | TACTACTGTCAACAGAGTTACAA |
| | TGCAACCCTAGGGTTGGGTTACTA | CGCCCCACCTACATTCGGTGGCG |
| | TTATTACTTTGATGTATGGGGCCA | GGACCAAAGTAGAAATTAAG |
| | GGGAACATTGGTGACTGTTAGCTC | |
| | G | |
| | SEQ ID NO | |
| | 99 | 100 |
| AVE-06- | GAGGTGCAGCTCCTCGAAAGCGGC | GACATTCAAATGACACAGAGCCC |
| A09 | GGCGGATTAGTCCAGCCTGGGGGA | ATCTAGTTTGAGCGCATCCGTAG |
| | TCGCTGAGGTTGTCATGCGCCGCC | GCGATAGAGTGACGATCACTTGT |
| | TCAGGCTTTCCCTTCGCATCCCACG | CGGGCTTCACAGACCATATCTTC |
| | CAATGACTTGGGTGAGACAGGCGC | CTTCGTGTCTTGGTATCAGCAGA |
| | CAGGGAAGGGGCTTGAGTGGGTGT | AACCGGGTAAGGCGCCCAAATT |
| | CTACGATTACCGGCAGCGGGCGGT | ACTCATCTACGCCGCCACAACCC |
| | CCACATATTATGCCGATAGTGTTA | TGCAAAAGGGCGTTCCTTCACGC |
| | AGGGCCGATTCACTATCTCACGTG | TTTTCAGGGAGCGGGTCCGGAAC |
| | ATAACTCCAAAAACACCCTGTACC | AGACTTCACACTTACCATTAGTT |
| | TGCAGATGAATAGTCTACGCGCTG | CGCTGCAGCCCGAGGATTTCGCT |
| | AGGACACCGCTGTATACTATTGTG | ACTTATTACTGCCAGCAGTCCTA |
| | CTTCTGCCTCTAGCTACTACGAAC | CCACACTAGGAGCTTTGGCGGAG |
| | CCGGTGACCCGTGGGGACAAGGTA | GGACCAAAGTCGAAATCAAG |
| | CACTGGTGACAGTCTCCAGC | |
| | SEQ ID NO | |
| | 109 | 110 |
| AVE-06- | GAAGTGCAGCTCCTAGAGAGCGG | GATATACAGATGACACAATCACC |
| B09 | GGGCGGGCTGGTGCAGCCCGGAG | CTCCAGCCTTTCGGCCTCAGTGG |
| | GGTCACTGAGGCTGTCTTGCGCTG | GTGACAGAGTTACCATTACCTGC |
| | CCTCTGGCTTAACATTCTCCAATTT | CGGGCTTCACAGAGCATCAGCTC |
| | CGCAATGACATGGGTGCGCCAGGC | CTACTTGAACTGGTACCAGCAAA |
| | CCCTGGGAAAGGTCTTGAGTGGGT | AGCCCGGAAAAGCTCCAAAGCT |
| | GTCCTCGATTAGGGGATCTGGTGC | CTTAATCTACGCCGCATCTTCTCT |
| | CACCACTTACTACGCTGACTCCGT | GCAGTCTGGGGTGCCTTCTAGGT |
| | CAAGGGCAGATTTACGATCAGCAG | TTAGCGGAAGTGGCAGCGGCAC |
| | AGATAACAGTAAGAATACCCTGTA | CGACTTTACACTGACTATTAGTT |
| | CTTGCAGATGAACAGTCTTCGGGC | CCCTGCAGCCTGAAGATTTCGCG |
| | CGAAGATACAGCTGTCTATTATTG | ACATATTACTGTCAGCAGTCCTA |
| | TGCACGAAGCCGTGAGGCGTATGG | TAGTACTCCATATACCTTCGGTG |
| | ATTTGACTACTGGGGCCAAGGCAC | GCGGGACGAAGGTCGAGATCAA |
| | CCTCGTAACTGTTAGCTCA | A |
| | SEQ ID NO | |
| | 119 | 120 |
| AVE-06- | GAAGTCCACCTGTTGGAGAGCGGC | GACATCCAGATGACGCAATCCCC |
| D09 | GGTGGCCTGGTTCAACCTGGGGGT | TTCTAGCCTGTCAGCGTCAGTGG |
| | TCTCTACGTTTGTCGTGTGCAGCCT | GCGATCGGGTCACAATCACTTGT |
| | CTGGGTTTACATTTAGCCATTATA | AGAGCTTCTCAGAGTATTAGTAC |
| | GCATGAATTGGGTGAGACAGGCAC | CCATCTGAACTGGTACCAGCAGA |
| | CCGGAAAGGGACTTGAGTGGGTTA | AACCAGGCAAGGCACCCAAACT |
| | GCGGGATCTCCGGGTCAGGCTCCG | CCTCATATATGCCGCTTCCAGCT |
| | CGACTTACTATGCCGATTCAGTGA | TACAGAGTGGTGTGCCATCGAGG |
| | AAGGCCGCTTCACCATTTCTCGGG | TTCAGCGGTTCCGGCAGCGGAAC |
| | ACAACAGTAAGAATACTCTGTACC | TGACTTTACCTTGACCATCTCCTC |
| | TCCAGATGAACAGTCTCCGAGCTG | TCTGCAGCCTGAAGATTTTGCCA |
| | AAGACACCGCCGTGTACTACTGCG | CATATTACTGCCAACAGTCACAC |
| | CTTCCGACAGGTATCTGACCTTCG | CGCACCCCCCTTATTTTCGGAGG |
| | ATGTCTGGGGACAGGGCACGTTAG | GGGGACAAAGGTTGAGATTAAG |
| | TGACAGTATCCTCA | |
| | SEQ ID NO | |
| | 129 | 130 |
| AVE-06-F09 | GAGGTGCAGCTGCTGGAGTCTGGC | GATATTCAGATGACACAGAGCCC |
| | GGCGGTCTTGTCCAGCCCGGGGGG | CTCGTCACTGTCTGCGTCCGTCG |
| | TCACTCCGACTAAGCTGCGCTGCG | GTGATCGGGTTACAATCACCTGT |
| | AGTGGATTTGACTTCTCCAAGTTT | AGGGCTTCTCAAGCTATCAAGAA |
| | GCCATGTCCTGGGTGAGACAGGCA | CTACCTCAATTGGTACCAGCAAA |
| | CCAGGAAAGGGACTTGAGTGGGT | AACCTGGCAAGGCCCCAAAACT |
| | GTCACACATATCTGGGAGCGGCGG | GTTAATTTATGCAGGGTCTACAT |
| | AACTATTTACTACGCTGATAGCGT | TGCAGAGTGGCGTGCCTTCAAGA |
| | TAAAGGCCGCTTCACAATCTCACG | TTCAGCGGCTCCGGGTCCGGAAC |
| | GGACAACAGTAAAAACACCCTCTA | TGACTTCACCCTTACAATCAGCA |
| | TTTGCAAATGAATAGTCTGAGGGC | GTCTGCAGCCCGAGGACTTTGCC |
| | CGAAGATACAGCAGTCTATTACTG | ACCTATTATTGCCAGCAGACGTA |
| | TGCCTCCGCCTCTAGCTACTATGA | CAGCACCCCACTCACTTTTGGGG |
| | ACCTGGCGACCCGTGGGGGCAGG | GTGGAACTAAAGGAGAAATAAA |
| | GTACGCTGGTGACCGTATCGTCC | G |
| | SEQ ID NO | |
| | 139 | 140 |
| AVE-06- | GAGGTGCAGTTCCTCGAATCCGGT | GACATCCAAATGACACAGTCCCC |
| G09 | GGGGGTTTAGTGCAGCCCGGGGGC | AAGTTCTCTTTCCGCCTCTGTGG |
| | AGCCTTCGGCTGTCCTGCGCCGCC | GTGACAGAGTGACAATCACCTGT |
| | TCTGGGTTTAGCTTCGACAATTAC | CGCGCATCCCAGTCAATTAATAA |
| | GGTATCAACTGGGTTCGACAAGCA | CTATCTCAACTGGTACCAGCAGA |
| | CCAGGGAAGGGACTGGAATGGGT | AGCCTGGGAAGGCGCCCAAACT |
| | GTCATCAATTTCCTCGTCTGGAGG | GCTGATTTATGCCGCTTCCAGCT |
| | CTCCGCGTATTACGCAGATTCAGT | TACAAAGTGGTGTCCCATCAAGG |
| | GAAAGGCCGTTTTACCATAAGTAG | TTTTCAGGCAGCGGAAGCGGGAC |
| | GGACAATAGCAAGAACACACTGT | CGATTTCACTCTGACGATAAGCT |
| | ACCTCCAGATGAACAGTCTGCGCG | CGTTGCAGCCTGAAGATTTCGCT |
| | CTGAGGACACCGCCGTCTATTACT | ACCTACTACTGCCAGCAGTCTCG |
| | GTGCTAGAGGCGTAGTCCCTGGAG | GACTACCCCCTGGACTTTTGGCG |
| | GATTCGATTATTGGGGCCAGGGCA | GCGGAACAAAGGTTGAGATCAA |
| | CGTTGGTTACTGTGTCTAGC | A |
| | SEQ ID NO | |
| | 149 | 150 |
| AVE-06- | GAAGTGCAGCTTCTTGAGTCCGGC | GACATCCAGATGACGCAAAGTCC |
| C10 | GGGGGACTAGTTCAGCCCGGCGGC | TAGCAGCCTGTCCGCCTCAGTGG |
| | TCACTGCGACTGAGTTGTGCGGCT | GCGACCGGGTGACTATCACATGT |
| | TCAGGGTTTGATTTCAGCAAATTC | AGGGCTTCTCAGACCATTGGCAT |
| | GCAATGTCTTGGGTGAGACAGGCC | ATATCTGAACTGGTACCAGCAGA |
| | CCAGGAAAAGGTTTGGAGTGGGTG | AGCCCGGGAAAGGTCCCAAACC |
| | AGCTCTATTACCGGGACCTCGGGC | GGCGATCTACTCCGCATCCCCAC |
| | GCCACCTACTATGCTGATTCCGTT | TTCAGCGCGGCGTCCCTTCCAGA |
| | AAGGGTAGATTTACTATCTCTAGG | TTCTCGGGGTGCGGCTCAGGTAC |
| | GACAATAGCAAGAACACACTCTAC | AGATTTTACTTTGACAATTTCTA |
| | CTCCAAATGAACAGTCTGCGGGCC | GTCTCCAGCCCGAAGATTTCGCC |
| | GAAGACACTGCCGTATACTATTGC | ACTTACTATTGCCAACAGAGCTA |
| | GCTAGGGATATACGCGTGCGGCGT | TTCTACCCCACACATTACCTTTG |
| | AGCTCCTGGGCAATGGACCCTTGG | GAGGGGGAACCAAGGTTGAGAT |
| | GGACAGGGCACGCTGGTCACAGTC | CAAG |
| | TCCTCA | |
| | SEQ ID NO | |
| | 159 | 160 |
| AVE-06- | GAGGTACAACTCCTGGAGTCAGGC | GACATTCAAATGACACAGAGTCC |
| G10 | GGAGGGCTCGTCCAGCCAGGAGGT | CAGTAGCCTCAGCGCTTCCGTTG |
| | TCCCTAAGACTTTCTTGTGCCGCCT | GCGATAGAGTGACAATTACCTGT |
| | CAGGCTTTGACTTCAGCAAGTTCG | AGGGCCTCCCAGGCAATAAAGA |
| | CAATGAGCTGGGTGAGGCAGGCG | ACTATCTTAACTGGTACCAGCAG |
| | CCTGGGAAGGGGTTAGAATGGGTG | AAACCAGGTAAGGCCCCTAAACT |
| | TCTCACATTAGTGGCTCTGGAGGA | CTTAATCTATGCTGGCTCAACCC |
| | ACTATCTACTATGCCGATTCGGTG | TGCAATCTGGGGTCCCCTCGCGG |
| | AAAGGGCGATTTACAATATCCCGC | TTCTCAGGGTCTGGAAGCGGCAC |
| | GACAATTCAAAAAACACACTGTAC | AGACTTCACTTTGACCATCTCTA |
| | CTGCAGATGAACAGTTTGCGGGCT | GCCTGCAGCCAGAGGAGTTTGCG |
| | GAAGATACCGCAGTCTACTATTGC | ACTTACTATTGCCAACAGACATA |
| | GCTTCCGCCTCCAGCTATTACGAG | CTCCACCCCTCTGACGTTTGGAG |
| | CCCGGTGACCCGTGGGGCCAGGGC | GGGGTACTAAGGGAGAAAATCA |
| | ACCCTGGTTACGGTGAGTAGC | G |
| | SEQ ID NO | |
| | 169 | 170 |
| AVE-06- | GAAGTGCAGCTGCTAGAATCCGGA | GATATCCAAATGACACAGTCTCC |
| H10 | GGGGGCTTAGTCCAGCCTGGCGGC | CAGTTCATTGTCCGCTTCCGTTG |
| | TCCCTTCGGCTCTCTTGTGCCGCTT | GAGATAGAGTCACCATTACCTGC |
| | CCGGATTTACGTTCACTAACTATG | AGGGCCAGTCAGGCGATAAAAA |
| | CACTGGCCTGGGTGCGACAGGCGC | ACTACCTTAATTGGTATCAGCAG |
| | CAGGGAAGGGGCTGGAGTGGGTA | AAGCCTGGGAAAGCTCCCAAGCT |
| | AGCCACATCTCTGGATCAGGAGGT | GCTGATCTACGCCGGCTCAACTC |
| | ACAATTTACTATGCTGACAGCGTG | TGCAGTCTGGGGTGCCTTCTCGG |
| | AAGGGGCGCTTCACCATAAGCAGA | TTTTCCGGTTCGGGCAGCGGGAC |
| | GATAACTCTAAAAATACCCTTTAT | GGACTTTACACTCACAATTAGCA |
| | CTGCAAATGAATAGTCTCAGGGCC | GCTTACAACCAGAGGACTTCGCA |
| | GAGGATACTGCAGTGTATTACTGC | ACTTATTACTGTCAGCAGACCTA |
| | GCTTCGGCCTCAAGCTACTACGAG | TAGCACACCACTCACTTTCGGCG |
| | CCCGGCGACCCGTGGGGTCAGGGC | GAGGTACCAAGGTGGAAATCAA |
| | ACATTGGTCACCGTTAGTTCC | A |
| | SEQ ID NO | |
| | 179 | 180 |
| AVE-06- | GAGGTGCAGCTGCTGGAGTCTGGG | GATATCCAGATGACCCAGTCGCC |
| B11 | GGCGGGCTAGTCCAACCCGGAGG | AAGCTCTCTCTCCGCTTCTGTTGG |
| | ATCGCTGAGGCTTTCCTGCGCCGC | AGACAGGGTCACAATTACTTGCC |
| | GTCAGGGTTTGACTTCAGCAAGTT | GGGCATCCCAAGCGATCAAGAA |
| | TGCCATGTCTTGGGTGCGACAGGC | CTATTTAAATTGGTACCAGCAGA |
| | ACCTGGAAAAGGATTGGAATGGGT | AGCCTGGGAAAGCTCCTAAACTT |
| | GTCACACATAAGCGGTTCTGGCGG | CTGATATATGCCGGCTCTACCCT |
| | CACTATCTATTACGCCGACTCCGT | GCAGTCAGGGGTGCCCAGTAGAT |
| | CAAAGGCCGCTTCACAATTAGCCG | TCTCAGGGAGCGGCAGTGGTACC |
| | GGATAACAGCAAGAATACGCTCTA | GATTTCACACTGACCATTAGCAG |
| | CTTACAGATGAACAGTCTCAGAGC | CTTGCAACCAGAGGACTTTGCCA |
| | CGAGGATACCGCTGTTTACTATTG | CATACTATTGTCAGCAGACGTAC |
| | TGCATCAGCTAGTAGTTACTATGA | TCCACTCCCCTCACATTTGGTGG |
| | ACCAGGTGACCCGTGGGGGCAGG | CGGAACTAAAGTGGAAATCAAG |
| | GCACCCTGGTGACAGTATCCTCC | |
| | SEQ ID NO | |
| | 189 | 190 |
| AVE-06- | GAGGTCCAGCTGCTAGAGTCTGGA | GATATTCAGATGACTCAGAGCCC |
| E11 | GGCGGGCTGGTGCAGCCGGGAGG | ATCTAGCCTCAGCGCTTCTGTGG |
| | TAGTTTACGCCTGTCCTGTGCAGCT | GGGACAGGGTTACGATTACATGT |
| | TCGGGCATTAGGTTTTCAAGCTAC | AGAGCAAGTCAGGCCATCAAGA |
| | GCAATGTCTTGGGTGCGACAAGCG | ACTACCTTAATTGGTACCAGCAA |
| | CCTGGTAAGGGACTGGAATGGGTG | AAGCCTGGCAAAGCTCCTAAACT |
| | AGCCACATCAGCGGCAGCGGCGG | GCTCATCTACGCGGGGTCCACAT |
| | AACTATATATTATGCCGACTCCGT | TGCAATCTGGAGTCCCCTCGCGG |
| | AAAGGGGCGGTTCACGATCTCACG | TTTAGTGGAAGCGGCTCCGGTAC |
| | TGATAACTCAAAAAACACATTGTA | AGACTTCACTCTGACCATCTCAT |
| | TCTCCAGATGAATAGTCTTAGAGC | CATTACAGCCAGAGGATTTCGCC |
| | CGAGGATACCGCCGTTTACTACTG | ACATATTATTGCCAGCAGACCTA |
| | CGCTTCCGCCTCCAGTTACTATGA | TTCCACTCCCCTGACCTTTGGCG |
| | ACCAGGGGACCCCTGGGGCCAGG | GCGGGACCAAAGTGGAAATAAA |
| | GGACACTCGTCACCGTGTCTTCT | G |
| | SEQ ID NO | |
| | 199 | 200 |
| AVE-06- | GAAGTGCAGCTCTTAGAGAGCGGG | GACATCCAGATGACCCAGTCTCC |
| H11 | GGCGGACTGGTCCAGCCAGGCGGT | TTCCTCCCTTAGTGCTTCGGTGG |
| | AGCCTGCGCCTAAGTTGCGCCGTT | GTGATAGGGTCACCATTACATGC |
| | TCTGGGTTCGATTTTTCTAAGTTCG | AGAGCCAGTCAGGCAATAAAGA |
| | TGATGAGTGGCGTGCGGCAGGCTC | ACTACTTGAATTGGTACCAGCAA |
| | CTGGCAAAGGTCTGGAGTGGGTTT | AAACCCGGCAAGGCCCCCAAAC |
| | CACACATATCCGGGTCTGGGGGAA | TGCTGATCTATGCTGGTTCCACG |
| | CGATTTACTACGCTGATTCAGTGA | TTACAGTCTGGAGTGCCAAGCCG |
| | AAGGAAGATTTACCATCTCCAGGG | GTTTAGCGGCTCAGGGTCTGGAA |
| | ACAACAGCAAGAATACTCTCTATC | CTGATTTCACACTGACAATTAGC |
| | TGCAAATGAACAGTCTTCGAGCGG | TCACTCCAGCCAGAAGACTTCGC |
| | AAGACACAGCCGTCTACTATTGTG | GACTTATTATTGTCAGCAAACCT |
| | CATCCGCCTCGTCATATTACGAGC | ACAGCACCCCTCTCACTTTTGGG |
| | CGGGTGACCCCTGGGGACAGGGC | GGCGGGACAAAGGTTGAGATCA |
| | ACATTGGTGACCGTAAGCTCC | AA |
| | SEQ ID NO | |
| | 209 | 210 |
| AVE-06- | GAAGTGCAGCTCCTAGAGTCCGGA | GATATACAGATGACCCAGTCACC |
| A12 | GGTGGCCTCGTGCAGCCGGGAGGC | CTCCAGCTTGTCAGCTAGTGTTG |
| | TCCCTGCGCCTGTCCTGCGCGGCC | GGGACCGGGTCACCATCACATGC |
| | TCTGGCTTTGACTTTAGTAAGTTCG | AGAGCCTCTCAGGCGATCAAGA |
| | CTATGAGCTGGGTGAGACAGGCTC | ATTACCTTAACTGGTATCAGCAG |
| | CAGGTAAGGGCCTGGAGTGGGTAT | AAACCAGGAAAAGCTCCGAAGT |
| | CTCACATCTCCGGGTCAGGAGGGA | TACTGATTTACGCAGGTAGCACA |
| | CCATATATTACGCCGACTCTGTCA | CTGCAGTCGGGCGTGCCCTCTAG |
| | AAGGCCGGTTCACTATTTCAAGGG | GTTCAGTGGGAGCGGCTCCGGAA |
| | ATAATTCAAAAAACACATTATACC | CAGACTTTACTCTGACCATTTCTT |
| | TTCAAATGAACAGTCTGCGAGCCG | CCCTCCAACCAGAAGATTTCCCT |
| | AAGACACAGCAGTGTACTATTGTG | ACTTATTATTTTCAGCAAAACTA |
| | CCAGCGCAAGCAGCTACTATGAGC | CAGCACGCCTTTCGCCTTTGGCG |
| | CTGGAGATCCCTGGGGGCAGGGG | GTGGGACCAAAGTGGAGATCAA |
| | ACCTTGGTCACGGTTAGTTCG | G |
| | SEQ ID NO | |
| | 219 | 220 |
| AVE-06- | GAGGTGCAGCTTCTGGAGAGCGGA | GACATCCAGATGACACAGAGTCC |
| B12 | GGGGGACTGGTCCAGCCTGGCGGC | ATCTAGCTTATCCGCGAGTGTTG |
| | AGTCTAAGATTGTCCTGCGCCGCG | GGGACAGGGTCACCATCACATGT |
| | TCAGGGTTCGATTTTTCCAAATTTG | AGAGCCTCTCAGGCAATTAAAAA |
| | CCATGTCATGGGTGCGCCCAGCTC | CTACCTGAATTGGTATCAACAAA |
| | CCTGTAAGGGATTAGAATGGGTGA | AGCCCGGAAAGGCCCCAAAGCT |
| | GTCACATCTCAGGTTCTGGGGGCA | GCTCATCTACGCCGGCTCCACCC |
| | CAATTTATTACGCAGACTCCGTCA | TGCAGAGCGGAGTGCCTAGCCG |
| | AGGGGCGATTCACGATATCCCGGG | GTTTAGCGGCTCTGGCTCGGGGA |
| | ACAACTCTAAAAACACTCTGTACC | CTGATTTTACATTGACTATTTCAT |
| | TCCAAATGAATAGTCTCAGGGCCG | CACTTCAGCCCGAAGATTTCGCT |
| | AGGACACCGCTGTGTACTATTGCG | ACATATTATTGCCAGCAGACCTA |
| | CCTCGGCAAGCAGCTATTACGAAC | CTCCACTCCTCTCACGTTCGGTG |
| | CCGGCGATCCGTGGGGCCAGGGTA | GTGGGACCAAAGTGGAGATAAA |
| | CCCTGGTAACAGTTAGCTCT | A |
| | SEQ ID NO | |
| | 229 | 230 |
| AVE-06- | GAGGTGCAGCTGCTGGAAAGCGG | GATATACAAATGACACAGAGTCC |
| C12 | AGGAGGGTTAGTGCAACCAGGAG | CTCATCCCTGTCAGCTTCTGTGG |
| | GATCTCTCCGGCTGAGCTGCGCTG | GTGATAGGGTCACAATCACATGC |
| | CCTCAGGCTTTGATTTCAGCAAGT | AGAGCCAGCCAGGCGATTAAAA |
| | TCGCCATGTCCTGGGTCCGACAGG | ATTACCTCAACTGGTATCAGCAG |
| | CACCGGGGAAAGGCCTTGAGTGG | AAGCCTGGCAAGGCTCCAAAATT |
| | GTGAGCCACATATCCGGCTCAGGT | ACTTATCTACGCAGGTAGCACCC |
| | GGGACCATTTACTACGCTGATTCT | TGCAGTCCGGAGTGCCTTCTCGG |
| | GTAAAAGGGAGATTTACGATCTCA | TTCAGCGGGTCCGGCTCTGGGAC |
| | CGCGACAACAGTAAGAATACACTC | CGACTTTACCCTCACCATCAGTA |
| | TACCTGCAGATGAACAGTTTGAGG | GCCTGCAGCCAGAAGACTTCGCC |
| | GCCGAAGACACAGCCGTGTATTAT | ACTTATTACTGTCAGCAAAACTA |
| | TGTGCGTCTGCATCCTCGTACTATG | TTCGACTCCCTTGACGTTTGGAG |
| | AGCCTGGCGACCCCTGGGGCCAGG | GCGGGACTAAAGTTGAGATTAA |
| | GTACCCTAGTCACTGTTTCCAGT | G |
| | SEQ ID NO | |
| | 239 | 240 |
| AVE-06- | GAGGTCCAATTTCTGAAAAGCGGT | GACATACAGATGACACAGTCTCC |
| E12 | GGCGGGCTGGTTAATCCCGCGGGC | CAGCTCTTTATCCGCTTCCGTGG |
| | ATCCTTGAGCTCTCTTGTGCAGCCT | GCGATAGAGTGACTATTACATGC |
| | CCGGGTTCCCGTTTAGCAGTCACG | CGCGCAAGTCACTCCATCGCCAG |
| | GTATGTCATGGGTGAGGCAGGCTC | CTATCTCCACTGGTACCAACAAA |
| | CAGGAAAGGGACTAGAATGGGTA | AGCCAGGTAAGGCGCCCAAACT |
| | TCTGTGATTTCAGGCTCCGGAGCC | GCTGATCTACAGGGCCAGCACGC |
| | ACCACCTATTACGCAGATTCCGTT | TGCATACAGGAGTCCCTTCTCGG |
| | AAGGGCCGCTTCACTATATCGAGA | TTTTCCGGAAGTGGCTCAGGGAC |
| | GACAACAGCAAAAATACTTTATAT | AGATTTCACTCTTACCATTTCATC |
| | TTGCAGATGAACAGTCTGCGAGCC | GTTGCAGCCTGAGGACTTTGCTA |
| | GAAGACACAGCTGTCTATTACTGC | CCTATTATTGTCAGCAGACCTAC |
| | GCCCGGGGGCAGCCTTACTATGGC | AGCATCCCGTGGACTTTCGGTGG |
| | TTCGATTACTGGGGACAGGGGACG | CGGGACCAAAGTTGAAATTAAG |
| | CTCGTGACAGTGAGCTCT | |
| | SEQ ID NO | |
| | 249 | 250 |
| AVE-06- | GAGGTGCAATTTCTGGAGTCAGGG | GACATTCAGATGACACAGTCACC |
| G12 | GGGGGTTTGGTTCAGCCGGGAGGC | AAGTAGTCTGTCCGCGTCTGTTG |
| | AGCCTTAGGTTATCCTGTGCCGCC | GGGATCGGGTGACGATTACATGC |
| | TCAGGGTTCGATTTCAGCAAATTT | AGGGCTTCGCAGGCCATCAAAA |
| | GCAATGAGTTGGGTCCGGCAGGCA | ACTACTTAAACTGGTATCAACAG |
| | CCCGGCAAGGGACTGGAATGGGT | AAGCCCGGCAAGGCCCCTAAGCT |
| | AAGCCACATTTCTGGCTCTGGAGG | GCTTATATACGGGGCTTCCAATC |
| | CACCATCTACTACGCCGACTCCGT | TGCAGTCAGGAGTCCCTTCCAGA |
| | GAAGGGACGATTCACGATATCGCG | TTTTCTGGATCTGGTAGCGGTAC |
| | CGACAATTCAAAAAACACCCTGTA | TGATTTTACACTCACCATCTCCA |
| | TCTCCAGATGAACAGTCTGAGAGC | GCCTCCAACCCGAGGACTTCGCA |
| | TGAGGATACTGCCGTCTATTACTG | ACCTATTACTGTCAGCAGAGCGA |
| | CGCTTCCGCGTCTAGTTACTATGA | AAGCACTCCATTGACCTTCGGGG |
| | ACCAGGTGACCCTTGGGGCCAGGG | GCGGCACCAAAGTGGAGATCAA |
| | GACACTCGTGACAGTGAGCTCC | A |
| | SEQ ID NO | |
| | 259 | 260 |
| AVE-07- | GAAGTGCAGCTTTTAGAATCAGGC | GATATCCAAATGACCCAGTCTCC |
| A01 | GGTGGACTCGTACAGCCTGGGGGC | TTCCTCGCTCAGCGCAAGTGTGG |
| | TCCCTGAGACTGAGCTGCGCTGCC | GCGACAGGGTGACCATTACATGT |
| | TCAGGCTTCGCCCTCACAGACTTT | CGCGCCAGTGCTCCAGTCGGAAG |
| | GCCATGTCCTGGGTGCGGCAAGCT | ATATCTGAACTGGTATCAGCAGA |
| | CCCGGGAAGGGACTAGAGTGGGT | AACCTGGGAAGGCCCCCAAGCT |
| | GTCCCAGATCTCGGTGTCTGGTGG | GCTTATCTATGCAGCCTCATCCTT |
| | CGTTGGGTACTACGCCGACAGCGT | ACAGTCTGGGGTGCCCTCCCGGT |
| | CAAAGGGAGGTTCACGATTTCCAG | TCTCAGGTAGCGGTAGCGGGACT |
| | GGATAACAGTAAGAATACCCTGTA | GACTTTACACTGACCATTTCATC |
| | CCTGCAAATGAACAGTTTGCGCGC | TTTGCAGCCAGAGGATTTTGCTA |
| | GGAGGATACAGCAGTTTATTATTG | CGTACTACTGCCAGCAAAGCTAC |
| | TGCACGAAGCCGTTCTTATTACGC | TCCGCGGTTACATTCGGAGGCGG |
| | TGGAGCATTTGACGTGTGGGGCCA | CACTAAAGTAGAAATAAAG |
| | GGGAACCCTTGTCACTGTCAGCTC | |
| | T | |
| | SEQ ID NO | |
| | 269 | 270 |
| AVE-07- | GAGGTCCAGCTACTGGAAAGCGG | GATATTCAGATGACACAGAGCCC |
| B01 | GGGCGGGTTAGTTCAGCCCGGGGG | CTCTAGTCTCAGCGCCTCCGTGG |
| | TAGCTTGCGCCTCTCATGCGCCGC | GTGACAGAGTTACCATTACCTGC |
| | CTCCGGATTCACCTTCGGCTCTTAT | AGGGCTTCGCAGACTCTGCGGAA |
| | GCCATGACCTGGGTGAGACAGGCA | CTACTTGAACTGGTACCAACAAA |
| | CCTGGCAAGGGACTGGAATGGGTG | AACCAGGGAAAGCGCCGAAGCT |
| | TCTGCTATTGGCACTACTGACAGA | CCTTATCTACGCAGCCTCCAATT |
| | TACACGTACTACGCTGATAGCGTG | TACAGACTGGCGTCCCCTCTCGC |
| | AAAGGCCGTTTTACCATCTCCCGA | TTTAGTGGCTCAGGATCAGGCAC |
| | GACAACAGTAAGAACACACTGTAT | GGACTTCACTCTGACCATAAGCT |
| | CTCCAGATGAATAGTCTGAGGGCT | CTCTGCAGCCAGAGGATTTCGCT |
| | GAGGATACAGCCGTTTATTACTGT | ACCTATTATTGTCAGCAGCGATT |
| | GCAAGGTCGCGGTCCTACTATGCG | TTCCCCTCCTTGGACATTCGGAG |
| | GGTGCATTTGACGTCTGGGGACAA | GTGGGACAAAGGTGGAAATCAA |
| | GGGACCCTTGTAACAGTGTCTTCA | G |
| | SEQ ID NO | |
| | 279 | 280 |
| AVE-07- | GAGGTACAGCTCCTCGAAAGTGGA | GATATTCAGATGACTCAATCCCC |
| C01 | GGAGGGCTGGTCCAGCCCGGCGGC | TAGCAGCCTGTCTGCGAGTGTGG |
| | TCACTAAGACTTTCATGTGCGGCA | GCGACAGAGTTACCATCACTTGC |
| | TCGGGGTATACTTTCAGCAATTTC | AGGGCTTCACAATCCATAGGGTC |
| | GCTATTTCCTGGGTGCGGCAAGCC | ATCCGTCAATTGGTATCAGCAGA |
| | CCTGGCAAAGGGCTGGAATGGGTG | AACCTGGGAAAGCTCCCAAGTTG |
| | AGCAGCATAACTGGTTCTGGTGTG | CTTATCTATGCCGCCTCTTCTCTG |
| | CGTACATTCTACGCCGATTCTGTTA | CAGTCCGGCGTGCCATCTCGGTT |
| | AGGGAAGATTTACCATCTCCAGGG | CAGCGGCAGCGGAAGTGGGACA |
| | ACAATAGCAAGAACACATTATATC | GACTTTACACTCACCATCTCGTC |
| | TGCAGATGAACAGTTTGCGCGCCG | ATTACAGCCAGAAGATTTCGCAA |
| | AGGATACCGCTGTCTACTATTGCG | CGTACTACTGTCAGCAGAGTGAC |
| | CAAGGTCACGATCCTACTACGCTG | AACAACCCCTGGACCTTTGGAGG |
| | GCGCCTTTGACGTTTGGGGGCAGG | TGGTACAAAGGTAGAGATTAAG |
| | GCACCCTGGTGACGGTGTCTTCC | |
| | SEQ ID NO | |
| | 289 | 290 |
| AVE-07- | GAGGTACAACTCTTAAAGTCCGGA | GATATTCAGATGACACAGTCCCC |
| D01 | TCTGGATTGGTCCAGCCTGGCGGC | AAGCTCGCTGTCTGCTTCCGTTG |
| | AGCCTCAGACTGTCATGCGCAGCC | GCGATAGAGTAACTATCACATGC |
| | AGCGGATTTACCTTCAACAATGCC | CGCGCCAGCGCTCCAGTGGGGA |
| | TGGATGCACTGGGTGAGGCAGGCA | GGTACCTCAACTGGTACCAGCAG |
| | CCCGGCAAAGGTCTTGAGTGGGTG | AAACCCGGGAAGGCACCTAAAT |
| | TCGGCCATCTCAAGCTCCGGGGGC | TGCTGATCTATGCCGCCTCTTCA |
| | TATACATATTATGCAGATTCAGTT | CTGCAGTCAGGTGTCCCCTCTCG |
| | AAGGGGCGGTTCACCATTTCTCGT | GTTTAGTGGGAGCGGTAGTGGAA |
| | GACAACTCCAAAAATACTCTGTAC | CTGACTTTACACTTACCATTTCCT |
| | CTGCAGATGAACAGTCTTCGAGCT | CCTTACAGCCTGAAGACTTCGCA |
| | GAAGATACAGCTGTGTATTACTGT | ACGTATTACTGTCAACAAGCGTA |
| | GCTAGGTCCCGCTCTTACTACGCG | TAGCATCCCCATAACCTTCGGAG |
| | GGCGCCTTTGACGTTTGGGGGCAG | GCGGCACCAAGGTGGAGATTAA |
| | GGTACTCTGGTCACGGTGAGCAGT | G |
| | SEQ ID NO | |
| | 299 | 300 |
| AVE-07- | GAGGTTCAGCTGTTAGAATCAGGA | GATATACAGATGACCCAGTCTCC |
| E01 | GGGGGGTTGGTGCAGCCTGGAGGC | CAGCAGCCTTTCTGCCTCAGTCG |
| | TCTCTCCGGCTGTCCTGCGCCGCAT | GGGATAGAGTTACGATCACATGT |
| | CGGGCTTCTCTGTCCGTGACTTCGC | CGGGCTCCACAGAATATCTTCAG |
| | CATGAATTGGGTTCGGCAGGCCCC | CTACATTAACTGGTACCAGCAGA |
| | CGGTAAGGGGCTGGAGTGGGTGA | AGCCGGGCAAGGCACCTAAGTT |
| | GCACCATCACAGGTACTGATAGAA | GCTGATCTATGCGGCTAGTTCCT |
| | CACCATACTACGCAGATTCAGTAA | TACAATCCGGAGTGCCATCGAGG |
| | AGGGAAGATTTACTATTAGCAGGG | TTTTCCGGCTCTGGGTCCGGTAC |
| | ACAATTCTAAAAACACGCTTTACC | AGACTTTACCCTGACTATTAGCT |
| | TCCAGATGAACAGTCTAAGGGCTG | CACTCCAGCCTGAGGACTTCGCC |
| | AAGATACAGCCGTGTATTATTGTG | ACCTATTACTGCCAGCAAACACA |
| | CTCGCAGCCGATCCTACTATGCTG | CAGTATCCCCCAAACTTTCGGCG |
| | GCGCGTTTGACGTGTGGGGCCAAG | GTGGGACCAAAGGAGAAATTAA |
| | GCACCCTGGTCACCGTGAGTTCC | A |
| | SEQ ID NO | |
| | 309 | 310 |
| AVE-07-F01 | GAAGTGCAATTGCTCGAAAGCGGC | GATATTCAGATGACCCAATCACC |
| | GGAGGCCTTGTTCAGCCTGGAGGT | AAGCAGCCTTAGCGCATCCGTTG |
| | AGCCTGAGGCTGTCTTGTGCGGCT | GTGATCGGGTGACAATTACCTGC |
| | TCCGGGTTCACCTTCAGCTCACAC | AGGGCCTCTCAGAGAATATCCAC |
| | AACATGGCTTGGGTGCGGCAGGCA | GTACATCAACTGGTACCAGCAGA |
| | CCAGGAAAGGGGCTAGAGTGGGT | AGCCTGGGAAAGCGCCTAAGCTC |
| | GAGTTCCATCGGTGGCGGTGGAAG | CTGATTTATGGCGCCAGTTATTT |
| | GACTACGAACTATGCCGACTCAGT | GCACTCTGGCGTGCCCTCGCGCT |
| | CAAAGGGCGTTTTACAATTTCCCG | TTAGCGGTAGTGGATCAGGGACC |
| | CGATAATTCTAAGAATACTCTTTA | GACTTTACTCTGACTATCTCTTCC |
| | CCTCCAGATGAACAGTCTGCGAGC | TTACAGCCCGAAGACTTCGCTAC |
| | CGAGGATACCGCTGTCTATTACTG | ATATTACTGTCAACAGACATACT |
| | CGCCAGAGCTGCACCCGGCGCCTA | CCACACCCCCAACTTTCGGGGGC |
| | CGCCTATGCATTAGACTACTGGGG | GGAACCAAAGTCGAGATCAAG |
| | CCAGGGGACCCTGGTGACAGTATC | |
| | GTCT | |
| | SEQ ID NO | |
| | 319 | 320 |
| AVE-07- | GAAGTACAGCTTCTGGAGTCTGGC | GACATTCAGATGACCCAGTCACC |
| C02 | GGAGGACTAGTTCAGCCCGGCGGG | GTCTTCACTCTCCGCCAGTGTGG |
| | TCACTCCGGCTGAGCTGTGCTGCA | GTGACAGAGTGACTATCACCTGT |
| | TCAGGGTTTAGCGTCCGTGATTTC | CGAGCCAGCCAAAGGATATCTAC |
| | GCCATGACATGGGTCAGACAGGCT | TTATATCAACTGGTATCAGCAGA |
| | CCTGGTAAGGGCCTTGAATGGGTG | AACCAGGGAAGGCACCTAAGTT |
| | TCCGCCATCAGAATATCGGGGGGG | ATTGATCTACTCGGCGTCCGTTC |
| | GGAACCTTTTATGCCGACAGCGTG | TGGAAAATGGAGTCCCCTCTCGG |
| | AAAGGCCGATTCACTATTTCCCGC | TTTAGTGGGAGCGGATTCGGCAC |
| | GACAATTCTAAGAACACTCTGTAT | GGATTTTACACTTACCATTAGCT |
| | TTGCAAATGAACAGTCTGAGGGCC | CCCTGCAGCCAGAGGATTTCGCT |
| | GAGGATACAGCCGTCTACTACTGC | ACCTACTACTGCCAACAGAATTA |
| | GCAAGGGCGGCACCAGGTGCTTAC | TCGCACTCCTCCCACATTCGGTA |
| | GCTTATGCCCTCGACTACTGGGGA | GCGGCACAAAAGGCGAGAACAA |
| | CAGGGCACGTTAGTGACCGTGAGT | G |
| | TCC | |
| | SEQ ID NO | |
| | 329 | 330 |
| AVE-07-F02 | GAGGTCCAGCTGCTGGAATCAGGC | GACATCCAGATGACCCAGTCTCC |
| | GGAGGGCTGGTTCAGCCCGGCGGC | TAGTAGCCTTTCTGCAAGCGTTG |
| | TCCCTCAGGTTAAGCTGCGCGGCA | GAGATCGGGTGACCATCACATGC |
| | AGCGGGTTTACCTTCTCTAATTAC | AGAGCCAGCCAAAGTATAAGCT |
| | GCCATGACATGGGTGAGACAAGCC | CATACTTGAACTGGTACCAGCAG |
| | CCTGGAAAAGGTTTGGAGTGGGTG | AAGCCAGGTAAGGCTCCAAAAC |
| | TCGACCATCACGAGCTCTGCTTCC | TGTTAATTTACGCTGCCTCTTCCC |
| | AAGACTAACTATGCAGATTCTGTG | TGCAGTCGGGCGTGCCTAGTAGG |
| | AAAGGCCGGTTCACAATTTCCAGA | TTTTCAGGGTCAGGGTCCGGAAC |
| | GATAACTCAAAGAATACTCTTTAC | TGACTTCACCCTCACAATCTCCT |
| | CTACAGATGAACAGTCTGCGAGCC | CCCTGCAGCCCGAGGATTTCGCC |
| | GAAGACACTGCTGTCTATTACTGT | ACCTATTATTGTCAACAGTCTTTT |
| | GCCCGTACCAGGTATCTTGAGCGC | ACGGCGCCCCTCACTTTCGGCGG |
| | TTTGCTGGCGGTCTGGACATATGG | TGGCACAAAGGTCGAAATTAAA |
| | GGACAGGGGACACTCGTGACCGTA | |
| | AGCAGT | |
| | SEQ ID NO | |
| | 339 | 340 |
| AVE-07- | GAGGTGCAACTGCTGGAATCTGGT | GATATTCAGATGACTCAATCACC |
| G02 | GGAGGTCTGGTCCAGCCCGGAGGC | CTCATCGCTGTCTGCGAGTGTAG |
| | AGTCTTAGGCTCAGCTGTGCCGCA | GGGACCGCGTCACAATCACCTGT |
| | TCCGGCCTAACTTTTAGCAATTAC | AGAGCCTCCGTGAGCATCAGTAC |
| | ACCATGACATGGGTTCGTCAGGCC | GTTTTTAAACTGGTATCAACAGA |
| | CCTGGGAAAGGGTTGGAATGGGTT | AACCCGGGAAGGCTCCTAAGCTG |
| | TCTGGCATCTCAGGAACCGGGGGT | CTCATATACGCAGCTTCAACACT |
| | TCGACATGGTACGCTGATTCAGTG | TCAGAGCGGTGTTCCAAGCAGGT |
| | AAGGGCAGATTCACCATTTCCCGA | TTTCTGGGTCCGGATCTGGAACT |
| | GACAACAGCAAGAATACGCTGTAT | GACTTCACATTGACTATCAGCTC |
| | CTTCAGATGAACAGTTTAAGGGCG | CCTCCAGCCAGAAGATTTCGCCA |
| | GAGGACACTGCAGTCTATTATTGC | CCTATTACTGCCAGCAGTCCCTG |
| | GCCCGCTCCCGGTCCTACTACGCT | CGGACCCCTATTACCTTCGGCGG |
| | GGGGCCTTCGATGTGTGGGGCCAG | CGGCACAAAGGTGGAGATTAAA |
| | GGAACCCTCGTAACAGTGTCTAGC | |
| | SEQ ID NO | |
| | 349 | 350 |
| AVE-07- | GAAGTCCAGCTCTTGGAATCCGGA | GATATCCAAATGACACAGTCGCC |
| H03 | GGCGGCCTGGTGCAGCCCGGGGG | TTCTTCCCTTAGCGCATCAGTCG |
| | GTCATTGCGACTGAGTTGCGCCGC | GGGACCGCGTGACAATTACCTGT |
| | ATCTGGTTTCCCTTTTTCTAGCTTC | AGGACCTCCCAGAGCGTGAGTAC |
| | GTGATGTCCTGGGTTAGACAGGCC | TTACTTTAACTGGTACCAACAGA |
| | CCGGGGAAGGGTTTAGAGTGGGTG | AGCCCGGCAAAGCCCCCAAGCT |
| | AGCTCGCTGTCCGGAAGCGGGGAC | GCTGATCTATGCCGCGTCTAGTT |
| | ATTACTTATTATGCAGATTCTGTTA | TGCAGAGTGGTGTTCCTTCACGG |
| | AGGGCCGCTTTACCATCAGCCGGG | TTCTCAGGGTCCGGCTCTGGCAC |
| | ACAACTCCAAAAATACACTCTACC | AGATTTCACCTTAACTATTTCCA |
| | TTCAAATGAACAGTCTACGTGCCG | GCCTCCAGCCAGAGGACTTTGCT |
| | AGGACACCGCCGTGTACTACTGTG | ACGTATTACTGCCAGCAGAGCTA |
| | CTAGGGCTGCCCCAGGCGCGTATG | TAGAACTCCGCCAACATTCGGAG |
| | CATATGCTCTGGATTACTGGGGAC | GAGGTACCAAGGTAGAAATAAA |
| | AGGGCACGCTGGTCACAGTATCAT | A |
| | CA | |
| | SEQ ID NO | |
| | 359 | 360 |
| AVE-07- | GAGGTGCAGCTACTGGAGTCAGGA | GATATCCAGATGACACAGTCGCC |
| D04 | GGCGGGCTGGTTCAGCCTGGGGGG | CAGCTCCCTGAGCGCTTCAGTGG |
| | TCATTGCGACTGAGCTGTGCCGCA | GGGACCGCGTAACTATTACATGT |
| | TCTGGCCTCACCTTTAACGCCTAC | AGAGCCAGTCGAACCGTCACCTC |
| | GCCATGAGCTGGGTCCGTCAGGCG | CTATCTCAACTGGTACCAACAGA |
| | CCCGGCAAAGGCTTGGAATGGGTG | AGCCTGGCAAGGCGCCAAAATT |
| | TCCAGCTTAACTGGGTCTGGAGCC | ACTGATATACGGAGCATCTTATT |
| | TCCACCTTCTACGCTGACTCGGTC | TGCACTCTGGTGTGCCGTCTAGG |
| | AAGGGTAGATTCACAATCTCTCGG | TTCTCCGGCAGTGGGAGCGGAAC |
| | GACAATTCAAAGAATACACTTTAT | CGACTTCACTCTTACTATCTCCA |
| | CTGCAGATGAACAGTCTGAGGGCT | GCCTGCAGCCAGAAGATTTTGCC |
| | GAAGATACGGCTGTGTACTATTGC | ACATATTACTGCCAACAGTCATA |
| | GCACGCTCCAGGTCCTACTATGCC | CCGGACCCCTCCCACGTTTGGGG |
| | GGTGCATTTGATGTTTGGGGCCAA | GTGGCACAAAGGTTGAGATTAA |
| | GGAACCCTCGTAACTGTGAGCAGT | A |
| | SEQ ID NO | |
| | 369 | 370 |
| AVE-07- | GAGGTCCAGCTCCTGGAATCAGGT | GATATTCAGATGACGCAGTCCCC |
| E06 | GGGGGCCTGGTGCAGCCCGGCGG | CAGTAGCTTGAGTGCCAGCGTGG |
| | GAGCTTGCGGCTTTCTTGTGCTGC | GTGACCGGGTTACTATCACATGT |
| | AAGCGGATTTTCAGTGAGAGACTT | AGAGCGAACCAGAATATCGGCA |
| | CGCCATGAATTGGGTCCGTCAAGC | ACTACCTTAATTGGTACCAACAG |
| | CCCTGGAAAGGGGTTAGAATGGGT | AAGCCTGGGAAGGCTCCCAAACT |
| | GTCTAGCATTCACAGGACAGGCGG | CCTGATCTATGCAGCCTCATCCC |
| | GGGCACATACTACGCAGATAGTGT | TGCAGTCAGGTGTGCCGTCCAGG |
| | TAAAGGCAGGTTCACGATCTCCAG | TTTTCGGGGTCCGGGAGCGGAAC |
| | AGACAATTCCAAGAACACCCTCTA | CGATTTCACCCTGACAATTTCTTC |
| | CCTACAGATGAACAGTCTGCGAGC | TTTACAACCAGAAGACTTTGCTA |
| | GGAGGACACCGCCGTTTATTACTG | CCTACTATTGCCAGCAGTCATAT |
| | CGCCCGGAGCCGCTCCTATTATGC | AGCACCTCTACTTTCGGCGGCGG |
| | TGGAGCTTTTGATGTGTGGGGTCA | AACAAAGGTCGAGATAAAA |
| | GGGAACTCTGGTGACTGTATCTTC | |
| | G | |
| | SEQ ID NO | |
| | 379 | 380 |
| AVE-07-F06 | GAGGTCCAGCTGTTGGAAAGCGGG | GACATACAGATGACACAGTCTCC |
| | GGGGGCTTAGTACAGCCTGGAGGT | ATCTAGCCTTAGTGCCTCCGTTG |
| | TCCCTCCGTCTAAGCTGCGCCGCA | GAGATCGGGTGACCATCACATGC |
| | TCGGGATTCGACTTTCGCTCCTACC | CGCGCCTCACAGAAGATTGCAAG |
| | CAATGGCTTGGGTGCGGCAGGCCC | ATATGTCAACTGGTACCAGCAGA |
| | CCGGCAAGGGCCTGGAATGGGTTA | AACCTGGTAAGGCACCAAAGCTC |
| | GTGTTATTTCTGGTGGAGGCGGTT | TTAATCTATGCTGCGAGCGACTT |
| | CAACCAACTACGCGGACAGCGTCA | GCAAAGCGGCGTGCCCTCTAGGT |
| | AAGGGAGATTTACTATCTCTAGGG | TTTCCGGCAGCGGTTCAGGCACC |
| | ACAATTCCAAGAACACGCTGTACC | GATTTCACCCTGACGATCTCCTC |
| | TGCAGATGAATAGTCTCCGAGCAG | GCTGCAGCCTGAGGACTTTGCTA |
| | AGGATACAGCCGTGTATTATTGTG | CTTATTACTGTCAGCAAGCCTAC |
| | CCAGGTCAAGATCCTACTATGCTG | AGTATTCCCATTACATTCGGGGG |
| | GAGCTTTCGATGTGTGGGGCCAAG | AGGGACTAAAGTAGAAATCAAA |
| | GGACACTTGTGACCGTGAGCTCT | |
| | SEQ ID NO | |
| | 389 | 390 |
| AVE-07- | GAGGTGCAACTCCTTGAGTCAGGT | GACATCCAGATGACCCAGTCACC |
| A07 | GGCGGACTCGTGCAGCCTGGCGGA | CTCTTCCCTGTCCGCAAGTGTGG |
| | TCGCTGAGACTGTCATGTGCGGCT | GCGACCGGGTGACTATTACTTGT |
| | TCTGGGTTTCGTTTCTCCAACTACG | CGCGCCAGCCAGCCCATCAGTAG |
| | CAATGACATGGGTGAGGCAGGCCC | ATACTTAAACTGGTATCAGCAGA |
| | CCGGGAAAGGCCTGGAGTGGGTAT | AGCCGGGCAAAGCGCCAAAGCT |
| | CCGGAATCAGCGGCGGCGGGGGC | GTTGATATATGACGCTTCTCGAC |
| | AGGACCTTCTATGCAGATAGTGTC | TTCAAGTCGGCGTACCTAGCAGG |
| | AAGGGTAGATTTACCATTAGCCGG | TTCTCAGGGAGCGGTTCTGGAAC |
| | GACAATTCCAAGAATACATTATAC | GGATTTCACACTGACCATCTCGT |
| | CTACAGATGAACAGTCTGCGCGCC | CCCTCCAACCAGAGGATTTTGCC |
| | GAAGATACGGCTGTCTATTACTGC | ACATACTACTGCCAGCAGAGCCA |
| | GCCCGAGGTTACGGGGCCGAATAT | CTCCATTCCTTGGACATTTGGGG |
| | TTCGACCCGTGGGGGCAGGGAACT | GTGGAACCAAGGTTGAAATTAA |
| | TTGGTTACTGTGAGCTCT | A |
| | SEQ ID NO | |
| | 399 | 400 |
| AVE-07- | GAGGTGCAGCTGCTCGAATCGGGC | GACATCCAAATGACCCAGAGCCC |
| E07 | GGGGGCCTGGTCCAGCCCGGGGG | ATCATCCCTGAGCGCATCAGTGG |
| | GTCTTTGAGGCTCTCCTGTGCTGCC | GAGATAGAGTCACTATCACCTGT |
| | TCAGGGTTTAGGTTCAGTAATTAC | CGCGCTTCTCAACCAATATCCCG |
| | GCGATGACTTGGGTGCGACAGGCC | ACACCTCAACTGGTACCAGCAGA |
| | CCAGGAAAGGGCCTAGAGTGGGT | AACCCGGAAAGGCCCCTAAGCT |
| | GAGCGGCATTTCAGGTGGAGGAG | GTTGATTTATAGTGCCAGTTCCC |
| | GCAGAACATTTTATGCAGATAGCG | TGCAGTCAGGTGTTCCTAGCAGG |
| | TTAAAGGTAGATTCACGATCTCTC | TTTTCTGGCTCTGGGTCCGGCAC |
| | GGGACAACTCCAAGAATACCCTTT | AGACTTCACGCTTACTATTTCTA |
| | ACTTACAAATGAACAGTCTGCGCG | GCTTACAGCCCGAAGATTTTGCG |
| | CAGAGGATACCGCCGTATACTATT | ACATACTACTGCCAGCAGAGTTA |
| | GCGCTCGTGGGTATGGAGCCGAAT | TGACCGGACATGGACCTTCGGTG |
| | ACTTCGACCCTTGGGGCCAGGGTA | GGGGCACCAAGGGGGAGTCGAA |
| | CCCTGGTCACAGTGAGCTCC | A |
| | SEQ ID NO | |
| | 409 | 410 |
| AVE-07- | GAAGTCCAGCTCTTGGAATCCGGG | GACATTCAGATGACCCAGTCACC |
| D08 | GGGGGGTTAGTGCAGCCTGGAGGC | CAGTAGCTTGTCTGCCTCTGTGG |
| | TCTCTCAGACTGAGTTGCGCCGCC | GAGATCGCGTCACAATAACTTGC |
| | AGCGGATTTAGGTTCTCCAATTAT | AGGGCTTCCCAACGGATCGCCAC |
| | GCCATGACGTGGGTGAGACAGGCC | CTACCTGAACTGGTACCAGCAAA |
| | CCCGGGAAGGGGCTGGAGTGGGT | AACCTGGTAAGGCACCAAAGCT |
| | GTCTGGCATTTCCGGCGGAGGCGG | GTTAATTTACGCGGCCTCTCACC |
| | ACGGACTTTCTATGCAGACTCAGT | TTCATGGAGGCGTGCCATCCAGA |
| | AAAGGGTAGGTTCACAATCAGCCG | TTCTCCGGCAGCGGGTCAGGCAC |
| | AGACAACTCGAAAAATACCCTTTA | AGATTTTACCCTGACTATCTCCTC |
| | CCTGCAAATGAACAGTCTGCGCGC | GCTCCAGCCCGAAGACTTCGCTA |
| | GGAGGATACCGCTGTGTACTACTG | CGTATTATTGTCAGCAGAGTTAT |
| | TGCACGTGGTTACGGCGCTGAGTA | AGCATCCCTCTCACATTTGGTGG |
| | TTTTGATCCGTGGGGCCAGGGTAC | GGGGACCAAGGTTGAGATTAAA |
| | ACTAGTCACTGTTAGCTCA | |
| | SEQ ID NO | |
| | 419 | 420 |
| AVE-07- | GGAGGCGCGGCCTTAAAGTCCGGA | GATATCCAAATGACTCAGAGCCC |
| E08 | GGTGGCCTGGTCCAGCCCGGAGGG | TTCCAGCTTGAGTGCGTCCGTGG |
| | TCCCTCAGATTGAGCTGTGCTGCC | GGGATAGGGTAACTATCACATGT |
| | TCTGGCTTTACAGTGTCACGGGAT | AGAACAAGCCAGACCATATCTAT |
| | TATATGTCGTGGGTCCGCCAAGCT | TTACCTCAACTGGTATCAGCAGA |
| | CCGGGCAAGGGACTGGAATGGGT | AGCCCGGAAAGGCCCCAAAATT |
| | GAGTGTGATTAGCACCGGGGGTGG | ACTGATTTATGCAGCCTCAATCC |
| | GTCCACGTACTATGCCGACTCTGT | TGCACGGAGGTGTTCCGTCACGG |
| | TAAGGGGAGGTTCACAATCTCCAG | TTCTCCGGGTCGGGCTCTGGTAC |
| | GGATAATTCAAAAAACACCCTGTA | CGACTTTACCCTTACGATTAGTA |
| | CCTTCAGATGAACAGTCTCCGTGC | GCCTGCAGCCTGAGGACTTCGCT |
| | TGAGGACACCGCAGTTTATTATTG | ACCTACTACTGCCAACAGAATTA |
| | CGCAAAACCCCGACCATACAGCAT | TTCTGTCCCACCCACTTTTGGGG |
| | AGCATGGTTCGCCGACCCTTTTGA | GCGGCACAAAAGTGGAAATCAA |
| | TTACTGGGGCCAGGGTACTCTGGT | G |
| | GACTGTAAGCTCT | |
| | SEQ ID NO | |
| | 429 | 430 |
| AVE-07- | GAGCTGCAACTCTTAGAATCTGGC | GACATTCAGATGACCCAGTCACC |
| H08 | GGAGGCCTGGTTCAGCCCGGAGGC | ATCCAGTCTGTCAGCTTCGGTGG |
| | AGTCTGCGTCTATCCTGTGCCGCA | GCGATAGGGTAACAATCACTTGC |
| | TCTGGGTTCCCTTTCTCCAACAACG | AGAGCGTCCCAGCCCATCTCTCG |
| | CCATGTCATGGGTCCGCCAGGCCC | CTATCTTAACTGGTACCAGCAAA |
| | CAGGTAAAGGTCTTGAATGGGTTT | AGCCGGGGAAAGCACCAAAGCT |
| | CTGTGATCAGCGGCAGCTACGGGA | GTTAATTTACGACGCCTCTCGGC |
| | CTACATACTATGCCGATTCCGTGA | TGCAGGTCGGCGTTCCTAGCCGA |
| | AGGGCAGATTTACCATTAGCCGGG | TTCAGCGGTTCTGGGTCCGGAAC |
| | ATAATTCGAAGAACACGCTTTATC | AGATTTTACCCTCACAATTAGCT |
| | TGCAGATGAATAGTTTGAGGGCTG | CCTTGCAGCCCGAAGACTTTGCC |
| | AGGACACCGCAGTGTATTACTGCG | ACCTATTACTGTCAGCAAAGTCA |
| | CCCGAGTGGCTGACGGGGCAGCTG | CAGCATCCCTTGGACGTTCGGTG |
| | CTTACGCGATGGACTATTGGGGGC | GAGGCACTAAGGTGGAGATAAA |
| | AGGGAACTCTCGTCACAGTAAGCT | A |
| | CA | |
| | SEQ ID NO | |
| | 439 | 440 |
| AVE-07- | GAGGTGCAACTGCTTGAGAGTGGC | GACATCCAGATGACCCAAAGTCC |
| C10 | GGAGGCCTGGTGCAGCCTGGGGGC | CTCTTCTCTTAGCGCTTCGGTGG |
| | AGCCTCAGATTATCTTGCGCGGCC | GCGATCGGGTGACCATTACTTGT |
| | TCAGGTTTTCGTTTCTCTAACTATG | AGAGCGTCACAGCCAATAAGCA |
| | CTATGACATGGGTCCGACAGGCTC | GGTATCTCAATTGGTATCAGCAG |
| | CAGGGAAAGGGCTAGAATGGGTA | AAGCCCGGAAAAGCACCCAAGT |
| | TCGGGAATTTCCGGTGGCGGGGGC | TGCTGATCTACGCCGCCTCCAAC |
| | CGGACTTTCTACGCCGATAGCGTC | TTAAAGAAGGGGGTTCCTAGCCG |
| | AAGGGAAGGTTCACAATCAGCCGC | CTTTAGTGGGTCCGGCTTCGGAA |
| | GACAATTCAAAGAATACCCTCTAC | CCGATTTCACACTCACAATCTCA |
| | CTGCAGATGAACAGTTTGAGGGCC | TCCCTGCAGCCGGAGGACTTTGC |
| | GAGGACACTGCAGTGTATTACTGT | TACGTACTATTGCCAACAGTCTT |
| | GCCAGAGGCTACGGAGCAGAATA | ACAACCCTCCACTGACTTTCGGT |
| | TTTTGATCCCTGGGGGCAGGGTAC | GGCGGGACAAAAGTCGAAATTA |
| | CCTGGTGACGGTTTCCTCC | AA |
| | SEQ ID NO | |
| | 449 | 450 |
| AVE-07- | GAGGTGCAGCTGCTGGAGAGCGG | GACATTCAAATGACACAGAGTCC |
| D11 | AGGCGGACTAGTGCAGCCAGGTG | TTCTTCCTTGTCTGCATCCGTCGG |
| | GGTCCCTGCGACTTAGCTGCGCCG | TGATCGCGTTACCATCACTTGCC |
| | CTTCGGGGTTTACGTTTCCGCACTC | GGTCGAGCCAGAACATAATCACT |
| | TGCAATGTCTTGGGTGCGTCAGGC | TATCTCAATTGGTACCAGCAGAA |
| | ACCTGGAAAGGGCTTAGAATGGGT | ACCCGGAAAAGCCCCAAAGTTA |
| | CAGCAGCATCGCTGGCAGAGGTGG | GCGATTTACGGTGCCTCCAGGGT |
| | CTCTCCCAATTACGCCGACTCCGT | GCAGAGCGGCGTGCCCAGCAGA |
| | GAAGGGTAGGTTCACTATTTCCCG | TTCAGCGGGAGTGGGTCAGGGG |
| | CGATAACTCAAAAAATACACTCTA | CTGATTTCACACTGACCATCAGT |
| | TCTGCAGATGAACAGTTTGAGGGC | TCTCTTCAGCCTGAGGACTTTTC |
| | CGAAGACACCGCGGTATATTATTG | AACCTATTACTGTCAACAGTCCT |
| | TGCACGGGTTGCCGATGGCGGGGC | TCTCAACGCCACTGACCTTTGGC |
| | TGCCTACGCTTTCGACTACTGGGG | GGAGGCACAAAGGTAGAAATTA |
| | GCAAGGAACCCTCGTTACAGTCAG | AG |
| | TTCA | |
| | SEQ ID NO | |
| | 459 | 460 |
| AVE-07-F11 | GAGGTGCAGCTCCTGGAAAGCGG | GACATCCAGATGACACAGTCACC |
| | AGGGGGCTTGGTGCAGCCCGGAG | CTCCAGCCTGTCTGCCTCTGTGG |
| | GGTCCCTCCGATTATCCTGTGCTGC | GGGACAGGGTCACCATTACTTGT |
| | CTCAGGGTTTCGTTTTTCAAATTAC | CGGGCATCGCAACCAATTAGCAG |
| | GCAATGACATGGGTCCGGCAGGCC | ATACTTGAACTGGTATCAGCAGA |
| | CCTGGCAAGGGGCTGGAGTGGGTG | AGCCAGGAAAGGCCCCCAAGTT |
| | AGCGGGATTTCCGGAGGTGGTGGC | ACTCATTTACGACGCTAGTCGAC |
| | AGAACGTTCTACGCAGATTCGGTG | TTCAAGTGGGTGTTCCTTCTCGCT |
| | AAGGGTAGATTCACCATCAGTAGG | TCTCCGGGTCCGGAAGCGGCACT |
| | GACAATTCTAAAAACACTCTATAT | GATTTTACCCTGACCATCTCAAG |
| | CTGCAGATGAACAGTCTGAGGGCT | TCCCCAGCCTGAAGATTTTGCGA |
| | GAAGACACTGCCGTTTATTATTGC | CATATTACTGCCAGCAGTCCCAC |
| | GCGCGCGGATACGGCGCCGAGTAC | AGCATACCGTGGACGTTCGGTGG |
| | TTCGATCCGTGGGGCCAAGGCACC | GGGCACAAAAGGCGAGATCAAA |
| | CTTGTCACAGTAAGCTCT | |
| | SEQ ID NO | |
| | 469 | 470 |
| AVE-07- | GAAGTGCAGCTCCTGGAGTCAGGC | GACATACAGATGACCCAGTCCCC |
| G11 | GGGGGACTGGTCCAGCCCGGAGGT | GTCAAGTCTCAGCGCCTCGGTCG |
| | TCACTGCGTCTAAGCTGCGCTGCA | GGGACAGAGTTACAATCACGTGT |
| | TCTGGCTTCACATTCCCACACTCCG | CGGGCATCTCAATCCATTTCCAG |
| | CCATGTCTTGGGTTCGCCAGGCCC | CTATCTGAATTGGTACCAGCAGA |
| | CTGGGAAGGGATTGGAATGGGTGT | AACCTGGGAAGGCTCCAAAGCT |
| | CCACCGTTACAGGTTCCGGCAGCC | GCTTATCTACGATGCCAGCAACC |
| | CGACATATTATGCCGATAGCGTGA | TGCAGAGCGGAGTGCCCTCAAG |
| | AAGGGCGATTTACCATCAGCAGGG | GTTCTCCGGCAGTGGCTCTGGGA |
| | ACAACAGTAAGAATACGCTGTATC | CTGATTTTACCCTCACAATTTCTT |
| | TTCAGATGAACAGTCTCCGGGCTG | CTTTACAGCCCGAAGACTTTGCG |
| | AGGATACTGCGGTCTACTACTGTG | ACTTACTATTGCCAGCAATCATT |
| | CAAGAGTGGCTGGAGGGGCCTAC | CAGTAACTTGTATACCTTCGGTG |
| | GGTTATGCAATGGACTACTGGGGC | GAGGCACAAAGGTGGAGATCAA |
| | CAAGGCACTTTAGTGACCGTATCG | A |
| | TCT | |
| | SEQ ID NO | |
| | 479 | 480 |
| AVE-07-F12 | GAGGTGCAGCTTCTGGAATCAGGG | GATATCCAGATGACTCAGAGCCC |
| | GGTGGACTGGTTCAGCCTGGGGGT | ATCATCGCTCAGCGCATCTGTCG |
| | TCCCTCCGCCTGTCGTGTGCCGCTT | GGGACAGGGTTACCATAACCTGC |
| | CTGGCTTTCCATTCTCCGTATATGC | AGAAGTTCACAGAACATCATCAC |
| | AATGACATGGGTGAGGCAGGCCCC | CTACCTTAATTGGTACCAGCAAA |
| | GGGCAAGGGATTGGAGTGGGTGA | AGCCTGGCAAAGCCCCTAAACTG |
| | GTTCTTTTGGCGGCAGCGGGCACT | CTCATTTATGGAGCCTCCCGGCT |
| | CCCCCTATTACGCAGATTCCGTGA | GCAGAGCGGCGTGCCCAGTCGCT |
| | AGGGGCGTTTCACAATCTCAAGAG | TCTCCGGTTCTGGATCAGGTACC |
| | ATAATTCTAAAAACACGCTGTATC | GATTTCACGCTGACAATTAGCTC |
| | TACAGATGAACAGTCTCCGGGCTG | CTTACAACCCGAAGACTTTGCTA |
| | AAGACACCGCCGTTTACTACTGCG | CTTACTATTGTCAGCAGTCCTTCT |
| | CGCGAGTGGCCGCTGGCAGCTACG | CTACTCCATTGACATTTGGCGGG |
| | CCTATGCAATGGACTACTGGGGAC | GGGACAAAGGAGGTGGACAAG |
| | AAGGAACCTTAGTCACTGTCAGCA | |
| | GC | |
| | SEQ ID NO | |
| | 489 | 490 |
| AVE-07- | GAAGTCCAGCTTCTGGAGAGCGGA | GATATTCAGATGACACAGAGCCC |
| G12 | GGCGGACTGGTGCAGCCAGGCGG | ATCTTCACTGTCCGCTTCGGTAG |
| | GAGCTTGCGTCTGTCCTGTGCGGC | GGGATAGAGTGACAATAACATG |
| | AAGCGGGTTCCGCTTCTCTAACTA | CCGCGCAAGCCAGCCTATCTCAC |
| | TGCCATGACCTGGGTAAGGCAAGC | GGTACCTCAACTGGTACCAACAG |
| | ACCTGGCAAAGGTCTTGAGTGGGT | AAACCCGGGAAAGCCCCGAAGC |
| | GTCCGGAATTTCCGGCGGGGGCGG | TGTTAATCTATGACGCGAGTAGG |
| | ACGGACGTTCTACGCCGATTCAGT | CTGCAAGTGGGTGTCCCCAGCCG |
| | GAAGGGTCGATTTACCATCTCTAG | ATTCAGCGGTTCCGGATCTGGCA |
| | GGATAATAGTAAGAACACTCTCTA | CCGACTTCACCCTCACTATCTCTT |
| | CCTCCAGATGAATAGTTTGAGAGC | CCTTGCAGCCAGAAGACTTTGCC |
| | TGAAGACACAGCCGTGTACTATTG | ACGTATTACTGTCAGCAGTCCTT |
| | CGCTAGAGGGTACGGTGCCGAGTA | TAGTACCCCTCTTACCTTCGGCG |
| | TTTTGACCCCTGGGGGCAGGGCAC | GAGGCACTAAGGTTGAGATTAA |
| | TCTGGTTACAGTCTCATCG | G |
| | SEQ ID NO | |
| | 499 | 500 |

In some embodiments, provided herein is an anti-Activin E antibody, wherein the antibody comprises at least 90, 95, 96, 97, 98, 99, or 100% sequence identity to the VH and VL pair amino acid sequences selected from SEQ ID NOS: 17, 8; 17, 18; 27, 28; 37, 38; 47, 48; 57, 58; 67, 68; 77, 78; 87, 88; 97, 98; 107, 108; 117, 118; 127, 128; 137, 138; 147, 148; 157, 158; 167, 168; 177, 178; 187, 188; 197, 198; 207, 208; 217, 218; 227, 228; 237, 238; 247, 248; 257, 258; 267, 268; 277, 278; 287, 288; 297, 298; 307, 308; 317, 318; 327, 328; 337, 338; 347, 348; 357, 358; 367, 368; 377, 378; 387, 388; 397, 398; 407, 408; 417, 418; 427, 428; 437, 438; 447, 448; 457, 458; 467, 468; 477, 478; 487, 488; or 497, 498.

In some embodiments, provided herein is an anti-Activin E antibody, wherein the antibody encoded by the polynucleotide comprises at least 90, 95, 96, 97, 98, 99, or 100% sequence identity to the VH and VL pair nucleic acid sequences selected from SEQ ID NOS: 9, 10; 19, 20; 29, 30; 39, 40; 49, 50; 59, 60; 69, 70; 79, 80; 89, 90; 99, 100; 109, 110; 119, 120; 129, 130; 139, 140; 149, 150; 159, 160; 169, 170; 179, 180; 189, 190; 199, 200; 209, 210; 219, 220; 229, 230; 239, 240; 249, 250; 259, 260; 269, 270; 279, 280; 289, 290; 299, 300; 309, 310; 319, 320; 329, 330; 339, 340; 349, 350; 359, 360; 369, 370; 379, 380; 389, 390; 399, 400; 409, 410; 419, 420; 429, 430; 439, 440; 449, 450; 459, 460; 469, 470; 479, 480; 489, 490; or 499, 500.

TABLE 4
Anti-Activin E scFv amino acid and Nucleic Acid Sequences.

| CloneID | scFv AA | scFv NT |
|---|---|---|
| AVE-01-D07 | EVQLLESGGGLV | GAAGTGCAGCTCCTAGAATCCGGCGGTGGGCTGG |
| | QPGGSLRLSCAA | TTCAGCCTGGAGGGTCCCTTAGACTGTCTTGTGCC |
| | SGFAFNNYAMT | GCGAGTGGCTTCGCCTTTAACAATTATGCAATGA |
| | WVRQAPGKGLE | CCTGGGTGAGGCAGGCACCCGGCAAGGGACTGG |
| | WVSVISGSGTSK | AGTGGGTAAGCGTCATCAGCGGGAGCGGCACTTC |
| | YYADSVKGRFTI | TAAGTACTACGCCGATTCTGTTAAAGGTCGATTC |
| | SRDNSKNTLYLQ | ACCATATCACGGGACAACTCCAAGAACACTTTGT |
| | MNSLRAEDTAV | ATCTGCAAATGAATAGTCTCCGCGCCGAGGATAC |
| | YYCAKPRPGSIF | GGCTGTCTACTACTGCGCTAAACCGCGTCCAGGT |
| | WGAGPFDYWGQ | TCAATTTTTTGGGGAGCTGGACCCTTCGACTATTG |
| | GTLVTVSSGGGG | GGGGCAGGGCACATTAGTGACAGTGTCGAGCGGT |
| | SGGGGSGGGGSD | GGCGGTGGATCGGGCGGTGGTGGATCTGGAGGA |
| | IQMTQSPSSLSAS | GGTGGCTCGGACATCCAGATGACACAGTCTCCTT |
| | VGDRVTITCRAS | CTTCTCTGTCCGCTAGTGTGGGCGACAGAGTGAC |
| | QSISSYLHWYQQ | GATCACCTGTAGGGCGAGTCAAAGCATTTCATCC |
| | KPGKAPKLLIYA | TACTTGCACTGGTACCAGCAGAAGCCCGGAAAAG |
| | ASSLQSGVPSRFS | CACCTAAACTGTTAATTTACGCCGCCAGTTCCCTC |
| | GSGSGTDFTLTIS | CAAAGCGGTGTCCCATCTCGGTTTTCGGGGTCAG |
| | SLQPEDFATYYC | GGAGCGGCACTGATTTCACCCTTACCATCTCATCC |
| | QQSYGSPTFGGG | CTGCAGCCCGAAGATTTTGCTACATATTATTGCCA |
| | TKVEIK | GCAGAGCTATGGGAGCCCAACATTCGGTGGAGGC |
| | | ACTAAGGTTGAGATAAAG |
| SEQ ID NO | 501 | 502 |
| AVE-01-B08 | EVQLLESGGGLV | GAAGTGCAATTGCTTGAGAGTGGCGGCGGCCTGG |
| | QPGGSLRLSCAA | TTCAGCCTGGGGGCTCCCTGAGACTTAGCTGTGCT |
| | SGFTFGASAMTW | GCCTCTGGCTTCACCTTCGGAGCTTCAGCAATGAC |
| | VRQAPGKGLEW | CTGGGTGCGTCAGGCTCCAGGTAAGGGCCTGGAG |
| | VSGISGLGRTTD | TGGGTGTCAGGTATCAGCGGACTAGGGCGGACAA |
| | YADSVKGRFTIS | CCGATTACGCTGATAGCGTCAAAGGGCGCTTTAC |
| | RDNSKNTLYLQ | GATTTCGAGGGACAACTCTAAGAACACTCTGTAT |
| | MNSLRAEDTAV | CTCCAGATGAATAGTTTAAGGGCAGAAGACACAG |
| | YYCARVAPGAY | CCGTGTACTACTGCGCCCGAGTAGCCCCCGGAGC |
| | AYAMDYWGQG | GTATGCCTATGCAATGGACTACTGGGGGCAGGGA |
| | TLVTVSSGGGGS | ACTCTCGTCACAGTTTCCTCCGGTGGCGGTGGATC |
| | GGGGSGGGGSDI | GGGCGGTGGTGGATCTGGAGGAGGTGGCTCGGAC |
| | QMTQSPSSLSAS | ATACAGATGACACAGTCTCCTTCCTCACTTAGCGC |
| | VGDRVTITCRAS | GTCGGTTGGCGACCGCGTGACCATCACATGTAGA |
| | QNIGHYLNWYQ | GCATCACAGAACATTGGTCACTACCTGAATTGGT |
| | QKPGKAPKLLIY | ACCAGCAGAAACCCGGAAAGGCCCCAAAATTATT |
| | DASRLQVGVPSR | GATCTATGACGCCTCCAGGCTGCAAGTGGGCGTA |
| | FSGSGSGTDFTLT | CCATCTCGGTTCTCCGGTAGCGGGAGCGGCACCG |
| | ISSLQPEDFATYY | ATTTTACACTGACTATCAGTTCTCTCCAGCCCGAA |
| | CQQSYSTPPTFG | GATTTTGCTACGTATTATTGCCAACAGAGTTACAG |
| | GGTKVEIK | CACCCCGCCTACCTTCGGGGGAGGGACTAAGGTC |
| | | GAGATTAAG |
| SEQ ID NO | 503 | 504 |
| AVE-02-A03 | EVQLLESGGGLV | GAGGTCCAGCTGCTGGAGTCAGGCGGTGGCCTTG |
| | QPGGSLRLSCAA | TTCAGCCCGGCGGGTCTTTGCGGCTGTCCTGCGCC |
| | SGFNFRSYVMN | GCCAGCGGATTCAACTTCAGAAGCTACGTTATGA |
| | WVRQAPGKGLE | ACTGGGTGCGCCAGGCCCCTGGCAAGGGCTTAGA |
| | WVSAISDVGRRT | ATGGGTGTCCGCTATCTCCGATGTCGGACGTAGG |
| | YYADSVKGRFTI | ACTTACTATGCAGACTCTGTGAAAGGGAGATTTA |
| | SRDNSKNTLYLQ | CCATTAGTCGAGATAACTCAAAAAATACACTGTA |
| | MNSLRAEDTAV | TCTCCAGATGAATAGTTTGAGGGCCGAGGACACC |
| | YYCAKAEILGDY | GCAGTATATTACTGTGCTAAGGCTGAAATACTCG |
| | AYMDYWGQGTL | GTGACTATGCGTACATGGATTACTGGGGGCAAGG |
| | VTVSSGGGGSGG | AACACTAGTGACGGTGAGCTCGGGTGGCGGTGGA |
| | GGSGGGGSDIQM | TCGGGCGGTGGTGGATCTGGAGGAGGTGGCTCGG |
| | TQSPSSLSASVGD | ACATCCAGATGACCCAGAGTCCCTCATCTCTGAG |
| | RVTITCRASESIG | TGCATCAGTGGGGGACCGCGTGACTATTACCTGC |
| | NYLSWYQQKPG | AGAGCTTCTGAGTCCATAGGTAACTATCTGTCCTG |
| | KAPKLLIYAASR | GTACCAGCAAAAACCTGGCAAGGCGCCCAAGTTG |
| | LQRGVPSRFSGS | CTGATTTACGCCGCTTCGCGATTACAGAGGGGCG |
| | GSGTDFTLTISSL | TCCCATCCCGGTTTAGCGGGTCAGGGTCCGGTAC |
| | QPEDFATYYCQQ | TGATTTCACACTTACAATCAGCAGCCTCCAACCA |
| | SYSTPTFGGGTK | GAAGATTTTGCCACCTATTACTGTCAGCAGAGCT |
| | VEIK | ATTCTACACCTACCTTCGGAGGAGGCACGAAGGT |
| | | TGAGATCAAA |
| SEQ ID NO | 505 | 506 |
| AVE-02-B04 | EVQLLESGGGLV | GAAGTGCAGCTCCTAGAGTCGGGCGGCGGGCTGG |
| | QPGGSLRLSCAA | TGCAGCCCGGTGGCTCCCTCCGGCTGAGCTGCGC |
| | SGFAFSAYAMN | CGCGAGCGGTTTCGCTTTCAGTGCCTACGCCATG |
| | WVRQAPGKGLE | AACTGGGTTCGTCAAGCCCCTGGAAAAGGCCTGG |
| | WVSSITESGAAT | AGTGGGTATCAAGCATCACGGAGTCTGGGGCTGC |
| | YYADSVKGRFTI | TACATATTATGCAGATTCTGTGAAGGGAAGATTT |
| | SRDNSKNTLYLQ | ACTATTTCCCGCGACAATAGCAAGAATACCCTGT |
| | MNSLRAEDTAV | ATCTGCAGATGAACAGTTTGCGAGCAGAAGACAC |
| | YYCARGLLASYT | CGCCGTCTACTACTGTGCAAGGGGGCTTCTTGCTT |
| | GDVWGQGTLVT | CATACACAGGGGATGTGTGGGGCCAGGGAACCTT |
| | VSSGGGGSGGGG | AGTCACTGTTTCCTCTGGTGGCGGTGGATCGGGC |
| | SGGGGSDIQMTQ | GGTGGTGGATCTGGAGGAGGTGGCTCGGACATTC |
| | SPSSLSASVGDR | AGATGACACAAAGCCCCTCCAGCCTGAGCGCATC |
| | VTITCRASQSISS | GGTGGGGGACCGCGTCACCATAACTTGTCGGGCC |
| | YLNWYQQKPGK | AGTCAGTCTATCAGCTCATATCTGAACTGGTACC |
| | APKLLIYATSTLQ | AGCAGAAGCCTGGCAAAGCTCCAAAGCTGCTTAT |
| | SGVPSRFSGSGSG | CTACGCTACTTCCACTTTACAGTCCGGTGTTCCAT |
| | TDFTLTISSLQPE | CCAGGTTCTCAGGATCTGGCTCTGGTACAGATTTC |
| | DFATYYCQQRD | ACCTTGACCATTTCAAGTCTCCAGCCTGAAGATTT |
| | NAPWTFGGGTK | TGCCACGTACTATTGCCAACAGAGAGACAATGCG |
| | VEIK | CCCTGGACATTTGGGGGCGGAACCAAAGTGGAGA |
| | | TCAAG |
| SEQ ID NO | 507 | 508 |
| AVE-06-B07 | EVQLLESGGGLV | GAAGTGCAGCTCCTAGAGTCGGGTGGCGGGCTGG |
| | QPGGSLRLSCAA | TGCAGCCGGGCGGCTCCCTGCGCCTGTCTTGCGC |
| | SGFDFSSFAMTW | GGCTAGTGGATTTGACTTTTCTAGCTTCGCAATGA |
| | VRQAPGKGLEW | CCTGGGTCAGACAGGCCCCAGGGAAGGGATTGG |
| | VSHITGSGGTIYY | AGTGGGTGTCCCACATAACAGGTTCAGGAGGGAC |
| | ADSVKGRFTISR | AATCTATTACGCCGATAGCGTTAAGGGACGGTTC |
| | DNSKNTLYLQM | ACTATTAGCAGGGATAATTCAAAAAACACGCTTT |
| | NSLRAEDTAVYY | ACTTACAAATGAACAGTCTGCGAGCCGAGGACAC |
| | CASASSYYEPGD | CGCAGTATACTATTGTGCTTCCGCCAGCTCCTATT |
| | PWGQGTLVTVSS | ACGAACCCGGGGACCCTTGGGGCCAGGGCACTCT |
| | GGGGSGGGGSG | CGTCACCGTGTCATCTGGTGGCGGTGGATCGGGC |
| | GGGSDIQMTQSP | GGTGGTGGATCTGGAGGAGGTGGCTCGGACATCC |
| | SSLSASVGDRVTI | AGATGACTCAGAGTCCCAGCTCACTCTCCGCTTC |
| | TCRASQAIKNYL | AGTCGGTGATCGGGTTACTATAACATGTAGAGCC |
| | NWYQQKPGKAP | AGCCAAGCTATTAAGAATTACCTGAACTGGTATC |
| | KLLIYAGSTLQS | AGCAAAAACCCGGGAAGGCACCTAAACTGTTGAT |
| | GVPSRFSGSGSG | TTATGCGGGGTCCACACTCCAGAGCGGGGTGCCT |
| | TDFTLTISSLQPE | AGTAGGTTCTCTGGCTCCGGATCTGGCACTGACTT |
| | DFATYYCQQTYS | TACCTTAACCATCTCTTCGCTTCAGCCAGAAGATT |
| | TPLTFGGGTKVEI | TTGCCACATACTATTGCCAGCAGACATACAGCAC |
| | K | GCCACTGACCTTCGGTGGCGGAACCAAGGTGGAG |
| | | ATCAAA |
| SEQ ID NO | 509 | 510 |
| AVE-06-F07 | EVQLLESGGGLV | GAAGTTCAGCTGTTAGAAAGCGGGGGGGGCCTGG |
| | QPGGSLRLSCAA | TGCAGCCTGGCGGCTCACTGAGATTGTCCTGCGC |
| | SGFPFSSHGMSW | TGCATCCGGATTCCCGTTTTCCTCACACGGAATGA |
| | VRQAPGKGLEW | GCTGGGTTAGGCAGGCTCCCGGGAAGGGCCTAGA |
| | VSTITGSGRSTYY | GTGGGTCAGTACCATTACAGGAAGCGGTAGGAGC |
| | ADSVKGRFTISR | ACGTACTACGCCGATTCAGTAAAGGGCCGATTCA |
| | DNSKNTLYLQM | CAATCTCCCGTGACAACTCTAAAAATACTCTTTAT |
| | NSLRAEDTAVYY | CTCCAGATGAACAGTCTGCGGGCAGAGGATACAG |
| | CASDYRDAPGTF | CCGTGTATTATTGTGCGTCGGACTACCGCGATGCC |
| | DVWGQGTLVTV | CCAGGAACCTTTGACGTGTGGGGTCAAGGGACTC |
| | SSGGGGSGGGGS | TCGTCACCGTGTCTTCTGGTGGCGGTGGATCGGG |
| | GGGGSDIQMTQS | CGGTGGTGGATCTGGAGGAGGTGGCTCGGACATC |
| | PSSLSASVGDRV | CAAATGACCCAGTCTCCCTCCTCGCTGAGCGCAA |
| | TITCRASQSISSY | GCGTAGGGGACCGGGTCACCATAACGTGTAGAGC |
| | LNWYQQKPGKA | TTCTCAGTCAATCTCCTCATACTTGAACTGGTATC |
| | PKLLIYDASHLQS | AGCAGAAGCCCGGCAAAGCCCCTAAGCTCCTGAT |
| | GVPSRFSGSGSG | TTACGATGCCAGCCACCTACAAAGCGGAGTTCCA |
| | TDFTLTISSLQPE | TCCAGGTTTAGTGGCTCTGGGTCAGGAACAGATT |
| | DFATYYCQQSYS | TCACACTGACAATCAGTTCTCTTCAGCCTGAAGA |
| | TPVFGGGTKVEI | CTTCGCGACTTACTATTGCCAGCAGTCCTATAGTA |
| | K | CCCCAGTGTTTGGTGGGGGCACTAAAGTGGAGAT |
| | | TAAG |
| SEQ ID NO | 511 | 512 |
| AVE-06-A08 | EVQLLESGGGLV | GAGGTCCAGCTGTTGGAATCCGGGGGAGGCTTAG |
| | QPGGSLRLSCAA | TGCAACCGGGCGGCTCCCTGCGACTGAGCTGTGC |
| | SGFPFASHAMTW | AGCCTCGGGATTCCCCTTTGCTTCTCACGCAATGA |
| | VRQAPGKGLEW | CCTGGGTTCGTCAGGCCCCAGGGAAAGGCCTGGA |
| | VSTITGSGRSTYY | GTGGGTTTCTACTATCACCGGTTCAGGTCGGAGC |
| | ADSVKGRFTISR | ACATATTACGCTGACAGCGTGAAGGGGAGATTTA |
| | DNSKNTLYLQM | CCATTTCACGCGACAATAGCAAGAACACTCTCTA |
| | NSLRAEDTAVYY | TCTTCAGATGAACAGTCTCAGGGCCGAAGATACA |
| | CASDYRDAPGTF | GCCGTCTATTACTGCGCTAGTGACTACAGGGATG |
| | DVWGQGTLVTV | CGCCTGGAACGTTCGATGTGTGGGGCCAGGGGAC |
| | SSGGGGSGGGGS | ACTAGTGACCGTATCCTCTGGTGGCGGTGGATCG |
| | GGGGSDIQMTQS | GGCGGTGGTGGATCTGGAGGAGGTGGCTCGGATA |
| | PSSLSASVGDRV | TACAGATGACTCAATCTCCCAGCAGTCTGTCCGC |
| | TITCRASQSISSY | ATCCGTCGGAGATAGGGTAACAATTACCTGCAGA |
| | LNWYQQKPGKA | GCGTCCCAATCGATCAGTTCATATCTTAACTGGTA |
| | PKLLIYDASHLQS | CCAGCAGAAGCCTGGCAAAGCTCCAAAGCTGTTA |
| | GVPSRFSGSGSG | ATTTACGACGCCTCTCACCTCCAGAGTGGCGTTCC |
| | TDFTLTISSLQPE | TTCTCGGTTTTCTGGCAGCGGTTCCGGGACCGACT |
| | DFATYYCQQSYS | TCACGTTGACAATCTCAAGCCTGCAGCCAGAAGA |
| | TPVFGGGTKVEI | CTTTGCCACCTACTATTGTCAGCAGTCATATAGCA |
| | K | CTCCCGTGTTCGGGGGTGGAACAAAAGTGGAGAT |
| | | CAAG |
| SEQ ID NO | 513 | 514 |
| AVE-06-D08 | EVQLLESGGGLV | GAAGTGCAACTGCTGGAATCGGGCGGTGGACTGG |
| | QPGGSLRLSCAA | TCCAGCCTGGAGGCTCTTTGAGGCTTTCCTGTGCC |
| | SGFDFSKFAMSW | GCCTCAGGGTTTGATTTCAGTAAATTCGCTATGTC |
| | VRQAPGKGLEW | CTGGGTTCGCCAGGCTCCCGGGAAAGGACTTGAG |
| | VSHISGSGGTIYY | TGGGTGTCCCACATCAGCGGTAGCGGCGGAACGA |
| | ADSVKGRFTISR | TTTATTACGCAGACTCAGTAAAGGGCAGATTTAC |
| | DNSKNTLYLQM | TATATCTCGGGACAATTCAAAGAACACACTCTAC |
| | NSLRAEDTAVYY | CTGCAGATGAACAGTTTACGAGCAGAGGATACCG |
| | CASASSYYEPGD | CGGTCTATTACTGCGCCAGTGCCAGCTCCTACTAT |
| | PWGQGTLVTVSS | GAGCCAGGCGACCCGTGGGGGCAGGGGACACTC |
| | GGGGSGGGGSG | GTGACCGTGAGCTCTGGTGGCGGTGGATCGGGCG |
| | GGGSDIQMTQSP | GTGGTGGATCTGGAGGAGGTGGCTCGGATATACA |
| | SSLSASVGDRVTI | GATGACTCAATCCCCTTCTTCTCTGTCCGCGTCTG |
| | TCRASQAIKNYL | TTGGGGACAGGGTGACAATCACCTGTCGGGCTTC |
| | NWYQQKPGKAP | GCAGGCTATTAAAAATTATCTTAACTGGTATCAG |
| | KLLIYDASHPQS | CAGAAGCCCGGTAAGGCCCCAAAGTTGCTCATCT |
| | GVPSRFRGSGSG | ATGATGCCAGCCACCCACAGTCAGGAGTACCCAG |
| | TDFPFTISSLQPEE | TCGCTTCAGAGGCTCCGGAAGCGGCACCGACTTC |
| | FATYYCQQSYST | CCTTTCACCATCTCAAGCCTGCAGCCGGAGGAAT |
| | PVFGGGTKVEIK | TTGCAACATACTACTGCCAGCAAAGCTACAGTAC |
| | | GCCCGTCTTTGGCGGTGGGACTAAAGTGGAGATT |
| | | AAA |
| SEQ ID NO | 515 | 516 |
| AVE-06-G08 | EVQLLESGGGLV | GAGGTACAACTCCTAGAGAGTGGCGGAGGCCTTG |
| | QPGGSLRLSCAA | TGCAGCCCGGAGGATCGCTGCGACTGTCATGCGC |
| | SGFDFSKFAMSW | TGCATCCGGCTTTGATTTCTCTAAATTTGCCATGA |
| | VRQAPGKGLEW | GCTGGGTGAGACAGGCCCCGGGTAAGGGCCTGG |
| | VSHISGSGGTIYY | AATGGGTGTCTCACATATCAGGGAGCGGGGGAAC |
| | ADSVKGRFTISR | AATTTACTATGCCGACTCAGTTAAGGGGCGCTTC |
| | DNSKNTLYLQM | ACCATCAGCAGGGACAATTCCAAAAACACCCTGT |
| | NSLRAEDTAVYY | ATTTACAGATGAACAGTTTGCGGGCGGAGGATAC |
| | CASDSSYYEPGD | GGCAGTCTACTATTGTGCTTCCGACAGCAGTTACT |
| | PWGQGTLVTVSS | ACGAACCAGGTGATCCTTGGGGCCAGGGGACTCT |
| | GGGGSGGGGSG | CGTGACAGTCTCCTCTGGTGGCGGTGGATCGGGC |
| | GGGSDIQMTQSP | GGTGGTGGATCTGGAGGAGGTGGCTCGGACATCC |
| | SSLSASVGDRVTI | AGATGACACAGAGCCCGTCTTCACTCAGTGCAAG |
| | TCRAGQAIKNYL | TGTTGGGGACAGAGTGACCATTACATGCAGGGCC |
| | NWYQQKPGKAP | GGACAGGCTATCAAAAACTATCTGAATTGGTACC |
| | KLLIYAGSPLQSG | AACAAAAACCTGGCAAGGCGCCCAAATTGCTCAT |
| | VPSRFSGSGSGT | CTATGCCGGTTCACCACTGCAGAGCGGTGTGCCA |
| | DFTLTISSLQPED | TCGCGGTTTAGCGGATCTGGCTCCGGCACTGATTT |
| | FATYYCQQTYST | CACCCTTACCATTTCCTCTTTACAGCCCGAGGATT |
| | PLTFGGGTKVEI | TCGCTACCTACTATTGTCAGCAGACTTACTCCACG |
| | K | CCTCTGACATTTGGAGGGGGGACTAAGGTCGAAA |
| | | TAAAG |
| SEQ ID NO | 517 | 518 |
| AVE-06-H08 | EVQLLESGGGLV | GAGGTGCAGCTGCTGGAGAGCGGCGGAGGGCTG |
| | QPGGSLRLSCAA | GTGCAACCTGGGGGCTCACTGCGACTTTCCTGCG |
| | SGFDFSKFAMSW | CTGCCTCAGGTTTCGATTTCTCTAAGTTCGCTATG |
| | VRQAPGKGLEW | AGCTGGGTCAGACAGGCACCCGGGAAAGGCTTA |
| | VSSITRGSETTYY | GAGTGGGTGTCTTCTATTACGCGTGGCAGTGAAA |
| | ADSVKGRFTISR | CTACCTACTACGCCGACAGCGTTAAAGGACGGTT |
| | DNSKNTLYLQM | TACAATCTCCAGGGACAATTCCAAGAACACCCTC |
| | NSLRAEDTAVYY | TATCTGCAGATGAACAGTCTCCGCGCGGAAGACA |
| | CATLGLGYYYYF | CAGCCGTCTATTACTGTGCAACCCTAGGGTTGGG |
| | DVWGQGTLVTV | TTACTATTATTACTTTGATGTATGGGGCCAGGGAA |
| | SSGGGGSGGGGS | CATTGGTGACTGTTAGCTCGGGTGGCGGTGGATC |
| | GGGGSDIQMTQS | GGGCGGTGGTGGATCTGGAGGAGGTGGCTCGGAC |
| | PSSLSASVGDRV | ATCCAGATGACTCAGTCGCCCTCCTCACTGTCTGC |
| | TITCRASQPISSY | TTCAGTTGGAGATCGGGTGACCATTACCTGCCGC |
| | VTWYQQKPGKA | GCTAGTCAACCGATCAGCTCCTATGTGACGTGGT |
| | PKLLIYSASHLRS | ATCAGCAGAAGCCAGGCAAGGCCCCTAAATTGTT |
| | GVPSRFSGSGSG | AATCTATTCAGCGTCCCACCTCAGGAGCGGCGTC |
| | TDFTLTISSLQPE | CCCAGCAGATTTTCTGGAAGCGGGAGTGGTACCG |
| | DFATYYCQQSYN | ATTTCACTCTGACAATATCCTCTCTTCAGCCCGAG |
| | APPTFGGGTKVEI | GACTTTGCAACATACTACTGTCAACAGAGTTACA |
| | K | ACGCCCCACCTACATTCGGTGGCGGGACCAAAGT |
| | | AGAAATTAAG |
| SEQ ID NO | 519 | 520 |
| AVE-06-A09 | EVQLLESGGGLV | GAGGTGCAGCTCCTCGAAAGCGGCGGCGGATTAG |
| | QPGGSLRLSCAA | TCCAGCCTGGGGGATCGCTGAGGTTGTCATGCGC |
| | SGFPFASHAMTW | CGCCTCAGGCTTTCCCTTCGCATCCCACGCAATGA |
| | VRQAPGKGLEW | CTTGGGTGAGACAGGCGCCAGGGAAGGGGCTTG |
| | VSTITGSGRSTYY | AGTGGGTGTCTACGATTACCGGCAGCGGGCGGTC |
| | ADSVKGRFTISR | CACATATTATGCCGATAGTGTTAAGGGCCGATTC |
| | DNSKNTLYLQM | ACTATCTCACGTGATAACTCCAAAAACACCCTGT |
| | NSLRAEDTAVYY | ACCTGCAGATGAATAGTCTACGCGCTGAGGACAC |
| | CASASSYYEPGD | CGCTGTATACTATTGTGCTTCTGCCTCTAGCTACT |
| | PWGQGTLVTVSS | ACGAACCCGGTGACCCGTGGGGACAAGGTACACT |
| | GGGGSGGGGSG | GGTGACAGTCTCCAGCGGTGGCGGTGGATCGGGC |
| | GGGSDIQMTQSP | GGTGGTGGATCTGGAGGAGGTGGCTCGGACATTC |
| | SSLSASVGDRVTI | AAATGACACAGAGCCCATCTAGTTTGAGCGCATC |
| | TCRASQTISSFVS | CGTAGGCGATAGAGTGACGATCACTTGTCGGGCT |
| | WYQQKPGKAPK | TCACAGACCATATCTTCCTTCGTGTCTTGGTATCA |
| | LLIYAATTLQKG | GCAGAAACCGGGTAAGGCGCCCAAATTACTCATC |
| | VPSRFSGSGSGT | TACGCCGCCACAACCCTGCAAAAGGGCGTTCCTT |
| | DFTLTISSLQPED | CACGCTTTTCAGGGAGCGGGTCCGGAACAGACTT |
| | FATYYCQQSYHT | CACACTTACCATTAGTTCGCTGCAGCCCGAGGAT |
| | RSFGGGTKVEIK | TTCGCTACTTATTACTGCCAGCAGTCCTACCACAC |
| | | TAGGAGCTTTGGCGGAGGGACCAAAGTCGAAATC |
| | | AAG |
| SEQ ID NO | 521 | 522 |
| AVE-06-B09 | EVQLLESGGGLV | GAAGTGCAGCTCCTAGAGAGCGGGGGCGGGCTG |
| | QPGGSLRLSCAA | GTGCAGCCCGGAGGGTCACTGAGGCTGTCTTGCG |
| | SGLTFSNFAMTW | CTGCCTCTGGCTTAACATTCTCCAATTTCGCAATG |
| | VRQAPGKGLEW | ACATGGGTGCGCCAGGCCCCTGGGAAAGGTCTTG |
| | VSSIRGSGATTY | AGTGGGTGTCCTCGATTAGGGGATCTGGTGCCAC |
| | YADSVKGRFTIS | CACTTACTACGCTGACTCCGTCAAGGGCAGATTT |
| | RDNSKNTLYLQ | ACGATCAGCAGAGATAACAGTAAGAATACCCTGT |
| | MNSLRAEDTAV | ACTTGCAGATGAACAGTCTTCGGGCCGAAGATAC |
| | YYCARSREAYGF | AGCTGTCTATTATTGTGCACGAAGCCGTGAGGCG |
| | DYWGQGTLVTV | TATGGATTTGACTACTGGGGCCAAGGCACCCTCG |
| | SSGGGGSGGGGS | TAACTGTTAGCTCAGGTGGCGGTGGATCGGGCGG |
| | GGGGSDIQMTQS | TGGTGGATCTGGAGGAGGTGGCTCGGATATACAG |
| | PSSLSASVGDRV | ATGACACAATCACCCTCCAGCCTTTCGGCCTCAGT |
| | TITCRASQSISSY | GGGTGACAGAGTTACCATTACCTGCCGGGCTTCA |
| | LNWYQQKPGKA | CAGAGCATCAGCTCCTACTTGAACTGGTACCAGC |
| | PKLLIYAASSLQS | AAAAGCCCGGAAAAGCTCCAAAGCTCTTAATCTA |
| | GVPSRFSGSGSG | CGCCGCATCTTCTCTGCAGTCTGGGGTGCCTTCTA |
| | TDFTLTISSLQPE | GGTTTAGCGGAAGTGGCAGCGGCACCGACTTTAC |
| | DFATYYCQQSYS | ACTGACTATTAGTTCCCTGCAGCCTGAAGATTTCG |
| | TPYTFGGGTKVE | CGACATATTACTGTCAGCAGTCCTATAGTACTCCA |
| | IK | TATACCTTCGGTGGCGGGACGAAGGTCGAGATCA |
| | | AA |
| SEQ ID NO | 523 | 524 |
| AVE-06-D09 | EVHLLESGGGLV | GAAGTCCACCTGTTGGAGAGCGGCGGTGGCCTGG |
| | QPGGSLRLSCAA | TTCAACCTGGGGGTTCTCTACGTTTGTCGTGTGCA |
| | SGFTFSHYSMNW | GCCTCTGGGTTTACATTTAGCCATTATAGCATGAA |
| | VRQAPGKGLEW | TTGGGTGAGACAGGCACCCGGAAAGGGACTTGA |
| | VSGISGSGSATY | GTGGGTTAGCGGGATCTCCGGGTCAGGCTCCGCG |
| | YADSVKGRFTIS | ACTTACTATGCCGATTCAGTGAAAGGCCGCTTCA |
| | RDNSKNTLYLQ | CCATTTCTCGGGACAACAGTAAGAATACTCTGTA |
| | MNSLRAEDTAV | CCTCCAGATGAACAGTCTCCGAGCTGAAGACACC |
| | YYCASDRYLTFD | GCCGTGTACTACTGCGCTTCCGACAGGTATCTGA |
| | VWGQGTLVTVS | CCTTCGATGTCTGGGGACAGGGCACGTTAGTGAC |
| | SGGGGSGGGGSG | AGTATCCTCAGGTGGCGGTGGATCGGGCGGTGGT |
| | GGGSDIQMTQSP | GGATCTGGAGGAGGTGGCTCGGACATCCAGATGA |
| | SSLSASVGDRVTI | CGCAATCCCCTTCTAGCCTGTCAGCGTCAGTGGG |
| | TCRASQSISTHLN | CGATCGGGTCACAATCACTTGTAGAGCTTCTCAG |
| | WYQQKPGKAPK | AGTATTAGTACCCATCTGAACTGGTACCAGCAGA |
| | LLIYAASSLQSGV | AACCAGGCAAGGCACCCAAACTCCTCATATATGC |
| | PSRFSGSGSGTDF | CGCTTCCAGCTTACAGAGTGGTGTGCCATCGAGG |
| | TLTISSLQPEDFA | TTCAGCGGTTCCGGCAGCGGAACTGACTTTACCTT |
| | TYYCQQSHRTPL | GACCATCTCCTCTCTGCAGCCTGAAGATTTTGCCA |
| | IFGGGTKVEIK | CATATTACTGCCAACAGTCACACCGCACCCCCCTT |
| | | ATTTTCGGAGGGGGGACAAAGGTTGAGATTAAG |
| SEQ ID NO | 525 | 526 |
| AVE-06-F09 | EVQLLESGGGLV | GAGGTGCAGCTGCTGGAGTCTGGCGGCGGTCTTG |
| | QPGGSLRLSCAA | TCCAGCCCGGGGGGTCACTCCGACTAAGCTGCGC |
| | SGFDFSKFAMSW | TGCGAGTGGATTTGACTTCTCCAAGTTTGCCATGT |
| | VRQAPGKGLEW | CCTGGGTGAGACAGGCACCAGGAAAGGGACTTG |
| | VSHISGSGGTIYY | AGTGGGTGTCACACATATCTGGGAGCGGCGGAAC |
| | ADSVKGRFTISR | TATTTACTACGCTGATAGCGTTAAAGGCCGCTTCA |
| | DNSKNTLYLQM | CAATCTCACGGGACAACAGTAAAAACACCCTCTA |
| | NSLRAEDTAVYY | TTTGCAAATGAATAGTCTGAGGGCCGAAGATACA |
| | CASASSYYEPGD | GCAGTCTATTACTGTGCCTCCGCCTCTAGCTACTA |
| | PWGQGTLVTVSS | TGAACCTGGCGACCCGTGGGGGCAGGGTACGCTG |
| | GGGGSGGGGSG | GTGACCGTATCGTCCGGTGGCGGTGGATCGGGCG |
| | GGGSDIQMTQSP | GTGGTGGATCTGGAGGAGGTGGCTCGGATATTCA |
| | SSLSASVGDRVTI | GATGACACAGAGCCCCTCGTCACTGTCTGCGTCC |
| | TCRASQAIKNYL | GTCGGTGATCGGGTTACAATCACCTGTAGGGCTT |
| | NWYQQKPGKAP | CTCAAGCTATCAAGAACTACCTCAATTGGTACCA |
| | KLLIYAGSTLQS | GCAAAAACCTGGCAAGGCCCCAAAACTGTTAATT |
| | GVPSRFSGSGSG | TATGCAGGGTCTACATTGCAGAGTGGCGTGCCTT |
| | TDFTLTISSLQPE | CAAGATTCAGCGGCTCCGGGTCCGGAACTGACTT |
| | DFATYYCQQTYS | CACCCTTACAATCAGCAGTCTGCAGCCCGAGGAC |
| | TPLTFGGGTKGEI | TTTGCCACCTATTATTGCCAGCAGACGTACAGCA |
| | K | CCCCACTCACTTTTGGGGGTGGAACTAAAGGAGA |
| | | AATAAAG |
| SEQ ID NO | 527 | 528 |
| AVE-06-G09 | EVQFLESGGGLV | GAGGTGCAGTTCCTCGAATCCGGTGGGGGTTTAG |
| | QPGGSLRLSCAA | TGCAGCCCGGGGGCAGCCTTCGGCTGTCCTGCGC |
| | SGFSFDNYGINW | CGCCTCTGGGTTTAGCTTCGACAATTACGGTATCA |
| | VRQAPGKGLEW | ACTGGGTTCGACAAGCACCAGGGAAGGGACTGG |
| | VSSISSSGGSAYY | AATGGGTGTCATCAATTTCCTCGTCTGGAGGCTCC |
| | ADSVKGRFTISR | GCGTATTACGCAGATTCAGTGAAAGGCCGTTTTA |
| | DNSKNTLYLQM | CCATAAGTAGGGACAATAGCAAGAACACACTGTA |
| | NSLRAEDTAVYY | CCTCCAGATGAACAGTCTGCGCGCTGAGGACACC |
| | CARGVVPGGFD | GCCGTCTATTACTGTGCTAGAGGCGTAGTCCCTG |
| | YWGQGTLVTVS | GAGGATTCGATTATTGGGGCCAGGGCACGTTGGT |
| | SGGGGSGGGGSG | TACTGTGTCTAGCGGTGGCGGTGGATCGGGCGGT |
| | GGGSDIQMTQSP | GGTGGATCTGGAGGAGGTGGCTCGGACATCCAAA |
| | SSLSASVGDRVTI | TGACACAGTCCCCAAGTTCTCTTTCCGCCTCTGTG |
| | TCRASQSINNYL | GGTGACAGAGTGACAATCACCTGTCGCGCATCCC |
| | NWYQQKPGKAP | AGTCAATTAATAACTATCTCAACTGGTACCAGCA |
| | KLLIYAASSLQSG | GAAGCCTGGGAAGGCGCCCAAACTGCTGATTTAT |
| | VPSRFSGSGSGT | GCCGCTTCCAGCTTACAAAGTGGTGTCCCATCAA |
| | DFTLTISSLQPED | GGTTTTCAGGCAGCGGAAGCGGGACCGATTTCAC |
| | FATYYCQQSRTT | TCTGACGATAAGCTCGTTGCAGCCTGAAGATTTC |
| | PWTFGGGTKVEI | GCTACCTACTACTGCCAGCAGTCTCGGACTACCC |
| | K | CCTGGACTTTTGGCGGCGGAACAAAGGTTGAGAT |
| | | CAAA |
| SEQ ID NO | 529 | 530 |
| AVE-06-C10 | EVQLLESGGGLV | GAAGTGCAGCTTCTTGAGTCCGGCGGGGGACTAG |
| | QPGGSLRLSCAA | TTCAGCCCGGCGGCTCACTGCGACTGAGTTGTGC |
| | SGFDFSKFAMSW | GGCTTCAGGGTTTGATTTCAGCAAATTCGCAATGT |
| | VRQAPGKGLEW | CTTGGGTGAGACAGGCCCCAGGAAAAGGTTTGGA |
| | VSSITGTSGATYY | GTGGGTGAGCTCTATTACCGGGACCTCGGGCGCC |
| | ADSVKGRFTISR | ACCTACTATGCTGATTCCGTTAAGGGTAGATTTAC |
| | DNSKNTLYLQM | TATCTCTAGGGACAATAGCAAGAACACACTCTAC |
| | NSLRAEDTAVYY | CTCCAAATGAACAGTCTGCGGGCCGAAGACACTG |
| | CARDIRVRRSSW | CCGTATACTATTGCGCTAGGGATATACGCGTGCG |
| | AMDPWGQGTLV | GCGTAGCTCCTGGGCAATGGACCCTTGGGGACAG |
| | TVSSGGGGSGGG | GGCACGCTGGTCACAGTCTCCTCAGGTGGCGGTG |
| | GSGGGGSDIQMT | GATCGGGCGGTGGTGGATCTGGAGGAGGTGGCTC |
| | QSPSSLSASVGD | GGACATCCAGATGACGCAAAGTCCTAGCAGCCTG |
| | RVTITCRASQTIG | TCCGCCTCAGTGGGCGACCGGGTGACTATCACAT |
| | IYLNWYQQKPG | GTAGGGCTTCTCAGACCATTGGCATATATCTGAA |
| | KGPKPAIYSASPL | CTGGTACCAGCAGAAGCCCGGGAAAGGTCCCAA |
| | QRGVPSRFSGCG | ACCGGCGATCTACTCCGCATCCCCACTTCAGCGC |
| | SGTDFTLTISSLQ | GGCGTCCCTTCCAGATTCTCGGGGTGCGGCTCAG |
| | PEDFATYYCQQS | GTACAGATTTTACTTTGACAATTTCTAGTCTCCAG |
| | YSTPHITFGGGTK | CCCGAAGATTTCGCCACTTACTATTGCCAACAGA |
| | VEIK | GCTATTCTACCCCACACATTACCTTTGGAGGGGG |
| | | AACCAAGGTTGAGATCAAG |
| SEQ ID NO | 531 | 532 |
| AVE-06-G10 | EVQLLESGGGLV | GAGGTACAACTCCTGGAGTCAGGCGGAGGGCTCG |
| | QPGGSLRLSCAA | TCCAGCCAGGAGGTTCCCTAAGACTTTCTTGTGCC |
| | SGFDFSKFAMSW | GCCTCAGGCTTTGACTTCAGCAAGTTCGCAATGA |
| | VRQAPGKGLEW | GCTGGGTGAGGCAGGCGCCTGGGAAGGGGTTAG |
| | VSHISGSGGTIYY | AATGGGTGTCTCACATTAGTGGCTCTGGAGGAAC |
| | ADSVKGRFTISR | TATCTACTATGCCGATTCGGTGAAAGGGCGATTT |
| | DNSKNTLYLQM | ACAATATCCCGCGACAATTCAAAAAACACACTGT |
| | NSLRAEDTAVYY | ACCTGCAGATGAACAGTTTGCGGGCTGAAGATAC |
| | CASASSYYEPGD | CGCAGTCTACTATTGCGCTTCCGCCTCCAGCTATT |
| | PWGQGTLVTVSS | ACGAGCCCGGTGACCCGTGGGGCCAGGGCACCCT |
| | GGGGSGGGGSG | GGTTACGGTGAGTAGCGGTGGCGGTGGATCGGGC |
| | GGGSDIQMTQSP | GGTGGTGGATCTGGAGGAGGTGGCTCGGACATTC |
| | SSLSASVGDRVTI | AAATGACACAGAGTCCCAGTAGCCTCAGCGCTTC |
| | TCRASQAIKNYL | CGTTGGCGATAGAGTGACAATTACCTGTAGGGCC |
| | NWYQQKPGKAP | TCCCAGGCAATAAAGAACTATCTTAACTGGTACC |
| | KLLIYAGSTLQS | AGCAGAAACCAGGTAAGGCCCCTAAACTCTTAAT |
| | GVPSRFSGSGSG | CTATGCTGGCTCAACCCTGCAATCTGGGGTCCCCT |
| | TDFTLTISSLQPE | CGCGGTTCTCAGGGTCTGGAAGCGGCACAGACTT |
| | EFATYYCQQTYS | CACTTTGACCATCTCTAGCCTGCAGCCAGAGGAG |
| | TPLTFGGGTKGE | TTTGCGACTTACTATTGCCAACAGACATACTCCAC |
| | NQ | CCCTCTGACGTTTGGAGGGGGTACTAAGGGAGAA |
| | | AATCAG |
| SEQ ID NO | 533 | 534 |
| AVE-06-H10 | EVQLLESGGGLV | GAAGTGCAGCTGCTAGAATCCGGAGGGGGCTTAG |
| | QPGGSLRLSCAA | TCCAGCCTGGCGGCTCCCTTCGGCTCTCTTGTGCC |
| | SGFTFTNYALAW | GCTTCCGGATTTACGTTCACTAACTATGCACTGGC |
| | VRQAPGKGLEW | CTGGGTGCGACAGGCGCCAGGGAAGGGGCTGGA |
| | VSHISGSGGTIYY | GTGGGTAAGCCACATCTCTGGATCAGGAGGTACA |
| | ADSVKGRFTISR | ATTTACTATGCTGACAGCGTGAAGGGGCGCTTCA |
| | DNSKNTLYLQM | CCATAAGCAGAGATAACTCTAAAAATACCCTTTA |
| | NSLRAEDTAVYY | TCTGCAAATGAATAGTCTCAGGGCCGAGGATACT |
| | CASASSYYEPGD | GCAGTGTATTACTGCGCTTCGGCCTCAAGCTACTA |
| | PWGQGTLVTVSS | CGAGCCCGGCGACCCGTGGGGTCAGGGCACATTG |
| | GGGGSGGGGSG | GTCACCGTTAGTTCCGGTGGCGGTGGATCGGGCG |
| | GGGSDIQMTQSP | GTGGTGGATCTGGAGGAGGTGGCTCGGATATCCA |
| | SSLSASVGDRVTI | AATGACACAGTCTCCCAGTTCATTGTCCGCTTCCG |
| | TCRASQAIKNYL | TTGGAGATAGAGTCACCATTACCTGCAGGGCCAG |
| | NWYQQKPGKAP | TCAGGCGATAAAAAACTACCTTAATTGGTATCAG |
| | KLLIYAGSTLQS | CAGAAGCCTGGGAAAGCTCCCAAGCTGCTGATCT |
| | GVPSRFSGSGSG | ACGCCGGCTCAACTCTGCAGTCTGGGGTGCCTTCT |
| | TDFTLTISSLQPE | CGGTTTTCCGGTTCGGGCAGCGGGACGGACTTTA |
| | DFATYYCQQTYS | CACTCACAATTAGCAGCTTACAACCAGAGGACTT |
| | TPLTFGGGTKVEI | CGCAACTTATTACTGTCAGCAGACCTATAGCACA |
| | K | CCACTCACTTTCGGCGGAGGTACCAAGGTGGAAA |
| | | TCAAA |
| SEQ ID NO | 535 | 536 |
| AVE-06-B11 | EVQLLESGGGLV | GAGGTGCAGCTGCTGGAGTCTGGGGGCGGGCTAG |
| | QPGGSLRLSCAA | TCCAACCCGGAGGATCGCTGAGGCTTTCCTGCGC |
| | SGFDFSKFAMSW | CGCGTCAGGGTTTGACTTCAGCAAGTTTGCCATGT |
| | VRQAPGKGLEW | CTTGGGTGCGACAGGCACCTGGAAAAGGATTGGA |
| | VSHISGSGGTIYY | ATGGGTGTCACACATAAGCGGTTCTGGCGGCACT |
| | ADSVKGRFTISR | ATCTATTACGCCGACTCCGTCAAAGGCCGCTTCA |
| | DNSKNTLYLQM | CAATTAGCCGGGATAACAGCAAGAATACGCTCTA |
| | NSLRAEDTAVYY | CTTACAGATGAACAGTCTCAGAGCCGAGGATACC |
| | CASASSYYEPGD | GCTGTTTACTATTGTGCATCAGCTAGTAGTTACTA |
| | PWGQGTLVTVSS | TGAACCAGGTGACCCGTGGGGGCAGGGCACCCTG |
| | GGGGSGGGGSG | GTGACAGTATCCTCCGGTGGCGGTGGATCGGGCG |
| | GGGSDIQMTQSP | GTGGTGGATCTGGAGGAGGTGGCTCGGATATCCA |
| | SSLSASVGDRVTI | GATGACCCAGTCGCCAAGCTCTCTCTCCGCTTCTG |
| | TCRASQAIKNYL | TTGGAGACAGGGTCACAATTACTTGCCGGGCATC |
| | NWYQQKPGKAP | CCAAGCGATCAAGAACTATTTAAATTGGTACCAG |
| | KLLIYAGSTLQS | CAGAAGCCTGGGAAAGCTCCTAAACTTCTGATAT |
| | GVPSRFSGSGSG | ATGCCGGCTCTACCCTGCAGTCAGGGGTGCCCAG |
| | TDFTLTISSLQPE | TAGATTCTCAGGGAGCGGCAGTGGTACCGATTTC |
| | DFATYYCQQTYS | ACACTGACCATTAGCAGCTTGCAACCAGAGGACT |
| | TPLTFGGGTKVEI | TTGCCACATACTATTGTCAGCAGACGTACTCCACT |
| | K | CCCCTCACATTTGGTGGCGGAACTAAAGTGGAAA |
| | | TCAAG |
| SEQ ID NO | 537 | 538 |
| AVE-06-E11 | EVQLLESGGGLV | GAGGTCCAGCTGCTAGAGTCTGGAGGCGGGCTGG |
| | QPGGSLRLSCAA | TGCAGCCGGGAGGTAGTTTACGCCTGTCCTGTGC |
| | SGIRFSSYAMSW | AGCTTCGGGCATTAGGTTTTCAAGCTACGCAATG |
| | VRQAPGKGLEW | TCTTGGGTGCGACAAGCGCCTGGTAAGGGACTGG |
| | VSHISGSGGTIYY | AATGGGTGAGCCACATCAGCGGCAGCGGCGGAA |
| | ADSVKGRFTISR | CTATATATTATGCCGACTCCGTAAAGGGGCGGTT |
| | DNSKNTLYLQM | CACGATCTCACGTGATAACTCAAAAAACACATTG |
| | NSLRAEDTAVYY | TATCTCCAGATGAATAGTCTTAGAGCCGAGGATA |
| | CASASSYYEPGD | CCGCCGTTTACTACTGCGCTTCCGCCTCCAGTTAC |
| | PWGQGTLVTVSS | TATGAACCAGGGGACCCCTGGGGCCAGGGGACA |
| | GGGGSGGGGSG | CTCGTCACCGTGTCTTCTGGTGGCGGTGGATCGG |
| | GGGSDIQMTQSP | GCGGTGGTGGATCTGGAGGAGGTGGCTCGGATAT |
| | SSLSASVGDRVTI | TCAGATGACTCAGAGCCCATCTAGCCTCAGCGCT |
| | TCRASQAIKNYL | TCTGTGGGGGACAGGGTTACGATTACATGTAGAG |
| | NWYQQKPGKAP | CAAGTCAGGCCATCAAGAACTACCTTAATTGGTA |
| | KLLIYAGSTLQS | CCAGCAAAAGCCTGGCAAAGCTCCTAAACTGCTC |
| | GVPSRFSGSGSG | ATCTACGCGGGGTCCACATTGCAATCTGGAGTCC |
| | TDFTLTISSLQPE | CCTCGCGGTTTAGTGGAAGCGGCTCCGGTACAGA |
| | DFATYYCQQTYS | CTTCACTCTGACCATCTCATCATTACAGCCAGAGG |
| | TPLTFGGGTKVEI | ATTTCGCCACATATTATTGCCAGCAGACCTATTCC |
| | K | ACTCCCCTGACCTTTGGCGGCGGGACCAAAGTGG |
| | | AAATAAAG |
| SEQ ID NO | 539 | 540 |
| AVE-06-H11 | EVQLLESGGGLV | GAAGTGCAGCTCTTAGAGAGCGGGGGCGGACTG |
| | QPGGSLRLSCAV | GTCCAGCCAGGCGGTAGCCTGCGCCTAAGTTGCG |
| | SGFDFSKFVMSG | CCGTTTCTGGGTTCGATTTTTCTAAGTTCGTGATG |
| | VRQAPGKGLEW | AGTGGCGTGCGGCAGGCTCCTGGCAAAGGTCTGG |
| | VSHISGSGGTIYY | AGTGGGTTTCACACATATCCGGGTCTGGGGGAAC |
| | ADSVKGRFTISR | GATTTACTACGCTGATTCAGTGAAAGGAAGATTT |
| | DNSKNTLYLQM | ACCATCTCCAGGGACAACAGCAAGAATACTCTCT |
| | NSLRAEDTAVYY | ATCTGCAAATGAACAGTCTTCGAGCGGAAGACAC |
| | CASASSYYEPGD | AGCCGTCTACTATTGTGCATCCGCCTCGTCATATT |
| | PWGQGTLVTVSS | ACGAGCCGGGTGACCCCTGGGGACAGGGCACATT |
| | GGGGSGGGGSG | GGTGACCGTAAGCTCCGGTGGCGGTGGATCGGGC |
| | GGGSDIQMTQSP | GGTGGTGGATCTGGAGGAGGTGGCTCGGACATCC |
| | SSLSASVGDRVTI | AGATGACCCAGTCTCCTTCCTCCCTTAGTGCTTCG |
| | TCRASQAIKNYL | GTGGGTGATAGGGTCACCATTACATGCAGAGCCA |
| | NWYQQKPGKAP | GTCAGGCAATAAAGAACTACTTGAATTGGTACCA |
| | KLLIYAGSTLQS | GCAAAAACCCGGCAAGGCCCCCAAACTGCTGATC |
| | GVPSRFSGSGSG | TATGCTGGTTCCACGTTACAGTCTGGAGTGCCAA |
| | TDFTLTISSLQPE | GCCGGTTTAGCGGCTCAGGGTCTGGAACTGATTT |
| | DFATYYCQQTYS | CACACTGACAATTAGCTCACTCCAGCCAGAAGAC |
| | TPLTFGGGTKVEI | TTCGCGACTTATTATTGTCAGCAAACCTACAGCAC |
| | K | CCCTCTCACTTTTGGGGGCGGGACAAAGGTTGAG |
| | | ATCAAA |
| SEQ ID NO | 541 | 542 |
| AVE-06-A12 | EVQLLESGGGLV | GAAGTGCAGCTCCTAGAGTCCGGAGGTGGCCTCG |
| | QPGGSLRLSCAA | TGCAGCCGGGAGGCTCCCTGCGCCTGTCCTGCGC |
| | SGFDFSKFAMSW | GGCCTCTGGCTTTGACTTTAGTAAGTTCGCTATGA |
| | VRQAPGKGLEW | GCTGGGTGAGACAGGCTCCAGGTAAGGGCCTGGA |
| | VSHISGSGGTIYY | GTGGGTATCTCACATCTCCGGGTCAGGAGGGACC |
| | ADSVKGRFTISR | ATATATTACGCCGACTCTGTCAAAGGCCGGTTCA |
| | DNSKNTLYLQM | CTATTTCAAGGGATAATTCAAAAAACACATTATA |
| | NSLRAEDTAVYY | CCTTCAAATGAACAGTCTGCGAGCCGAAGACACA |
| | CASASSYYEPGD | GCAGTGTACTATTGTGCCAGCGCAAGCAGCTACT |
| | PWGQGTLVTVSS | ATGAGCCTGGAGATCCCTGGGGGCAGGGGACCTT |
| | GGGGSGGGGSG | GGTCACGGTTAGTTCGGGTGGCGGTGGATCGGGC |
| | GGGSDIQMTQSP | GGTGGTGGATCTGGAGGAGGTGGCTCGGATATAC |
| | SSLSASVGDRVTI | AGATGACCCAGTCACCCTCCAGCTTGTCAGCTAG |
| | TCRASQAIKNYL | TGTTGGGGACCGGGTCACCATCACATGCAGAGCC |
| | NWYQQKPGKAP | TCTCAGGCGATCAAGAATTACCTTAACTGGTATC |
| | KLLIYAGSTLQS | AGCAGAAACCAGGAAAAGCTCCGAAGTTACTGAT |
| | GVPSRFSGSGSG | TTACGCAGGTAGCACACTGCAGTCGGGCGTGCCC |
| | TDFTLTISSLQPE | TCTAGGTTCAGTGGGAGCGGCTCCGGAACAGACT |
| | DFPTYYFQQNYS | TTACTCTGACCATTTCTTCCCTCCAACCAGAAGAT |
| | TPFAFGGGTKVEI | TTCCCTACTTATTATTTTCAGCAAAACTACAGCAC |
| | K | GCCTTTCGCCTTTGGCGGTGGGACCAAAGTGGAG |
| | | ATCAAG |
| SEQ ID NO | 543 | 544 |
| AVE-06-B12 | EVQLLESGGGLV | GAGGTGCAGCTTCTGGAGAGCGGAGGGGGACTG |
| | QPGGSLRLSCAA | GTCCAGCCTGGCGGCAGTCTAAGATTGTCCTGCG |
| | SGFDFSKFAMSW | CCGCGTCAGGGTTCGATTTTTCCAAATTTGCCATG |
| | VRPAPCKGLEW | TCATGGGTGCGCCCAGCTCCCTGTAAGGGATTAG |
| | VSHISGSGGTIYY | AATGGGTGAGTCACATCTCAGGTTCTGGGGGCAC |
| | ADSVKGRFTISR | AATTTATTACGCAGACTCCGTCAAGGGGCGATTC |
| | DNSKNTLYLQM | ACGATATCCCGGGACAACTCTAAAAACACTCTGT |
| | NSLRAEDTAVYY | ACCTCCAAATGAATAGTCTCAGGGCCGAGGACAC |
| | CASASSYYEPGD | CGCTGTGTACTATTGCGCCTCGGCAAGCAGCTATT |
| | PWGQGTLVTVSS | ACGAACCCGGCGATCCGTGGGGCCAGGGTACCCT |
| | GGGGSGGGGSG | GGTAACAGTTAGCTCTGGTGGCGGTGGATCGGGC |
| | GGGSDIQMTQSP | GGTGGTGGATCTGGAGGAGGTGGCTCGGACATCC |
| | SSLSASVGDRVTI | AGATGACACAGAGTCCATCTAGCTTATCCGCGAG |
| | TCRASQAIKNYL | TGTTGGGGACAGGGTCACCATCACATGTAGAGCC |
| | NWYQQKPGKAP | TCTCAGGCAATTAAAAACTACCTGAATTGGTATC |
| | KLLIYAGSTLQS | AACAAAAGCCCGGAAAGGCCCCAAAGCTGCTCAT |
| | GVPSRFSGSGSG | CTACGCCGGCTCCACCCTGCAGAGCGGAGTGCCT |
| | TDFTLTISSLQPE | AGCCGGTTTAGCGGCTCTGGCTCGGGGACTGATT |
| | DFATYYCQQTYS | TTACATTGACTATTTCATCACTTCAGCCCGAAGAT |
| | TPLTFGGGTKVEI | TTCGCTACATATTATTGCCAGCAGACCTACTCCAC |
| | K | TCCTCTCACGTTCGGTGGTGGGACCAAAGTGGAG |
| | | ATAAAA |
| SEQ ID NO | 545 | 546 |
| AVE-06-C12 | EVQLLESGGGLV | GAGGTGCAGCTGCTGGAAAGCGGAGGAGGGTTA |
| | QPGGSLRLSCAA | GTGCAACCAGGAGGATCTCTCCGGCTGAGCTGCG |
| | SGFDFSKFAMSW | CTGCCTCAGGCTTTGATTTCAGCAAGTTCGCCATG |
| | VRQAPGKGLEW | TCCTGGGTCCGACAGGCACCGGGGAAAGGCCTTG |
| | VSHISGSGGTIYY | AGTGGGTGAGCCACATATCCGGCTCAGGTGGGAC |
| | ADSVKGRFTISR | CATTTACTACGCTGATTCTGTAAAAGGGAGATTT |
| | DNSKNTLYLQM | ACGATCTCACGCGACAACAGTAAGAATACACTCT |
| | NSLRAEDTAVYY | ACCTGCAGATGAACAGTTTGAGGGCCGAAGACAC |
| | CASASSYYEPGD | AGCCGTGTATTATTGTGCGTCTGCATCCTCGTACT |
| | PWGQGTLVTVSS | ATGAGCCTGGCGACCCCTGGGGCCAGGGTACCCT |
| | GGGGSGGGGSG | AGTCACTGTTTCCAGTGGTGGCGGTGGATCGGGC |
| | GGGSDIQMTQSP | GGTGGTGGATCTGGAGGAGGTGGCTCGGATATAC |
| | SSLSASVGDRVTI | AAATGACACAGAGTCCCTCATCCCTGTCAGCTTCT |
| | TCRASQAIKNYL | GTGGGTGATAGGGTCACAATCACATGCAGAGCCA |
| | NWYQQKPGKAP | GCCAGGCGATTAAAAATTACCTCAACTGGTATCA |
| | KLLIYAGSTLQS | GCAGAAGCCTGGCAAGGCTCCAAAATTACTTATC |
| | GVPSRFSGSGSG | TACGCAGGTAGCACCCTGCAGTCCGGAGTGCCTT |
| | TDFTLTISSLQPE | CTCGGTTCAGCGGGTCCGGCTCTGGGACCGACTT |
| | DFATYYCQQNYS | TACCCTCACCATCAGTAGCCTGCAGCCAGAAGAC |
| | TPLTFGGGTKVEI | TTCGCCACTTATTACTGTCAGCAAAACTATTCGAC |
| | K | TCCCTTGACGTTTGGAGGCGGGACTAAAGTTGAG |
| | | ATTAAG |
| SEQ ID NO | 547 | 548 |
| AVE-06-E12 | EVQFLKSGGGLV | GAGGTCCAATTTCTGAAAAGCGGTGGCGGGCTGG |
| | NPAGILELSCAAS | TTAATCCCGCGGGCATCCTTGAGCTCTCTTGTGCA |
| | GFPFSSHGMSWV | GCCTCCGGGTTCCCGTTTAGCAGTCACGGTATGTC |
| | RQAPGKGLEWV | ATGGGTGAGGCAGGCTCCAGGAAAGGGACTAGA |
| | SVISGSGATTYY | ATGGGTATCTGTGATTTCAGGCTCCGGAGCCACC |
| | ADSVKGRFTISR | ACCTATTACGCAGATTCCGTTAAGGGCCGCTTCA |
| | DNSKNTLYLQM | CTATATCGAGAGACAACAGCAAAAATACTTTATA |
| | NSLRAEDTAVYY | TTTGCAGATGAACAGTCTGCGAGCCGAAGACACA |
| | CARGQPYYGFD | GCTGTCTATTACTGCGCCCGGGGGCAGCCTTACT |
| | YWGQGTLVTVS | ATGGCTTCGATTACTGGGGACAGGGGACGCTCGT |
| | SGGGGSGGGGSG | GACAGTGAGCTCTGGTGGCGGTGGATCGGGCGGT |
| | GGGSDIQMTQSP | GGTGGATCTGGAGGAGGTGGCTCGGACATACAGA |
| | SSLSASVGDRVTI | TGACACAGTCTCCCAGCTCTTTATCCGCTTCCGTG |
| | TCRASHSIASYLH | GGCGATAGAGTGACTATTACATGCCGCGCAAGTC |
| | WYQQKPGKAPK | ACTCCATCGCCAGCTATCTCCACTGGTACCAACA |
| | LLIYRASTLHTG | AAAGCCAGGTAAGGCGCCCAAACTGCTGATCTAC |
| | VPSRFSGSGSGT | AGGGCCAGCACGCTGCATACAGGAGTCCCTTCTC |
| | DFTLTISSLQPED | GGTTTTCCGGAAGTGGCTCAGGGACAGATTTCAC |
| | FATYYCQQTYSI | TCTTACCATTTCATCGTTGCAGCCTGAGGACTTTG |
| | PWTFGGGTKVEI | CTACCTATTATTGTCAGCAGACCTACAGCATCCCG |
| | K | TGGACTTTCGGTGGCGGGACCAAAGTTGAAATTA |
| | | AG |
| SEQ ID NO | 549 | 550 |
| AVE-06-G12 | EVQFLESGGGLV | GAGGTGCAATTTCTGGAGTCAGGGGGGGGTTTGG |
| | QPGGSLRLSCAA | TTCAGCCGGGAGGCAGCCTTAGGTTATCCTGTGC |
| | SGFDFSKFAMSW | CGCCTCAGGGTTCGATTTCAGCAAATTTGCAATG |
| | VRQAPGKGLEW | AGTTGGGTCCGGCAGGCACCCGGCAAGGGACTGG |
| | VSHISGSGGTIYY | AATGGGTAAGCCACATTTCTGGCTCTGGAGGCAC |
| | ADSVKGRFTISR | CATCTACTACGCCGACTCCGTGAAGGGACGATTC |
| | DNSKNTLYLQM | ACGATATCGCGCGACAATTCAAAAAACACCCTGT |
| | NSLRAEDTAVYY | ATCTCCAGATGAACAGTCTGAGAGCTGAGGATAC |
| | CASASSYYEPGD | TGCCGTCTATTACTGCGCTTCCGCGTCTAGTTACT |
| | PWGQGTLVTVSS | ATGAACCAGGTGACCCTTGGGGCCAGGGGACACT |
| | GGGGSGGGGSG | CGTGACAGTGAGCTCCGGTGGCGGTGGATCGGGC |
| | GGGSDIQMTQSP | GGTGGTGGATCTGGAGGAGGTGGCTCGGACATTC |
| | SSLSASVGDRVTI | AGATGACACAGTCACCAAGTAGTCTGTCCGCGTC |
| | TCRASQAIKNYL | TGTTGGGGATCGGGTGACGATTACATGCAGGGCT |
| | NWYQQKPGKAP | TCGCAGGCCATCAAAAACTACTTAAACTGGTATC |
| | KLLIYGASNLQS | AACAGAAGCCCGGCAAGGCCCCTAAGCTGCTTAT |
| | GVPSRFSGSGSG | ATACGGGGCTTCCAATCTGCAGTCAGGAGTCCCT |
| | TDFTLTISSLQPE | TCCAGATTTTCTGGATCTGGTAGCGGTACTGATTT |
| | DFATYYCQQSES | TACACTCACCATCTCCAGCCTCCAACCCGAGGAC |
| | TPLTFGGGTKVEI | TTCGCAACCTATTACTGTCAGCAGAGCGAAAGCA |
| | K | CTCCATTGACCTTCGGGGGCGGCACCAAAGTGGA |
| | | GATCAAA |
| SEQ ID NO | 551 | 552 |
| AVE-07-A01 | EVQLLESGGGLV | GAAGTGCAGCTTTTAGAATCAGGCGGTGGACTCG |
| | QPGGSLRLSCAA | TACAGCCTGGGGGCTCCCTGAGACTGAGCTGCGC |
| | SGFALTDFAMSW | TGCCTCAGGCTTCGCCCTCACAGACTTTGCCATGT |
| | VRQAPGKGLEW | CCTGGGTGCGGCAAGCTCCCGGGAAGGGACTAGA |
| | VSQISVSGGVGY | GTGGGTGTCCCAGATCTCGGTGTCTGGTGGCGTT |
| | YADSVKGRFTIS | GGGTACTACGCCGACAGCGTCAAAGGGAGGTTCA |
| | RDNSKNTLYLQ | CGATTTCCAGGGATAACAGTAAGAATACCCTGTA |
| | MNSLRAEDTAV | CCTGCAAATGAACAGTTTGCGCGCGGAGGATACA |
| | YYCARSRSYYAG | GCAGTTTATTATTGTGCACGAAGCCGTTCTTATTA |
| | AFDVWGQGTLV | CGCTGGAGCATTTGACGTGTGGGGCCAGGGAACC |
| | TVSSGGGGSGGG | CTTGTCACTGTCAGCTCTGGTGGCGGTGGATCGG |
| | GSGGGGSDIQMT | GCGGTGGTGGATCTGGAGGAGGTGGCTCGGATAT |
| | QSPSSLSASVGD | CCAAATGACCCAGTCTCCTTCCTCGCTCAGCGCA |
| | RVTITCRASAPV | AGTGTGGGCGACAGGGTGACCATTACATGTCGCG |
| | GRYLNWYQQKP | CCAGTGCTCCAGTCGGAAGATATCTGAACTGGTA |
| | GKAPKLLIYAAS | TCAGCAGAAACCTGGGAAGGCCCCCAAGCTGCTT |
| | SLQSGVPSRFSGS | ATCTATGCAGCCTCATCCTTACAGTCTGGGGTGCC |
| | GSGTDFTLTISSL | CTCCCGGTTCTCAGGTAGCGGTAGCGGGACTGAC |
| | QPEDFATYYCQQ | TTTACACTGACCATTTCATCTTTGCAGCCAGAGGA |
| | SYSAVTFGGGTK | TTTTGCTACGTACTACTGCCAGCAAAGCTACTCCG |
| | VEIK | CGGTTACATTCGGAGGCGGCACTAAAGTAGAAAT |
| | | AAAG |
| SEQ ID NO | 553 | 554 |
| AVE-07-B01 | EVQLLESGGGLV | GAGGTCCAGCTACTGGAAAGCGGGGGCGGGTTA |
| | QPGGSLRLSCAA | GTTCAGCCCGGGGGTAGCTTGCGCCTCTCATGCG |
| | SGFTFGSYAMTW | CCGCCTCCGGATTCACCTTCGGCTCTTATGCCATG |
| | VRQAPGKGLEW | ACCTGGGTGAGACAGGCACCTGGCAAGGGACTG |
| | VSAIGTTDRYTY | GAATGGGTGTCTGCTATTGGCACTACTGACAGAT |
| | YADSVKGRFTIS | ACACGTACTACGCTGATAGCGTGAAAGGCCGTTT |
| | RDNSKNTLYLQ | TACCATCTCCCGAGACAACAGTAAGAACACACTG |
| | MNSLRAEDTAV | TATCTCCAGATGAATAGTCTGAGGGCTGAGGATA |
| | YYCARSRSYYAG | CAGCCGTTTATTACTGTGCAAGGTCGCGGTCCTAC |
| | AFDVWGQGTLV | TATGCGGGTGCATTTGACGTCTGGGGACAAGGGA |
| | TVSSGGGGSGGG | CCCTTGTAACAGTGTCTTCAGGTGGCGGTGGATC |
| | GSGGGGSDIQMT | GGGCGGTGGTGGATCTGGAGGAGGTGGCTCGGAT |
| | QSPSSLSASVGD | ATTCAGATGACACAGAGCCCCTCTAGTCTCAGCG |
| | RVTITCRASQTLR | CCTCCGTGGGTGACAGAGTTACCATTACCTGCAG |
| | NYLNWYQQKPG | GGCTTCGCAGACTCTGCGGAACTACTTGAACTGG |
| | KAPKLLIYAASN | TACCAACAAAAACCAGGGAAAGCGCCGAAGCTC |
| | LQTGVPSRFSGS | CTTATCTACGCAGCCTCCAATTTACAGACTGGCGT |
| | GSGTDFTLTISSL | CCCCTCTCGCTTTAGTGGCTCAGGATCAGGCACG |
| | QPEDFATYYCQQ | GACTTCACTCTGACCATAAGCTCTCTGCAGCCAG |
| | RFSPPWTFGGGT | AGGATTTCGCTACCTATTATTGTCAGCAGCGATTT |
| | KVEIK | TCCCCTCCTTGGACATTCGGAGGTGGGACAAAGG |
| | | TGGAAATCAAG |
| SEQ ID NO | 555 | 556 |
| AVE-07-C01 | EVQLLESGGGLV | GAGGTACAGCTCCTCGAAAGTGGAGGAGGGCTG |
| | QPGGSLRLSCAA | GTCCAGCCCGGCGGCTCACTAAGACTTTCATGTG |
| | SGYTFSNFAISW | CGGCATCGGGGTATACTTTCAGCAATTTCGCTATT |
| | VRQAPGKGLEW | TCCTGGGTGCGGCAAGCCCCTGGCAAAGGGCTGG |
| | VSSITGSGVRTFY | AATGGGTGAGCAGCATAACTGGTTCTGGTGTGCG |
| | ADSVKGRFTISR | TACATTCTACGCCGATTCTGTTAAGGGAAGATTTA |
| | DNSKNTLYLQM | CCATCTCCAGGGACAATAGCAAGAACACATTATA |
| | NSLRAEDTAVYY | TCTGCAGATGAACAGTTTGCGCGCCGAGGATACC |
| | CARSRSYYAGAF | GCTGTCTACTATTGCGCAAGGTCACGATCCTACTA |
| | DVWGQGTLVTV | CGCTGGCGCCTTTGACGTTTGGGGGCAGGGCACC |
| | SSGGGGSGGGGS | CTGGTGACGGTGTCTTCCGGTGGCGGTGGATCGG |
| | GGGGSDIQMTQS | GCGGTGGTGGATCTGGAGGAGGTGGCTCGGATAT |
| | PSSLSASVGDRV | TCAGATGACTCAATCCCCTAGCAGCCTGTCTGCG |
| | TITCRASQSIGSS | AGTGTGGGCGACAGAGTTACCATCACTTGCAGGG |
| | VNWYQQKPGKA | CTTCACAATCCATAGGGTCATCCGTCAATTGGTAT |
| | PKLLIYAASSLQS | CAGCAGAAACCTGGGAAAGCTCCCAAGTTGCTTA |
| | GVPSRFSGSGSG | TCTATGCCGCCTCTTCTCTGCAGTCCGGCGTGCCA |
| | TDFTLTISSLQPE | TCTCGGTTCAGCGGCAGCGGAAGTGGGACAGACT |
| | DFATYYCQQSDN | TTACACTCACCATCTCGTCATTACAGCCAGAAGA |
| | NPWTFGGGTKV | TTTCGCAACGTACTACTGTCAGCAGAGTGACAAC |
| | EIK | AACCCCTGGACCTTTGGAGGTGGTACAAAGGTAG |
| | | AGATTAAG |
| SEQ ID NO | 557 | 558 |
| AVE-07-D01 | EVQLLKSGSGLV | GAGGTACAACTCTTAAAGTCCGGATCTGGATTGG |
| | QPGGSLRLSCAA | TCCAGCCTGGCGGCAGCCTCAGACTGTCATGCGC |
| | SGFTFNNAWMH | AGCCAGCGGATTTACCTTCAACAATGCCTGGATG |
| | WVRQAPGKGLE | CACTGGGTGAGGCAGGCACCCGGCAAAGGTCTTG |
| | WVSAISSSGGYT | AGTGGGTGTCGGCCATCTCAAGCTCCGGGGGCTA |
| | YYADSVKGRFTI | TACATATTATGCAGATTCAGTTAAGGGGCGGTTC |
| | SRDNSKNTLYLQ | ACCATTTCTCGTGACAACTCCAAAAATACTCTGTA |
| | MNSLRAEDTAV | CCTGCAGATGAACAGTCTTCGAGCTGAAGATACA |
| | YYCARSRSYYAG | GCTGTGTATTACTGTGCTAGGTCCCGCTCTTACTA |
| | AFDVWGQGTLV | CGCGGGCGCCTTTGACGTTTGGGGGCAGGGTACT |
| | TVSSGGGGSGGG | CTGGTCACGGTGAGCAGTGGTGGCGGTGGATCGG |
| | GSGGGGSDIQMT | GCGGTGGTGGATCTGGAGGAGGTGGCTCGGATAT |
| | QSPSSLSASVGD | TCAGATGACACAGTCCCCAAGCTCGCTGTCTGCTT |
| | RVTITCRASAPV | CCGTTGGCGATAGAGTAACTATCACATGCCGCGC |
| | GRYLNWYQQKP | CAGCGCTCCAGTGGGGAGGTACCTCAACTGGTAC |
| | GKAPKLLIYAAS | CAGCAGAAACCCGGGAAGGCACCTAAATTGCTGA |
| | SLQSGVPSRFSGS | TCTATGCCGCCTCTTCACTGCAGTCAGGTGTCCCC |
| | GSGTDFTLTISSL | TCTCGGTTTAGTGGGAGCGGTAGTGGAACTGACT |
| | QPEDFATYYCQQ | TTACACTTACCATTTCCTCCTTACAGCCTGAAGAC |
| | AYSIPITFGGGTK | TTCGCAACGTATTACTGTCAACAAGCGTATAGCA |
| | VEIK | TCCCCATAACCTTCGGAGGCGGCACCAAGGTGGA |
| | | GATTAAG |
| SEQ ID NO | 559 | 560 |
| AVE-07-E01 | EVQLLESGGGLV | GAGGTTCAGCTGTTAGAATCAGGAGGGGGGTTGG |
| | QPGGSLRLSCAA | TGCAGCCTGGAGGCTCTCTCCGGCTGTCCTGCGCC |
| | SGFSVRDFAMN | GCATCGGGCTTCTCTGTCCGTGACTTCGCCATGAA |
| | WVRQAPGKGLE | TTGGGTTCGGCAGGCCCCCGGTAAGGGGCTGGAG |
| | WVSTITGTDRTP | TGGGTGAGCACCATCACAGGTACTGATAGAACAC |
| | YYADSVKGRFTI | CATACTACGCAGATTCAGTAAAGGGAAGATTTAC |
| | SRDNSKNTLYLQ | TATTAGCAGGGACAATTCTAAAAACACGCTTTAC |
| | MNSLRAEDTAV | CTCCAGATGAACAGTCTAAGGGCTGAAGATACAG |
| | YYCARSRSYYAG | CCGTGTATTATTGTGCTCGCAGCCGATCCTACTAT |
| | AFDVWGQGTLV | GCTGGCGCGTTTGACGTGTGGGGCCAAGGCACCC |
| | TVSSGGGGSGGG | TGGTCACCGTGAGTTCCGGTGGCGGTGGATCGGG |
| | GSGGGGSDIQMT | CGGTGGTGGATCTGGAGGAGGTGGCTCGGATATA |
| | QSPSSLSASVGD | CAGATGACCCAGTCTCCCAGCAGCCTTTCTGCCTC |
| | RVTITCRAPQNIF | AGTCGGGGATAGAGTTACGATCACATGTCGGGCT |
| | SYINWYQQKPGK | CCACAGAATATCTTCAGCTACATTAACTGGTACC |
| | APKLLIYAASSLQ | AGCAGAAGCCGGGCAAGGCACCTAAGTTGCTGAT |
| | SGVPSRFSGSGSG | CTATGCGGCTAGTTCCTTACAATCCGGAGTGCCAT |
| | TDFTLTISSLQPE | CGAGGTTTTCCGGCTCTGGGTCCGGTACAGACTTT |
| | DFATYYCQQTHS | ACCCTGACTATTAGCTCACTCCAGCCTGAGGACTT |
| | IPQTFGGGTKGEI | CGCCACCTATTACTGCCAGCAAACACACAGTATC |
| | K | CCCCAAACTTTCGGCGGTGGGACCAAAGGAGAAA |
| | | TTAAA |
| SEQ ID NO | 561 | 562 |
| AVE-07-F01 | EVQLLESGGGLV | GAAGTGCAATTGCTCGAAAGCGGCGGAGGCCTTG |
| | QPGGSLRLSCAA | TTCAGCCTGGAGGTAGCCTGAGGCTGTCTTGTGC |
| | SGFTFSSHNMAW | GGCTTCCGGGTTCACCTTCAGCTCACACAACATG |
| | VRQAPGKGLEW | GCTTGGGTGCGGCAGGCACCAGGAAAGGGGCTA |
| | VSSIGGGGRTTN | GAGTGGGTGAGTTCCATCGGTGGCGGTGGAAGGA |
| | YADSVKGRFTIS | CTACGAACTATGCCGACTCAGTCAAAGGGCGTTT |
| | RDNSKNTLYLQ | TACAATTTCCCGCGATAATTCTAAGAATACTCTTT |
| | MNSLRAEDTAV | ACCTCCAGATGAACAGTCTGCGAGCCGAGGATAC |
| | YYCARAAPGAY | CGCTGTCTATTACTGCGCCAGAGCTGCACCCGGC |
| | AYALDYWGQGT | GCCTACGCCTATGCATTAGACTACTGGGGCCAGG |
| | LVTVSSGGGGSG | GGACCCTGGTGACAGTATCGTCTGGTGGCGGTGG |
| | GGGSGGGGSDIQ | ATCGGGCGGTGGTGGATCTGGAGGAGGTGGCTCG |
| | MTQSPSSLSASV | GATATTCAGATGACCCAATCACCAAGCAGCCTTA |
| | GDRVTITCRASQ | GCGCATCCGTTGGTGATCGGGTGACAATTACCTG |
| | RISTYINWYQQK | CAGGGCCTCTCAGAGAATATCCACGTACATCAAC |
| | PGKAPKLLIYGA | TGGTACCAGCAGAAGCCTGGGAAAGCGCCTAAGC |
| | SYLHSGVPSRFS | TCCTGATTTATGGCGCCAGTTATTTGCACTCTGGC |
| | GSGSGTDFTLTIS | GTGCCCTCGCGCTTTAGCGGTAGTGGATCAGGGA |
| | SLQPEDFATYYC | CCGACTTTACTCTGACTATCTCTTCCTTACAGCCC |
| | QQTYSTPPTFGG | GAAGACTTCGCTACATATTACTGTCAACAGACAT |
| | GTKVEIK | ACTCCACACCCCCAACTTTCGGGGGCGGAACCAA |
| | | AGTCGAGATCAAG |
| SEQ ID NO | 563 | 564 |
| AVE-07-C02 | EVQLLESGGGLV | GAAGTACAGCTTCTGGAGTCTGGCGGAGGACTAG |
| | QPGGSLRLSCAA | TTCAGCCCGGCGGGTCACTCCGGCTGAGCTGTGC |
| | SGFSVRDFAMT | TGCATCAGGGTTTAGCGTCCGTGATTTCGCCATGA |
| | WVRQAPGKGLE | CATGGGTCAGACAGGCTCCTGGTAAGGGCCTTGA |
| | WVSAIRISGGGTF | ATGGGTGTCCGCCATCAGAATATCGGGGGGGGGA |
| | YADSVKGRFTIS | ACCTTTTATGCCGACAGCGTGAAAGGCCGATTCA |
| | RDNSKNTLYLQ | CTATTTCCCGCGACAATTCTAAGAACACTCTGTAT |
| | MNSLRAEDTAV | TTGCAAATGAACAGTCTGAGGGCCGAGGATACAG |
| | YYCARAAPGAY | CCGTCTACTACTGCGCAAGGGCGGCACCAGGTGC |
| | AYALDYWGQGT | TTACGCTTATGCCCTCGACTACTGGGGACAGGGC |
| | LVTVSSGGGGSG | ACGTTAGTGACCGTGAGTTCCGGTGGCGGTGGAT |
| | GGGSGGGGSDIQ | CGGGCGGTGGTGGATCTGGAGGAGGTGGCTCGGA |
| | MTQSPSSLSASV | CATTCAGATGACCCAGTCACCGTCTTCACTCTCCG |
| | GDRVTITCRASQ | CCAGTGTGGGTGACAGAGTGACTATCACCTGTCG |
| | RISTYINWYQQK | AGCCAGCCAAAGGATATCTACTTATATCAACTGG |
| | PGKAPKLLIYSAS | TATCAGCAGAAACCAGGGAAGGCACCTAAGTTAT |
| | VLENGVPSRFSG | TGATCTACTCGGCGTCCGTTCTGGAAAATGGAGT |
| | SGFGTDFTLTISS | CCCCTCTCGGTTTAGTGGGAGCGGATTCGGCACG |
| | LQPEDFATYYCQ | GATTTTACACTTACCATTAGCTCCCTGCAGCCAGA |
| | QNYRTPPTFGSG | GGATTTCGCTACCTACTACTGCCAACAGAATTATC |
| | TKGENK | GCACTCCTCCCACATTCGGTAGCGGCACAAAAGG |
| | | CGAGAACAAG |
| SEQ ID NO | 565 | 566 |
| AVE-07-F02 | EVQLLESGGGLV | GAGGTCCAGCTGCTGGAATCAGGCGGAGGGCTGG |
| | QPGGSLRLSCAA | TTCAGCCCGGCGGCTCCCTCAGGTTAAGCTGCGC |
| | SGFTFSNYAMTW | GGCAAGCGGGTTTACCTTCTCTAATTACGCCATG |
| | VRQAPGKGLEW | ACATGGGTGAGACAAGCCCCTGGAAAAGGTTTGG |
| | VSTITSSASKTNY | AGTGGGTGTCGACCATCACGAGCTCTGCTTCCAA |
| | ADSVKGRFTISR | GACTAACTATGCAGATTCTGTGAAAGGCCGGTTC |
| | DNSKNTLYLQM | ACAATTTCCAGAGATAACTCAAAGAATACTCTTT |
| | NSLRAEDTAVYY | ACCTACAGATGAACAGTCTGCGAGCCGAAGACAC |
| | CARTRYLERFAG | TGCTGTCTATTACTGTGCCCGTACCAGGTATCTTG |
| | GLDIWGQGTLVT | AGCGCTTTGCTGGCGGTCTGGACATATGGGGACA |
| | VSSGGGGSGGGG | GGGGACACTCGTGACCGTAAGCAGTGGTGGCGGT |
| | SGGGGSDIQMTQ | GGATCGGGCGGTGGTGGATCTGGAGGAGGTGGCT |
| | SPSSLSASVGDR | CGGACATCCAGATGACCCAGTCTCCTAGTAGCCT |
| | VTITCRASQSISS | TTCTGCAAGCGTTGGAGATCGGGTGACCATCACA |
| | YLNWYQQKPGK | TGCAGAGCCAGCCAAAGTATAAGCTCATACTTGA |
| | APKLLIYAASSLQ | ACTGGTACCAGCAGAAGCCAGGTAAGGCTCCAAA |
| | SGVPSRFSGSGSG | ACTGTTAATTTACGCTGCCTCTTCCCTGCAGTCGG |
| | TDFTLTISSLQPE | GCGTGCCTAGTAGGTTTTCAGGGTCAGGGTCCGG |
| | DFATYYCQQSFT | AACTGACTTCACCCTCACAATCTCCTCCCTGCAGC |
| | APLTFGGGTKVE | CCGAGGATTTCGCCACCTATTATTGTCAACAGTCT |
| | IK | TTTACGGCGCCCCTCACTTTCGGCGGTGGCACAA |
| | | AGGTCGAAATTAAA |
| SEQ ID NO | 567 | 568 |
| AVE-07-G02 | EVQLLESGGGLV | GAGGTGCAACTGCTGGAATCTGGTGGAGGTCTGG |
| | QPGGSLRLSCAA | TCCAGCCCGGAGGCAGTCTTAGGCTCAGCTGTGC |
| | SGLTFSNYTMTW | CGCATCCGGCCTAACTTTTAGCAATTACACCATG |
| | VRQAPGKGLEW | ACATGGGTTCGTCAGGCCCCTGGGAAAGGGTTGG |
| | VSGISGTGGSTW | AATGGGTTTCTGGCATCTCAGGAACCGGGGGTTC |
| | YADSVKGRFTIS | GACATGGTACGCTGATTCAGTGAAGGGCAGATTC |
| | RDNSKNTLYLQ | ACCATTTCCCGAGACAACAGCAAGAATACGCTGT |
| | MNSLRAEDTAV | ATCTTCAGATGAACAGTTTAAGGGCGGAGGACAC |
| | YYCARSRSYYAG | TGCAGTCTATTATTGCGCCCGCTCCCGGTCCTACT |
| | AFDVWGQGTLV | ACGCTGGGGCCTTCGATGTGTGGGGCCAGGGAAC |
| | TVSSGGGGSGGG | CCTCGTAACAGTGTCTAGCGGTGGCGGTGGATCG |
| | GSGGGGSDIQMT | GGCGGTGGTGGATCTGGAGGAGGTGGCTCGGATA |
| | QSPSSLSASVGD | TTCAGATGACTCAATCACCCTCATCGCTGTCTGCG |
| | RVTITCRASVSIS | AGTGTAGGGGACCGCGTCACAATCACCTGTAGAG |
| | TFLNWYQQKPG | CCTCCGTGAGCATCAGTACGTTTTTAAACTGGTAT |
| | KAPKLLIYAAST | CAACAGAAACCCGGGAAGGCTCCTAAGCTGCTCA |
| | LQSGVPSRFSGS | TATACGCAGCTTCAACACTTCAGAGCGGTGTTCC |
| | GSGTDFTLTISSL | AAGCAGGTTTTCTGGGTCCGGATCTGGAACTGAC |
| | QPEDFATYYCQQ | TTCACATTGACTATCAGCTCCCTCCAGCCAGAAG |
| | SLRTPITFGGGTK | ATTTCGCCACCTATTACTGCCAGCAGTCCCTGCGG |
| | VEIK | ACCCCTATTACCTTCGGCGGCGGCACAAAGGTGG |
| | | AGATTAAA |
| SEQ ID NO | 569 | 570 |
| AVE-07-H03 | EVQLLESGGGLV | GAAGTCCAGCTCTTGGAATCCGGAGGCGGCCTGG |
| | QPGGSLRLSCAA | TGCAGCCCGGGGGGTCATTGCGACTGAGTTGCGC |
| | SGFPFSSFVMSW | CGCATCTGGTTTCCCTTTTTCTAGCTTCGTGATGT |
| | VRQAPGKGLEW | CCTGGGTTAGACAGGCCCCGGGGAAGGGTTTAGA |
| | VSSLSGSGDITYY | GTGGGTGAGCTCGCTGTCCGGAAGCGGGGACATT |
| | ADSVKGRFTISR | ACTTATTATGCAGATTCTGTTAAGGGCCGCTTTAC |
| | DNSKNTLYLQM | CATCAGCCGGGACAACTCCAAAAATACACTCTAC |
| | NSLRAEDTAVYY | CTTCAAATGAACAGTCTACGTGCCGAGGACACCG |
| | CARAAPGAYAY | CCGTGTACTACTGTGCTAGGGCTGCCCCAGGCGC |
| | ALDYWGQGTLV | GTATGCATATGCTCTGGATTACTGGGGACAGGGC |
| | TVSSGGGGSGGG | ACGCTGGTCACAGTATCATCAGGTGGCGGTGGAT |
| | GSGGGGSDIQMT | CGGGCGGTGGTGGATCTGGAGGAGGTGGCTCGGA |
| | QSPSSLSASVGD | TATCCAAATGACACAGTCGCCTTCTTCCCTTAGCG |
| | RVTITCRTSQSVS | CATCAGTCGGGGACCGCGTGACAATTACCTGTAG |
| | TYFNWYQQKPG | GACCTCCCAGAGCGTGAGTACTTACTTTAACTGG |
| | KAPKLLIYAASSL | TACCAACAGAAGCCCGGCAAAGCCCCCAAGCTGC |
| | QSGVPSRFSGSGS | TGATCTATGCCGCGTCTAGTTTGCAGAGTGGTGTT |
| | GTDFTLTISSLQP | CCTTCACGGTTCTCAGGGTCCGGCTCTGGCACAG |
| | EDFATYYCQQSY | ATTTCACCTTAACTATTTCCAGCCTCCAGCCAGAG |
| | RTPPTFGGGTKV | GACTTTGCTACGTATTACTGCCAGCAGAGCTATA |
| | EIK | GAACTCCGCCAACATTCGGAGGAGGTACCAAGGT |
| | | AGAAATAAAA |
| SEQ ID NO | 571 | 572 |
| AVE-07-D04 | EVQLLESGGGLV | GAGGTGCAGCTACTGGAGTCAGGAGGCGGGCTG |
| | QPGGSLRLSCAA | GTTCAGCCTGGGGGGTCATTGCGACTGAGCTGTG |
| | SGLTFNAYAMS | CCGCATCTGGCCTCACCTTTAACGCCTACGCCATG |
| | WVRQAPGKGLE | AGCTGGGTCCGTCAGGCGCCCGGCAAAGGCTTGG |
| | WVSSLTGSGAST | AATGGGTGTCCAGCTTAACTGGGTCTGGAGCCTC |
| | FYADSVKGRFTIS | CACCTTCTACGCTGACTCGGTCAAGGGTAGATTC |
| | RDNSKNTLYLQ | ACAATCTCTCGGGACAATTCAAAGAATACACTTT |
| | MNSLRAEDTAV | ATCTGCAGATGAACAGTCTGAGGGCTGAAGATAC |
| | YYCARSRSYYAG | GGCTGTGTACTATTGCGCACGCTCCAGGTCCTACT |
| | AFDVWGQGTLV | ATGCCGGTGCATTTGATGTTTGGGGCCAAGGAAC |
| | TVSSGGGGSGGG | CCTCGTAACTGTGAGCAGTGGTGGCGGTGGATCG |
| | GSGGGGSDIQMT | GGCGGTGGTGGATCTGGAGGAGGTGGCTCGGATA |
| | QSPSSLSASVGD | TCCAGATGACACAGTCGCCCAGCTCCCTGAGCGC |
| | RVTITCRASRTVT | TTCAGTGGGGGACCGCGTAACTATTACATGTAGA |
| | SYLNWYQQKPG | GCCAGTCGAACCGTCACCTCCTATCTCAACTGGT |
| | KAPKLLIYGASY | ACCAACAGAAGCCTGGCAAGGCGCCAAAATTACT |
| | LHSGVPSRFSGS | GATATACGGAGCATCTTATTTGCACTCTGGTGTGC |
| | GSGTDFTLTISSL | CGTCTAGGTTCTCCGGCAGTGGGAGCGGAACCGA |
| | QPEDFATYYCQQ | CTTCACTCTTACTATCTCCAGCCTGCAGCCAGAAG |
| | SYRTPPTFGGGT | ATTTTGCCACATATTACTGCCAACAGTCATACCGG |
| | KVEIK | ACCCCTCCCACGTTTGGGGGTGGCACAAAGGTTG |
| | | AGATTAAA |
| SEQ ID NO | 573 | 574 |
| AVE-07-E06 | EVQLLESGGGLV | GAGGTCCAGCTCCTGGAATCAGGTGGGGGCCTGG |
| | QPGGSLRLSCAA | TGCAGCCCGGCGGGAGCTTGCGGCTTTCTTGTGCT |
| | SGFSVRDFAMN | GCAAGCGGATTTTCAGTGAGAGACTTCGCCATGA |
| | WVRQAPGKGLE | ATTGGGTCCGTCAAGCCCCTGGAAAGGGGTTAGA |
| | WVSSIHRTGGGT | ATGGGTGTCTAGCATTCACAGGACAGGCGGGGGC |
| | YYADSVKGRFTI | ACATACTACGCAGATAGTGTTAAAGGCAGGTTCA |
| | SRDNSKNTLYLQ | CGATCTCCAGAGACAATTCCAAGAACACCCTCTA |
| | MNSLRAEDTAV | CCTACAGATGAACAGTCTGCGAGCGGAGGACACC |
| | YYCARSRSYYAG | GCCGTTTATTACTGCGCCCGGAGCCGCTCCTATTA |
| | AFDVWGQGTLV | TGCTGGAGCTTTTGATGTGTGGGGTCAGGGAACT |
| | TVSSGGGGSGGG | CTGGTGACTGTATCTTCGGGTGGCGGTGGATCGG |
| | GSGGGGSDIQMT | GCGGTGGTGGATCTGGAGGAGGTGGCTCGGATAT |
| | QSPSSLSASVGD | TCAGATGACGCAGTCCCCCAGTAGCTTGAGTGCC |
| | RVTITCRANQNI | AGCGTGGGTGACCGGGTTACTATCACATGTAGAG |
| | GNYLNWYQQKP | CGAACCAGAATATCGGCAACTACCTTAATTGGTA |
| | GKAPKLLIYAAS | CCAACAGAAGCCTGGGAAGGCTCCCAAACTCCTG |
| | SLQSGVPSRFSGS | ATCTATGCAGCCTCATCCCTGCAGTCAGGTGTGCC |
| | GSGTDFTLTISSL | GTCCAGGTTTTCGGGGTCCGGGAGCGGAACCGAT |
| | QPEDFATYYCQQ | TTCACCCTGACAATTTCTTCTTTACAACCAGAAGA |
| | SYSTSTFGGGTK | CTTTGCTACCTACTATTGCCAGCAGTCATATAGCA |
| | VEIK | CCTCTACTTTCGGCGGCGGAACAAAGGTCGAGAT |
| | | AAAA |
| SEQ ID NO | 575 | 576 |
| AVE-07-F06 | EVQLLESGGGLV | GAGGTCCAGCTGTTGGAAAGCGGGGGGGGCTTAG |
| | QPGGSLRLSCAA | TACAGCCTGGAGGTTCCCTCCGTCTAAGCTGCGC |
| | SGFDFRSYPMAW | CGCATCGGGATTCGACTTTCGCTCCTACCCAATGG |
| | VRQAPGKGLEW | CTTGGGTGCGGCAGGCCCCCGGCAAGGGCCTGGA |
| | VSVISGGGGSTN | ATGGGTTAGTGTTATTTCTGGTGGAGGCGGTTCA |
| | YADSVKGRFTIS | ACCAACTACGCGGACAGCGTCAAAGGGAGATTTA |
| | RDNSKNTLYLQ | CTATCTCTAGGGACAATTCCAAGAACACGCTGTA |
| | MNSLRAEDTAV | CCTGCAGATGAATAGTCTCCGAGCAGAGGATACA |
| | YYCARSRSYYAG | GCCGTGTATTATTGTGCCAGGTCAAGATCCTACTA |
| | AFDVWGQGTLV | TGCTGGAGCTTTCGATGTGTGGGGCCAAGGGACA |
| | TVSSGGGGSGGG | CTTGTGACCGTGAGCTCTGGTGGCGGTGGATCGG |
| | GSGGGGSDIQMT | GCGGTGGTGGATCTGGAGGAGGTGGCTCGGACAT |
| | QSPSSLSASVGD | ACAGATGACACAGTCTCCATCTAGCCTTAGTGCC |
| | RVTITCRASQKIA | TCCGTTGGAGATCGGGTGACCATCACATGCCGCG |
| | RYVNWYQQKPG | CCTCACAGAAGATTGCAAGATATGTCAACTGGTA |
| | KAPKLLIYAASD | CCAGCAGAAACCTGGTAAGGCACCAAAGCTCTTA |
| | LQSGVPSRFSGS | ATCTATGCTGCGAGCGACTTGCAAAGCGGCGTGC |
| | GSGTDFTLTISSL | CCTCTAGGTTTTCCGGCAGCGGTTCAGGCACCGA |
| | QPEDFATYYCQQ | TTTCACCCTGACGATCTCCTCGCTGCAGCCTGAGG |
| | AYSIPITFGGGTK | ACTTTGCTACTTATTACTGTCAGCAAGCCTACAGT |
| | VEIK | ATTCCCATTACATTCGGGGGAGGGACTAAAGTAG |
| | | AAATCAAA |
| SEQ ID NO | 577 | 578 |
| AVE-07-A07 | EVQLLESGGGLV | GAGGTGCAACTCCTTGAGTCAGGTGGCGGACTCG |
| | QPGGSLRLSCAA | TGCAGCCTGGCGGATCGCTGAGACTGTCATGTGC |
| | SGFRFSNYAMT | GGCTTCTGGGTTTCGTTTCTCCAACTACGCAATGA |
| | WVRQAPGKGLE | CATGGGTGAGGCAGGCCCCCGGGAAAGGCCTGG |
| | WVSGISGGGGRT | AGTGGGTATCCGGAATCAGCGGCGGCGGGGGCA |
| | FYADSVKGRFTIS | GGACCTTCTATGCAGATAGTGTCAAGGGTAGATT |
| | RDNSKNTLYLQ | TACCATTAGCCGGGACAATTCCAAGAATACATTA |
| | MNSLRAEDTAV | TACCTACAGATGAACAGTCTGCGCGCCGAAGATA |
| | YYCARGYGAEY | CGGCTGTCTATTACTGCGCCCGAGGTTACGGGGC |
| | FDPWGQGTLVT | CGAATATTTCGACCCGTGGGGGCAGGGAACTTTG |
| | VSSGGGGSGGGG | GTTACTGTGAGCTCTGGTGGCGGTGGATCGGGCG |
| | SGGGGSDIQMTQ | GTGGTGGATCTGGAGGAGGTGGCTCGGACATCCA |
| | SPSSLSASVGDR | GATGACCCAGTCACCCTCTTCCCTGTCCGCAAGTG |
| | VTITCRASQPISR | TGGGCGACCGGGTGACTATTACTTGTCGCGCCAG |
| | YLNWYQQKPGK | CCAGCCCATCAGTAGATACTTAAACTGGTATCAG |
| | APKLLIYDASRL | CAGAAGCCGGGCAAAGCGCCAAAGCTGTTGATAT |
| | QVGVPSRFSGSG | ATGACGCTTCTCGACTTCAAGTCGGCGTACCTAG |
| | SGTDFTLTISSLQ | CAGGTTCTCAGGGAGCGGTTCTGGAACGGATTTC |
| | PEDFATYYCQQS | ACACTGACCATCTCGTCCCTCCAACCAGAGGATT |
| | HSIPWTFGGGTK | TTGCCACATACTACTGCCAGCAGAGCCACTCCAT |
| | VEIK | TCCTTGGACATTTGGGGGTGGAACCAAGGTTGAA |
| | | ATTAAA |
| SEQ ID NO | 579 | 580 |
| AVE-07-E07 | EVQLLESGGGLV | GAGGTGCAGCTGCTCGAATCGGGCGGGGGCCTGG |
| | QPGGSLRLSCAA | TCCAGCCCGGGGGGTCTTTGAGGCTCTCCTGTGCT |
| | SGFRFSNYAMT | GCCTCAGGGTTTAGGTTCAGTAATTACGCGATGA |
| | WVRQAPGKGLE | CTTGGGTGCGACAGGCCCCAGGAAAGGGCCTAGA |
| | WVSGISGGGGRT | GTGGGTGAGCGGCATTTCAGGTGGAGGAGGCAG |
| | FYADSVKGRFTIS | AACATTTTATGCAGATAGCGTTAAAGGTAGATTC |
| | RDNSKNTLYLQ | ACGATCTCTCGGGACAACTCCAAGAATACCCTTT |
| | MNSLRAEDTAV | ACTTACAAATGAACAGTCTGCGCGCAGAGGATAC |
| | YYCARGYGAEY | CGCCGTATACTATTGCGCTCGTGGGTATGGAGCC |
| | FDPWGQGTLVT | GAATACTTCGACCCTTGGGGCCAGGGTACCCTGG |
| | VSSGGGGSGGGG | TCACAGTGAGCTCCGGTGGCGGTGGATCGGGCGG |
| | SGGGGSDIQMTQ | TGGTGGATCTGGAGGAGGTGGCTCGGACATCCAA |
| | SPSSLSASVGDR | ATGACCCAGAGCCCATCATCCCTGAGCGCATCAG |
| | VTITCRASQPISR | TGGGAGATAGAGTCACTATCACCTGTCGCGCTTC |
| | HLNWYQQKPGK | TCAACCAATATCCCGACACCTCAACTGGTACCAG |
| | APKLLIYSASSLQ | CAGAAACCCGGAAAGGCCCCTAAGCTGTTGATTT |
| | SGVPSRFSGSGSG | ATAGTGCCAGTTCCCTGCAGTCAGGTGTTCCTAGC |
| | TDFTLTISSLQPE | AGGTTTTCTGGCTCTGGGTCCGGCACAGACTTCAC |
| | DFATYYCQQSYD | GCTTACTATTTCTAGCTTACAGCCCGAAGATTTTG |
| | RTWTFGGGTKG | CGACATACTACTGCCAGCAGAGTTATGACCGGAC |
| | ESK | ATGGACCTTCGGTGGGGGCACCAAGGGGGAGTCG |
| | | AAA |
| SEQ ID NO | 581 | 582 |
| AVE-07-D08 | EVQLLESGGGLV | GAAGTCCAGCTCTTGGAATCCGGGGGGGGGTTAG |
| | QPGGSLRLSCAA | TGCAGCCTGGAGGCTCTCTCAGACTGAGTTGCGC |
| | SGFRFSNYAMT | CGCCAGCGGATTTAGGTTCTCCAATTATGCCATG |
| | WVRQAPGKGLE | ACGTGGGTGAGACAGGCCCCCGGGAAGGGGCTG |
| | WVSGISGGGGRT | GAGTGGGTGTCTGGCATTTCCGGCGGAGGCGGAC |
| | FYADSVKGRFTIS | GGACTTTCTATGCAGACTCAGTAAAGGGTAGGTT |
| | RDNSKNTLYLQ | CACAATCAGCCGAGACAACTCGAAAAATACCCTT |
| | MNSLRAEDTAV | TACCTGCAAATGAACAGTCTGCGCGCGGAGGATA |
| | YYCARGYGAEY | CCGCTGTGTACTACTGTGCACGTGGTTACGGCGCT |
| | FDPWGQGTLVT | GAGTATTTTGATCCGTGGGGCCAGGGTACACTAG |
| | VSSGGGGSGGGG | TCACTGTTAGCTCAGGTGGCGGTGGATCGGGCGG |
| | SGGGGSDIQMTQ | TGGTGGATCTGGAGGAGGTGGCTCGGACATTCAG |
| | SPSSLSASVGDR | ATGACCCAGTCACCCAGTAGCTTGTCTGCCTCTGT |
| | VTITCRASQRIAT | GGGAGATCGCGTCACAATAACTTGCAGGGCTTCC |
| | YLNWYQQKPGK | CAACGGATCGCCACCTACCTGAACTGGTACCAGC |
| | APKLLIYAASHL | AAAAACCTGGTAAGGCACCAAAGCTGTTAATTTA |
| | HGGVPSRFSGSG | CGCGGCCTCTCACCTTCATGGAGGCGTGCCATCC |
| | SGTDFTLTISSLQ | AGATTCTCCGGCAGCGGGTCAGGCACAGATTTTA |
| | PEDFATYYCQQS | CCCTGACTATCTCCTCGCTCCAGCCCGAAGACTTC |
| | YSIPLTFGGGTKV | GCTACGTATTATTGTCAGCAGAGTTATAGCATCCC |
| | EIK | TCTCACATTTGGTGGGGGGACCAAGGTTGAGATT |
| | | AAA |
| SEQ ID NO | 583 | 584 |
| AVE-07-E08 | GGAALKSGGGL | GGAGGCGCGGCCTTAAAGTCCGGAGGTGGCCTGG |
| | VQPGGSLRLSCA | TCCAGCCCGGAGGGTCCCTCAGATTGAGCTGTGC |
| | ASGFTVSRDYMS | TGCCTCTGGCTTTACAGTGTCACGGGATTATATGT |
| | WVRQAPGKGLE | CGTGGGTCCGCCAAGCTCCGGGCAAGGGACTGGA |
| | WVSVISTGGGST | ATGGGTGAGTGTGATTAGCACCGGGGGGGGTCC |
| | YYADSVKGRFTI | ACGTACTATGCCGACTCTGTTAAGGGGAGGTTCA |
| | SRDNSKNTLYLQ | CAATCTCCAGGGATAATTCAAAAAACACCCTGTA |
| | MNSLRAEDTAV | CCTTCAGATGAACAGTCTCCGTGCTGAGGACACC |
| | YYCAKPRPYSIA | GCAGTTTATTATTGCGCAAAACCCCGACCATACA |
| | WFADPFDYWGQ | GCATAGCATGGTTCGCCGACCCTTTTGATTACTGG |
| | GTLVTVSSGGGG | GGCCAGGGTACTCTGGTGACTGTAAGCTCTGGTG |
| | SGGGGSGGGGSD | GCGGTGGATCGGGCGGTGGTGGATCTGGAGGAG |
| | IQMTQSPSSLSAS | GTGGCTCGGATATCCAAATGACTCAGAGCCCTTC |
| | VGDRVTITCRTS | CAGCTTGAGTGCGTCCGTGGGGGATAGGGTAACT |
| | QTISIYLNWYQQ | ATCACATGTAGAACAAGCCAGACCATATCTATTT |
| | KPGKAPKLLIYA | ACCTCAACTGGTATCAGCAGAAGCCCGGAAAGGC |
| | ASILHGGVPSRFS | CCCAAAATTACTGATTTATGCAGCCTCAATCCTGC |
| | GSGSGTDFTLTIS | ACGGAGGTGTTCCGTCACGGTTCTCCGGGTCGGG |
| | SLQPEDFATYYC | CTCTGGTACCGACTTTACCCTTACGATTAGTAGCC |
| | QQNYSVPPTFGG | TGCAGCCTGAGGACTTCGCTACCTACTACTGCCA |
| | GTKVEIK | ACAGAATTATTCTGTCCCACCCACTTTTGGGGGCG |
| | | GCACAAAAGTGGAAATCAAG |
| SEQ ID NO | 585 | 586 |
| AVE-07-H08 | ELQLLESGGGLV | GAGCTGCAACTCTTAGAATCTGGCGGAGGCCTGG |
| | QPGGSLRLSCAA | TTCAGCCCGGAGGCAGTCTGCGTCTATCCTGTGCC |
| | SGFPFSNNAMSW | GCATCTGGGTTCCCTTTCTCCAACAACGCCATGTC |
| | VRQAPGKGLEW | ATGGGTCCGCCAGGCCCCAGGTAAAGGTCTTGAA |
| | VSVISGSYGTTY | TGGGTTTCTGTGATCAGCGGCAGCTACGGGACTA |
| | YADSVKGRFTIS | CATACTATGCCGATTCCGTGAAGGGCAGATTTAC |
| | RDNSKNTLYLQ | CATTAGCCGGGATAATTCGAAGAACACGCTTTAT |
| | MNSLRAEDTAV | CTGCAGATGAATAGTTTGAGGGCTGAGGACACCG |
| | YYCARVADGAA | CAGTGTATTACTGCGCCCGAGTGGCTGACGGGGC |
| | AYAMDYWGQG | AGCTGCTTACGCGATGGACTATTGGGGGCAGGGA |
| | TLVTVSSGGGGS | ACTCTCGTCACAGTAAGCTCAGGTGGCGGTGGAT |
| | GGGGSGGGGSDI | CGGGCGGTGGTGGATCTGGAGGAGGTGGCTCGGA |
| | QMTQSPSSLSAS | CATTCAGATGACCCAGTCACCATCCAGTCTGTCA |
| | VGDRVTITCRAS | GCTTCGGTGGGCGATAGGGTAACAATCACTTGCA |
| | QPISRYLNWYQQ | GAGCGTCCCAGCCCATCTCTCGCTATCTTAACTGG |
| | KPGKAPKLLIYD | TACCAGCAAAAGCCGGGGAAAGCACCAAAGCTG |
| | ASRLQVGVPSRF | TTAATTTACGACGCCTCTCGGCTGCAGGTCGGCGT |
| | SGSGSGTDFTLTI | TCCTAGCCGATTCAGCGGTTCTGGGTCCGGAACA |
| | SSLQPEDFATYY | GATTTTACCCTCACAATTAGCTCCTTGCAGCCCGA |
| | CQQSHSIPWTFG | AGACTTTGCCACCTATTACTGTCAGCAAAGTCAC |
| | GGTKVEIK | AGCATCCCTTGGACGTTCGGTGGAGGCACTAAGG |
| | | TGGAGATAAAA |
| SEQ ID NO | 587 | 588 |
| AVE-07-C10 | EVQLLESGGGLV | GAGGTGCAACTGCTTGAGAGTGGCGGAGGCCTGG |
| | QPGGSLRLSCAA | TGCAGCCTGGGGGCAGCCTCAGATTATCTTGCGC |
| | SGFRFSNYAMT | GGCCTCAGGTTTTCGTTTCTCTAACTATGCTATGA |
| | WVRQAPGKGLE | CATGGGTCCGACAGGCTCCAGGGAAAGGGCTAG |
| | WVSGISGGGGRT | AATGGGTATCGGGAATTTCCGGTGGCGGGGGCCG |
| | FYADSVKGRFTIS | GACTTTCTACGCCGATAGCGTCAAGGGAAGGTTC |
| | RDNSKNTLYLQ | ACAATCAGCCGCGACAATTCAAAGAATACCCTCT |
| | MNSLRAEDTAV | ACCTGCAGATGAACAGTTTGAGGGCCGAGGACAC |
| | YYCARGYGAEY | TGCAGTGTATTACTGTGCCAGAGGCTACGGAGCA |
| | FDPWGQGTLVT | GAATATTTTGATCCCTGGGGGCAGGGTACCCTGG |
| | VSSGGGGSGGGG | TGACGGTTTCCTCCGGTGGCGGTGGATCGGGCGG |
| | SGGGGSDIQMTQ | TGGTGGATCTGGAGGAGGTGGCTCGGACATCCAG |
| | SPSSLSASVGDR | ATGACCCAAAGTCCCTCTTCTCTTAGCGCTTCGGT |
| | VTITCRASQPISR | GGGCGATCGGGTGACCATTACTTGTAGAGCGTCA |
| | YLNWYQQKPGK | CAGCCAATAAGCAGGTATCTCAATTGGTATCAGC |
| | APKLLIYAASNL | AGAAGCCCGGAAAAGCACCCAAGTTGCTGATCTA |
| | KKGVPSRFSGSG | CGCCGCCTCCAACTTAAAGAAGGGGGTTCCTAGC |
| | FGTDFTLTISSLQ | CGCTTTAGTGGGTCCGGCTTCGGAACCGATTTCAC |
| | PEDFATYYCQQS | ACTCACAATCTCATCCCTGCAGCCGGAGGACTTT |
| | YNPPLTFGGGTK | GCTACGTACTATTGCCAACAGTCTTACAACCCTCC |
| | VEIK | ACTGACTTTCGGTGGCGGGACAAAAGTCGAAATT |
| | | AAA |
| SEQ ID NO | 589 | 590 |
| AVE-07-D11 | EVQLLESGGGLV | GAGGTGCAGCTGCTGGAGAGCGGAGGCGGACTA |
| | QPGGSLRLSCAA | GTGCAGCCAGGTGGGTCCCTGCGACTTAGCTGCG |
| | SGFTFPHSAMSW | CCGCTTCGGGGTTTACGTTTCCGCACTCTGCAATG |
| | VRQAPGKGLEW | TCTTGGGTGCGTCAGGCACCTGGAAAGGGCTTAG |
| | VSSIAGRGGSPN | AATGGGTCAGCAGCATCGCTGGCAGAGGTGGCTC |
| | YADSVKGRFTIS | TCCCAATTACGCCGACTCCGTGAAGGGTAGGTTC |
| | RDNSKNTLYLQ | ACTATTTCCCGCGATAACTCAAAAAATACACTCT |
| | MNSLRAEDTAV | ATCTGCAGATGAACAGTTTGAGGGCCGAAGACAC |
| | YYCARVADGGA | CGCGGTATATTATTGTGCACGGGTTGCCGATGGC |
| | AYAFDYWGQGT | GGGGCTGCCTACGCTTTCGACTACTGGGGGCAAG |
| | LVTVSSGGGGSG | GAACCCTCGTTACAGTCAGTTCAGGTGGCGGTGG |
| | GGGSGGGGSDIQ | ATCGGGCGGTGGTGGATCTGGAGGAGGTGGCTCG |
| | MTQSPSSLSASV | GACATTCAAATGACACAGAGTCCTTCTTCCTTGTC |
| | GDRVTITCRSSQ | TGCATCCGTCGGTGATCGCGTTACCATCACTTGCC |
| | NIITYLNWYQQK | GGTCGAGCCAGAACATAATCACTTATCTCAATTG |
| | PGKAPKLAIYGA | GTACCAGCAGAAACCCGGAAAAGCCCCAAAGTT |
| | SRVQSGVPSRFS | AGCGATTTACGGTGCCTCCAGGGTGCAGAGCGGC |
| | GSGSGADFTLTIS | GTGCCCAGCAGATTCAGCGGGAGTGGGTCAGGGG |
| | SLQPEDFSTYYC | CTGATTTCACACTGACCATCAGTTCTCTTCAGCCT |
| | QQSFSTPLTFGG | GAGGACTTTTCAACCTATTACTGTCAACAGTCCTT |
| | GTKVEIK | CTCAACGCCACTGACCTTTGGCGGAGGCACAAAG |
| | | GTAGAAATTAAG |
| SEQ ID NO | 591 | 592 |
| AVE-07-F11 | EVQLLESGGGLV | GAGGTGCAGCTCCTGGAAAGCGGAGGGGGCTTG |
| | QPGGSLRLSCAA | GTGCAGCCCGGAGGGTCCCTCCGATTATCCTGTG |
| | SGFRFSNYAMT | CTGCCTCAGGGTTTCGTTTTTCAAATTACGCAATG |
| | WVRQAPGKGLE | ACATGGGTCCGGCAGGCCCCTGGCAAGGGGCTGG |
| | WVSGISGGGGRT | AGTGGGTGAGCGGGATTTCCGGAGGTGGTGGCAG |
| | FYADSVKGRFTIS | AACGTTCTACGCAGATTCGGTGAAGGGTAGATTC |
| | RDNSKNTLYLQ | ACCATCAGTAGGGACAATTCTAAAAACACTCTAT |
| | MNSLRAEDTAV | ATCTGCAGATGAACAGTCTGAGGGCTGAAGACAC |
| | YYCARGYGAEY | TGCCGTTTATTATTGCGCGCGCGGATACGGCGCC |
| | FDPWGQGTLVT | GAGTACTTCGATCCGTGGGGCCAAGGCACCCTTG |
| | VSSGGGGSGGGG | TCACAGTAAGCTCTGGTGGCGGTGGATCGGGCGG |
| | SGGGGSDIQMTQ | TGGTGGATCTGGAGGAGGTGGCTCGGACATCCAG |
| | SPSSLSASVGDR | ATGACACAGTCACCCTCCAGCCTGTCTGCCTCTGT |
| | VTITCRASQPISR | GGGGGACAGGGTCACCATTACTTGTCGGGCATCG |
| | YLNWYQQKPGK | CAACCAATTAGCAGATACTTGAACTGGTATCAGC |
| | APKLLIYDASRL | AGAAGCCAGGAAAGGCCCCCAAGTTACTCATTTA |
| | QVGVPSRFSGSG | CGACGCTAGTCGACTTCAAGTGGGTGTTCCTTCTC |
| | SGTDFTLTISSPQ | GCTTCTCCGGGTCCGGAAGCGGCACTGATTTTAC |
| | PEDFATYYCQQS | CCTGACCATCTCAAGTCCCCAGCCTGAAGATTTTG |
| | HSIPWTFGGGTK | CGACATATTACTGCCAGCAGTCCCACAGCATACC |
| | GEIK | GTGGACGTTCGGTGGGGGCACAAAAGGCGAGAT |
| | | CAAA |
| SEQ ID NO | 593 | 594 |
| AVE-07-G11 | EVQLLESGGGLV | GAAGTGCAGCTCCTGGAGTCAGGCGGGGGACTGG |
| | QPGGSLRLSCAA | TCCAGCCCGGAGGTTCACTGCGTCTAAGCTGCGC |
| | SGFTFPHSAMSW | TGCATCTGGCTTCACATTCCCACACTCCGCCATGT |
| | VRQAPGKGLEW | CTTGGGTTCGCCAGGCCCCTGGGAAGGGATTGGA |
| | VSTVTGSGSPTY | ATGGGTGTCCACCGTTACAGGTTCCGGCAGCCCG |
| | YADSVKGRFTIS | ACATATTATGCCGATAGCGTGAAAGGGCGATTTA |
| | RDNSKNTLYLQ | CCATCAGCAGGGACAACAGTAAGAATACGCTGTA |
| | MNSLRAEDTAV | TCTTCAGATGAACAGTCTCCGGGCTGAGGATACT |
| | YYCARVAGGAY | GCGGTCTACTACTGTGCAAGAGTGGCTGGAGGGG |
| | GYAMDYWGQG | CCTACGGTTATGCAATGGACTACTGGGGCCAAGG |
| | TLVTVSSGGGGS | CACTTTAGTGACCGTATCGTCTGGTGGCGGTGGA |
| | GGGGSGGGGSDI | TCGGGCGGTGGTGGATCTGGAGGAGGTGGCTCGG |
| | QMTQSPSSLSAS | ACATACAGATGACCCAGTCCCCGTCAAGTCTCAG |
| | VGDRVTITCRAS | CGCCTCGGTCGGGGACAGAGTTACAATCACGTGT |
| | QSISSYLNWYQQ | CGGGCATCTCAATCCATTTCCAGCTATCTGAATTG |
| | KPGKAPKLLIYD | GTACCAGCAGAAACCTGGGAAGGCTCCAAAGCTG |
| | ASNLQSGVPSRF | CTTATCTACGATGCCAGCAACCTGCAGAGCGGAG |
| | SGSGSGTDFTLTI | TGCCCTCAAGGTTCTCCGGCAGTGGCTCTGGGAC |
| | SSLQPEDFATYY | TGATTTTACCCTCACAATTTCTTCTTTACAGCCCG |
| | CQQSFSNLYTFG | AAGACTTTGCGACTTACTATTGCCAGCAATCATTC |
| | GGTKVEIK | AGTAACTTGTATACCTTCGGTGGAGGCACAAAGG |
| | | TGGAGATCAAA |
| SEQ ID NO | 595 | 596 |
| AVE-07-F12 | EVQLLESGGGLV | GAGGTGCAGCTTCTGGAATCAGGGGGTGGACTGG |
| | QPGGSLRLSCAA | TTCAGCCTGGGGGTTCCCTCCGCCTGTCGTGTGCC |
| | SGFPFSVYAMTW | GCTTCTGGCTTTCCATTCTCCGTATATGCAATGAC |
| | VRQAPGKGLEW | ATGGGTGAGGCAGGCCCCGGGCAAGGGATTGGA |
| | VSSFGGSGHSPY | GTGGGTGAGTTCTTTTGGCGGCAGCGGGCACTCC |
| | YADSVKGRFTIS | CCCTATTACGCAGATTCCGTGAAGGGGCGTTTCA |
| | RDNSKNTLYLQ | CAATCTCAAGAGATAATTCTAAAAACACGCTGTA |
| | MNSLRAEDTAV | TCTACAGATGAACAGTCTCCGGGCTGAAGACACC |
| | YYCARVAAGSY | GCCGTTTACTACTGCGCGCGAGTGGCCGCTGGCA |
| | AYAMDYWGQG | GCTACGCCTATGCAATGGACTACTGGGGACAAGG |
| | TLVTVSSGGGGS | AACCTTAGTCACTGTCAGCAGCGGTGGCGGTGGA |
| | GGGGSGGGGSDI | TCGGGCGGTGGTGGATCTGGAGGAGGTGGCTCGG |
| | QMTQSPSSLSAS | ATATCCAGATGACTCAGAGCCCATCATCGCTCAG |
| | VGDRVTITCRSS | CGCATCTGTCGGGGACAGGGTTACCATAACCTGC |
| | QNIITYLNWYQQ | AGAAGTTCACAGAACATCATCACCTACCTTAATT |
| | KPGKAPKLLIYG | GGTACCAGCAAAAGCCTGGCAAAGCCCCTAAACT |
| | ASRLQSGVPSRFS | GCTCATTTATGGAGCCTCCCGGCTGCAGAGCGGC |
| | GSGSGTDFTLTIS | GTGCCCAGTCGCTTCTCCGGTTCTGGATCAGGTAC |
| | SLQPEDFATYYC | CGATTTCACGCTGACAATTAGCTCCTTACAACCCG |
| | QQSFSTPLTFGG | AAGACTTTGCTACTTACTATTGTCAGCAGTCCTTC |
| | GTKEVDK | TCTACTCCATTGACATTTGGCGGGGGGACAAAGG |
| | | AGGTGGACAAG |
| SEQ ID NO | 597 | 598 |
| AVE-07-G12 | EVQLLESGGGLV | GAAGTCCAGCTTCTGGAGAGCGGAGGCGGACTGG |
| | QPGGSLRLSCAA | TGCAGCCAGGCGGGAGCTTGCGTCTGTCCTGTGC |
| | SGFRFSNYAMT | GGCAAGCGGGTTCCGCTTCTCTAACTATGCCATG |
| | WVRQAPGKGLE | ACCTGGGTAAGGCAAGCACCTGGCAAAGGTCTTG |
| | WVSGISGGGGRT | AGTGGGTGTCCGGAATTTCCGGCGGGGGCGGACG |
| | FYADSVKGRFTIS | GACGTTCTACGCCGATTCAGTGAAGGGTCGATTT |
| | RDNSKNTLYLQ | ACCATCTCTAGGGATAATAGTAAGAACACTCTCT |
| | MNSLRAEDTAV | ACCTCCAGATGAATAGTTTGAGAGCTGAAGACAC |
| | YYCARGYGAEY | AGCCGTGTACTATTGCGCTAGAGGGTACGGTGCC |
| | FDPWGQGTLVT | GAGTATTTTGACCCCTGGGGGCAGGGCACTCTGG |
| | VSSGGGGSGGGG | TTACAGTCTCATCGGGTGGCGGTGGATCGGGCGG |
| | SGGGGSDIQMTQ | TGGTGGATCTGGAGGAGGTGGCTCGGATATTCAG |
| | SPSSLSASVGDR | ATGACACAGAGCCCATCTTCACTGTCCGCTTCGGT |
| | VTITCRASQPISR | AGGGGATAGAGTGACAATAACATGCCGCGCAAG |
| | YLNWYQQKPGK | CCAGCCTATCTCACGGTACCTCAACTGGTACCAA |
| | APKLLIYDASRL | CAGAAACCCGGGAAAGCCCCGAAGCTGTTAATCT |
| | QVGVPSRFSGSG | ATGACGCGAGTAGGCTGCAAGTGGGTGTCCCCAG |
| | SGTDFTLTISSLQ | CCGATTCAGCGGTTCCGGATCTGGCACCGACTTC |
| | PEDFATYYCQQS | ACCCTCACTATCTCTTCCTTGCAGCCAGAAGACTT |
| | FSTPLTFGGGTK | TGCCACGTATTACTGTCAGCAGTCCTTTAGTACCC |
| | VEIK | CTCTTACCTTCGGCGGAGGCACTAAGGTTGAGAT |
| | | TAAG |
| SEQ ID NO | 599 | 600 |

In some embodiments, provided herein is an anti-Activin E scFV antibody, wherein the amino acids have at least 95, 96, 97, 98, 99, or 100% sequence identity to SEQ ID NOS: 501; 503; 505; 507; 509; 511; 513; 515; 517; 519; 521; 523; 525; 527; 529; 531; 533; 535; 537; 539; 541; 543; 545; 547; 549; 551; 553; 555; 557; 559; 561; 563; 565; 567; 569; 571; 573; 575; 577; 579; 581; 583; 585; 587; 589; 591; 593; 595; 597; or 599.

In some embodiments, provided herein is an anti-Activin E scFV antibody encoded by a polynucleotide having at least 90, 91, 92, 93, 94, 95, 96, 97, 98, 99, or 100% sequence identity to SEQ ID NOS: 502; 504; 506; 508; 510; 512; 514; 516; 518; 520; 522; 524; 526; 528; 530; 532; 534; 536; 538; 540; 542; 544; 546; 548; 550; 552; 554; 556; 558; 560; 562; 564; 566; 568; 570; 572; 574; 576; 578; 580; 582; 584; 586; 588; 590; 592; 594; 596; 598; or 600.

TABLE 5
Activity of the anti-Activin Antibodies as Measured by SPR.

| Clone ID | Activin E Binding KD (M) |
|---|---|
| AVE-01-D07 | 2.20E−08 |
| AVE-01-B08 | 2.90E−08 |
| AVE-02-A03 | 9.30E−09 |
| AVE-02-B04 | 3.10E−08 |
| AVE-06-B07 | 5.00E−08 |
| AVE-06-F07 | 4.38E−07 |
| AVE-06-A08 | 5.48E−09 |
| AVE-06-D08 | 3.57E−08 |
| AVE-06-G08 | 4.38E−08 |
| AVE-06-H08 | 7.69E−11 |
| AVE-06-A09 | 3.13E−07 |
| AVE-06-B09 | 9.38E−09 |
| AVE-06-D09 | 1.16E−07 |
| AVE-06-F09 | 3.83E−08 |
| AVE-06-G09 | 6.67E−09 |
| AVE-06-C10 | 1.18E−08 |
| AVE-06-G10 | 1.17E−08 |
| AVE-06-H10 | 3.75E−08 |
| AVE-06-B11 | 3.75E−08 |
| AVE-06-E11 | 3.00E−08 |
| AVE-06-H11 | 3.75E−08 |
| AVE-06-A12 | 3.75E−07 |
| AVE-06-B12 | 2.19E−08 |
| AVE-06-C12 | 2.13E−08 |
| AVE-06-E12 | 2.91E−08 |
| AVE-06-G12 | 2.20E−08 |
| AVE-07-A01 | 3.67E−08 |
| AVE-07-B01 | 9.69E−08 |
| AVE-07-C01 | 8.75E−09 |
| AVE-07-D01 | 2.91E−08 |
| AVE-07-E01 | 7.78E−09 |
| AVE-07-F01 | 2.56E−07 |
| AVE-07-C02 | 1.13E−07 |
| AVE-07-F02 | 8.44E−08 |
| AVE-07-G02 | 1.22E−08 |
| AVE-07-H03 | 2.00E−08 |
| AVE-07-D04 | 7.81E−08 |
| AVE-07-E06 | 1.84E−08 |
| AVE-07-F06 | 1.39E−07 |
| AVE-07-A07 | 1.50E−08 |
| AVE-07-E07 | 1.63E−07 |
| AVE-07-D08 | 9.20E−09 |
| AVE-07-E08 | 1.04E−08 |
| AVE-07-H08 | 1.75E−08 |
| AVE-07-C10 | 1.55E−08 |
| AVE-07-D11 | 1.04E−08 |
| AVE-07-F11 | 2.29E−08 |
| AVE-07-G11 | 8.57E−09 |
| AVE-07-F12 | 9.23E−09 |
| AVE-07-G12 | 8.15E−09 |

TABLE 6
IC₅₀ for each tested antibody.

| | IC50 (nM) | IC50 (nM) |
|---|---|---|
| Antibody | 0.2 nM Activin E | 0.02 nM Activin E |
| hIgG1 Isotype | nd | nd |
| J02 IgG1 (anti-ALK7) | 0.3139 | 0.3057 |
| AVE-06-H08 scFv-Fc | 0.2703 | 0.04758 |
| AVE-06-A08 scFv-Fc | nd | nd |
| AVE-06-G09 scFv-Fc | nd | nd |
| AVE-07-E01 scFv-Fc | nd | nd |
| AVE-07-G12 scFv-Fc | nd | nd |
| AVE-07-G11 scFv-Fc | nd | nd |
| AVE-07-C01 scFv-Fc | nd | nd |
| AVE-07-D08 scFv-Fc | nd | nd |
| No treatment | nd | nd |

### Uses of Anti-Activin E Antibodies.

### Therapeutic Anti-Activin E Antibodies.

Male C57BL/6J mice with diet-induced obesity (DIO) were used in this study. Obesity was induced over 12-16 weeks through ad libitum access to a high-fat diet (HFD, 60% kcal from fat). Water was provided ad libitum throughout the study, and mice continued on HFD for the duration of the study.

Mice were randomized into four treatment groups: Group 1: Vehicle/Vehicle, Group 2: AVE-06-H08 mouse IgG1 10 mg/kg/Vehicle, Group 3: Vehicle/Semaglutide 40 μg/kg, and Group 4: AVE-06-H08 mouse IgG1 10 mg/kg/Semaglutide 40 nmol/kg.

All dosing was administered for the duration of the study. AVE-06-H08 mouse IgG1 and vehicle were administered twice weekly via subcutaneous (SC) injection at a dose volume of 5 mL/kg. Semaglutide was administered SC once daily.

Dual-energy X-ray absorptiometry (DEXA) scans were performed at baseline (week 0) and at week 2 to assess body composition. For each DEXA scan, mice were anesthetized with ketamine/xylazine. Body weight was recorded twice weekly for all animals throughout the study.

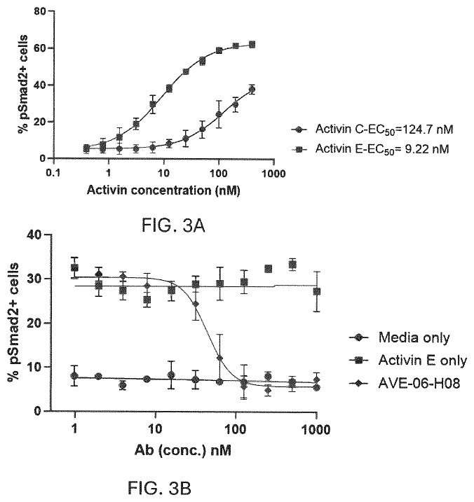

FIG. 3A is a graph that shows the establishment of Activin E signaling assay in differentiated human adipocytes, and FIG. 3B is a graph that shows the antagonism of Activin E signaling in human adipocytes by AVE-06-H08 at 100 nM Activin E using the antibodies disclosed herein.

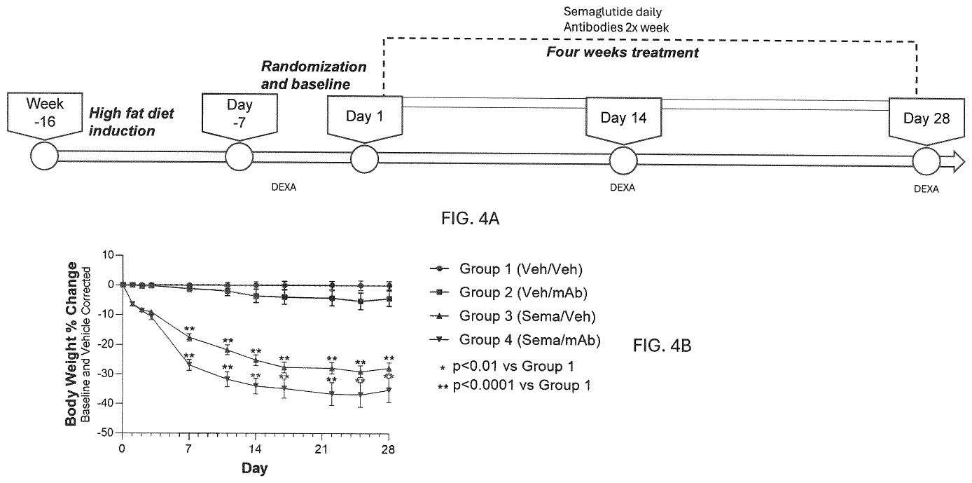

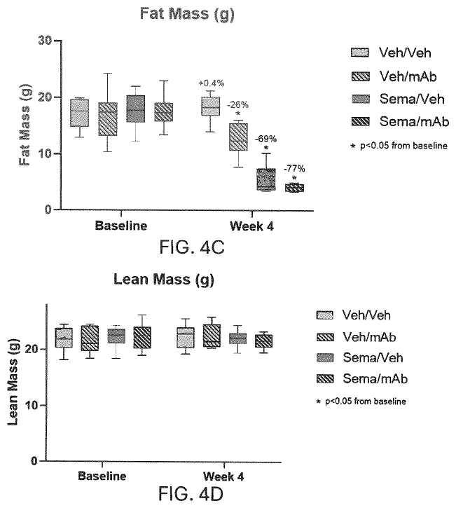

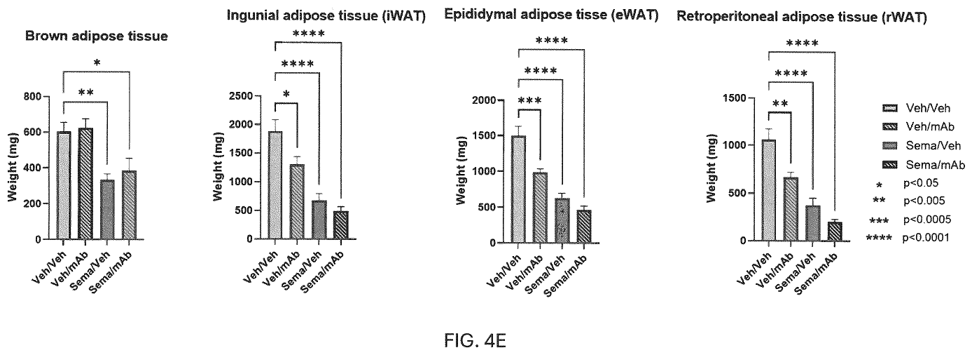

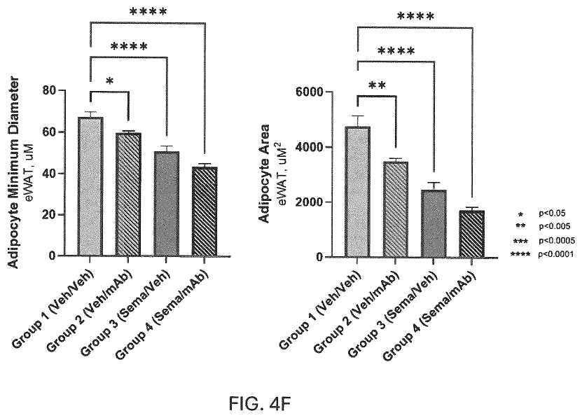

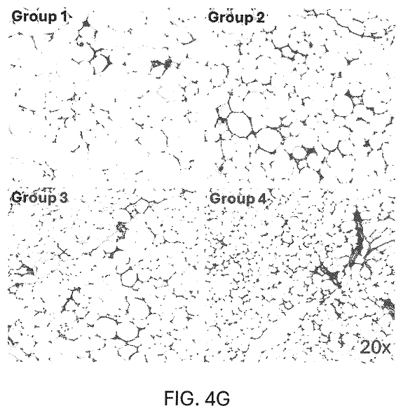

FIG. 4A to 4G show a Diet Induced Obesity (DIO) mouse model study plan and results. FIG. 4A shows the DIO mouse model study plan. FIG. 4B is a graph that shows baseline and vehicle corrected change in body weight post dose. 2-way ANOVA used for statistical measures to compare mean body weight at each timepoint to the vehicle only group. FIG. 4C is a graph that shows body composition analysis of total fat mass. Statistics calculated using 2-way ANOVA, and comparing to baseline value. FIG. 4D is a graph that shows body composition analysis of total lean mass. Statistics calculated using 2-way ANOVA, and comparing to baseline value. FIG. 4E is a graph that shows weight of various fat depots at terminal end point. One-way ANOVA used for statistical analysis. FIG. 4F is a graph that shows a histological analysis of eWAT adipose tissue from terminal endpoint, measuring adipocyte minimum diameter and area. One-way ANOVA used for statistical analysis. FIG. 4G shows representative images of adipocyte histology. One-way ANOVA used for statistical analysis. Prevention of weight and fat regain in diet-induced obesity (DIO) mouse model. Male C57BL/6J mice with diet-induced obesity were used for this study. Obesity induction occurred over 12-16 weeks; during this time mice were allowed ad libitum access to a high-fat diet (HFD, 60% kcal from fat) and water. Mice were fed HFD for the entire duration of the study.

Mice were randomized into 3 weight-matched treatment groups: Group 1: Vehicle/Vehicle, Group 2: Semaglutide 40 μg per kg/Vehicle, Group 3: Semaglutide 40 μg per kg/AVE-06-H08 mouse IgG1 10 mg per kg.

Semaglutide was administered daily for the first 14 consecutive days of the study via subcutaneous (SC) injection at a dose volume of 2 μL per gram of body weight. AVE-06-H08 was administered SC at a volume of 2.8 μL per gram of body weight twice weekly beginning at day 10 through the end of the study, for a total of 8. Semaglutide treatment was stopped after day 14 and was replaced with vehicle treatments for groups 1 and 2 on the same days as AVE-06-H08 treatment for group 3.

Body weights were collected daily for the first 14 consecutive days, and then Monday-Friday for the remainder of the study. Food intake measurements were collected twice per week for each cage and then divided by the number of mice per cage to obtain individual food intake values.

On the first day of the study (day 1), animals were fasted for 4 hours at the start of the light phase. Upon euthanasia, epididymal (bilateral), mesenteric, and inguinal (bilateral) fat depots were collected and weighed. The liver was inspected for fatty liver characteristics and weighed. The left quadriceps muscle was collected for measurement of weight.

Data were analyzed using one- or two-way ANOVA (when appropriate) using time and treatment as factors. Holm-Sidak's multiple comparisons post hoc analysis was used, when appropriate, to identify treatment group differences. All data are presented as mean±S.E.M., and p values of <0.05 were considered statistically significant.

FcRn Binding Affinity. FcRn binding affinity. FcRn binding affinity was measured using biolayer interferometry (BLI) on a ForteBio Octet Red384 instrument. Biotinylated FcRn:beta-2-microglobulin heterodimer (Acro Biosystems, Cat #FCM-H5283) was prepared at 1 μg/mL in PBS+0.01% Tween-20 pH 7.4, then captured on SA Biosensors (Sartorius, Cat #18-5019) for 180 seconds to ≈7 nm. Biosensors were then baselined in acetate buffer at pH 6 for 180 seconds followed by 5-minute sample association and dissociation phases in the same buffer. Regeneration was performed by dipping biosensors in PBS+0.01% Tween-20, pH 7.4 for 30 seconds 3 times. The resulting kinetic profiles were fit using a 1:1 binding model with the Data Analysis HT software.

TABLE 7
Affinity of tested antibodies to human FcRn, as measured
by biolayer interferometry. Alternate #1 and Alternate
#2 refer to non-Activin E binding antibodies expressed
in same IgG4 or IgG4 LS Fc as AVE-06-H08.

| | | | | Ratio |
|---|---|---|---|---|
| Clone | ka (1/Ms) | kd (1/s) | KD (M) | IgG4/IgG4 LS |
| AVE-06-H08 IgG4 | 7.02E+05 | 1.95E−03 | 2.78E−09 | 4.3 |
| AVE-06-H08 IgG4 | 4.09E+05 | 2.68E−04 | 6.54E−10 | n/a |
| [LS] | | | | |
| Alternate #1 IgG4 | 8.90E+05 | 1.27E−03 | 1.42E−09 | 2.1 |
| Alternate #1 IgG4 | 6.47E+05 | 4.40E−04 | 6.79E−10 | n/a |
| [LS] | | | | |
| Alternate #2 IgG4 | 8.34E+05 | 8.05E−04 | 9.66E−10 | 1.9 |
| Alternate #2 IgG4 | 6.34E+05 | 3.18E−04 | 5.01E−10 | n/a |
| [LS] | | | | |

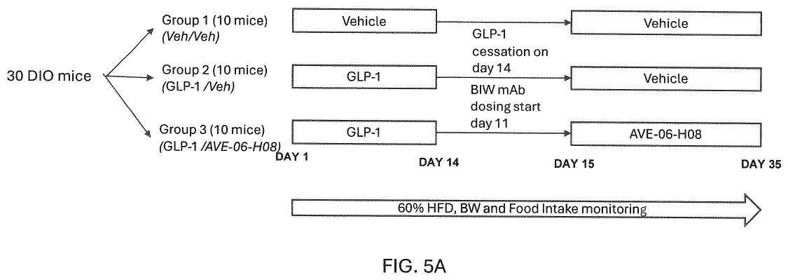

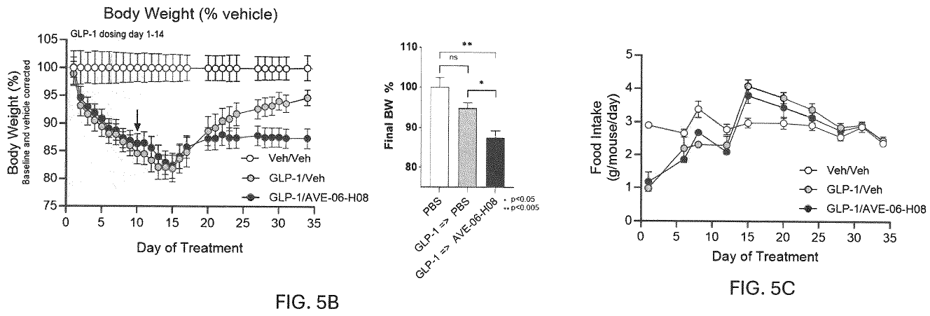

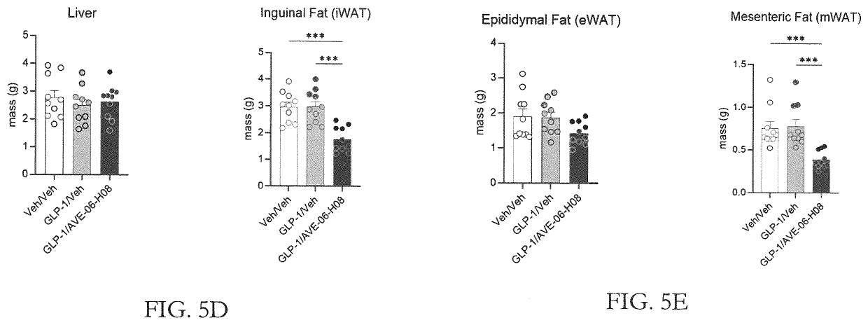

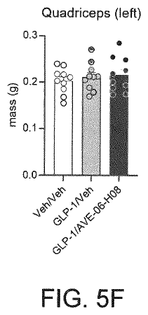

FIGS. 5A to 5F show the study design and results for weight regain prevention in DIO mice. Semaglutide dosed daily, mAbs dosed twice per week. FIG. 5B are graphs that show the baseline and vehicle-corrected body weights. Arrow indicates initiation of BIW mAb dosing. 2-way ANOVA used for statistical analysis. FIG. 5C is a graph that shows the food intake, reported on per-mouse basis. FIG. 5D is a graph that shows the terminal liver weight. FIG. 5E is a graph that shows the terminal adipose depot weights. FIG. 5F is a graph that shows the Terminal quadriceps weight.

TABLE 8
Modified Fc region AVE-06-H08_IgG4.

| Name | Amino Acid sequence | SEQ ID NO: |
|---|---|---|
| >AVE-06- | EVQLLESGGGLVQPGGSLRLSCAASGFDFSKFAMSW | 601 |
| H08_IgG4_LS_HC | VRQAPGKGLEWVSSITRGSETTYYADSVKGRFTISRD | |
| | NSKNTLYLQMNSLRAEDTAVYYCATLGLGYYYYFD | |
| | VWGQGTLVTVSSASTKGPSVFPLAPCSRSTSESTAAL | |
| | GCLVKDYFPEPVTVSWNSGALTSGVHTFPAVLQSSG | |
| | LYSLSSVVTVPSSSLGTKTYTCNVDHKPSNTKVDKR | |
| | VESKYGPPCPPCPAPEFLGGPSVFLFPPKPKDTLMISR | |
| | TPEVTCVVVDVSQEDPEVQFNWYVDGVEVHNAKTK | |
| | PREEQFNSTYRVVSVLTVLHQDWLNGKEYKCKVSN | |
| | KGLPSSIEKTISKAKGQPREPQVYTLPPSQEEMTKNQ | |
| | VSLTCLVKGFYPSDIAVEWESNGQPENNYKTTPPVLD | |
| | SDGSFFLYSRLTVDKSRWQEGNVFSCSVLHEALHSH | |
| | YTQKSLSLSLG | |
| >AVE-06- | DIQMTQSPSSLSASVGDRVTITCRASQPISSYVTWYQ | 602 |
| H08_IgG4_LS_LC | QKPGKAPKLLIYSASHLRSGVPSRFSGSGSGTDFTLTI | |
| | SSLQPEDFATYYCQQSYNAPPTFGGGTKVEIKRTVAA | |
| | PSVFIFPPSDEQLKSGTASVVCLLNNFYPREAKVQWK | |
| | VDNALQSGNSQESVTEQDSKDSTYSLSSTLTLSKADY | |
| | EKHKVYACEVTHQGLSSPVTKSFNRGEC | |

Obese Non-human Primate Pharmacokinetic Study. Male diet-induced obese (DIO) cynomolgus monkeys (Macaca fascicularis), aged 8-15 years, with a body weight >7.5 kg, BMI 35-70 kg/m², blood glucose <120 mg/dL, and blood insulin <300 μU/mL were used in this study. Animals were housed under standard conditions with ad libitum access to water and a controlled diet. Food and water intake were monitored throughout the study, and daily cage-side clinical observations were performed.

A total of 6 animals were enrolled in the pharmacokinetics study. The animals received a single intravenous (IV) dose of AVE-06-H08_IgG4_LS at 10 mg/kg.

Serum samples were collected at predefined time points following dosing for a total of 8 weeks. At each time point, 4 mL of whole blood was collected from a peripheral vein and transferred into serum separation tubes (SST). Samples were centrifuged at 2500×g for 10 minutes at 4° C., and the resulting serum was aliquoted into three 400 μL fractions and stored at −80° C. until analysis.

Serum concentrations of AVE-06-H08_IgG4_LS were determined using a Human IgG4 ELISA kit. Monkey serum samples were diluted 1:10 in assay buffer, and a standard curve was generated to quantify antibody concentrations at each time point. Antibody half-life was calculated based on a linear elimination model, using the serum concentration data obtained from the ELISA assay.

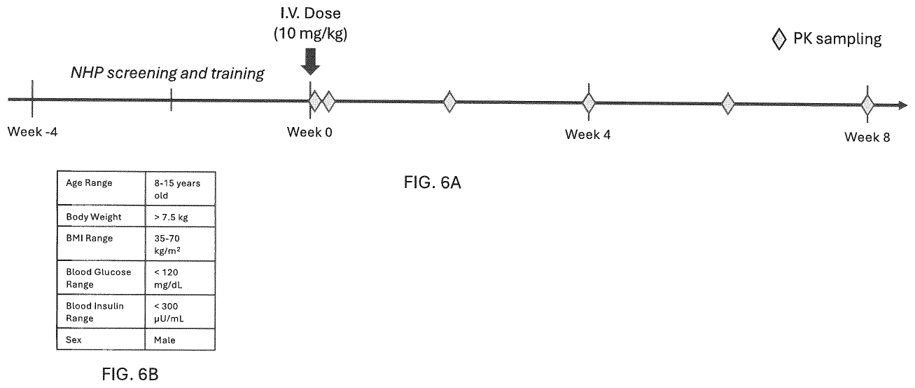

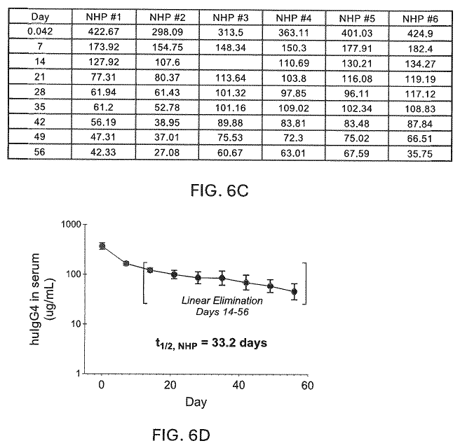

FIG. 6A shows the study design for PK study in obese mature nonhuman primates (NHPs). FIG. 6B shows the non-human primate (NHP) selection criteria. FIG. 6C shows the serum concentration of AVE-06-H08_IgG4 at various timepoints. FIG. 6D shows the average serum concentration of AVE-06-H08_IgG4 with half-life calculated using linear elimination from days 14-56.

In some embodiments, the Anti-Activin E antibodies provided herein are useful for the treatment of a disease or condition involving an immune response.

The present disclosure also includes methods of treating a subject with a therapeutic agent that treats or inhibits a metabolic disorder comprising administering or continuing to administer to the subject the therapeutic agent that treats or inhibits the metabolic disorder in a standard dosage amount, and administering to the subject an inhibitory anti-Activin E antibody, scFv, or binding fragment thereof.

The present disclosure also includes methods of treating a subject with a therapeutic agent that treats or inhibits a cardiovascular disease, wherein the subject is suffering from a cardiovascular disease, comprising administering or continuing to administer to the subject the therapeutic agent that treats or inhibits the cardiovascular disease in a standard dosage amount, and administering to the subject an inhibitory anti-Activin E antibody, scFv, or binding fragment thereof.

For subjects that have an increased risk of developing a metabolic disorder, such as type 2 diabetes, lipodystrophy, liver inflammation, fatty liver disease, hypercholesterolemia, elevated liver enzymes (such as, for example, ALT and/or AST), obesity, high blood pressure, and/or elevated triglyceride level (hypertriglyceridemia), and/or a cardiovascular disease, such as cardiomyopathy, heart failure, and high blood pressure. These subjects can be treated with an inhibitory anti-Activin E antibody, scFv, or binding fragment thereof of the present disclosure.

In certain embodiments of the disclosure, the metabolic disorder is type 2 diabetes, obesity, NASH, and/or elevated triglyceride level. In any of the embodiments disclosed herein, the metabolic disorder is type 2 diabetes. In any of the embodiments disclosed herein, the metabolic disorder is obesity. In any of the embodiments disclosed herein, the metabolic disorder is NASH. In any of the embodiments disclosed herein, the metabolic disorder is elevated triglyceride level. In any of the embodiments disclosed herein, the metabolic disorder is lipodystrophy. In any of the embodiments disclosed herein, the metabolic disorder is liver inflammation. In any of the embodiments disclosed herein, the metabolic disorder is fatty liver disease. In any of the embodiments disclosed herein, the metabolic disorder is hypercholesterolemia. In any of the embodiments disclosed herein, the metabolic disorder is elevated liver enzymes (such as, for example, ALT and/or AST).

In addition, other metabolic disorders/conditions associated with body fat distribution also include, but are not limited to: type 2 diabetes, hyperlipidemia or dyslipidemia (high or altered circulating levels of low-density lipoprotein cholesterol (LDL-C), triglycerides, very low-density lipoprotein cholesterol (VLDL-C), apolipoprotein B or other lipid fractions), obesity (particularly abdominal obesity), lipodystrophy (such as an inability to deposit fat in adipose depots regionally (partial lipodystrophy) or in the whole body (lipoatrophy)), insulin resistance or higher or altered insulin levels at fasting or during a glucose or insulin challenge, liver fat deposition or fatty liver disease and their complications (such as, for example, cirrhosis, fibrosis, or inflammation of the liver), higher or elevated or altered liver enzyme levels or other markers of liver damage, inflammation or fat deposition, higher blood pressure and/or hypertension, higher blood sugar or glucose or hyperglycemia, metabolic syndrome, coronary artery disease, and other atherosclerotic conditions, and the complications of each of the aforementioned conditions.

### Administration of Therapeutic Anti-Activin E Antibodies.

The in vivo administration of the therapeutic Anti-Activin E antibodies described herein may be carried out intravenously, intramuscularly, subcutaneously, topically, orally, transdermally, intraperitoneally, intraorbitally, intrathecally, intraventricularly, intranasally, transmucosally, through implantation, or through inhalation. Intravenous administration may be carried out via injection or infusion. In some embodiments, the Anti-Activin E antibodies of the disclosure are administered intravenously. In some embodiments, the Anti-Activin E antibodies of the disclosure are administered subcutaneously. Administration of the therapeutic Anti-Activin E antibodies may be performed with any suitable excipients, carriers, or other agents to provide suitable or improved tolerance, transfer, delivery, and the like.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. An anti-Activin E antibody or antigen binding domain thereof comprising comprises:
a heavy chain variable domain (VH) complementarity determining region 1 (CDR1), VH CDR2, and VH CDR3 comprising an amino acid sequence of any one of the following SEQ ID NOs: 1, 2, 3; 11, 12, 13; 21, 22, 23; 31, 32, 33; 41, 42, 43; 51, 52, 53; 61, 62, 63; 71, 72, 73; 81, 82, 83; 91, 92, 93; 101, 102, 103; 111, 112, 113; 121, 122, 123; 131, 132, 133; 141, 142, 143; 151, 152, 153; 161, 162, 163; 171, 172, 173; 181, 182, 183; 191, 192, 193; 201, 202, 203; 211, 212, 213; 221, 222, 223; 231, 232, 233; 241, 242, 243; 251, 252, 253; 261, 262, 263; 271, 272, 273; 281, 282, 283; 291, 292, 293; 301, 302, 303; 311, 312, 313; 321, 322, 323; 331, 332, 333; 341, 342, 343; 351, 352, 353; 361, 362, 363; 371, 372, 373; 381, 382, 383; 391, 392, 393; 401, 402, 403; 411, 412, 413; 421, 422, 423; 431, 432, 433; 441, 442, 443; 451, 452, 453; 461, 462, 463; 471, 472, 473; 481, 482, 483; or 491, 492, 493, respectively; and
a light chain variable domain (VL) CDR1, VL CDR2, and VL CDR3 comprising the amino acid sequence of any one of the following SEQ ID NOs: 4, 5, 6; 14, 15, 16; 24, 25, 26; 34, 35, 36; 44, 45, 46; 54, 55, 56; 64, 65, 66; 74, 75, 76; 84, 85, 86; 94, 95, 96; 104, 105, 106; 114, 115, 116; 124, 125, 126; 134, 135, 136; 144, 145, 146; 154, 155, 156; 164, 165, 166; 174, 175, 176; 184, 185, 186; 194, 195, 196; 204, 205, 206; 214, 215, 216; 224, 225, 226; 234, 235, 236; 244, 245, 246; 254, 255, 256; 264, 265, 266; 274, 275, 276; 284, 285, 286; 294, 295, 296; 304, 305, 306; 314, 315, 316; 324, 325, 326; 334, 335, 336; 344, 345, 346; 354, 355, 356; 364, 365, 366; 374, 375, 376; 384, 385, 386; 394, 395, 396; 404, 405, 406; 414, 415, 416; 424, 425, 426; 434, 435, 436; 444, 445, 446; 454, 455, 456; 464, 465, 466; 474, 475, 476; 484, 485, 486; or 494, 495, 496, respectively.

2. The antibody of claim 1, wherein the antibody comprises:
a VH and VL pair comprising at least 95, 96, 97, 98, 99, or 100% sequence identify to SEQ ID NOS; 7, 8; 17, 18; 27, 28; 37, 38; 47, 48; 57, 58; 67, 68; 77, 78; 87, 88; 97, 98; 107, 108; 117, 118; 127, 128; 137, 138; 147, 148; 157, 158; 167, 168; 177, 178; 187, 188; 197, 198; 207, 208; 217, 218; 227, 228; 237, 238; 247, 248; 257, 258; 267, 268; 277, 278; 287, 288; 297, 298; 307, 308; 317, 318; 327, 328; 337, 338; 347, 348; 357, 358; 367, 368; 377, 378; 387, 388; 397, 398; 407, 408; 417, 418; 427, 428; 437, 438; 447, 448; 457, 458; 467, 468; 477, 478; 487, 488; 497, 498; or 601, 602.

3. The antibody of claim 1, wherein the antibody comprises:
a heavy chain and light chain variable domains are encoded by a polynucleotide or polynucleotides comprising at least 95, 96, 97, 98, 99, or 100% sequence identify to SEQ ID NOS: 9, 10; 19, 20; 29, 30; 39, 40; 49, 50; 59, 60; 69, 70; 79, 80; 89, 90; 99, 100; 109, 110; 119, 120; 129, 130; 139, 140; 149, 150; 159, 160; 169, 170; 179, 180; 189, 190; 199, 200; 209, 210; 219, 220; 229, 230; 239, 240; 249, 250; 259, 260; 269, 270; 279, 280; 289, 290; 299, 300; 309, 310; 319, 320; 329, 330; 339, 340; 349, 350; 359, 360; 369, 370; 379, 380; 389, 390; 399, 400; 409, 410; 419, 420; 429, 430; 439, 440; 449, 450; 459, 460; 469, 470; 479, 480; 489, 490; or 499, 500.

4. The antibody of claim 1, wherein the antibody is a monoclonal antibody.

5. The antibody of claim 1, wherein the antibody is a full-length antibody.

6. The antibody of claim 1, wherein the antibody is an antibody fragment selected from F(ab′)₂, Fab, Fab′, Fv, or scFv.

7. The antibody fragment of claim 6, wherein the antibody comprises an Fc domain selected from one of the following: human IgG1, human IgG2, human IgG3, and human IgG4.

8. A method of treating a disease in a subject in need thereof, comprising administering to the subject a therapeutically effective amount of the antibody of claim 1.

9. A single chain fragment variable (scFv) Activin E antagonist comprising:
a heavy chain variable domain (VH) complementarity determining region 1 (CDR1), VH CDR2, and VH CDR3 comprising an amino acid sequence of any one of the following SEQ ID NOs: 1, 2, 3; 11, 12, 13; 21, 22, 23; 31, 32, 33; 41, 42, 43; 51, 52, 53; 61, 62, 63; 71, 72, 73; 81, 82, 83; 91, 92, 93; 101, 102, 103; 111, 112, 113; 121, 122, 123; 131, 132, 133; 141, 142, 143; 151, 152, 153; 161, 162, 163; 171, 172, 173; 181, 182, 183; 191, 192, 193; 201, 202, 203; 211, 212, 213; 221, 222, 223; 231, 232, 233; 241, 242, 243; 251, 252, 253; 261, 262, 263; 271, 272, 273; 281, 282, 283; 291, 292, 293; 301, 302, 303; 311, 312, 313; 321, 322, 323; 331, 332, 333; 341, 342, 343; 351, 352, 353; 361, 362, 363; 371, 372, 373; 381, 382, 383; 391, 392, 393; 401, 402, 403; 411, 412, 413; 421, 422, 423; 431, 432, 433; 441, 442, 443; 451, 452, 453; 461, 462, 463; 471, 472, 473; 481, 482, 483; or 491, 492, 493; and
a light chain variable domain (VL) CDR1, VL CDR2, and VL CDR3 comprising the amino acid sequence of any one of the following SEQ ID NOs: 4, 5, 6; 14, 15, 16; 24, 25, 26; 34, 35, 36; 44, 45, 46; 54, 55, 56; 64, 65, 66; 74, 75, 76; 84, 85, 86; 94, 95, 96; 104, 105, 106; 114, 115, 116; 124, 125, 126; 134, 135, 136; 144, 145, 146; 154, 155, 156; 164, 165, 166; 174, 175, 176; 184, 185, 186; 194, 195, 196; 204, 205, 206; 214, 215, 216; 224, 225, 226; 234, 235, 236; 244, 245, 246; 254, 255, 256; 264, 265, 266; 274, 275, 276; 284, 285, 286; 294, 295, 296; 304, 305, 306; 314, 315, 316; 324, 325, 326; 334, 335, 336; 344, 345, 346; 354, 355, 356; 364, 365, 366; 374, 375, 376; 384, 385, 386; 394, 395, 396; 404, 405, 406; 414, 415, 416; 424, 425, 426; 434, 435, 436; 444, 445, 446; 454, 455, 456; 464, 465, 466; 474, 475, 476; 484, 485, 486; or 494, 495, 496, respectively.

10. The scFv of claim 9, wherein the scFv comprises at least 95, 96, 97, 98, 99, or 100% amino acid sequence identify to: 501; 503; 505; 507; 509; 511; 513; 515; 517; 519; 521; 523; 525; 527; 529; 531; 533; 535; 537; 539; 541; 543; 545; 547; 549; 551; 553; 555; 557; 559; 561; 563; 565; 567; 569; 571; 573; 575; 577; 579; 581; 583; 585; 587; 589; 591; 593; 595; 597; or 599.

11. The scFv of claim 9, wherein the scFv comprises at least 95, 96, 97, 98, 99, or 100% nucleic sequence identify to: 502; 504; 506; 508; 510; 512; 514; 516; 518; 520; 522; 524; 526; 528; 530; 532; 534; 536; 538; 540; 542; 544; 546; 548; 550; 552; 554; 556; 558; 560; 562; 564; 566; 568; 570; 572; 574; 576; 578; 580; 582; 584; 586; 588; 590; 592; 594; 596; 598; or 600.

12. The scFv of claim 9, wherein the scFv is a tandem scFv.

13. A method of treating a disease in a subject in need thereof, comprising administering to the subject a therapeutically effective amount of the antibody of claim 1.

14. The method of claim 13, where the disease is selected from at least one of: a metabolic disorder, type 2 diabetes, obesity, an elevated triglyceride level, lipodystrophy, liver inflammation, fatty liver disease, hypercholesterolemia, an elevated liver enzyme, nonalcoholic steatohepatitis (NASH), a cardiovascular disease, cardiomyopathy, high blood pressure, or heart failure.

15. The method of claim 13, wherein the subject is human.

16. A nucleic acid comprising an anti-Activin E antibody or antigen binding domain thereof, comprising:
a heavy chain variable domain (VH) complementarity determining region 1 (CDR1), VH CDR2, and VH CDR3 comprising an amino acid sequence of any one of the following SEQ ID NOs: 1, 2, 3; 11, 12, 13; 21, 22, 23; 31, 32, 33; 41, 42, 43; 51, 52, 53; 61, 62, 63; 71, 72, 73; 81, 82, 83; 91, 92, 93; 101, 102, 103; 111, 112, 113; 121, 122, 123; 131, 132, 133; 141, 142, 143; 151, 152, 153; 161, 162, 163; 171, 172, 173; 181, 182, 183; 191, 192, 193; 201, 202, 203; 211, 212, 213; 221, 222, 223; 231, 232, 233; 241, 242, 243; 251, 252, 253; 261, 262, 263; 271, 272, 273; 281, 282, 283; 291, 292, 293; 301, 302, 303; 311, 312, 313; 321, 322, 323; 331, 332, 333; 341, 342, 343; 351, 352, 353; 361, 362, 363; 371, 372, 373; 381, 382, 383; 391, 392, 393; 401, 402, 403; 411, 412, 413; 421, 422, 423; 431, 432, 433; 441, 442, 443; 451, 452, 453; 461, 462, 463; 471, 472, 473; 481, 482, 483; or 491, 492, 493; and
a light chain variable domain (VL) CDR1, VL CDR2, and VL CDR3 comprising the amino acid sequence of any one of the following SEQ ID NOs: 4, 5, 6; 14, 15, 16; 24, 25, 26; 34, 35, 36; 44, 45, 46; 54, 55, 56; 64, 65, 66; 74, 75, 76; 84, 85, 86; 94, 95, 96; 104, 105, 106; 114, 115, 116; 124, 125, 126; 134, 135, 136; 144, 145, 146; 154, 155, 156; 164, 165, 166; 174, 175, 176; 184, 185, 186; 194, 195, 196; 204, 205, 206; 214, 215, 216; 224, 225, 226; 234, 235, 236; 244, 245, 246; 254, 255, 256; 264, 265, 266; 274, 275, 276; 284, 285, 286; 294, 295, 296; 304, 305, 306; 314, 315, 316; 324, 325, 326; 334, 335, 336; 344, 345, 346; 354, 355, 356; 364, 365, 366; 374, 375, 376; 384, 385, 386; 394, 395, 396; 404, 405, 406; 414, 415, 416; 424, 425, 426; 434, 435, 436; 444, 445, 446; 454, 455, 456; 464, 465, 466; 474, 475, 476; 484, 485, 486; or 494, 495, 496, respectively.

17. The nucleic acid of claim 16, wherein the antibody or antigen binding domain thereof comprises a heavy chain and light chain variable domain encoding polynucleotide having at least 95, 96, 97, 98, 99, or 100% sequence identify to SEQ ID NOS: 9, 10; 19, 20; 29, 30; 39, 40; 49, 50; 59, 60; 69, 70; 79, 80; 89, 90; 99, 100; 109, 110; 119, 120; 129, 130; 139, 140; 149, 150; 159, 160; 169, 170; 179, 180; 189, 190; 199, 200; 209, 210; 219, 220; 229, 230; 239, 240; 249, 250; 259, 260; 269, 270; 279, 280; 289, 290; 299, 300; 309, 310; 319, 320; 329, 330; 339, 340; 349, 350; 359, 360; 369, 370; 379, 380; 389, 390; 399, 400; 409, 410; 419, 420; 429, 430; 439, 440; 449, 450; 459, 460; 469, 470; 479, 480; 489, 490; 499, 500; or 601, 602.

18. The nucleic acid of claim 16, wherein the antibody is a monoclonal, bispecific, multivalent, multi-specific, diabody, chimeric, scFv antibody, or domain thereof.

19. The nucleic acid of claim 18, wherein the scFv encodes a polypeptide that comprises at least 95, 96, 97, 98, 99, or 100% amino acid sequence identify to: 501; 503; 505; 507; 509; 511; 513; 515; 517; 519; 521; 523; 525; 527; 529; 531; 533; 535; 537; 539; 541; 543; 545; 547; 549; 551; 553; 555; 557; 559; 561; 563; 565; 567; 569; 571; 573; 575; 577; 579; 581; 583; 585; 587; 589; 591; 593; 595; 597; or 599.

20. The nucleic acid of claim 18, wherein the scFv comprises at least 90, 91, 92, 93, 94, 95, 96, 97, 98, 99, or 100% nucleic sequence identify to: 502; 504; 506; 508; 510; 512; 514; 516; 518; 520; 522; 524; 526; 528; 530; 532; 534; 536; 538; 540; 542; 544; 546; 548; 550; 552; 554; 556; 558; 560; 562; 564; 566; 568; 570; 572; 574; 576; 578; 580; 582; 584; 586; 588; 590; 592; 594; 596; 598; or 600.

21. The nucleic acid of claim 16, wherein an antibody binding domain is fused to an Fc domain of any one of the following: human IgG1, human IgG2, human IgG3, and human IgG4.

22. The nucleic acid of claim 16, wherein the nucleic acid sequence is optimized for expression in a bacterial, fungal, mammalian, insect, or plant cell.

23. A vector comprising the nucleic acid of claim 16.

24. A host cell comprising nucleic acid the vector of claim 23.

25. A method of treating a subject having or at risk of developing a metabolic disorder, type 2 diabetes, obesity, an elevated triglyceride level, lipodystrophy, liver inflammation, fatty liver disease, hypercholesterolemia, an elevated liver enzyme, nonalcoholic steatohepatitis (NASH), a cardiovascular disease, cardiomyopathy, high blood pressure, and/or heart failure, the method comprising administering an anti-Inhibin E antibody administering an antibody, scFv, or antigen binding domain thereof.

26. The method of claim 25, wherein the anti-Activin antibody or binding domain thereof comprises a variable heavy chain and light chain nucleic acid sequence having at least 95, 96, 97, 98, 99, or 100% sequence identify to SEQ ID NOS: 9, 10; 19, 20; 29, 30; 39, 40; 49, 50; 59, 60; 69, 70; 79, 80; 89, 90; 99, 100; 109, 110; 119, 120; 129, 130; 139, 140; 149, 150; 159, 160; 169, 170; 179, 180; 189, 190; 199, 200; 209, 210; 219, 220; 229, 230; 239, 240; 249, 250; 259, 260; 269, 270; 279, 280; 289, 290; 299, 300; 309, 310; 319, 320; 329, 330; 339, 340; 349, 350; 359, 360; 369, 370; 379, 380; 389, 390; 399, 400; 409, 410; 419, 420; 429, 430; 439, 440; 449, 450; 459, 460; 469, 470; 479, 480; 489, 490; 499, 500; or 601, 602.

27. The method of claim 25, wherein the anti-Activin antibody or binding domain thereof comprises an scFv comprising at least 95, 96, 97, 98, 99, or 100% amino acid sequence identify to: 501; 503; 505; 507; 509; 511; 513; 515; 517; 519; 521; 523; 525; 527; 529; 531; 533; 535; 537; 539; 541; 543; 545; 547; 549; 551; 553; 555; 557; 559; 561; 563; 565; 567; 569; 571; 573; 575; 577; 579; 581; 583; 585; 587; 589; 591; 593; 595; 597; or 599.

28. The method of claim 25, wherein when the subject is administered a therapeutic agent that treats or inhibits metabolic disorders or cardiovascular diseases in a standard dosage amount.

29. The method of claim 25, wherein when the subject is administered a therapeutic agent that treats or inhibits metabolic disorders or cardiovascular diseases that is the same as or lower than a standard dosage amount.

30. A method of treating a subject with a therapeutic agent that treats or inhibits a metabolic disorder, type 2 diabetes, obesity, an elevated triglyceride level, lipodystrophy, liver inflammation, fatty liver disease, hypercholesterolemia, an elevated liver enzyme, nonalcoholic steatohepatitis (NASH), a cardiovascular disease, cardiomyopathy, high blood pressure, or heart failure, the method comprising administering an antibody or antigen binding domain thereof to the subject.

31. The method of claim 30, wherein the subject is administered or continued to be administered a therapeutic agent that treats or inhibits the metabolic disorder, type 2 diabetes, obesity, an elevated triglyceride level, lipodystrophy, liver inflammation, fatty liver disease, hypercholesterolemia, an elevated liver enzyme, nonalcoholic steatohepatitis (NASH), a cardiovascular disease, cardiomyopathy, high blood pressure, or heart failure in a standard dosage amount; or the subject is administered or continued to be administered the therapeutic agent that treats or inhibits the metabolic disorder, type 2 diabetes, obesity, an elevated triglyceride level, lipodystrophy, liver inflammation, fatty liver disease, hypercholesterolemia, an elevated liver enzyme, nonalcoholic steatohepatitis (NASH), a cardiovascular disease, cardiomyopathy, high blood pressure, or heart failure in an amount that is the same as or lower than a standard dosage amount.

32. The method of claim 30, wherein the metabolic disorder is selected from at least one of:
type 2 diabetes, and the therapeutic agent is chosen from metformin, insulin, glyburide, glipizide, glimepiride, repaglinide, nateglinide, thiazolidinediones, rosiglitazone, pioglitazone, sitagliptin, saxagliptin, linagliptin, exenatide, liraglutide, semaglutide, canagliflozin, dapagliflozin, and empagliflozin, or any combination thereof;
obesity, and the therapeutic agent is chosen from orlistat, phentermine, topiramate, bupropion, naltrexone, and liraglutide, or any combination thereof:
elevated triglyceride, and the therapeutic agent is chosen from rosuvastatin, simvastatin, atorvastatin, fenofibrate, gemfibrozil, fenofibric acid, niacin, and an omega-3 fatty acid, or any combination thereof,
lipodystrophy, and the therapeutic agent is chosen from tesamorelin, metformin, poly-L-lactic acid, calcium hydroxyapatite, polymethylmethacrylate, bovine collagens, human collagens, silicone, and hyaluronic acid, or any combination thereof;
liver inflammation, and the therapeutic agent is a hepatitis therapeutic or a hepatitis vaccine;
fatty liver disease include, and the subject is administered bariatric surgery and/or dietary intervention;
hypercholesterolemia, and the therapeutic agent is chosen from: atorvastatin, fluvastatin, lovastatin, pitavastatin, pravastatin, rosuvastatin calcium, simvastatin, cholestyramine, colesevelam, and colestipol, alirocumab, evolocumab, niaspan, niacor, fenofibrate, gemfibrozil, and bempedoic, or any combination thereof;
an elevated liver enzyme, and the therapeutic agent is chosen from coffee, folic acid, potassium, vitamin B6, a statin, and fiber, or any combination thereof; or
nonalcoholic steatohepatitis (NASH) and the therapeutic agent is obeticholic acid, Selonsertib, Elafibranor, Cenicriviroc, GR MD 02, MGL 3196, IMM124E, arachidyl amido cholanoic acid, GS0976, Emricasan, Volixibat, NGM282, GS9674, Tropifexor, MN 001, LMB763, BI 1467335, MSDC 0602, PF 05221304, DF102, Saroglitazar, BMS986036, Lanifibranor, Semaglutide, Nitazoxanide, GRI 0621, EYP001, VK2809, Nalmefene, LIK066, MT 3995, Elobixibat, Namodenoson, Foralumab, SAR425899, Sotagliflozin, EDP 305, Isosabutate, Gemcabene, TERN 101, KBP 042, PF 06865571, DUR928, PF 06835919, NGM313, BMS 986171, Namacizumab, CER 209, ND L02 s0201, RTU 1096, DRX 065, IONIS DGAT2Rx, INT 767, NC 001, Seladepar, PXL770, TERN 201, NV556, AZD2693, SP 1373, VK0214, Hepastem, TGFTX4, RLBN1127, GKT 137831, RYI 018, CB4209-CB4211, and JH 0920.

33. The method of claim 30, wherein the cardiovascular disease is selected from at least one of:
high blood pressure, and the therapeutic agent is chosen from chlorthalidone, chlorothiazide, hydrochlorothiazide, indapamide, metolazone, acebutolol, atenolol, betaxolol, bisoprolol fumarate, carteolol hydrochloride, metoprolol tartrate, metoprolol succinate, nadolol, benazepril hydrochloride, captopril, enalapril maleate, fosinopril sodium, lisinopril, moexipril, perindopril, quinapril hydrochloride, ramipril, trandolapril, candesartan, eprosartan mesylate, irbesartan, losartan potassium, telmisartan, valsartan, amlodipine besylate, bepridil, diltiazem hydrochloride, felodipine, isradipine, nicardipine, nifedipine, nisoldipine, verapamil hydrochloride, doxazosin mesylate, prazosin hydrochloride, terazosin hydrochloride, methyldopa, carvedilol labetalol hydrochloride, alpha methyldopa, clonidine hydrochloride, guanabenz acetate, guanfacine hydrochloride, guanadrel, guanethidine monosulfate, reserpine, hydralazine hydrochloride, and minoxidil, or any combination thereof;
cardiomyopathy, and the therapeutic agent is an ACE inhibitor, an angiotensin II receptor blocker, a beta blocker, a calcium channel blocker, digoxin, an antiarrhythmic, an aldosterone blocker, a diuretic, an anticoagulant, a blood thinner, and a corticosteroid; or
heart failure, and the therapeutic agent is an ACE inhibitor, an angiotensin-2 receptor blocker, a beta blocker, a mineralocorticoid receptor antagonist, a diuretic, ivabradine, sacubitril valsartan, hydralazine with nitrate, and digoxin.


SEQUENCE LISTING (602 sequences)

SEQ ID NO: 1 - protein, 8 aa, synthetic construct
GFAFNNYA

SEQ ID NO: 2 - protein, 8 aa, synthetic construct
ISGSGTSK

SEQ ID NO: 3 - protein, 17 aa, synthetic construct
AKPRPGSIFWGAGPFDY

SEQ ID NO: 4 - protein, 6 aa, synthetic construct
QSISSY

SEQ ID NO: 5 - skipped

SEQ ID NO: 6 - protein, 8 aa, synthetic construct
QQSYGSPT

SEQ ID NO: 7 - protein, 124 aa, synthetic construct
EVQLLESGGGLVQPGGSLRLSCAASGFAFNNYAMTWVRQAPGKGLEWVSVISGSGTSKYYADSVKGRFTISRDNSKNTLYLQMNSLRAEDTAVYYCAKPRPGSIFWGAGPFDYWGQGTLVTVSS

SEQ ID NO: 8 - protein, 106 aa, synthetic construct
DIQMTQSPSSLSASVGDRVTITCRASQSISSYLHWYQQKPGKAPKLLIYAASSLQSGVPSRFSGSGSGTDFTLTISSLQPEDFATYYCQQSYGSPTFGGGTKVEIK

SEQ ID NO: 9 - DNA, 249 bp, synthetic construct
  source 1..249: mol_type = other DNA
gaagtgcagctcctagaatccggcggtgggctggttcagcctggagggtcccttagactgtcttgtgccgcgagtggcttcgcctttaacaattatgcaatgacctgggtgaggcaggcacccggcaagggactggagtgggtaagcgtcatcagcgggagcggcacttctaagtactacgccgattctgttaaaggtcgattcaccatatcacgggacaactccaagaacactttgtatctgcaaatg

SEQ ID NO: 10 - DNA, 318 bp, synthetic construct
  source 1..318: mol_type = other DNA
gacatccagatgacacagtctccttcttctctgtccgctagtgtgggcgacagagtgacgatcacctgtagggcgagtcaaagcatttcatcctacttgcactggtaccagcagaagcccggaaaagcacctaaactgttaatttacgccgccagttccctccaaagcggtgtcccatctcggttttcggggtcagggagcggcactgatttcacccttaccatctcatccctgcagcccgaagattttgctacatattattgccagcagagctatgggagcccaacattcggtggaggcactaaggttgagataaag

SEQ ID NO: 11 - protein, 8 aa, synthetic construct
GFTFGASA

SEQ ID NO: 12 - protein, 8 aa, synthetic construct
ISGLGRTT

SEQ ID NO: 13 - protein, 14 aa, synthetic construct
ARVAPGAYAYAMDY

SEQ ID NO: 14 - protein, 6 aa, synthetic construct
QNIGHY

SEQ ID NO: 15 - skipped

SEQ ID NO: 16 - protein, 9 aa, synthetic construct
QQSYSTPPT

SEQ ID NO: 17 - protein, 121 aa, synthetic construct
EVQLLESGGGLVQPGGSLRLSCAASGFTFGASAMTWVRQAPGKGLEWVSGISGLGRTTDYADSVKGRFTISRDNSKNTLYLQMNSLRAEDTAVYYCARVAPGAYAYAMDYWGQGTLVTVSS

SEQ ID NO: 18 - protein, 107 aa, synthetic construct
DIQMTQSPSSLSASVGDRVTITCRASQNIGHYLNWYQQKPGKAPKLLIYDASRLQVGVPSRFSGSGSGTDFTLTISSLQPEDFATYYCQQSYSTPPTFGGGTKVEIK

SEQ ID NO: 19 - DNA, 363 bp, synthetic construct
  source 1..363: mol_type = other DNA
gaagtgcaattgcttgagagtggcggcggcctggttcagcctgggggctccctgagacttagctgtgctgcctctggcttcaccttcggagcttcagcaatgacctgggtgcgtcaggctccaggtaagggcctggagtgggtgtcaggtatcagcggactagggcggacaaccgattacgctgatagcgtcaaagggcgctttacgatttcgagggacaactctaagaacactctgtatctccagatgaatagtttaagggcagaagacacagccgtgtactactgcgcccgagtagcccccggagcgtatgcctatgcaatggactactgggggcagggaactctcgtcacagtttcctcc

SEQ ID NO: 20 - DNA, 321 bp, synthetic construct
  source 1..321: mol_type = other DNA
gacatacagatgacacagtctccttcctcacttagcgcgtcggttggcgaccgcgtgaccatcacatgtagagcatcacagaacattggtcactacctgaattggtaccagcagaaacccggaaaggccccaaaattattgatctatgacgcctccaggctgcaagtgggcgtaccatctcggttctccggtagcgggagcggcaccgattttacactgactatcagttctctccagcccgaagattttgctacgtattattgccaacagagttacagcaccccgcctaccttcgggggagggactaaggtcgagattaag

SEQ ID NO: 21 - protein, 8 aa, synthetic construct
GFNFRSYV

SEQ ID NO: 22 - protein, 8 aa, synthetic construct
ISDVGRRT

SEQ ID NO: 23 - protein, 14 aa, synthetic construct
AKAEILGDYAYMDY

SEQ ID NO: 24 - protein, 6 aa, synthetic construct
ESIGNY

SEQ ID NO: 25 - skipped

SEQ ID NO: 26 - protein, 8 aa, synthetic construct
QQSYSTPT

SEQ ID NO: 27 - protein, 121 aa, synthetic construct
EVQLLESGGGLVQPGGSLRLSCAASGFNFRSYVMNWVRQAPGKGLEWVSAISDVGRRTYYADSVKGRFTISRDNSKNTLYLQMNSLRAEDTAVYYCAKAEILGDYAYMDYWGQGTLVTVSS

SEQ ID NO: 28 - protein, 106 aa, synthetic construct
DIQMTQSPSSLSASVGDRVTITCRASESIGNYLSWYQQKPGKAPKLLIYAASRLQRGVPSRFSGSGSGTDFTLTISSLQPEDFATYYCQQSYSTPTFGGGTKVEIK

SEQ ID NO: 29 - DNA, 363 bp, synthetic construct
  source 1..363: mol_type = other DNA
gaggtccagctgctggagtcaggcggtggccttgttcagcccggcgggtctttgcggctgtcctgcgccgccagcggattcaacttcagaagctacgttatgaactgggtgcgccaggcccctggcaagggcttagaatgggtgtccgctatctccgatgtcggacgtaggacttactatgcagactctgtgaaagggagatttaccattagtcgagataactcaaaaaatacactgtatctccagatgaatagtttgagggccgaggacaccgcagtatattactgtgctaaggctgaaatactcggtgactatgcgtacatggattactgggggcaaggaacactagtgacggtgagctcg

SEQ ID NO: 30 - DNA, 318 bp, synthetic construct
  source 1..318: mol_type = other DNA
gacatccagatgacccagagtccctcatctctgagtgcatcagtgggggaccgcgtgactattacctgcagagcttctgagtccataggtaactatctgtcctggtaccagcaaaaacctggcaaggcgcccaagttgctgatttacgccgcttcgcgattacagaggggcgtcccatcccggtttagcgggtcagggtccggtactgatttcacacttacaatcagcagcctccaaccagaagattttgccacctattactgtcagcagagctattctacacctaccttcggaggaggcacgaaggttgagatcaaa

SEQ ID NO: 31 - protein, 8 aa, synthetic construct
GFAFSAYA

SEQ ID NO: 32 - protein, 8 aa, synthetic construct
ITESGAAT

SEQ ID NO: 33 - protein, 12 aa, synthetic construct
ARGLLASYTGDV

SEQ ID NO: 34 - protein, 6 aa, synthetic construct
QSISSY

SEQ ID NO: 35 - skipped

SEQ ID NO: 36 - protein, 9 aa, synthetic construct
QQRDNAPWT

SEQ ID NO: 37 - protein, 119 aa, synthetic construct
EVQLLESGGGLVQPGGSLRLSCAASGFAFSAYAMNWVRQAPGKGLEWVSSITESGAATYYADSVKGRFTISRDNSKNTLYLQMNSLRAEDTAVYYCARGLLASYTGDVWGQGTLVTVSS

SEQ ID NO: 38 - protein, 107 aa, synthetic construct
DIQMTQSPSSLSASVGDRVTITCRASQSISSYLNWYQQKPGKAPKLLIYATSTLQSGVPSRFSGSGSGTDFTLTISSLQPEDFATYYCQQRDNAPWTFGGGTKVEIK

SEQ ID NO: 39 - DNA, 357 bp, synthetic construct
  source 1..357: mol_type = other DNA
gaagtgcagctcctagagtcgggcggcgggctggtgcagcccggtggctccctccggctgagctgcgccgcgagcggtttcgctttcagtgcctacgccatgaactgggttcgtcaagcccctggaaaaggcctggagtgggtatcaagcatcacggagtctggggctgctacatattatgcagattctgtgaagggaagatttactatttcccgcgacaatagcaagaataccctgtatctgcagatgaacagtttgcgagcagaagacaccgccgtctactactgtgcaagggggcttcttgcttcatacacaggggatgtgtggggccagggaaccttagtcactgtttcctct

SEQ ID NO: 40 - DNA, 321 bp, synthetic construct
  source 1..321: mol_type = other DNA
gacattcagatgacacaaagcccctccagcctgagcgcatcggtgggggaccgcgtcaccataacttgtcgggccagtcagtctatcagctcatatctgaactggtaccagcagaagcctggcaaagctccaaagctgcttatctacgctacttccactttacagtccggtgttccatccaggttctcaggatctggctctggtacagatttcaccttgaccatttcaagtctccagcctgaagattttgccacgtactattgccaacagagagacaatgcgccctggacatttgggggcggaaccaaagtggagatcaag

SEQ ID NO: 41 - protein, 8 aa, synthetic construct
GFDFSSFA

SEQ ID NO: 42 - protein, 8 aa, synthetic construct
ITGSGGTI

SEQ ID NO: 43 - protein, 12 aa, synthetic construct
ASASSYYEPGDP

SEQ ID NO: 44 - protein, 6 aa, synthetic construct
QAIKNY

SEQ ID NO: 45 - skipped

SEQ ID NO: 46 - protein, 9 aa, synthetic construct
QQTYSTPLT

SEQ ID NO: 47 - protein, 119 aa, synthetic construct
EVQLLESGGGLVQPGGSLRLSCAASGFDFSSFAMTWVRQAPGKGLEWVSHITGSGGTIYYADSVKGRFTISRDNSKNTLYLQMNSLRAEDTAVYYCASASSYYEPGDPWGQGTLVTVSS

SEQ ID NO: 48 - protein, 107 aa, synthetic construct
DIQMTQSPSSLSASVGDRVTITCRASQAIKNYLNWYQQKPGKAPKLLIYAGSTLQSGVPSRFSGSGSGTDFTLTISSLQPEDFATYYCQQTYSTPLTFGGGTKVEIK

SEQ ID NO: 49 - DNA, 357 bp, synthetic construct
  source 1..357: mol_type = other DNA
gaagtgcagctcctagagtcgggtggcgggctggtgcagccgggcggctccctgcgcctgtcttgcgcggctagtggatttgacttttctagcttcgcaatgacctgggtcagacaggccccagggaagggattggagtgggtgtcccacataacaggttcaggagggacaatctattacgccgatagcgttaagggacggttcactattagcagggataattcaaaaaacacgctttacttacaaatgaacagtctgcgagccgaggacaccgcagtatactattgtgcttccgccagctcctattacgaacccggggacccttggggccagggcactctcgtcaccgtgtcatct

SEQ ID NO: 50 - DNA, 321 bp, synthetic construct
  source 1..321: mol_type = other DNA
gacatccagatgactcagagtcccagctcactctccgcttcagtcggtgatcgggttactataacatgtagagccagccaagctattaagaattacctgaactggtatcagcaaaaacccgggaaggcacctaaactgttgatttatgcggggtccacactccagagcggggtgcctagtaggttctctggctccggatctggcactgactttaccttaaccatctcttcgcttcagccagaagattttgccacatactattgccagcagacatacagcacgccactgaccttcggtggcggaaccaaggtggagatcaaa

SEQ ID NO: 51 - protein, 8 aa, synthetic construct
GFPFSSHG

SEQ ID NO: 52 - protein, 8 aa, synthetic construct
ITGSGRST

SEQ ID NO: 53 - protein, 13 aa, synthetic construct
ASDYRDAPGTFDV

SEQ ID NO: 54 - protein, 6 aa, synthetic construct
QSISSY

SEQ ID NO: 55 - skipped

SEQ ID NO: 56 - protein, 8 aa, synthetic construct
QQSYSTPV

SEQ ID NO: 57 - protein, 120 aa, synthetic construct
EVQLLESGGGLVQPGGSLRLSCAASGFPFSSHGMSWVRQAPGKGLEWVSTITGSGRSTYYADSVKGRFTISRDNSKNTLYLQMNSLRAEDTAVYYCASDYRDAPGTFDVWGQGTLVTVSS

SEQ ID NO: 58 - protein, 106 aa, synthetic construct
DIQMTQSPSSLSASVGDRVTITCRASQSISSYLNWYQQKPGKAPKLLIYDASHLQSGVPSRFSGSGSGTDFTLTISSLQPEDFATYYCQQSYSTPVFGGGTKVEIK

SEQ ID NO: 59 - DNA, 360 bp, synthetic construct
  source 1..360: mol_type = other DNA
gaagttcagctgttagaaagcggggggggcctggtgcagcctggcggctcactgagattgtcctgcgctgcatccggattcccgttttcctcacacggaatgagctgggttaggcaggctcccgggaagggcctagagtgggtcagtaccattacaggaagcggtaggagcacgtactacgccgattcagtaaagggccgattcacaatctcccgtgacaactctaaaaatactctttatctccagatgaacagtctgcgggcagaggatacagccgtgtattattgtgcgtcggactaccgcgatgccccaggaacctttgacgtgtggggtcaagggactctcgtcaccgtgtcttct

SEQ ID NO: 60 - DNA, 318 bp, synthetic construct
  source 1..318: mol_type = other DNA
gacatccaaatgacccagtctccctcctcgctgagcgcaagcgtaggggaccgggtcaccataacgtgtagagcttctcagtcaatctcctcatacttgaactggtatcagcagaagcccggcaaagcccctaagctcctgatttacgatgccagccacctacaaagcggagttccatccaggtttagtggctctgggtcaggaacagatttcacactgacaatcagttctcttcagcctgaagacttcgcgacttactattgccagcagtcctatagtaccccagtgtttggtgggggcactaaagtggagattaag

SEQ ID NO: 61 - protein, 8 aa, synthetic construct
GFPFASHA

SEQ ID NO: 62 - protein, 8 aa, synthetic construct
ITGSGRST

SEQ ID NO: 63 - protein, 13 aa, synthetic construct
ASDYRDAPGTFDV

SEQ ID NO: 64 - protein, 6 aa, synthetic construct
QSISSY

SEQ ID NO: 65 - skipped

SEQ ID NO: 66 - protein, 8 aa, synthetic construct
QQSYSTPV

SEQ ID NO: 67 - protein, 120 aa, synthetic construct
EVQLLESGGGLVQPGGSLRLSCAASGFPFASHAMTWVRQAPGKGLEWVSTITGSGRSTYYADSVKGRFTISRDNSKNTLYLQMNSLRAEDTAVYYCASDYRDAPGTFDVWGQGTLVTVSS

SEQ ID NO: 68 - protein, 106 aa, synthetic construct; identical to SEQ ID NO: 58

SEQ ID NO: 69 - DNA, 360 bp, synthetic construct
  source 1..360: mol_type = other DNA
gaggtccagctgttggaatccgggggaggcttagtgcaaccgggcggctccctgcgactgagctgtgcagcctcgggattcccctttgcttctcacgcaatgacctgggttcgtcaggccccagggaaaggcctggagtgggtttctactatcaccggttcaggtcggagcacatattacgctgacagcgtgaaggggagatttaccatttcacgcgacaatagcaagaacactctctatcttcagatgaacagtctcagggccgaagatacagccgtctattactgcgctagtgactacagggatgcgcctggaacgttcgatgtgtggggccaggggacactagtgaccgtatcctct

SEQ ID NO: 70 - DNA, 318 bp, synthetic construct
  source 1..318: mol_type = other DNA
gatatacagatgactcaatctcccagcagtctgtccgcatccgtcggagatagggtaacaattacctgcagagcgtcccaatcgatcagttcatatcttaactggtaccagcagaagcctggcaaagctccaaagctgttaatttacgacgcctctcacctccagagtggcgttccttctcggttttctggcagcggttccgggaccgacttcacgttgacaatctcaagcctgcagccagaagactttgccacctactattgtcagcagtcatatagcactcccgtgttcgggggtggaacaaaagtggagatcaag

SEQ ID NO: 71 - protein, 8 aa, synthetic construct
GFDFSKFA

SEQ ID NO: 72 - protein, 8 aa, synthetic construct
ISGSGGTI

SEQ ID NO: 73 - protein, 12 aa, synthetic construct
ASASSYYEPGDP

SEQ ID NO: 74 - protein, 6 aa, synthetic construct
QAIKNY

SEQ ID NO: 75 - skipped

SEQ ID NO: 76 - protein, 8 aa, synthetic construct
QQSYSTPV

SEQ ID NO: 77 - protein, 119 aa, synthetic construct
EVQLLESGGGLVQPGGSLRLSCAASGFDFSKFAMSWVRQAPGKGLEWVSHISGSGGTIYYADSVKGRFTISRDNSKNTLYLQMNSLRAEDTAVYYCASASSYYEPGDPWGQGTLVTVSS

SEQ ID NO: 78 - protein, 106 aa, synthetic construct
DIQMTQSPSSLSASVGDRVTITCRASQAIKNYLNWYQQKPGKAPKLLIYDASHPQSGVPSRFRGSGSGTDFPFTISSLQPEEFATYYCQQSYSTPVFGGGTKVEIK

SEQ ID NO: 79 - DNA, 357 bp, synthetic construct
  source 1..357: mol_type = other DNA
gaagtgcaactgctggaatcgggcggtggactggtccagcctggaggctctttgaggctttcctgtgccgcctcagggtttgatttcagtaaattcgctatgtcctgggttcgccaggctcccgggaaaggacttgagtgggtgtcccacatcagcggtagcggcggaacgatttattacgcagactcagtaaagggcagatttactatatctcgggacaattcaaagaacacactctacctgcagatgaacagtttacgagcagaggataccgcggtctattactgcgccagtgccagctcctactatgagccaggcgacccgtgggggcaggggacactcgtgaccgtgagctct

SEQ ID NO: 80 - DNA, 318 bp, synthetic construct
  source 1..318: mol_type = other DNA
gatatacagatgactcaatccccttcttctctgtccgcgtctgttggggacagggtgacaatcacctgtcgggcttcgcaggctattaaaaattatcttaactggtatcagcagaagcccggtaaggccccaaagttgctcatctatgatgccagccacccacagtcaggagtacccagtcgcttcagaggctccggaagcggcaccgacttccctttcaccatctcaagcctgcagccggaggaatttgcaacatactactgccagcaaagctacagtacgcccgtctttggcggtgggactaaagtggagattaaa

SEQ ID NO: 81 - protein, 8 aa, synthetic construct
GFDFSKFA

SEQ ID NO: 82 - protein, 8 aa, synthetic construct
ISGSGGTI

SEQ ID NO: 83 - protein, 12 aa, synthetic construct
ASDSSYYEPGDP

SEQ ID NO: 84 - protein, 6 aa, synthetic construct
QAIKNY

SEQ ID NO: 85 - skipped

SEQ ID NO: 86 - protein, 9 aa, synthetic construct
QQTYSTPLT

SEQ ID NO: 87 - protein, 119 aa, synthetic construct
EVQLLESGGGLVQPGGSLRLSCAASGFDFSKFAMSWVRQAPGKGLEWVSHISGSGGTIYYADSVKGRFTISRDNSKNTLYLQMNSLRAEDTAVYYCASDSSYYEPGDPWGQGTLVTVSS

SEQ ID NO: 88 - protein, 107 aa, synthetic construct
DIQMTQSPSSLSASVGDRVTITCRAGQAIKNYLNWYQQKPGKAPKLLIYAGSPLQSGVPSRFSGSGSGTDFTLTISSLQPEDFATYYCQQTYSTPLTFGGGTKVEIK

SEQ ID NO: 89 - DNA, 357 bp, synthetic construct
  source 1..357: mol_type = other DNA
gaggtacaactcctagagagtggcggaggccttgtgcagcccggaggatcgctgcgactgtcatgcgctgcatccggctttgatttctctaaatttgccatgagctgggtgagacaggccccgggtaagggcctggaatgggtgtctcacatatcagggagcgggggaacaatttactatgccgactcagttaaggggcgcttcaccatcagcagggacaattccaaaaacaccctgtatttacagatgaacagtttgcgggcggaggatacggcagtctactattgtgcttccgacagcagttactacgaaccaggtgatccttggggccaggggactctcgtgacagtctcctct

SEQ ID NO: 90 - DNA, 321 bp, synthetic construct
  source 1..321: mol_type = other DNA
gacatccagatgacacagagcccgtcttcactcagtgcaagtgttggggacagagtgaccattacatgcagggccggacaggctatcaaaaactatctgaattggtaccaacaaaaacctggcaaggcgcccaaattgctcatctatgccggttcaccactgcagagcggtgtgccatcgcggtttagcggatctggctccggcactgatttcacccttaccatttcctctttacagcccgaggatttcgctacctactattgtcagcagacttactccacgcctctgacatttggaggggggactaaggtcgaaataaag

SEQ ID NO: 91 - protein, 8 aa, synthetic construct
GFDFSKFA

SEQ ID NO: 92 - protein, 8 aa, synthetic construct
ITRGSETT

SEQ ID NO: 93 - protein, 13 aa, synthetic construct
ATLGLGYYYYFDV

SEQ ID NO: 94 - protein, 6 aa, synthetic construct
QPISSY

SEQ ID NO: 95 - skipped

SEQ ID NO: 96 - protein, 9 aa, synthetic construct
QQSYNAPPT

SEQ ID NO: 97 - protein, 120 aa, synthetic construct
EVQLLESGGGLVQPGGSLRLSCAASGFDFSKFAMSWVRQAPGKGLEWVSSITRGSETTYYADSVKGRFTISRDNSKNTLYLQMNSLRAEDTAVYYCATLGLGYYYYFDVWGQGTLVTVSS

SEQ ID NO: 98 - protein, 107 aa, synthetic construct
DIQMTQSPSSLSASVGDRVTITCRASQPISSYVTWYQQKPGKAPKLLIYSASHLRSGVPSRFSGSGSGTDFTLTISSLQPEDFATYYCQQSYNAPPTFGGGTKVEIK

SEQ ID NO: 99 - DNA, 360 bp, synthetic construct
  source 1..360: mol_type = other DNA
gaggtgcagctgctggagagcggcggagggctggtgcaacctgggggctcactgcgactttcctgcgctgcctcaggtttcgatttctctaagttcgctatgagctgggtcagacaggcacccgggaaaggcttagagtgggtgtcttctattacgcgtggcagtgaaactacctactacgccgacagcgttaaaggacggtttacaatctccagggacaattccaagaacaccctctatctgcagatgaacagtctccgcgcggaagacacagccgtctattactgtgcaaccctagggttgggttactattattactttgatgtatggggccagggaacattggtgactgttagctcg

SEQ ID NO: 100 - DNA, 321 bp, synthetic construct
  source 1..321: mol_type = other DNA
gacatccagatgactcagtcgccctcctcactgtctgcttcagttggagatcgggtgaccattacctgccgcgctagtcaaccgatcagctcctatgtgacgtggtatcagcagaagccaggcaaggcccctaaattgttaatctattcagcgtcccacctcaggagcggcgtccccagcagattttctggaagcgggagtggtaccgatttcactctgacaatatcctctcttcagcccgaggactttgcaacatactactgtcaacagagttacaacgccccacctacattcggtggcgggaccaaagtagaaattaag

SEQ ID NO: 101 - protein, 8 aa, synthetic construct
GFPFASHA

SEQ ID NO: 102 - protein, 8 aa, synthetic construct
ITGSGRST

SEQ ID NO: 103 - protein, 12 aa, synthetic construct
ASASSYYEPGDP

SEQ ID NO: 104 - protein, 6 aa, synthetic construct
QTISSF

SEQ ID NO: 105 - skipped

SEQ ID NO: 106 - protein, 8 aa, synthetic construct
QQSYHTRS

SEQ ID NO: 107 - protein, 119 aa, synthetic construct
EVQLLESGGGLVQPGGSLRLSCAASGFPFASHAMTWVRQAPGKGLEWVSTITGSGRSTYYADSVKGRFTISRDNSKNTLYLQMNSLRAEDTAVYYCASASSYYEPGDPWGQGTLVTVSS

SEQ ID NO: 108 - protein, 106 aa, synthetic construct
DIQMTQSPSSLSASVGDRVTITCRASQTISSFVSWYQQKPGKAPKLLIYAATTLQKGVPSRFSGSGSGTDFTLTISSLQPEDFATYYCQQSYHTRSFGGGTKVEIK

SEQ ID NO: 109 - DNA, 357 bp, synthetic construct
  source 1..357: mol_type = other DNA
gaggtgcagctcctcgaaagcggcggcggattagtccagcctgggggatcgctgaggttgtcatgcgccgcctcaggctttcccttcgcatcccacgcaatgacttgggtgagacaggcgccagggaaggggcttgagtgggtgtctacgattaccggcagcgggcggtccacatattatgccgatagtgttaagggccgattcactatctcacgtgataactccaaaaacaccctgtacctgcagatgaatagtctacgcgctgaggacaccgctgtatactattgtgcttctgcctctagctactacgaacccggtgacccgtggggacaaggtacactggtgacagtctccagc

SEQ ID NO: 110 - DNA, 318 bp, synthetic construct
  source 1..318: mol_type = other DNA
gacattcaaatgacacagagcccatctagtttgagcgcatccgtaggcgatagagtgacgatcacttgtcgggcttcacagaccatatcttccttcgtgtcttggtatcagcagaaaccgggtaaggcgcccaaattactcatctacgccgccacaaccctgcaaaagggcgttccttcacgcttttcagggagcgggtccggaacagacttcacacttaccattagttcgctgcagcccgaggatttcgctacttattactgccagcagtcctaccacactaggagctttggcggagggaccaaagtcgaaatcaag

SEQ ID NO: 111 - protein, 8 aa, synthetic construct
GLTFSNFA

SEQ ID NO: 112 - protein, 8 aa, synthetic construct
IRGSGATT

SEQ ID NO: 113 - protein, 11 aa, synthetic construct
ARSREAYGFDY

SEQ ID NO: 114 - protein, 6 aa, synthetic construct
QSISSY

SEQ ID NO: 115 - skipped

SEQ ID NO: 116 - protein, 9 aa, synthetic construct
QQSYSTPYT

SEQ ID NO: 117 - protein, 118 aa, synthetic construct
EVQLLESGGGLVQPGGSLRLSCAASGLTFSNFAMTWVRQAPGKGLEWVSSIRGSGATTYYADSVKGRFTISRDNSKNTLYLQMNSLRAEDTAVYYCARSREAYGFDYWGQGTLVTVSS

SEQ ID NO: 118 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGGGTKVEIK

SEQ ID NO: 119 - DNA, 354 bp, synthetic construct
  source 1..354: mol_type = other DNA
gaagtgcagctcctagagagcgggggcgggctggtgcagcccggagggtcactgaggctgtcttgcgctgcctctggcttaacattctccaatttcgcaatgacatgggtgcgccaggcccctgggaaaggtcttgagtgggtgtcctcgattaggggatctggtgccaccacttactacgctgactccgtcaagggcagatttacgatcagcagagataacagtaagaataccctgtacttgcagatgaacagtcttcgggccgaagatacagctgtctattattgtgcacgaagccgtgaggcgtatggatttgactactggggccaaggcaccctcgtaactgttagctca

SEQ ID NO: 120 - DNA, 321 bp, synthetic construct
  source 1..321: mol_type = other DNA
gatatacagatgacacaatcaccctccagcctttcggcctcagtgggtgacagagttaccattacctgccgggcttcacagagcatcagctcctacttgaactggtaccagcaaaagcccggaaaagctccaaagctcttaatctacgccgcatcttctctgcagtctggggtgccttctaggtttagcggaagtggcagcggcaccgactttacactgactattagttccctgcagcctgaagatttcgcgacatattactgtcagcagtcctatagtactccatataccttcggtggcgggacgaaggtcgagatcaaa

SEQ ID NO: 121 - protein, 8 aa, synthetic construct
GFTFSHYS

SEQ ID NO: 122 - protein, 8 aa, synthetic construct
ISGSGSAT

SEQ ID NO: 123 - protein, 10 aa, synthetic construct
ASDRYLTFDV

SEQ ID NO: 124 - protein, 6 aa, synthetic construct
QSISTH

SEQ ID NO: 125 - skipped

SEQ ID NO: 126 - protein, 9 aa, synthetic construct
QQSHRTPLI

SEQ ID NO: 127 - protein, 117 aa, synthetic construct
EVHLLESGGGLVQPGGSLRLSCAASGFTFSHYSMNWVRQAPGKGLEWVSGISGSGSATYYADSVKGRFTISRDNSKNTLYLQMNSLRAEDTAVYYCASDRYLTFDVWGQGTLVTVSS

SEQ ID NO: 128 - protein, 107 aa, synthetic construct
DIQMTQSPSSLSASVGDRVTITCRASQSISTHLNWYQQKPGKAPKLLIYAASSLQSGVPSRFSGSGSGTDFTLTISSLQPEDFATYYCQQSHRTPLIFGGGTKVEIK

SEQ ID NO: 129 - DNA, 351 bp, synthetic construct
  source 1..351: mol_type = other DNA
gaagtccacctgttggagagcggcggtggcctggttcaacctgggggttctctacgtttgtcgtgtgcagcctctgggtttacatttagccattatagcatgaattgggtgagacaggcacccggaaagggacttgagtgggttagcgggatctccgggtcaggctccgcgacttactatgccgattcagtgaaaggccgcttcaccatttctcgggacaacagtaagaatactctgtacctccagatgaacagtctccgagctgaagacaccgccgtgtactactgcgcttccgacaggtatctgaccttcgatgtctggggacagggcacgttagtgacagtatcctca

SEQ ID NO: 130 - DNA, 321 bp, synthetic construct
  source 1..321: mol_type = other DNA
gacatccagatgacgcaatccccttctagcctgtcagcgtcagtgggcgatcgggtcacaatcacttgtagagcttctcagagtattagtacccatctgaactggtaccagcagaaaccaggcaaggcacccaaactcctcatatatgccgcttccagcttacagagtggtgtgccatcgaggttcagcggttccggcagcggaactgactttaccttgaccatctcctctctgcagcctgaagattttgccacatattactgccaacagtcacaccgcaccccccttattttcggaggggggacaaaggttgagattaag

SEQ ID NO: 131 - protein, 8 aa, synthetic construct
GFDFSKFA

SEQ ID NO: 132 - protein, 8 aa, synthetic construct
ISGSGGTI

SEQ ID NO: 133 - protein, 12 aa, synthetic construct
ASASSYYEPGDP

SEQ ID NO: 134 - protein, 6 aa, synthetic construct
QAIKNY

SEQ ID NO: 135 - skipped

SEQ ID NO: 136 - protein, 9 aa, synthetic construct
QQTYSTPLT

SEQ ID NO: 137 - protein, 119 aa, synthetic construct; identical to SEQ ID NO: 77

SEQ ID NO: 138 - protein, 107 aa, synthetic construct
DIQMTQSPSSLSASVGDRVTITCRASQAIKNYLNWYQQKPGKAPKLLIYAGSTLQSGVPSRFSGSGSGTDFTLTISSLQPEDFATYYCQQTYSTPLTFGGGTKGEIK

SEQ ID NO: 139 - DNA, 357 bp, synthetic construct
  source 1..357: mol_type = other DNA
gaggtgcagctgctggagtctggcggcggtcttgtccagcccggggggtcactccgactaagctgcgctgcgagtggatttgacttctccaagtttgccatgtcctgggtgagacaggcaccaggaaagggacttgagtgggtgtcacacatatctgggagcggcggaactatttactacgctgatagcgttaaaggccgcttcacaatctcacgggacaacagtaaaaacaccctctatttgcaaatgaatagtctgagggccgaagatacagcagtctattactgtgcctccgcctctagctactatgaacctggcgacccgtgggggcagggtacgctggtgaccgtatcgtcc

SEQ ID NO: 140 - DNA, 321 bp, synthetic construct
  source 1..321: mol_type = other DNA
gatattcagatgacacagagcccctcgtcactgtctgcgtccgtcggtgatcgggttacaatcacctgtagggcttctcaagctatcaagaactacctcaattggtaccagcaaaaacctggcaaggccccaaaactgttaatttatgcagggtctacattgcagagtggcgtgccttcaagattcagcggctccgggtccggaactgacttcacccttacaatcagcagtctgcagcccgaggactttgccacctattattgccagcagacgtacagcaccccactcacttttgggggtggaactaaaggagaaataaag

SEQ ID NO: 141 - protein, 8 aa, synthetic construct
GFSFDNYG

SEQ ID NO: 142 - protein, 8 aa, synthetic construct
ISSSGGSA

SEQ ID NO: 143 - protein, 11 aa, synthetic construct
ARGVVPGGFDY

SEQ ID NO: 144 - protein, 6 aa, synthetic construct
QSINNY

SEQ ID NO: 145 - skipped

SEQ ID NO: 146 - protein, 9 aa, synthetic construct
QQSRTTPWT

SEQ ID NO: 147 - protein, 118 aa, synthetic construct
EVQFLESGGGLVQPGGSLRLSCAASGFSFDNYGINWVRQAPGKGLEWVSSISSSGGSAYYADSVKGRFTISRDNSKNTLYLQMNSLRAEDTAVYYCARGVVPGGFDYWGQGTLVTVSS

SEQ ID NO: 148 - protein, 107 aa, synthetic construct
DIQMTQSPSSLSASVGDRVTITCRASQSINNYLNWYQQKPGKAPKLLIYAASSLQSGVPSRFSGSGSGTDFTLTISSLQPEDFATYYCQQSRTTPWTFGGGTKVEIK

SEQ ID NO: 149 - DNA, 354 bp, synthetic construct
  source 1..354: mol_type = other DNA
gaggtgcagttcctcgaatccggtgggggtttagtgcagcccgggggcagccttcggctgtcctgcgccgcctctgggtttagcttcgacaattacggtatcaactgggttcgacaagcaccagggaagggactggaatgggtgtcatcaatttcctcgtctggaggctccgcgtattacgcagattcagtgaaaggccgttttaccataagtagggacaatagcaagaacacactgtacctccagatgaacagtctgcgcgctgaggacaccgccgtctattactgtgctagaggcgtagtccctggaggattcgattattggggccagggcacgttggttactgtgtctagc

SEQ ID NO: 150 - DNA, 321 bp, synthetic construct
  source 1..321: mol_type = other DNA
gacatccaaatgacacagtccccaagttctctttccgcctctgtgggtgacagagtgacaatcacctgtcgcgcatcccagtcaattaataactatctcaactggtaccagcagaagcctgggaaggcgcccaaactgctgatttatgccgcttccagcttacaaagtggtgtcccatcaaggttttcaggcagcggaagcgggaccgatttcactctgacgataagctcgttgcagcctgaagatttcgctacctactactgccagcagtctcggactaccccctggacttttggcggcggaacaaaggttgagatcaaa

SEQ ID NO: 151 - protein, 8 aa, synthetic construct
GFDFSKFA

SEQ ID NO: 152 - protein, 8 aa, synthetic construct
ITGTSGAT

SEQ ID NO: 153 - protein, 15 aa, synthetic construct
ARDIRVRRSSWAMDP

SEQ ID NO: 154 - protein, 6 aa, synthetic construct
QTIGIY

SEQ ID NO: 155 - skipped

SEQ ID NO: 156 - protein, 10 aa, synthetic construct
QQSYSTPHIT

SEQ ID NO: 157 - protein, 122 aa, synthetic construct
EVQLLESGGGLVQPGGSLRLSCAASGFDFSKFAMSWVRQAPGKGLEWVSSITGTSGATYYADSVKGRFTISRDNSKNTLYLQMNSLRAEDTAVYYCARDIRVRRSSWAMDPWGQGTLVTVSS

SEQ ID NO: 158 - protein, 108 aa, synthetic construct
DIQMTQSPSSLSASVGDRVTITCRASQTIGIYLNWYQQKPGKGPKPAIYSASPLQRGVPSRFSGCGSGTDFTLTISSLQPEDFATYYCQQSYSTPHITFGGGTKVEIK

SEQ ID NO: 159 - DNA, 366 bp, synthetic construct
  source 1..366: mol_type = other DNA
gaagtgcagcttcttgagtccggcgggggactagttcagcccggcggctcactgcgactgagttgtgcggcttcagggtttgatttcagcaaattcgcaatgtcttgggtgagacaggccccaggaaaaggtttggagtgggtgagctctattaccgggacctcgggcgccacctactatgctgattccgttaagggtagatttactatctctagggacaatagcaagaacacactctacctccaaatgaacagtctgcgggccgaagacactgccgtatactattgcgctagggatatacgcgtgcggcgtagctcctgggcaatggacccttggggacagggcacgctggtcacagtctcctca

SEQ ID NO: 160 - DNA, 324 bp, synthetic construct
  source 1..324: mol_type = other DNA
gacatccagatgacgcaaagtcctagcagcctgtccgcctcagtgggcgaccgggtgactatcacatgtagggcttctcagaccattggcatatatctgaactggtaccagcagaagcccgggaaaggtcccaaaccggcgatctactccgcatccccacttcagcgcggcgtcccttccagattctcggggtgcggctcaggtacagattttactttgacaatttctagtctccagcccgaagatttcgccacttactattgccaacagagctattctaccccacacattacctttggagggggaaccaaggttgagatcaag

SEQ ID NO: 161 - protein, 8 aa, synthetic construct
GFDFSKFA

SEQ ID NO: 162 - protein, 8 aa, synthetic construct
ISGSGGTI

SEQ ID NO: 163 - protein, 12 aa, synthetic construct
ASASSYYEPGDP

SEQ ID NO: 164 - protein, 6 aa, synthetic construct
QAIKNY

SEQ ID NO: 165 - skipped

SEQ ID NO: 166 - protein, 9 aa, synthetic construct
QQTYSTPLT

SEQ ID NO: 167 - protein, 119 aa, synthetic construct; identical to SEQ ID NO: 77

SEQ ID NO: 168 - protein, 107 aa, synthetic construct
DIQMTQSPSSLSASVGDRVTITCRASQAIKNYLNWYQQKPGKAPKLLIYAGSTLQSGVPSRFSGSGSGTDFTLTISSLQPEEFATYYCQQTYSTPLTFGGGTKGENQ

SEQ ID NO: 169 - DNA, 357 bp, synthetic construct
  source 1..357: mol_type = other DNA
gaggtacaactcctggagtcaggcggagggctcgtccagccaggaggttccctaagactttcttgtgccgcctcaggctttgacttcagcaagttcgcaatgagctgggtgaggcaggcgcctgggaaggggttagaatgggtgtctcacattagtggctctggaggaactatctactatgccgattcggtgaaagggcgatttacaatatcccgcgacaattcaaaaaacacactgtacctgcagatgaacagtttgcgggctgaagataccgcagtctactattgcgcttccgcctccagctattacgagcccggtgacccgtggggccagggcaccctggttacggtgagtagc

SEQ ID NO: 170 - DNA, 321 bp, synthetic construct
  source 1..321: mol_type = other DNA
gacattcaaatgacacagagtcccagtagcctcagcgcttccgttggcgatagagtgacaattacctgtagggcctcccaggcaataaagaactatcttaactggtaccagcagaaaccaggtaaggcccctaaactcttaatctatgctggctcaaccctgcaatctggggtcccctcgcggttctcagggtctggaagcggcacagacttcactttgaccatctctagcctgcagccagaggagtttgcgacttactattgccaacagacatactccacccctctgacgtttggagggggtactaagggagaaaatcag

SEQ ID NO: 171 - protein, 8 aa, synthetic construct
GFTFTNYA

SEQ ID NO: 172 - protein, 8 aa, synthetic construct
ISGSGGTI

SEQ ID NO: 173 - protein, 12 aa, synthetic construct
ASASSYYEPGDP

SEQ ID NO: 174 - protein, 6 aa, synthetic construct
QAIKNY

SEQ ID NO: 175 - skipped

SEQ ID NO: 176 - protein, 9 aa, synthetic construct
QQTYSTPLT

SEQ ID NO: 177 - protein, 119 aa, synthetic construct
EVQLLESGGGLVQPGGSLRLSCAASGFTFTNYALAWVRQAPGKGLEWVSHISGSGGTIYYADSVKGRFTISRDNSKNTLYLQMNSLRAEDTAVYYCASASSYYEPGDPWGQGTLVTVSS

SEQ ID NO: 178 - protein, 107 aa, synthetic construct; identical to SEQ ID NO: 48

SEQ ID NO: 179 - DNA, 357 bp, synthetic construct
  source 1..357: mol_type = other DNA
gaagtgcagctgctagaatccggagggggcttagtccagcctggcggctcccttcggctctcttgtgccgcttccggatttacgttcactaactatgcactggcctgggtgcgacaggcgccagggaaggggctggagtgggtaagccacatctctggatcaggaggtacaatttactatgctgacagcgtgaaggggcgcttcaccataagcagagataactctaaaaataccctttatctgcaaatgaatagtctcagggccgaggatactgcagtgtattactgcgcttcggcctcaagctactacgagcccggcgacccgtggggtcagggcacattggtcaccgttagttcc

SEQ ID NO: 180 - DNA, 321 bp, synthetic construct
  source 1..321: mol_type = other DNA
gatatccaaatgacacagtctcccagttcattgtccgcttccgttggagatagagtcaccattacctgcagggccagtcaggcgataaaaaactaccttaattggtatcagcagaagcctgggaaagctcccaagctgctgatctacgccggctcaactctgcagtctggggtgccttctcggttttccggttcgggcagcgggacggactttacactcacaattagcagcttacaaccagaggacttcgcaacttattactgtcagcagacctatagcacaccactcactttcggcggaggtaccaaggtggaaatcaaa

SEQ ID NO: 181 - protein, 8 aa, synthetic construct
GFDFSKFA

SEQ ID NO: 182 - protein, 8 aa, synthetic construct
ISGSGGTI

SEQ ID NO: 183 - protein, 12 aa, synthetic construct
ASASSYYEPGDP

SEQ ID NO: 184 - protein, 6 aa, synthetic construct
QAIKNY

SEQ ID NO: 185 - skipped

SEQ ID NO: 186 - protein, 9 aa, synthetic construct
QQTYSTPLT

SEQ ID NO: 187 - protein, 119 aa, synthetic construct; identical to SEQ ID NO: 77

SEQ ID NO: 188 - protein, 107 aa, synthetic construct; identical to SEQ ID NO: 48

SEQ ID NO: 189 - DNA, 357 bp, synthetic construct
  source 1..357: mol_type = other DNA
gaggtgcagctgctggagtctgggggcgggctagtccaacccggaggatcgctgaggctttcctgcgccgcgtcagggtttgacttcagcaagtttgccatgtcttgggtgcgacaggcacctggaaaaggattggaatgggtgtcacacataagcggttctggcggcactatctattacgccgactccgtcaaaggccgcttcacaattagccgggataacagcaagaatacgctctacttacagatgaacagtctcagagccgaggataccgctgtttactattgtgcatcagctagtagttactatgaaccaggtgacccgtgggggcagggcaccctggtgacagtatcctcc

SEQ ID NO: 190 - DNA, 321 bp, synthetic construct
  source 1..321: mol_type = other DNA
gatatccagatgacccagtcgccaagctctctctccgcttctgttggagacagggtcacaattacttgccgggcatcccaagcgatcaagaactatttaaattggtaccagcagaagcctgggaaagctcctaaacttctgatatatgccggctctaccctgcagtcaggggtgcccagtagattctcagggagcggcagtggtaccgatttcacactgaccattagcagcttgcaaccagaggactttgccacatactattgtcagcagacgtactccactcccctcacatttggtggcggaactaaagtggaaatcaag

SEQ ID NO: 191 - protein, 8 aa, synthetic construct
GIRFSSYA

SEQ ID NO: 192 - protein, 8 aa, synthetic construct
ISGSGGTI

SEQ ID NO: 193 - protein, 12 aa, synthetic construct
ASASSYYEPGDP

SEQ ID NO: 194 - protein, 6 aa, synthetic construct
QAIKNY

SEQ ID NO: 195 - skipped

SEQ ID NO: 196 - protein, 9 aa, synthetic construct
QQTYSTPLT

SEQ ID NO: 197 - protein, 119 aa, synthetic construct
EVQLLESGGGLVQPGGSLRLSCAASGIRFSSYAMSWVRQAPGKGLEWVSHISGSGGTIYYADSVKGRFTISRDNSKNTLYLQMNSLRAEDTAVYYCASASSYYEPGDPWGQGTLVTVSS

SEQ ID NO: 198 - protein, 107 aa, synthetic construct; identical to SEQ ID NO: 48

SEQ ID NO: 199 - DNA, 357 bp, synthetic construct
  source 1..357: mol_type = other DNA
gaggtccagctgctagagtctggaggcgggctggtgcagccgggaggtagtttacgcctgtcctgtgcagcttcgggcattaggttttcaagctacgcaatgtcttgggtgcgacaagcgcctggtaagggactggaatgggtgagccacatcagcggcagcggcggaactatatattatgccgactccgtaaaggggcggttcacgatctcacgtgataactcaaaaaacacattgtatctccagatgaatagtcttagagccgaggataccgccgtttactactgcgcttccgcctccagttactatgaaccaggggacccctggggccaggggacactcgtcaccgtgtcttct

SEQ ID NO: 200 - DNA, 321 bp, synthetic construct
  source 1..321: mol_type = other DNA
gatattcagatgactcagagcccatctagcctcagcgcttctgtgggggacagggttacgattacatgtagagcaagtcaggccatcaagaactaccttaattggtaccagcaaaagcctggcaaagctcctaaactgctcatctacgcggggtccacattgcaatctggagtcccctcgcggtttagtggaagcggctccggtacagacttcactctgaccatctcatcattacagccagaggatttcgccacatattattgccagcagacctattccactcccctgacctttggcggcgggaccaaagtggaaataaag

SEQ ID NO: 201 - protein, 8 aa, synthetic construct
GFDFSKFV

SEQ ID NO: 202 - protein, 8 aa, synthetic construct
ISGSGGTI

SEQ ID NO: 203 - protein, 12 aa, synthetic construct
ASASSYYEPGDP

SEQ ID NO: 204 - protein, 6 aa, synthetic construct
QAIKNY

SEQ ID NO: 205 - skipped

SEQ ID NO: 206 - protein, 9 aa, synthetic construct
QQTYSTPLT

SEQ ID NO: 207 - protein, 119 aa, synthetic construct
EVQLLESGGGLVQPGGSLRLSCAVSGFDFSKFVMSGVRQAPGKGLEWVSHISGSGGTIYYADSVKGRFTISRDNSKNTLYLQMNSLRAEDTAVYYCASASSYYEPGDPWGQGTLVTVSS

SEQ ID NO: 208 - protein, 107 aa, synthetic construct; identical to SEQ ID NO: 48

SEQ ID NO: 209 - DNA, 357 bp, synthetic construct
  source 1..357: mol_type = other DNA
gaagtgcagctcttagagagcgggggcggactggtccagccaggcggtagcctgcgcctaagttgcgccgtttctgggttcgatttttctaagttcgtgatgagtggcgtgcggcaggctcctggcaaaggtctggagtgggtttcacacatatccgggtctgggggaacgatttactacgctgattcagtgaaaggaagatttaccatctccagggacaacagcaagaatactctctatctgcaaatgaacagtcttcgagcggaagacacagccgtctactattgtgcatccgcctcgtcatattacgagccgggtgacccctggggacagggcacattggtgaccgtaagctcc

SEQ ID NO: 210 - DNA, 321 bp, synthetic construct
  source 1..321: mol_type = other DNA
gacatccagatgacccagtctccttcctcccttagtgcttcggtgggtgatagggtcaccattacatgcagagccagtcaggcaataaagaactacttgaattggtaccagcaaaaacccggcaaggcccccaaactgctgatctatgctggttccacgttacagtctggagtgccaagccggtttagcggctcagggtctggaactgatttcacactgacaattagctcactccagccagaagacttcgcgacttattattgtcagcaaacctacagcacccctctcacttttgggggcgggacaaaggttgagatcaaa

SEQ ID NO: 211 - protein, 8 aa, synthetic construct
GFDFSKFA

SEQ ID NO: 212 - protein, 8 aa, synthetic construct
ISGSGGTI

SEQ ID NO: 213 - protein, 12 aa, synthetic construct
ASASSYYEPGDP

SEQ ID NO: 214 - protein, 6 aa, synthetic construct
QAIKNY

SEQ ID NO: 215 - skipped

SEQ ID NO: 216 - protein, 9 aa, synthetic construct
QQNYSTPFA

SEQ ID NO: 217 - protein, 119 aa, synthetic construct; identical to SEQ ID NO: 77

SEQ ID NO: 218 - protein, 107 aa, synthetic construct
DIQMTQSPSSLSASVGDRVTITCRASQAIKNYLNWYQQKPGKAPKLLIYAGSTLQSGVPSRFSGSGSGTDFTLTISSLQPEDFPTYYFQQNYSTPFAFGGGTKVEIK

SEQ ID NO: 219 - DNA, 357 bp, synthetic construct
  source 1..357: mol_type = other DNA
gaagtgcagctcctagagtccggaggtggcctcgtgcagccgggaggctccctgcgcctgtcctgcgcggcctctggctttgactttagtaagttcgctatgagctgggtgagacaggctccaggtaagggcctggagtgggtatctcacatctccgggtcaggagggaccatatattacgccgactctgtcaaaggccggttcactatttcaagggataattcaaaaaacacattataccttcaaatgaacagtctgcgagccgaagacacagcagtgtactattgtgccagcgcaagcagctactatgagcctggagatccctgggggcaggggaccttggtcacggttagttcg

SEQ ID NO: 220 - DNA, 321 bp, synthetic construct
  source 1..321: mol_type = other DNA
gatatacagatgacccagtcaccctccagcttgtcagctagtgttggggaccgggtcaccatcacatgcagagcctctcaggcgatcaagaattaccttaactggtatcagcagaaaccaggaaaagctccgaagttactgatttacgcaggtagcacactgcagtcgggcgtgccctctaggttcagtgggagcggctccggaacagactttactctgaccatttcttccctccaaccagaagatttccctacttattattttcagcaaaactacagcacgcctttcgcctttggcggtgggaccaaagtggagatcaag

SEQ ID NO: 221 - protein, 8 aa, synthetic construct
GFDFSKFA

SEQ ID NO: 222 - protein, 8 aa, synthetic construct
ISGSGGTI

SEQ ID NO: 223 - protein, 12 aa, synthetic construct
ASASSYYEPGDP

SEQ ID NO: 224 - protein, 6 aa, synthetic construct
QAIKNY

SEQ ID NO: 225 - skipped

SEQ ID NO: 226 - protein, 9 aa, synthetic construct
QQTYSTPLT

SEQ ID NO: 227 - protein, 119 aa, synthetic construct
EVQLLESGGGLVQPGGSLRLSCAASGFDFSKFAMSWVRPAPCKGLEWVSHISGSGGTIYYADSVKGRFTISRDNSKNTLYLQMNSLRAEDTAVYYCASASSYYEPGDPWGQGTLVTVSS

SEQ ID NO: 228 - protein, 107 aa, synthetic construct; identical to SEQ ID NO: 48

SEQ ID NO: 229 - DNA, 357 bp, synthetic construct
  source 1..357: mol_type = other DNA
gaggtgcagcttctggagagcggagggggactggtccagcctggcggcagtctaagattgtcctgcgccgcgtcagggttcgatttttccaaatttgccatgtcatgggtgcgcccagctccctgtaagggattagaatgggtgagtcacatctcaggttctgggggcacaatttattacgcagactccgtcaaggggcgattcacgatatcccgggacaactctaaaaacactctgtacctccaaatgaatagtctcagggccgaggacaccgctgtgtactattgcgcctcggcaagcagctattacgaacccggcgatccgtggggccagggtaccctggtaacagttagctct

SEQ ID NO: 230 - DNA, 321 bp, synthetic construct
  source 1..321: mol_type = other DNA
gacatccagatgacacagagtccatctagcttatccgcgagtgttggggacagggtcaccatcacatgtagagcctctcaggcaattaaaaactacctgaattggtatcaacaaaagcccggaaaggccccaaagctgctcatctacgccggctccaccctgcagagcggagtgcctagccggtttagcggctctggctcggggactgattttacattgactatttcatcacttcagcccgaagatttcgctacatattattgccagcagacctactccactcctctcacgttcggtggtgggaccaaagtggagataaaa

SEQ ID NO: 231 - protein, 8 aa, synthetic construct
GFDFSKFA

SEQ ID NO: 232 - protein, 8 aa, synthetic construct
ISGSGGTI

SEQ ID NO: 233 - protein, 12 aa, synthetic construct
ASASSYYEPGDP

SEQ ID NO: 234 - protein, 6 aa, synthetic construct
QAIKNY

SEQ ID NO: 235 - skipped

SEQ ID NO: 236 - protein, 9 aa, synthetic construct
QQNYSTPLT

SEQ ID NO: 237 - protein, 119 aa, synthetic construct; identical to SEQ ID NO: 77

SEQ ID NO: 238 - protein, 107 aa, synthetic construct
DIQMTQSPSSLSASVGDRVTITCRASQAIKNYLNWYQQKPGKAPKLLIYAGSTLQSGVPSRFSGSGSGTDFTLTISSLQPEDFATYYCQQNYSTPLTFGGGTKVEIK

SEQ ID NO: 239 - DNA, 357 bp, synthetic construct
  source 1..357: mol_type = other DNA
gaggtgcagctgctggaaagcggaggagggttagtgcaaccaggaggatctctccggctgagctgcgctgcctcaggctttgatttcagcaagttcgccatgtcctgggtccgacaggcaccggggaaaggccttgagtgggtgagccacatatccggctcaggtgggaccatttactacgctgattctgtaaaagggagatttacgatctcacgcgacaacagtaagaatacactctacctgcagatgaacagtttgagggccgaagacacagccgtgtattattgtgcgtctgcatcctcgtactatgagcctggcgacccctggggccagggtaccctagtcactgtttccagt

SEQ ID NO: 240 - DNA, 321 bp, synthetic construct
  source 1..321: mol_type = other DNA
gatatacaaatgacacagagtccctcatccctgtcagcttctgtgggtgatagggtcacaatcacatgcagagccagccaggcgattaaaaattacctcaactggtatcagcagaagcctggcaaggctccaaaattacttatctacgcaggtagcaccctgcagtccggagtgccttctcggttcagcgggtccggctctgggaccgactttaccctcaccatcagtagcctgcagccagaagacttcgccacttattactgtcagcaaaactattcgactcccttgacgtttggaggcgggactaaagttgagattaag

SEQ ID NO: 241 - protein, 8 aa, synthetic construct
GFPFSSHG

SEQ ID NO: 242 - protein, 8 aa, synthetic construct
ISGSGATT

SEQ ID NO: 243 - protein, 11 aa, synthetic construct
ARGQPYYGFDY

SEQ ID NO: 244 - protein, 6 aa, synthetic construct
HSIASY

SEQ ID NO: 245 - skipped

SEQ ID NO: 246 - protein, 9 aa, synthetic construct
QQTYSIPWT

SEQ ID NO: 247 - protein, 118 aa, synthetic construct
EVQFLKSGGGLVNPAGILELSCAASGFPFSSHGMSWVRQAPGKGLEWVSVISGSGATTYYADSVKGRFTISRDNSKNTLYLQMNSLRAEDTAVYYCARGQPYYGFDYWGQGTLVTVSS

SEQ ID NO: 248 - protein, 107 aa, synthetic construct
DIQMTQSPSSLSASVGDRVTITCRASHSIASYLHWYQQKPGKAPKLLIYRASTLHTGVPSRFSGSGSGTDFTLTISSLQPEDFATYYCQQTYSIPWTFGGGTKVEIK

SEQ ID NO: 249 - DNA, 354 bp, synthetic construct
  source 1..354: mol_type = other DNA
gaggtccaatttctgaaaagcggtggcgggctggttaatcccgcgggcatccttgagctctcttgtgcagcctccgggttcccgtttagcagtcacggtatgtcatgggtgaggcaggctccaggaaagggactagaatgggtatctgtgatttcaggctccggagccaccacctattacgcagattccgttaagggccgcttcactatatcgagagacaacagcaaaaatactttatatttgcagatgaacagtctgcgagccgaagacacagctgtctattactgcgcccgggggcagccttactatggcttcgattactggggacaggggacgctcgtgacagtgagctct

SEQ ID NO: 250 - DNA, 321 bp, synthetic construct
  source 1..321: mol_type = other DNA
gacatacagatgacacagtctcccagctctttatccgcttccgtgggcgatagagtgactattacatgccgcgcaagtcactccatcgccagctatctccactggtaccaacaaaagccaggtaaggcgcccaaactgctgatctacagggccagcacgctgcatacaggagtcccttctcggttttccggaagtggctcagggacagatttcactcttaccatttcatcgttgcagcctgaggactttgctacctattattgtcagcagacctacagcatcccgtggactttcggtggcgggaccaaagttgaaattaag

SEQ ID NO: 251 - protein, 8 aa, synthetic construct
GFDFSKFA

SEQ ID NO: 252 - protein, 8 aa, synthetic construct
ISGSGGTI

SEQ ID NO: 253 - protein, 12 aa, synthetic construct
ASASSYYEPGDP

SEQ ID NO: 254 - protein, 6 aa, synthetic construct
QAIKNY

SEQ ID NO: 255 - skipped

SEQ ID NO: 256 - protein, 9 aa, synthetic construct
QQSESTPLT

SEQ ID NO: 257 - protein, 119 aa, synthetic construct
EVQFLESGGGLVQPGGSLRLSCAASGFDFSKFAMSWVRQAPGKGLEWVSHISGSGGTIYYADSVKGRFTISRDNSKNTLYLQMNSLRAEDTAVYYCASASSYYEPGDPWGQGTLVTVSS

SEQ ID NO: 258 - protein, 107 aa, synthetic construct
DIQMTQSPSSLSASVGDRVTITCRASQAIKNYLNWYQQKPGKAPKLLIYGASNLQSGVPSRFSGSGSGTDFTLTISSLQPEDFATYYCQQSESTPLTFGGGTKVEIK

SEQ ID NO: 259 - DNA, 357 bp, synthetic construct
  source 1..357: mol_type = other DNA
gaggtgcaatttctggagtcaggggggggtttggttcagccgggaggcagccttaggttatcctgtgccgcctcagggttcgatttcagcaaatttgcaatgagttgggtccggcaggcacccggcaagggactggaatgggtaagccacatttctggctctggaggcaccatctactacgccgactccgtgaagggacgattcacgatatcgcgcgacaattcaaaaaacaccctgtatctccagatgaacagtctgagagctgaggatactgccgtctattactgcgcttccgcgtctagttactatgaaccaggtgacccttggggccaggggacactcgtgacagtgagctcc

SEQ ID NO: 260 - DNA, 321 bp, synthetic construct
  source 1..321: mol_type = other DNA
gacattcagatgacacagtcaccaagtagtctgtccgcgtctgttggggatcgggtgacgattacatgcagggcttcgcaggccatcaaaaactacttaaactggtatcaacagaagcccggcaaggcccctaagctgcttatatacggggcttccaatctgcagtcaggagtcccttccagattttctggatctggtagcggtactgattttacactcaccatctccagcctccaacccgaggacttcgcaacctattactgtcagcagagcgaaagcactccattgaccttcgggggcggcaccaaagtggagatcaaa

SEQ ID NO: 261 - protein, 8 aa, synthetic construct
GFALTDFA

SEQ ID NO: 262 - protein, 8 aa, synthetic construct
ISVSGGVG

SEQ ID NO: 263 - protein, 13 aa, synthetic construct
ARSRSYYAGAFDV

SEQ ID NO: 264 - protein, 6 aa, synthetic construct
APVGRY

SEQ ID NO: 265 - skipped

SEQ ID NO: 266 - protein, 8 aa, synthetic construct
QQSYSAVT

SEQ ID NO: 267 - protein, 120 aa, synthetic construct
EVQLLESGGGLVQPGGSLRLSCAASGFALTDFAMSWVRQAPGKGLEWVSQISVSGGVGYYADSVKGRFTISRDNSKNTLYLQMNSLRAEDTAVYYCARSRSYYAGAFDVWGQGTLVTVSS

SEQ ID NO: 268 - protein, 106 aa, synthetic construct
DIQMTQSPSSLSASVGDRVTITCRASAPVGRYLNWYQQKPGKAPKLLIYAASSLQSGVPSRFSGSGSGTDFTLTISSLQPEDFATYYCQQSYSAVTFGGGTKVEIK

SEQ ID NO: 269 - DNA, 360 bp, synthetic construct
  source 1..360: mol_type = other DNA
gaagtgcagcttttagaatcaggcggtggactcgtacagcctgggggctccctgagactgagctgcgctgcctcaggcttcgccctcacagactttgccatgtcctgggtgcggcaagctcccgggaagggactagagtgggtgtcccagatctcggtgtctggtggcgttgggtactacgccgacagcgtcaaagggaggttcacgatttccagggataacagtaagaataccctgtacctgcaaatgaacagtttgcgcgcggaggatacagcagtttattattgtgcacgaagccgttcttattacgctggagcatttgacgtgtggggccagggaacccttgtcactgtcagctct

SEQ ID NO: 270 - DNA, 318 bp, synthetic construct
  source 1..318: mol_type = other DNA
gatatccaaatgacccagtctccttcctcgctcagcgcaagtgtgggcgacagggtgaccattacatgtcgcgccagtgctccagtcggaagatatctgaactggtatcagcagaaacctgggaaggcccccaagctgcttatctatgcagcctcatccttacagtctggggtgccctcccggttctcaggtagcggtagcgggactgactttacactgaccatttcatctttgcagccagaggattttgctacgtactactgccagcaaagctactccgcggttacattcggaggcggcactaaagtagaaataaag

SEQ ID NO: 271 - protein, 8 aa, synthetic construct
GFTFGSYA

SEQ ID NO: 272 - protein, 8 aa, synthetic construct
IGTTDRYT

SEQ ID NO: 273 - protein, 13 aa, synthetic construct
ARSRSYYAGAFDV

SEQ ID NO: 274 - protein, 6 aa, synthetic construct
QTLRNY

SEQ ID NO: 275 - skipped

SEQ ID NO: 276 - protein, 9 aa, synthetic construct
QQRFSPPWT

SEQ ID NO: 277 - protein, 120 aa, synthetic construct
EVQLLESGGGLVQPGGSLRLSCAASGFTFGSYAMTWVRQAPGKGLEWVSAIGTTDRYTYYADSVKGRFTISRDNSKNTLYLQMNSLRAEDTAVYYCARSRSYYAGAFDVWGQGTLVTVSS

SEQ ID NO: 278 - protein, 107 aa, synthetic construct
DIQMTQSPSSLSASVGDRVTITCRASQTLRNYLNWYQQKPGKAPKLLIYAASNLQTGVPSRFSGSGSGTDFTLTISSLQPEDFATYYCQQRFSPPWTFGGGTKVEIK

SEQ ID NO: 279 - DNA, 360 bp, synthetic construct
  source 1..360: mol_type = other DNA
gaggtccagctactggaaagcgggggcgggttagttcagcccgggggtagcttgcgcctctcatgcgccgcctccggattcaccttcggctcttatgccatgacctgggtgagacaggcacctggcaagggactggaatgggtgtctgctattggcactactgacagatacacgtactacgctgatagcgtgaaaggccgttttaccatctcccgagacaacagtaagaacacactgtatctccagatgaatagtctgagggctgaggatacagccgtttattactgtgcaaggtcgcggtcctactatgcgggtgcatttgacgtctggggacaagggacccttgtaacagtgtcttca

SEQ ID NO: 280 - DNA, 321 bp, synthetic construct
  source 1..321: mol_type = other DNA
gatattcagatgacacagagcccctctagtctcagcgcctccgtgggtgacagagttaccattacctgcagggcttcgcagactctgcggaactacttgaactggtaccaacaaaaaccagggaaagcgccgaagctccttatctacgcagcctccaatttacagactggcgtcccctctcgctttagtggctcaggatcaggcacggacttcactctgaccataagctctctgcagccagaggatttcgctacctattattgtcagcagcgattttcccctccttggacattcggaggtgggacaaaggtggaaatcaag

SEQ ID NO: 281 - protein, 8 aa, synthetic construct
GYTFSNFA

SEQ ID NO: 282 - protein, 8 aa, synthetic construct
ITGSGVRT

SEQ ID NO: 283 - protein, 13 aa, synthetic construct
ARSRSYYAGAFDV

SEQ ID NO: 284 - protein, 6 aa, synthetic construct
QSIGSS

SEQ ID NO: 285 - skipped

SEQ ID NO: 286 - protein, 9 aa, synthetic construct
QQSDNNPWT

SEQ ID NO: 287 - protein, 120 aa, synthetic construct
EVQLLESGGGLVQPGGSLRLSCAASGYTFSNFAISWVRQAPGKGLEWVSSITGSGVRTFYADSVKGRFTISRDNSKNTLYLQMNSLRAEDTAVYYCARSRSYYAGAFDVWGQGTLVTVSS

SEQ ID NO: 288 - protein, 107 aa, synthetic construct
DIQMTQSPSSLSASVGDRVTITCRASQSIGSSVNWYQQKPGKAPKLLIYAASSLQSGVPSRFSGSGSGTDFTLTISSLQPEDFATYYCQQSDNNPWTFGGGTKVEIK

SEQ ID NO: 289 - DNA, 360 bp, synthetic construct
  source 1..360: mol_type = other DNA
gaggtacagctcctcgaaagtggaggagggctggtccagcccggcggctcactaagactttcatgtgcggcatcggggtatactttcagcaatttcgctatttcctgggtgcggcaagcccctggcaaagggctggaatgggtgagcagcataactggttctggtgtgcgtacattctacgccgattctgttaagggaagatttaccatctccagggacaatagcaagaacacattatatctgcagatgaacagtttgcgcgccgaggataccgctgtctactattgcgcaaggtcacgatcctactacgctggcgcctttgacgtttgggggcagggcaccctggtgacggtgtcttcc

SEQ ID NO: 290 - DNA, 321 bp, synthetic construct
  source 1..321: mol_type = other DNA
gatattcagatgactcaatcccctagcagcctgtctgcgagtgtgggcgacagagttaccatcacttgcagggcttcacaatccatagggtcatccgtcaattggtatcagcagaaacctgggaaagctcccaagttgcttatctatgccgcctcttctctgcagtccggcgtgccatctcggttcagcggcagcggaagtgggacagactttacactcaccatctcgtcattacagccagaagatttcgcaacgtactactgtcagcagagtgacaacaacccctggacctttggaggtggtacaaaggtagagattaag

SEQ ID NO: 291 - protein, 8 aa, synthetic construct
GFTFNNAW

SEQ ID NO: 292 - protein, 8 aa, synthetic construct
ISSSGGYT

SEQ ID NO: 293 - protein, 13 aa, synthetic construct
ARSRSYYAGAFDV

SEQ ID NO: 294 - protein, 6 aa, synthetic construct
APVGRY

SEQ ID NO: 295 - skipped

SEQ ID NO: 296 - protein, 9 aa, synthetic construct
QQAYSIPIT

SEQ ID NO: 297 - protein, 120 aa, synthetic construct
EVQLLKSGSGLVQPGGSLRLSCAASGFTFNNAWMHWVRQAPGKGLEWVSAISSSGGYTYYADSVKGRFTISRDNSKNTLYLQMNSLRAEDTAVYYCARSRSYYAGAFDVWGQGTLVTVSS

SEQ ID NO: 298 - protein, 107 aa, synthetic construct
DIQMTQSPSSLSASVGDRVTITCRASAPVGRYLNWYQQKPGKAPKLLIYAASSLQSGVPSRFSGSGSGTDFTLTISSLQPEDFATYYCQQAYSIPITFGGGTKVEIK

SEQ ID NO: 299 - DNA, 360 bp, synthetic construct
  source 1..360: mol_type = other DNA
gaggtacaactcttaaagtccggatctggattggtccagcctggcggcagcctcagactgtcatgcgcagccagcggatttaccttcaacaatgcctggatgcactgggtgaggcaggcacccggcaaaggtcttgagtgggtgtcggccatctcaagctccgggggctatacatattatgcagattcagttaaggggcggttcaccatttctcgtgacaactccaaaaatactctgtacctgcagatgaacagtcttcgagctgaagatacagctgtgtattactgtgctaggtcccgctcttactacgcgggcgcctttgacgtttgggggcagggtactctggtcacggtgagcagt

SEQ ID NO: 300 - DNA, 321 bp, synthetic construct
  source 1..321: mol_type = other DNA
gatattcagatgacacagtccccaagctcgctgtctgcttccgttggcgatagagtaactatcacatgccgcgccagcgctccagtggggaggtacctcaactggtaccagcagaaacccgggaaggcacctaaattgctgatctatgccgcctcttcactgcagtcaggtgtcccctctcggtttagtgggagcggtagtggaactgactttacacttaccatttcctccttacagcctgaagacttcgcaacgtattactgtcaacaagcgtatagcatccccataaccttcggaggcggcaccaaggtggagattaag

SEQ ID NO: 301 - protein, 8 aa, synthetic construct
GFSVRDFA

SEQ ID NO: 302 - protein, 8 aa, synthetic construct
ITGTDRTP

SEQ ID NO: 303 - protein, 13 aa, synthetic construct
ARSRSYYAGAFDV

SEQ ID NO: 304 - protein, 6 aa, synthetic construct
QNIFSY

SEQ ID NO: 305 - skipped

SEQ ID NO: 306 - protein, 9 aa, synthetic construct
QQTHSIPQT

SEQ ID NO: 307 - protein, 120 aa, synthetic construct
EVQLLESGGGLVQPGGSLRLSCAASGFSVRDFAMNWVRQAPGKGLEWVSTITGTDRTPYYADSVKGRFTISRDNSKNTLYLQMNSLRAEDTAVYYCARSRSYYAGAFDVWGQGTLVTVSS

SEQ ID NO: 308 - protein, 107 aa, synthetic construct
DIQMTQSPSSLSASVGDRVTITCRAPQNIFSYINWYQQKPGKAPKLLIYAASSLQSGVPSRFSGSGSGTDFTLTISSLQPEDFATYYCQQTHSIPQTFGGGTKGEIK

SEQ ID NO: 309 - DNA, 360 bp, synthetic construct
  source 1..360: mol_type = other DNA
gaggttcagctgttagaatcaggaggggggttggtgcagcctggaggctctctccggctgtcctgcgccgcatcgggcttctctgtccgtgacttcgccatgaattgggttcggcaggcccccggtaaggggctggagtgggtgagcaccatcacaggtactgatagaacaccatactacgcagattcagtaaagggaagatttactattagcagggacaattctaaaaacacgctttacctccagatgaacagtctaagggctgaagatacagccgtgtattattgtgctcgcagccgatcctactatgctggcgcgtttgacgtgtggggccaaggcaccctggtcaccgtgagttcc

SEQ ID NO: 310 - DNA, 321 bp, synthetic construct
  source 1..321: mol_type = other DNA
gatatacagatgacccagtctcccagcagcctttctgcctcagtcggggatagagttacgatcacatgtcgggctccacagaatatcttcagctacattaactggtaccagcagaagccgggcaaggcacctaagttgctgatctatgcggctagttccttacaatccggagtgccatcgaggttttccggctctgggtccggtacagactttaccctgactattagctcactccagcctgaggacttcgccacctattactgccagcaaacacacagtatcccccaaactttcggcggtgggaccaaaggagaaattaaa

SEQ ID NO: 311 - protein, 8 aa, synthetic construct
GFTFSSHN

SEQ ID NO: 312 - protein, 8 aa, synthetic construct
IGGGGRTT

SEQ ID NO: 313 - protein, 14 aa, synthetic construct
ARAAPGAYAYALDY

SEQ ID NO: 314 - protein, 6 aa, synthetic construct
QRISTY

SEQ ID NO: 315 - skipped

SEQ ID NO: 316 - protein, 9 aa, synthetic construct
QQTYSTPPT

SEQ ID NO: 317 - protein, 121 aa, synthetic construct
EVQLLESGGGLVQPGGSLRLSCAASGFTFSSHNMAWVRQAPGKGLEWVSSIGGGGRTTNYADSVKGRFTISRDNSKNTLYLQMNSLRAEDTAVYYCARAAPGAYAYALDYWGQGTLVTVSS

SEQ ID NO: 318 - protein, 107 aa, synthetic construct
DIQMTQSPSSLSASVGDRVTITCRASQRISTYINWYQQKPGKAPKLLIYGASYLHSGVPSRFSGSGSGTDFTLTISSLQPEDFATYYCQQTYSTPPTFGGGTKVEIK

SEQ ID NO: 319 - DNA, 363 bp, synthetic construct
  source 1..363: mol_type = other DNA
gaagtgcaattgctcgaaagcggcggaggccttgttcagcctggaggtagcctgaggctgtcttgtgcggcttccgggttcaccttcagctcacacaacatggcttgggtgcggcaggcaccaggaaaggggctagagtgggtgagttccatcggtggcggtggaaggactacgaactatgccgactcagtcaaagggcgttttacaatttcccgcgataattctaagaatactctttacctccagatgaacagtctgcgagccgaggataccgctgtctattactgcgccagagctgcacccggcgcctacgcctatgcattagactactggggccaggggaccctggtgacagtatcgtct

SEQ ID NO: 320 - DNA, 321 bp, synthetic construct
  source 1..321: mol_type = other DNA
gatattcagatgacccaatcaccaagcagccttagcgcatccgttggtgatcgggtgacaattacctgcagggcctctcagagaatatccacgtacatcaactggtaccagcagaagcctgggaaagcgcctaagctcctgatttatggcgccagttatttgcactctggcgtgccctcgcgctttagcggtagtggatcagggaccgactttactctgactatctcttccttacagcccgaagacttcgctacatattactgtcaacagacatactccacacccccaactttcgggggcggaaccaaagtcgagatcaag

SEQ ID NO: 321 - protein, 8 aa, synthetic construct
GFSVRDFA

SEQ ID NO: 322 - protein, 8 aa, synthetic construct
IRISGGGT

SEQ ID NO: 323 - protein, 14 aa, synthetic construct
ARAAPGAYAYALDY

SEQ ID NO: 324 - protein, 6 aa, synthetic construct
QRISTY

SEQ ID NO: 325 - skipped

SEQ ID NO: 326 - protein, 9 aa, synthetic construct
QQNYRTPPT

SEQ ID NO: 327 - protein, 121 aa, synthetic construct
EVQLLESGGGLVQPGGSLRLSCAASGFSVRDFAMTWVRQAPGKGLEWVSAIRISGGGTFYADSVKGRFTISRDNSKNTLYLQMNSLRAEDTAVYYCARAAPGAYAYALDYWGQGTLVTVSS

SEQ ID NO: 328 - protein, 107 aa, synthetic construct
DIQMTQSPSSLSASVGDRVTITCRASQRISTYINWYQQKPGKAPKLLIYSASVLENGVPSRFSGSGFGTDFTLTISSLQPEDFATYYCQQNYRTPPTFGSGTKGENK

SEQ ID NO: 329 - DNA, 363 bp, synthetic construct
  source 1..363: mol_type = other DNA
gaagtacagcttctggagtctggcggaggactagttcagcccggcgggtcactccggctgagctgtgctgcatcagggtttagcgtccgtgatttcgccatgacatgggtcagacaggctcctggtaagggccttgaatgggtgtccgccatcagaatatcgggggggggaaccttttatgccgacagcgtgaaaggccgattcactatttcccgcgacaattctaagaacactctgtatttgcaaatgaacagtctgagggccgaggatacagccgtctactactgcgcaagggcggcaccaggtgcttacgcttatgccctcgactactggggacagggcacgttagtgaccgtgagttcc

SEQ ID NO: 330 - DNA, 321 bp, synthetic construct
  source 1..321: mol_type = other DNA
gacattcagatgacccagtcaccgtcttcactctccgccagtgtgggtgacagagtgactatcacctgtcgagccagccaaaggatatctacttatatcaactggtatcagcagaaaccagggaaggcacctaagttattgatctactcggcgtccgttctggaaaatggagtcccctctcggtttagtgggagcggattcggcacggattttacacttaccattagctccctgcagccagaggatttcgctacctactactgccaacagaattatcgcactcctcccacattcggtagcggcacaaaaggcgagaacaag

SEQ ID NO: 331 - protein, 8 aa, synthetic construct
GFTFSNYA

SEQ ID NO: 332 - protein, 8 aa, synthetic construct
ITSSASKT

SEQ ID NO: 333 - protein, 15 aa, synthetic construct
ARTRYLERFAGGLDI

SEQ ID NO: 334 - protein, 6 aa, synthetic construct
QSISSY

SEQ ID NO: 335 - skipped

SEQ ID NO: 336 - protein, 9 aa, synthetic construct
QQSFTAPLT

SEQ ID NO: 337 - protein, 122 aa, synthetic construct
EVQLLESGGGLVQPGGSLRLSCAASGFTFSNYAMTWVRQAPGKGLEWVSTITSSASKTNYADSVKGRFTISRDNSKNTLYLQMNSLRAEDTAVYYCARTRYLERFAGGLDIWGQGTLVTVSS

SEQ ID NO: 338 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSFTAPLTFGGGTKVEIK

SEQ ID NO: 339 - DNA, 366 bp, synthetic construct
  source 1..366: mol_type = other DNA
gaggtccagctgctggaatcaggcggagggctggttcagcccggcggctccctcaggttaagctgcgcggcaagcgggtttaccttctctaattacgccatgacatgggtgagacaagcccctggaaaaggtttggagtgggtgtcgaccatcacgagctctgcttccaagactaactatgcagattctgtgaaaggccggttcacaatttccagagataactcaaagaatactctttacctacagatgaacagtctgcgagccgaagacactgctgtctattactgtgcccgtaccaggtatcttgagcgctttgctggcggtctggacatatggggacaggggacactcgtgaccgtaagcagt

SEQ ID NO: 340 - DNA, 321 bp, synthetic construct
  source 1..321: mol_type = other DNA
gacatccagatgacccagtctcctagtagcctttctgcaagcgttggagatcgggtgaccatcacatgcagagccagccaaagtataagctcatacttgaactggtaccagcagaagccaggtaaggctccaaaactgttaatttacgctgcctcttccctgcagtcgggcgtgcctagtaggttttcagggtcagggtccggaactgacttcaccctcacaatctcctccctgcagcccgaggatttcgccacctattattgtcaacagtcttttacggcgcccctcactttcggcggtggcacaaaggtcgaaattaaa

SEQ ID NO: 341 - protein, 8 aa, synthetic construct
GLTFSNYT

SEQ ID NO: 342 - protein, 8 aa, synthetic construct
ISGTGGST

SEQ ID NO: 343 - protein, 13 aa, synthetic construct
ARSRSYYAGAFDV

SEQ ID NO: 344 - protein, 6 aa, synthetic construct
VSISTF

SEQ ID NO: 345 - skipped

SEQ ID NO: 346 - protein, 9 aa, synthetic construct
QQSLRTPIT

SEQ ID NO: 347 - protein, 120 aa, synthetic construct
EVQLLESGGGLVQPGGSLRLSCAASGLTFSNYTMTWVRQAPGKGLEWVSGISGTGGSTWYADSVKGRFTISRDNSKNTLYLQMNSLRAEDTAVYYCARSRSYYAGAFDVWGQGTLVTVSS

SEQ ID NO: 348 - protein, 107 aa, synthetic construct
DIQMTQSPSSLSASVGDRVTITCRASVSISTFLNWYQQKPGKAPKLLIYAASTLQSGVPSRFSGSGSGTDFTLTISSLQPEDFATYYCQQSLRTPITFGGGTKVEIK

SEQ ID NO: 349 - DNA, 360 bp, synthetic construct
  source 1..360: mol_type = other DNA
gaggtgcaactgctggaatctggtggaggtctggtccagcccggaggcagtcttaggctcagctgtgccgcatccggcctaacttttagcaattacaccatgacatgggttcgtcaggcccctgggaaagggttggaatgggtttctggcatctcaggaaccgggggttcgacatggtacgctgattcagtgaagggcagattcaccatttcccgagacaacagcaagaatacgctgtatcttcagatgaacagtttaagggcggaggacactgcagtctattattgcgcccgctcccggtcctactacgctggggccttcgatgtgtggggccagggaaccctcgtaacagtgtctagc

SEQ ID NO: 350 - DNA, 321 bp, synthetic construct
  source 1..321: mol_type = other DNA
gatattcagatgactcaatcaccctcatcgctgtctgcgagtgtaggggaccgcgtcacaatcacctgtagagcctccgtgagcatcagtacgtttttaaactggtatcaacagaaacccgggaaggctcctaagctgctcatatacgcagcttcaacacttcagagcggtgttccaagcaggttttctgggtccggatctggaactgacttcacattgactatcagctccctccagccagaagatttcgccacctattactgccagcagtccctgcggacccctattaccttcggcggcggcacaaaggtggagattaaa

SEQ ID NO: 351 - protein, 8 aa, synthetic construct
GFPFSSFV

SEQ ID NO: 352 - protein, 8 aa, synthetic construct
LSGSGDIT

SEQ ID NO: 353 - protein, 14 aa, synthetic construct
ARAAPGAYAYALDY

SEQ ID NO: 354 - protein, 6 aa, synthetic construct
QSVSTY

SEQ ID NO: 355 - skipped

SEQ ID NO: 356 - protein, 9 aa, synthetic construct
QQSYRTPPT

SEQ ID NO: 357 - protein, 121 aa, synthetic construct
EVQLLESGGGLVQPGGSLRLSCAASGFPFSSFVMSWVRQAPGKGLEWVSSLSGSGDITYYADSVKGRFTISRDNSKNTLYLQMNSLRAEDTAVYYCARAAPGAYAYALDYWGQGTLVTVSS

SEQ ID NO: 358 - protein, 107 aa, synthetic construct
DIQMTQSPSSLSASVGDRVTITCRTSQSVSTYFNWYQQKPGKAPKLLIYAASSLQSGVPSRFSGSGSGTDFTLTISSLQPEDFATYYCQQSYRTPPTFGGGTKVEIK

SEQ ID NO: 359 - DNA, 363 bp, synthetic construct
  source 1..363: mol_type = other DNA
gaagtccagctcttggaatccggaggcggcctggtgcagcccggggggtcattgcgactgagttgcgccgcatctggtttccctttttctagcttcgtgatgtcctgggttagacaggccccggggaagggtttagagtgggtgagctcgctgtccggaagcggggacattacttattatgcagattctgttaagggccgctttaccatcagccgggacaactccaaaaatacactctaccttcaaatgaacagtctacgtgccgaggacaccgccgtgtactactgtgctagggctgccccaggcgcgtatgcatatgctctggattactggggacagggcacgctggtcacagtatcatca

SEQ ID NO: 360 - DNA, 321 bp, synthetic construct
  source 1..321: mol_type = other DNA
gatatccaaatgacacagtcgccttcttcccttagcgcatcagtcggggaccgcgtgacaattacctgtaggacctcccagagcgtgagtacttactttaactggtaccaacagaagcccggcaaagcccccaagctgctgatctatgccgcgtctagtttgcagagtggtgttccttcacggttctcagggtccggctctggcacagatttcaccttaactatttccagcctccagccagaggactttgctacgtattactgccagcagagctatagaactccgccaacattcggaggaggtaccaaggtagaaataaaa

SEQ ID NO: 361 - protein, 8 aa, synthetic construct
GLTFNAYA

SEQ ID NO: 362 - protein, 8 aa, synthetic construct
LTGSGAST

SEQ ID NO: 363 - protein, 13 aa, synthetic construct
ARSRSYYAGAFDV

SEQ ID NO: 364 - protein, 6 aa, synthetic construct
RTVTSY

SEQ ID NO: 365 - skipped

SEQ ID NO: 366 - protein, 9 aa, synthetic construct
QQSYRTPPT

SEQ ID NO: 367 - protein, 120 aa, synthetic construct
EVQLLESGGGLVQPGGSLRLSCAASGLTFNAYAMSWVRQAPGKGLEWVSSLTGSGASTFYADSVKGRFTISRDNSKNTLYLQMNSLRAEDTAVYYCARSRSYYAGAFDVWGQGTLVTVSS

SEQ ID NO: 368 - protein, 107 aa, synthetic construct
DIQMTQSPSSLSASVGDRVTITCRASRTVTSYLNWYQQKPGKAPKLLIYGASYLHSGVPSRFSGSGSGTDFTLTISSLQPEDFATYYCQQSYRTPPTFGGGTKVEIK

SEQ ID NO: 369 - DNA, 360 bp, synthetic construct
  source 1..360: mol_type = other DNA
gaggtgcagctactggagtcaggaggcgggctggttcagcctggggggtcattgcgactgagctgtgccgcatctggcctcacctttaacgcctacgccatgagctgggtccgtcaggcgcccggcaaaggcttggaatgggtgtccagcttaactgggtctggagcctccaccttctacgctgactcggtcaagggtagattcacaatctctcgggacaattcaaagaatacactttatctgcagatgaacagtctgagggctgaagatacggctgtgtactattgcgcacgctccaggtcctactatgccggtgcatttgatgtttggggccaaggaaccctcgtaactgtgagcagt

SEQ ID NO: 370 - DNA, 321 bp, synthetic construct
  source 1..321: mol_type = other DNA
gatatccagatgacacagtcgcccagctccctgagcgcttcagtgggggaccgcgtaactattacatgtagagccagtcgaaccgtcacctcctatctcaactggtaccaacagaagcctggcaaggcgccaaaattactgatatacggagcatcttatttgcactctggtgtgccgtctaggttctccggcagtgggagcggaaccgacttcactcttactatctccagcctgcagccagaagattttgccacatattactgccaacagtcataccggacccctcccacgtttgggggtggcacaaaggttgagattaaa

SEQ ID NO: 371 - protein, 8 aa, synthetic construct
GFSVRDFA

SEQ ID NO: 372 - protein, 8 aa, synthetic construct
IHRTGGGT

SEQ ID NO: 373 - protein, 13 aa, synthetic construct
ARSRSYYAGAFDV

SEQ ID NO: 374 - protein, 6 aa, synthetic construct
QNIGNY

SEQ ID NO: 375 - skipped

SEQ ID NO: 376 - protein, 8 aa, synthetic construct
QQSYSTST

SEQ ID NO: 377 - protein, 120 aa, synthetic construct
EVQLLESGGGLVQPGGSLRLSCAASGFSVRDFAMNWVRQAPGKGLEWVSSIHRTGGGTYYADSVKGRFTISRDNSKNTLYLQMNSLRAEDTAVYYCARSRSYYAGAFDVWGQGTLVTVSS

SEQ ID NO: 378 - protein, 106 aa, synthetic construct
DIQMTQSPSSLSASVGDRVTITCRANQNIGNYLNWYQQKPGKAPKLLIYAASSLQSGVPSRFSGSGSGTDFTLTISSLQPEDFATYYCQQSYSTSTFGGGTKVEIK

SEQ ID NO: 379 - DNA, 360 bp, synthetic construct
  source 1..360: mol_type = other DNA
gaggtccagctcctggaatcaggtgggggcctggtgcagcccggcgggagcttgcggctttcttgtgctgcaagcggattttcagtgagagacttcgccatgaattgggtccgtcaagcccctggaaaggggttagaatgggtgtctagcattcacaggacaggcgggggcacatactacgcagatagtgttaaaggcaggttcacgatctccagagacaattccaagaacaccctctacctacagatgaacagtctgcgagcggaggacaccgccgtttattactgcgcccggagccgctcctattatgctggagcttttgatgtgtggggtcagggaactctggtgactgtatcttcg

SEQ ID NO: 380 - DNA, 318 bp, synthetic construct
  source 1..318: mol_type = other DNA
gatattcagatgacgcagtcccccagtagcttgagtgccagcgtgggtgaccgggttactatcacatgtagagcgaaccagaatatcggcaactaccttaattggtaccaacagaagcctgggaaggctcccaaactcctgatctatgcagcctcatccctgcagtcaggtgtgccgtccaggttttcggggtccgggagcggaaccgatttcaccctgacaatttcttctttacaaccagaagactttgctacctactattgccagcagtcatatagcacctctactttcggcggcggaacaaaggtcgagataaaa

SEQ ID NO: 381 - protein, 8 aa, synthetic construct
GFDFRSYP

SEQ ID NO: 382 - protein, 8 aa, synthetic construct
ISGGGGST

SEQ ID NO: 383 - protein, 13 aa, synthetic construct
ARSRSYYAGAFDV

SEQ ID NO: 384 - protein, 6 aa, synthetic construct
QKIARY

SEQ ID NO: 385 - skipped

SEQ ID NO: 386 - protein, 9 aa, synthetic construct
QQAYSIPIT

SEQ ID NO: 387 - protein, 120 aa, synthetic construct
EVQLLESGGGLVQPGGSLRLSCAASGFDFRSYPMAWVRQAPGKGLEWVSVISGGGGSTNYADSVKGRFTISRDNSKNTLYLQMNSLRAEDTAVYYCARSRSYYAGAFDVWGQGTLVTVSS

SEQ ID NO: 388 - protein, 107 aa, synthetic construct
DIQMTQSPSSLSASVGDRVTITCRASQKIARYVNWYQQKPGKAPKLLIYAASDLQSGVPSRFSGSGSGTDFTLTISSLQPEDFATYYCQQAYSIPITFGGGTKVEIK

SEQ ID NO: 389 - DNA, 360 bp, synthetic construct
  source 1..360: mol_type = other DNA
gaggtccagctgttggaaagcggggggggcttagtacagcctggaggttccctccgtctaagctgcgccgcatcgggattcgactttcgctcctacccaatggcttgggtgcggcaggcccccggcaagggcctggaatgggttagtgttatttctggtggaggcggttcaaccaactacgcggacagcgtcaaagggagatttactatctctagggacaattccaagaacacgctgtacctgcagatgaatagtctccgagcagaggatacagccgtgtattattgtgccaggtcaagatcctactatgctggagctttcgatgtgtggggccaagggacacttgtgaccgtgagctct

SEQ ID NO: 390 - DNA, 321 bp, synthetic construct
  source 1..321: mol_type = other DNA
gacatacagatgacacagtctccatctagccttagtgcctccgttggagatcgggtgaccatcacatgccgcgcctcacagaagattgcaagatatgtcaactggtaccagcagaaacctggtaaggcaccaaagctcttaatctatgctgcgagcgacttgcaaagcggcgtgccctctaggttttccggcagcggttcaggcaccgatttcaccctgacgatctcctcgctgcagcctgaggactttgctacttattactgtcagcaagcctacagtattcccattacattcgggggagggactaaagtagaaatcaaa

SEQ ID NO: 391 - protein, 8 aa, synthetic construct
GFRFSNYA

SEQ ID NO: 392 - protein, 8 aa, synthetic construct
ISGGGGRT

SEQ ID NO: 393 - protein, 11 aa, synthetic construct
ARGYGAEYFDP

SEQ ID NO: 394 - protein, 6 aa, synthetic construct
QPISRY

SEQ ID NO: 395 - skipped

SEQ ID NO: 396 - protein, 9 aa, synthetic construct
QQSHSIPWT

SEQ ID NO: 397 - protein, 118 aa, synthetic construct
EVQLLESGGGLVQPGGSLRLSCAASGFRFSNYAMTWVRQAPGKGLEWVSGISGGGGRTFYADSVKGRFTISRDNSKNTLYLQMNSLRAEDTAVYYCARGYGAEYFDPWGQGTLVTVSS

SEQ ID NO: 398 - protein, 107 aa, synthetic construct
DIQMTQSPSSLSASVGDRVTITCRASQPISRYLNWYQQKPGKAPKLLIYDASRLQVGVPSRFSGSGSGTDFTLTISSLQPEDFATYYCQQSHSIPWTFGGGTKVEIK

SEQ ID NO: 399 - DNA, 354 bp, synthetic construct
  source 1..354: mol_type = other DNA
gaggtgcaactccttgagtcaggtggcggactcgtgcagcctggcggatcgctgagactgtcatgtgcggcttctgggtttcgtttctccaactacgcaatgacatgggtgaggcaggcccccgggaaaggcctggagtgggtatccggaatcagcggcggcgggggcaggaccttctatgcagatagtgtcaagggtagatttaccattagccgggacaattccaagaatacattatacctacagatgaacagtctgcgcgccgaagatacggctgtctattactgcgcccgaggttacggggccgaatatttcgacccgtgggggcagggaactttggttactgtgagctct

SEQ ID NO: 400 - DNA, 321 bp, synthetic construct
  source 1..321: mol_type = other DNA
gacatccagatgacccagtcaccctcttccctgtccgcaagtgtgggcgaccgggtgactattacttgtcgcgccagccagcccatcagtagatacttaaactggtatcagcagaagccgggcaaagcgccaaagctgttgatatatgacgcttctcgacttcaagtcggcgtacctagcaggttctcagggagcggttctggaacggatttcacactgaccatctcgtccctccaaccagaggattttgccacatactactgccagcagagccactccattccttggacatttgggggtggaaccaaggttgaaattaaa

SEQ ID NO: 401 - protein, 8 aa, synthetic construct
GFRFSNYA

SEQ ID NO: 402 - protein, 8 aa, synthetic construct
ISGGGGRT

SEQ ID NO: 403 - protein, 11 aa, synthetic construct
ARGYGAEYFDP

SEQ ID NO: 404 - protein, 6 aa, synthetic construct
QPISRH

SEQ ID NO: 405 - skipped

SEQ ID NO: 406 - protein, 9 aa, synthetic construct
QQSYDRTWT

SEQ ID NO: 407 - protein, 118 aa, synthetic construct; identical to SEQ ID NO: 397

SEQ ID NO: 408 - protein, 107 aa, synthetic construct
DIQMTQSPSSLSASVGDRVTITCRASQPISRHLNWYQQKPGKAPKLLIYSASSLQSGVPSRFSGSGSGTDFTLTISSLQPEDFATYYCQQSYDRTWTFGGGTKGESK

SEQ ID NO: 409 - DNA, 354 bp, synthetic construct
  source 1..354: mol_type = other DNA
gaggtgcagctgctcgaatcgggcgggggcctggtccagcccggggggtctttgaggctctcctgtgctgcctcagggtttaggttcagtaattacgcgatgacttgggtgcgacaggccccaggaaagggcctagagtgggtgagcggcatttcaggtggaggaggcagaacattttatgcagatagcgttaaaggtagattcacgatctctcgggacaactccaagaataccctttacttacaaatgaacagtctgcgcgcagaggataccgccgtatactattgcgctcgtgggtatggagccgaatacttcgacccttggggccagggtaccctggtcacagtgagctcc

SEQ ID NO: 410 - DNA, 321 bp, synthetic construct
  source 1..321: mol_type = other DNA
gacatccaaatgacccagagcccatcatccctgagcgcatcagtgggagatagagtcactatcacctgtcgcgcttctcaaccaatatcccgacacctcaactggtaccagcagaaacccggaaaggcccctaagctgttgatttatagtgccagttccctgcagtcaggtgttcctagcaggttttctggctctgggtccggcacagacttcacgcttactatttctagcttacagcccgaagattttgcgacatactactgccagcagagttatgaccggacatggaccttcggtgggggcaccaagggggagtcgaaa

SEQ ID NO: 411 - protein, 8 aa, synthetic construct
GFRFSNYA

SEQ ID NO: 412 - protein, 8 aa, synthetic construct
ISGGGGRT

SEQ ID NO: 413 - protein, 11 aa, synthetic construct
ARGYGAEYFDP

SEQ ID NO: 414 - protein, 6 aa, synthetic construct
QRIATY

SEQ ID NO: 415 - skipped

SEQ ID NO: 416 - protein, 9 aa, synthetic construct
QQSYSIPLT

SEQ ID NO: 417 - protein, 118 aa, synthetic construct; identical to SEQ ID NO: 397

SEQ ID NO: 418 - protein, 107 aa, synthetic construct
DIQMTQSPSSLSASVGDRVTITCRASQRIATYLNWYQQKPGKAPKLLIYAASHLHGGVPSRFSGSGSGTDFTLTISSLQPEDFATYYCQQSYSIPLTFGGGTKVEIK

SEQ ID NO: 419 - DNA, 354 bp, synthetic construct
  source 1..354: mol_type = other DNA
gaagtccagctcttggaatccgggggggggttagtgcagcctggaggctctctcagactgagttgcgccgccagcggatttaggttctccaattatgccatgacgtgggtgagacaggcccccgggaaggggctggagtgggtgtctggcatttccggcggaggcggacggactttctatgcagactcagtaaagggtaggttcacaatcagccgagacaactcgaaaaataccctttacctgcaaatgaacagtctgcgcgcggaggataccgctgtgtactactgtgcacgtggttacggcgctgagtattttgatccgtggggccagggtacactagtcactgttagctca

SEQ ID NO: 420 - DNA, 321 bp, synthetic construct
  source 1..321: mol_type = other DNA
gacattcagatgacccagtcacccagtagcttgtctgcctctgtgggagatcgcgtcacaataacttgcagggcttcccaacggatcgccacctacctgaactggtaccagcaaaaacctggtaaggcaccaaagctgttaatttacgcggcctctcaccttcatggaggcgtgccatccagattctccggcagcgggtcaggcacagattttaccctgactatctcctcgctccagcccgaagacttcgctacgtattattgtcagcagagttatagcatccctctcacatttggtggggggaccaaggttgagattaaa

SEQ ID NO: 421 - protein, 8 aa, synthetic construct
GFTVSRDY

SEQ ID NO: 422 - protein, 8 aa, synthetic construct
ISTGGGST

SEQ ID NO: 423 - protein, 17 aa, synthetic construct
AKPRPYSIAWFADPFDY

SEQ ID NO: 424 - protein, 6 aa, synthetic construct
QTISIY

SEQ ID NO: 425 - skipped

SEQ ID NO: 426 - protein, 9 aa, synthetic construct
QQNYSVPPT

SEQ ID NO: 427 - protein, 124 aa, synthetic construct
GGAALKSGGGLVQPGGSLRLSCAASGFTVSRDYMSWVRQAPGKGLEWVSVISTGGGSTYYADSVKGRFTISRDNSKNTLYLQMNSLRAEDTAVYYCAKPRPYSIAWFADPFDYWGQGTLVTVSS

SEQ ID NO: 428 - protein, 107 aa, synthetic construct
DIQMTQSPSSLSASVGDRVTITCRTSQTISIYLNWYQQKPGKAPKLLIYAASILHGGVPSRFSGSGSGTDFTLTISSLQPEDFATYYCQQNYSVPPTFGGGTKVEIK

SEQ ID NO: 429 - DNA, 372 bp, synthetic construct
  source 1..372: mol_type = other DNA
ggaggcgcggccttaaagtccggaggtggcctggtccagcccggagggtccctcagattgagctgtgctgcctctggctttacagtgtcacgggattatatgtcgtgggtccgccaagctccgggcaagggactggaatgggtgagtgtgattagcaccgggggtgggtccacgtactatgccgactctgttaaggggaggttcacaatctccagggataattcaaaaaacaccctgtaccttcagatgaacagtctccgtgctgaggacaccgcagtttattattgcgcaaaaccccgaccatacagcatagcatggttcgccgacccttttgattactggggccagggtactctggtgactgtaagctct

SEQ ID NO: 430 - DNA, 321 bp, synthetic construct
  source 1..321: mol_type = other DNA
gatatccaaatgactcagagcccttccagcttgagtgcgtccgtgggggatagggtaactatcacatgtagaacaagccagaccatatctatttacctcaactggtatcagcagaagcccggaaaggccccaaaattactgatttatgcagcctcaatcctgcacggaggtgttccgtcacggttctccgggtcgggctctggtaccgactttacccttacgattagtagcctgcagcctgaggacttcgctacctactactgccaacagaattattctgtcccacccacttttgggggcggcacaaaagtggaaatcaag

SEQ ID NO: 431 - protein, 8 aa, synthetic construct
GFPFSNNA

SEQ ID NO: 432 - protein, 8 aa, synthetic construct
ISGSYGTT

SEQ ID NO: 433 - protein, 14 aa, synthetic construct
ARVADGAAAYAMDY

SEQ ID NO: 434 - protein, 6 aa, synthetic construct
QPISRY

SEQ ID NO: 435 - skipped

SEQ ID NO: 436 - protein, 9 aa, synthetic construct
QQSHSIPWT

SEQ ID NO: 437 - protein, 121 aa, synthetic construct
ELQLLESGGGLVQPGGSLRLSCAASGFPFSNNAMSWVRQAPGKGLEWVSVISGSYGTTYYADSVKGRFTISRDNSKNTLYLQMNSLRAEDTAVYYCARVADGAAAYAMDYWGQGTLVTVSS

SEQ ID NO: 438 - protein, 107 aa, synthetic construct; identical to SEQ ID NO: 398

SEQ ID NO: 439 - DNA, 363 bp, synthetic construct
  source 1..363: mol_type = other DNA
gagctgcaactcttagaatctggcggaggcctggttcagcccggaggcagtctgcgtctatcctgtgccgcatctgggttccctttctccaacaacgccatgtcatgggtccgccaggccccaggtaaaggtcttgaatgggtttctgtgatcagcggcagctacgggactacatactatgccgattccgtgaagggcagatttaccattagccgggataattcgaagaacacgctttatctgcagatgaatagtttgagggctgaggacaccgcagtgtattactgcgcccgagtggctgacggggcagctgcttacgcgatggactattgggggcagggaactctcgtcacagtaagctca

SEQ ID NO: 440 - DNA, 321 bp, synthetic construct
  source 1..321: mol_type = other DNA
gacattcagatgacccagtcaccatccagtctgtcagcttcggtgggcgatagggtaacaatcacttgcagagcgtcccagcccatctctcgctatcttaactggtaccagcaaaagccggggaaagcaccaaagctgttaatttacgacgcctctcggctgcaggtcggcgttcctagccgattcagcggttctgggtccggaacagattttaccctcacaattagctccttgcagcccgaagactttgccacctattactgtcagcaaagtcacagcatcccttggacgttcggtggaggcactaaggtggagataaaa

SEQ ID NO: 441 - protein, 8 aa, synthetic construct
GFRFSNYA

SEQ ID NO: 442 - protein, 8 aa, synthetic construct
ISGGGGRT

SEQ ID NO: 443 - protein, 11 aa, synthetic construct
ARGYGAEYFDP

SEQ ID NO: 444 - protein, 6 aa, synthetic construct
QPISRY

SEQ ID NO: 445 - skipped

SEQ ID NO: 446 - protein, 9 aa, synthetic construct
QQSYNPPLT

SEQ ID NO: 447 - protein, 118 aa, synthetic construct; identical to SEQ ID NO: 397

SEQ ID NO: 448 - protein, 107 aa, synthetic construct
DIQMTQSPSSLSASVGDRVTITCRASQPISRYLNWYQQKPGKAPKLLIYAASNLKKGVPSRFSGSGFGTDFTLTISSLQPEDFATYYCQQSYNPPLTFGGGTKVEIK

SEQ ID NO: 449 - DNA, 354 bp, synthetic construct
  source 1..354: mol_type = other DNA
gaggtgcaactgcttgagagtggcggaggcctggtgcagcctgggggcagcctcagattatcttgcgcggcctcaggttttcgtttctctaactatgctatgacatgggtccgacaggctccagggaaagggctagaatgggtatcgggaatttccggtggcgggggccggactttctacgccgatagcgtcaagggaaggttcacaatcagccgcgacaattcaaagaataccctctacctgcagatgaacagtttgagggccgaggacactgcagtgtattactgtgccagaggctacggagcagaatattttgatccctgggggcagggtaccctggtgacggtttcctcc

SEQ ID NO: 450 - DNA, 321 bp, synthetic construct
  source 1..321: mol_type = other DNA
gacatccagatgacccaaagtccctcttctcttagcgcttcggtgggcgatcgggtgaccattacttgtagagcgtcacagccaataagcaggtatctcaattggtatcagcagaagcccggaaaagcacccaagttgctgatctacgccgcctccaacttaaagaagggggttcctagccgctttagtgggtccggcttcggaaccgatttcacactcacaatctcatccctgcagccggaggactttgctacgtactattgccaacagtcttacaaccctccactgactttcggtggcgggacaaaagtcgaaattaaa

SEQ ID NO: 451 - protein, 8 aa, synthetic construct
GFTFPHSA

SEQ ID NO: 452 - protein, 8 aa, synthetic construct
IAGRGGSP

SEQ ID NO: 453 - protein, 14 aa, synthetic construct
ARVADGGAAYAFDY

SEQ ID NO: 454 - protein, 6 aa, synthetic construct
QNIITY

SEQ ID NO: 455 - skipped

SEQ ID NO: 456 - protein, 9 aa, synthetic construct
QQSFSTPLT

SEQ ID NO: 457 - protein, 121 aa, synthetic construct
EVQLLESGGGLVQPGGSLRLSCAASGFTFPHSAMSWVRQAPGKGLEWVSSIAGRGGSPNYADSVKGRFTISRDNSKNTLYLQMNSLRAEDTAVYYCARVADGGAAYAFDYWGQGTLVTVSS

SEQ ID NO: 458 - protein, 107 aa, synthetic construct
DIQMTQSPSSLSASVGDRVTITCRSSQNIITYLNWYQQKPGKAPKLAIYGASRVQSGVPSRFSGSGSGADFTLTISSLQPEDFSTYYCQQSFSTPLTFGGGTKVEIK

SEQ ID NO: 459 - DNA, 363 bp, synthetic construct
  source 1..363: mol_type = other DNA
gaggtgcagctgctggagagcggaggcggactagtgcagccaggtgggtccctgcgacttagctgcgccgcttcggggtttacgtttccgcactctgcaatgtcttgggtgcgtcaggcacctggaaagggcttagaatgggtcagcagcatcgctggcagaggtggctctcccaattacgccgactccgtgaagggtaggttcactatttcccgcgataactcaaaaaatacactctatctgcagatgaacagtttgagggccgaagacaccgcggtatattattgtgcacgggttgccgatggcggggctgcctacgctttcgactactgggggcaaggaaccctcgttacagtcagttca

SEQ ID NO: 460 - DNA, 321 bp, synthetic construct
  source 1..321: mol_type = other DNA
gacattcaaatgacacagagtccttcttccttgtctgcatccgtcggtgatcgcgttaccatcacttgccggtcgagccagaacataatcacttatctcaattggtaccagcagaaacccggaaaagccccaaagttagcgatttacggtgcctccagggtgcagagcggcgtgcccagcagattcagcgggagtgggtcaggggctgatttcacactgaccatcagttctcttcagcctgaggacttttcaacctattactgtcaacagtccttctcaacgccactgacctttggcggaggcacaaaggtagaaattaag

SEQ ID NO: 461 - protein, 8 aa, synthetic construct
GFRFSNYA

SEQ ID NO: 462 - protein, 8 aa, synthetic construct
ISGGGGRT

SEQ ID NO: 463 - protein, 11 aa, synthetic construct
ARGYGAEYFDP

SEQ ID NO: 464 - protein, 6 aa, synthetic construct
QPISRY

SEQ ID NO: 465 - skipped

SEQ ID NO: 466 - protein, 9 aa, synthetic construct
QQSHSIPWT

SEQ ID NO: 467 - protein, 118 aa, synthetic construct; identical to SEQ ID NO: 397

SEQ ID NO: 468 - protein, 107 aa, synthetic construct
DIQMTQSPSSLSASVGDRVTITCRASQPISRYLNWYQQKPGKAPKLLIYDASRLQVGVPSRFSGSGSGTDFTLTISSPQPEDFATYYCQQSHSIPWTFGGGTKGEIK

SEQ ID NO: 469 - DNA, 354 bp, synthetic construct
  source 1..354: mol_type = other DNA
gaggtgcagctcctggaaagcggagggggcttggtgcagcccggagggtccctccgattatcctgtgctgcctcagggtttcgtttttcaaattacgcaatgacatgggtccggcaggcccctggcaaggggctggagtgggtgagcgggatttccggaggtggtggcagaacgttctacgcagattcggtgaagggtagattcaccatcagtagggacaattctaaaaacactctatatctgcagatgaacagtctgagggctgaagacactgccgtttattattgcgcgcgcggatacggcgccgagtacttcgatccgtggggccaaggcacccttgtcacagtaagctct

SEQ ID NO: 470 - DNA, 321 bp, synthetic construct
  source 1..321: mol_type = other DNA
gacatccagatgacacagtcaccctccagcctgtctgcctctgtgggggacagggtcaccattacttgtcgggcatcgcaaccaattagcagatacttgaactggtatcagcagaagccaggaaaggcccccaagttactcatttacgacgctagtcgacttcaagtgggtgttccttctcgcttctccgggtccggaagcggcactgattttaccctgaccatctcaagtccccagcctgaagattttgcgacatattactgccagcagtcccacagcataccgtggacgttcggtgggggcacaaaaggcgagatcaaa

SEQ ID NO: 471 - protein, 8 aa, synthetic construct
GFTFPHSA

SEQ ID NO: 472 - protein, 8 aa, synthetic construct
VTGSGSPT

SEQ ID NO: 473 - protein, 14 aa, synthetic construct
ARVAGGAYGYAMDY

SEQ ID NO: 474 - protein, 6 aa, synthetic construct
QSISSY

SEQ ID NO: 475 - skipped

SEQ ID NO: 476 - protein, 9 aa, synthetic construct
QQSFSNLYT

SEQ ID NO: 477 - protein, 121 aa, synthetic construct
EVQLLESGGGLVQPGGSLRLSCAASGFTFPHSAMSWVRQAPGKGLEWVSTVTGSGSPTYYADSVKGRFTISRDNSKNTLYLQMNSLRAEDTAVYYCARVAGGAYGYAMDYWGQGTLVTVSS

SEQ ID NO: 478 - protein, 107 aa, synthetic construct
DIQMTQSPSSLSASVGDRVTITCRASQSISSYLNWYQQKPGKAPKLLIYDASNLQSGVPSRFSGSGSGTDFTLTISSLQPEDFATYYCQQSFSNLYTFGGGTKVEIK

SEQ ID NO: 479 - DNA, 363 bp, synthetic construct
  source 1..363: mol_type = other DNA
gaagtgcagctcctggagtcaggcgggggactggtccagcccggaggttcactgcgtctaagctgcgctgcatctggcttcacattcccacactccgccatgtcttgggttcgccaggcccctgggaagggattggaatgggtgtccaccgttacaggttccggcagcccgacatattatgccgatagcgtgaaagggcgatttaccatcagcagggacaacagtaagaatacgctgtatcttcagatgaacagtctccgggctgaggatactgcggtctactactgtgcaagagtggctggaggggcctacggttatgcaatggactactggggccaaggcactttagtgaccgtatcgtct

SEQ ID NO: 480 - DNA, 321 bp, synthetic construct
  source 1..321: mol_type = other DNA
gacatacagatgacccagtccccgtcaagtctcagcgcctcggtcggggacagagttacaatcacgtgtcgggcatctcaatccatttccagctatctgaattggtaccagcagaaacctgggaaggctccaaagctgcttatctacgatgccagcaacctgcagagcggagtgccctcaaggttctccggcagtggctctgggactgattttaccctcacaatttcttctttacagcccgaagactttgcgacttactattgccagcaatcattcagtaacttgtataccttcggtggaggcacaaaggtggagatcaaa

SEQ ID NO: 481 - protein, 8 aa, synthetic construct
GFPFSVYA

SEQ ID NO: 482 - protein, 8 aa, synthetic construct
FGGSGHSP

SEQ ID NO: 483 - protein, 14 aa, synthetic construct
ARVAAGSYAYAMDY

SEQ ID NO: 484 - protein, 6 aa, synthetic construct
QNIITY

SEQ ID NO: 485 - skipped

SEQ ID NO: 486 - protein, 9 aa, synthetic construct
QQSFSTPLT

SEQ ID NO: 487 - protein, 121 aa, synthetic construct
EVQLLESGGGLVQPGGSLRLSCAASGFPFSVYAMTWVRQAPGKGLEWVSSFGGSGHSPYYADSVKGRFTISRDNSKNTLYLQMNSLRAEDTAVYYCARVAAGSYAYAMDYWGQGTLVTVSS

SEQ ID NO: 488 - protein, 107 aa, synthetic construct
DIQMTQSPSSLSASVGDRVTITCRSSQNIITYLNWYQQKPGKAPKLLIYGASRLQSGVPSRFSGSGSGTDFTLTISSLQPEDFATYYCQQSFSTPLTFGGGTKEVDK

SEQ ID NO: 489 - DNA, 363 bp, synthetic construct
  source 1..363: mol_type = other DNA
gaggtgcagcttctggaatcagggggtggactggttcagcctgggggttccctccgcctgtcgtgtgccgcttctggctttccattctccgtatatgcaatgacatgggtgaggcaggccccgggcaagggattggagtgggtgagttcttttggcggcagcgggcactccccctattacgcagattccgtgaaggggcgtttcacaatctcaagagataattctaaaaacacgctgtatctacagatgaacagtctccgggctgaagacaccgccgtttactactgcgcgcgagtggccgctggcagctacgcctatgcaatggactactggggacaaggaaccttagtcactgtcagcagc

SEQ ID NO: 490 - DNA, 321 bp, synthetic construct
  source 1..321: mol_type = other DNA
gatatccagatgactcagagcccatcatcgctcagcgcatctgtcggggacagggttaccataacctgcagaagttcacagaacatcatcacctaccttaattggtaccagcaaaagcctggcaaagcccctaaactgctcatttatggagcctcccggctgcagagcggcgtgcccagtcgcttctccggttctggatcaggtaccgatttcacgctgacaattagctccttacaacccgaagactttgctacttactattgtcagcagtccttctctactccattgacatttggcggggggacaaaggaggtggacaag

SEQ ID NO: 491 - protein, 8 aa, synthetic construct
GFRFSNYA

SEQ ID NO: 492 - protein, 8 aa, synthetic construct
ISGGGGRT

SEQ ID NO: 493 - protein, 11 aa, synthetic construct
ARGYGAEYFDP

SEQ ID NO: 494 - protein, 6 aa, synthetic construct
QPISRY

SEQ ID NO: 495 - skipped

SEQ ID NO: 496 - protein, 9 aa, synthetic construct
QQSFSTPLT

SEQ ID NO: 497 - protein, 118 aa, synthetic construct; identical to SEQ ID NO: 397

SEQ ID NO: 498 - protein, 107 aa, synthetic construct
DIQMTQSPSSLSASVGDRVTITCRASQPISRYLNWYQQKPGKAPKLLIYDASRLQVGVPSRFSGSGSGTDFTLTISSLQPEDFATYYCQQSFSTPLTFGGGTKVEIK

SEQ ID NO: 499 - DNA, 354 bp, synthetic construct
  source 1..354: mol_type = other DNA
gaagtccagcttctggagagcggaggcggactggtgcagccaggcgggagcttgcgtctgtcctgtgcggcaagcgggttccgcttctctaactatgccatgacctgggtaaggcaagcacctggcaaaggtcttgagtgggtgtccggaatttccggcgggggcggacggacgttctacgccgattcagtgaagggtcgatttaccatctctagggataatagtaagaacactctctacctccagatgaatagtttgagagctgaagacacagccgtgtactattgcgctagagggtacggtgccgagtattttgacccctgggggcagggcactctggttacagtctcatcg

SEQ ID NO: 500 - DNA, 321 bp, synthetic construct
  source 1..321: mol_type = other DNA
gatattcagatgacacagagcccatcttcactgtccgcttcggtaggggatagagtgacaataacatgccgcgcaagccagcctatctcacggtacctcaactggtaccaacagaaacccgggaaagccccgaagctgttaatctatgacgcgagtaggctgcaagtgggtgtccccagccgattcagcggttccggatctggcaccgacttcaccctcactatctcttccttgcagccagaagactttgccacgtattactgtcagcagtcctttagtacccctcttaccttcggcggaggcactaaggttgagattaag

SEQ ID NO: 501 - protein, 245 aa, synthetic construct
EVQLLESGGGLVQPGGSLRLSCAASGFAFNNYAMTWVRQAPGKGLEWVSVISGSGTSKYYADSVKGRFTISRDNSKNTLYLQMNSLRAEDTAVYYCAKPRPGSIFWGAGPFDYWGQGTLVTVSSGGGGSGGGGSGGGGSDIQMTQSPSSLSASVGDRVTITCRASQSISSYLHWYQQKPGKAPKLLIYAASSLQSGVPSRFSGSGSGTDFTLTISSLQPEDFATYYCQQSYGSPTFGGGTKVEIK

SEQ ID NO: 502 - DNA, 735 bp, synthetic construct
  source 1..735: mol_type = other DNA
gaagtgcagctcctagaatccggcggtgggctggttcagcctggagggtcccttagactgtcttgtgccgcgagtggcttcgcctttaacaattatgcaatgacctgggtgaggcaggcacccggcaagggactggagtgggtaagcgtcatcagcgggagcggcacttctaagtactacgccgattctgttaaaggtcgattcaccatatcacgggacaactccaagaacactttgtatctgcaaatgaatagtctccgcgccgaggatacggctgtctactactgcgctaaaccgcgtccaggttcaattttttggggagctggacccttcgactattgggggcagggcacattagtgacagtgtcgagcggtggcggtggatcgggcggtggtggatctggaggaggtggctcggacatccagatgacacagtctccttcttctctgtccgctagtgtgggcgacagagtgacgatcacctgtagggcgagtcaaagcatttcatcctacttgcactggtaccagcagaagcccggaaaagcacctaaactgttaatttacgccgccagttccctccaaagcggtgtcccatctcggttttcggggtcagggagcggcactgatttcacccttaccatctcatccctgcagcccgaagattttgctacatattattgccagcagagctatgggagcccaacattcggtggaggcactaaggttgagataaag

SEQ ID NO: 503 - protein, 243 aa, synthetic construct
EVQLLESGGGLVQPGGSLRLSCAASGFTFGASAMTWVRQAPGKGLEWVSGISGLGRTTDYADSVKGRFTISRDNSKNTLYLQMNSLRAEDTAVYYCARVAPGAYAYAMDYWGQGTLVTVSSGGGGSGGGGSGGGGSDIQMTQSPSSLSASVGDRVTITCRASQNIGHYLNWYQQKPGKAPKLLIYDASRLQVGVPSRFSGSGSGTDFTLTISSLQPEDFATYYCQQSYSTPPTFGGGTKVEIK

SEQ ID NO: 504 - DNA, 729 bp, synthetic construct
  source 1..729: mol_type = other DNA
gaagtgcaattgcttgagagtggcggcggcctggttcagcctgggggctccctgagacttagctgtgctgcctctggcttcaccttcggagcttcagcaatgacctgggtgcgtcaggctccaggtaagggcctggagtgggtgtcaggtatcagcggactagggcggacaaccgattacgctgatagcgtcaaagggcgctttacgatttcgagggacaactctaagaacactctgtatctccagatgaatagtttaagggcagaagacacagccgtgtactactgcgcccgagtagcccccggagcgtatgcctatgcaatggactactgggggcagggaactctcgtcacagtttcctccggtggcggtggatcgggcggtggtggatctggaggaggtggctcggacatacagatgacacagtctccttcctcacttagcgcgtcggttggcgaccgcgtgaccatcacatgtagagcatcacagaacattggtcactacctgaattggtaccagcagaaacccggaaaggccccaaaattattgatctatgacgcctccaggctgcaagtgggcgtaccatctcggttctccggtagcgggagcggcaccgattttacactgactatcagttctctccagcccgaagattttgctacgtattattgccaacagagttacagcaccccgcctaccttcgggggagggactaaggtcgagattaag

SEQ ID NO: 505 - protein, 242 aa, synthetic construct
EVQLLESGGGLVQPGGSLRLSCAASGFNFRSYVMNWVRQAPGKGLEWVSAISDVGRRTYYADSVKGRFTISRDNSKNTLYLQMNSLRAEDTAVYYCAKAEILGDYAYMDYWGQGTLVTVSSGGGGSGGGGSGGGGSDIQMTQSPSSLSASVGDRVTITCRASESIGNYLSWYQQKPGKAPKLLIYAASRLQRGVPSRFSGSGSGTDFTLTISSLQPEDFATYYCQQSYSTPTFGGGTKVEIK

SEQ ID NO: 506 - DNA, 726 bp, synthetic construct
  source 1..726: mol_type = other DNA
gaggtccagctgctggagtcaggcggtggccttgttcagcccggcgggtctttgcggctgtcctgcgccgccagcggattcaacttcagaagctacgttatgaactgggtgcgccaggcccctggcaagggcttagaatgggtgtccgctatctccgatgtcggacgtaggacttactatgcagactctgtgaaagggagatttaccattagtcgagataactcaaaaaatacactgtatctccagatgaatagtttgagggccgaggacaccgcagtatattactgtgctaaggctgaaatactcggtgactatgcgtacatggattactgggggcaaggaacactagtgacggtgagctcgggtggcggtggatcgggcggtggtggatctggaggaggtggctcggacatccagatgacccagagtccctcatctctgagtgcatcagtgggggaccgcgtgactattacctgcagagcttctgagtccataggtaactatctgtcctggtaccagcaaaaacctggcaaggcgcccaagttgctgatttacgccgcttcgcgattacagaggggcgtcccatcccggtttagcgggtcagggtccggtactgatttcacacttacaatcagcagcctccaaccagaagattttgccacctattactgtcagcagagctattctacacctaccttcggaggaggcacgaaggttgagatcaaa

SEQ ID NO: 507 - protein, 241 aa, synthetic construct
EVQLLESGGGLVQPGGSLRLSCAASGFAFSAYAMNWVRQAPGKGLEWVSSITESGAATYYADSVKGRFTISRDNSKNTLYLQMNSLRAEDTAVYYCARGLLASYTGDVWGQGTLVTVSSGGGGSGGGGSGGGGSDIQMTQSPSSLSASVGDRVTITCRASQSISSYLNWYQQKPGKAPKLLIYATSTLQSGVPSRFSGSGSGTDFTLTISSLQPEDFATYYCQQRDNAPWTFGGGTKVEIK

SEQ ID NO: 508 - DNA, 723 bp, synthetic construct
  source 1..723: mol_type = other DNA
gaagtgcagctcctagagtcgggcggcgggctggtgcagcccggtggctccctccggctgagctgcgccgcgagcggtttcgctttcagtgcctacgccatgaactgggttcgtcaagcccctggaaaaggcctggagtgggtatcaagcatcacggagtctggggctgctacatattatgcagattctgtgaagggaagatttactatttcccgcgacaatagcaagaataccctgtatctgcagatgaacagtttgcgagcagaagacaccgccgtctactactgtgcaagggggcttcttgcttcatacacaggggatgtgtggggccagggaaccttagtcactgtttcctctggtggcggtggatcgggcggtggtggatctggaggaggtggctcggacattcagatgacacaaagcccctccagcctgagcgcatcggtgggggaccgcgtcaccataacttgtcgggccagtcagtctatcagctcatatctgaactggtaccagcagaagcctggcaaagctccaaagctgcttatctacgctacttccactttacagtccggtgttccatccaggttctcaggatctggctctggtacagatttcaccttgaccatttcaagtctccagcctgaagattttgccacgtactattgccaacagagagacaatgcgccctggacatttgggggcggaaccaaagtggagatcaag

SEQ ID NO: 509 - protein, 241 aa, synthetic construct
EVQLLESGGGLVQPGGSLRLSCAASGFDFSSFAMTWVRQAPGKGLEWVSHITGSGGTIYYADSVKGRFTISRDNSKNTLYLQMNSLRAEDTAVYYCASASSYYEPGDPWGQGTLVTVSSGGGGSGGGGSGGGGSDIQMTQSPSSLSASVGDRVTITCRASQAIKNYLNWYQQKPGKAPKLLIYAGSTLQSGVPSRFSGSGSGTDFTLTISSLQPEDFATYYCQQTYSTPLTFGGGTKVEIK

SEQ ID NO: 510 - DNA, 723 bp, synthetic construct
  source 1..723: mol_type = other DNA
gaagtgcagctcctagagtcgggtggcgggctggtgcagccgggcggctccctgcgcctgtcttgcgcggctagtggatttgacttttctagcttcgcaatgacctgggtcagacaggccccagggaagggattggagtgggtgtcccacataacaggttcaggagggacaatctattacgccgatagcgttaagggacggttcactattagcagggataattcaaaaaacacgctttacttacaaatgaacagtctgcgagccgaggacaccgcagtatactattgtgcttccgccagctcctattacgaacccggggacccttggggccagggcactctcgtcaccgtgtcatctggtggcggtggatcgggcggtggtggatctggaggaggtggctcggacatccagatgactcagagtcccagctcactctccgcttcagtcggtgatcgggttactataacatgtagagccagccaagctattaagaattacctgaactggtatcagcaaaaacccgggaaggcacctaaactgttgatttatgcggggtccacactccagagcggggtgcctagtaggttctctggctccggatctggcactgactttaccttaaccatctcttcgcttcagccagaagattttgccacatactattgccagcagacatacagcacgccactgaccttcggtggcggaaccaaggtggagatcaaa

SEQ ID NO: 511 - protein, 241 aa, synthetic construct
EVQLLESGGGLVQPGGSLRLSCAASGFPFSSHGMSWVRQAPGKGLEWVSTITGSGRSTYYADSVKGRFTISRDNSKNTLYLQMNSLRAEDTAVYYCASDYRDAPGTFDVWGQGTLVTVSSGGGGSGGGGSGGGGSDIQMTQSPSSLSASVGDRVTITCRASQSISSYLNWYQQKPGKAPKLLIYDASHLQSGVPSRFSGSGSGTDFTLTISSLQPEDFATYYCQQSYSTPVFGGGTKVEIK

SEQ ID NO: 512 - DNA, 723 bp, synthetic construct
  source 1..723: mol_type = other DNA
gaagttcagctgttagaaagcggggggggcctggtgcagcctggcggctcactgagattgtcctgcgctgcatccggattcccgttttcctcacacggaatgagctgggttaggcaggctcccgggaagggcctagagtgggtcagtaccattacaggaagcggtaggagcacgtactacgccgattcagtaaagggccgattcacaatctcccgtgacaactctaaaaatactctttatctccagatgaacagtctgcgggcagaggatacagccgtgtattattgtgcgtcggactaccgcgatgccccaggaacctttgacgtgtggggtcaagggactctcgtcaccgtgtcttctggtggcggtggatcgggcggtggtggatctggaggaggtggctcggacatccaaatgacccagtctccctcctcgctgagcgcaagcgtaggggaccgggtcaccataacgtgtagagcttctcagtcaatctcctcatacttgaactggtatcagcagaagcccggcaaagcccctaagctcctgatttacgatgccagccacctacaaagcggagttccatccaggtttagtggctctgggtcaggaacagatttcacactgacaatcagttctcttcagcctgaagacttcgcgacttactattgccagcagtcctatagtaccccagtgtttggtgggggcactaaagtggagattaag

SEQ ID NO: 513 - protein, 241 aa, synthetic construct
EVQLLESGGGLVQPGGSLRLSCAASGFPFASHAMTWVRQAPGKGLEWVSTITGSGRSTYYADSVKGRFTISRDNSKNTLYLQMNSLRAEDTAVYYCASDYRDAPGTFDVWGQGTLVTVSSGGGGSGGGGSGGGGSDIQMTQSPSSLSASVGDRVTITCRASQSISSYLNWYQQKPGKAPKLLIYDASHLQSGVPSRFSGSGSGTDFTLTISSLQPEDFATYYCQQSYSTPVFGGGTKVEIK

SEQ ID NO: 514 - DNA, 723 bp, synthetic construct
  source 1..723: mol_type = other DNA
gaggtccagctgttggaatccgggggaggcttagtgcaaccgggcggctccctgcgactgagctgtgcagcctcgggattcccctttgcttctcacgcaatgacctgggttcgtcaggccccagggaaaggcctggagtgggtttctactatcaccggttcaggtcggagcacatattacgctgacagcgtgaaggggagatttaccatttcacgcgacaatagcaagaacactctctatcttcagatgaacagtctcagggccgaagatacagccgtctattactgcgctagtgactacagggatgcgcctggaacgttcgatgtgtggggccaggggacactagtgaccgtatcctctggtggcggtggatcgggcggtggtggatctggaggaggtggctcggatatacagatgactcaatctcccagcagtctgtccgcatccgtcggagatagggtaacaattacctgcagagcgtcccaatcgatcagttcatatcttaactggtaccagcagaagcctggcaaagctccaaagctgttaatttacgacgcctctcacctccagagtggcgttccttctcggttttctggcagcggttccgggaccgacttcacgttgacaatctcaagcctgcagccagaagactttgccacctactattgtcagcagtcatatagcactcccgtgttcgggggtggaacaaaagtggagatcaag

SEQ ID NO: 515 - protein, 240 aa, synthetic construct
EVQLLESGGGLVQPGGSLRLSCAASGFDFSKFAMSWVRQAPGKGLEWVSHISGSGGTIYYADSVKGRFTISRDNSKNTLYLQMNSLRAEDTAVYYCASASSYYEPGDPWGQGTLVTVSSGGGGSGGGGSGGGGSDIQMTQSPSSLSASVGDRVTITCRASQAIKNYLNWYQQKPGKAPKLLIYDASHPQSGVPSRFRGSGSGTDFPFTISSLQPEEFATYYCQQSYSTPVFGGGTKVEIK

SEQ ID NO: 516 - DNA, 720 bp, synthetic construct
  source 1..720: mol_type = other DNA
gaagtgcaactgctggaatcgggcggtggactggtccagcctggaggctctttgaggctttcctgtgccgcctcagggtttgatttcagtaaattcgctatgtcctgggttcgccaggctcccgggaaaggacttgagtgggtgtcccacatcagcggtagcggcggaacgatttattacgcagactcagtaaagggcagatttactatatctcgggacaattcaaagaacacactctacctgcagatgaacagtttacgagcagaggataccgcggtctattactgcgccagtgccagctcctactatgagccaggcgacccgtgggggcaggggacactcgtgaccgtgagctctggtggcggtggatcgggcggtggtggatctggaggaggtggctcggatatacagatgactcaatccccttcttctctgtccgcgtctgttggggacagggtgacaatcacctgtcgggcttcgcaggctattaaaaattatcttaactggtatcagcagaagcccggtaaggccccaaagttgctcatctatgatgccagccacccacagtcaggagtacccagtcgcttcagaggctccggaagcggcaccgacttccctttcaccatctcaagcctgcagccggaggaatttgcaacatactactgccagcaaagctacagtacgcccgtctttggcggtgggactaaagtggagattaaa

SEQ ID NO: 517 - protein, 241 aa, synthetic construct
EVQLLESGGGLVQPGGSLRLSCAASGFDFSKFAMSWVRQAPGKGLEWVSHISGSGGTIYYADSVKGRFTISRDNSKNTLYLQMNSLRAEDTAVYYCASDSSYYEPGDPWGQGTLVTVSSGGGGSGGGGSGGGGSDIQMTQSPSSLSASVGDRVTITCRAGQAIKNYLNWYQQKPGKAPKLLIYAGSPLQSGVPSRFSGSGSGTDFTLTISSLQPEDFATYYCQQTYSTPLTFGGGTKVEIK

SEQ ID NO: 518 - DNA, 723 bp, synthetic construct
  source 1..723: mol_type = other DNA
gaggtacaactcctagagagtggcggaggccttgtgcagcccggaggatcgctgcgactgtcatgcgctgcatccggctttgatttctctaaatttgccatgagctgggtgagacaggccccgggtaagggcctggaatgggtgtctcacatatcagggagcgggggaacaatttactatgccgactcagttaaggggcgcttcaccatcagcagggacaattccaaaaacaccctgtatttacagatgaacagtttgcgggcggaggatacggcagtctactattgtgcttccgacagcagttactacgaaccaggtgatccttggggccaggggactctcgtgacagtctcctctggtggcggtggatcgggcggtggtggatctggaggaggtggctcggacatccagatgacacagagcccgtcttcactcagtgcaagtgttggggacagagtgaccattacatgcagggccggacaggctatcaaaaactatctgaattggtaccaacaaaaacctggcaaggcgcccaaattgctcatctatgccggttcaccactgcagagcggtgtgccatcgcggtttagcggatctggctccggcactgatttcacccttaccatttcctctttacagcccgaggatttcgctacctactattgtcagcagacttactccacgcctctgacatttggaggggggactaaggtcgaaataaag

SEQ ID NO: 519 - protein, 242 aa, synthetic construct
EVQLLESGGGLVQPGGSLRLSCAASGFDFSKFAMSWVRQAPGKGLEWVSSITRGSETTYYADSVKGRFTISRDNSKNTLYLQMNSLRAEDTAVYYCATLGLGYYYYFDVWGQGTLVTVSSGGGGSGGGGSGGGGSDIQMTQSPSSLSASVGDRVTITCRASQPISSYVTWYQQKPGKAPKLLIYSASHLRSGVPSRFSGSGSGTDFTLTISSLQPEDFATYYCQQSYNAPPTFGGGTKVEIK

SEQ ID NO: 520 - DNA, 726 bp, synthetic construct
  source 1..726: mol_type = other DNA
gaggtgcagctgctggagagcggcggagggctggtgcaacctgggggctcactgcgactttcctgcgctgcctcaggtttcgatttctctaagttcgctatgagctgggtcagacaggcacccgggaaaggcttagagtgggtgtcttctattacgcgtggcagtgaaactacctactacgccgacagcgttaaaggacggtttacaatctccagggacaattccaagaacaccctctatctgcagatgaacagtctccgcgcggaagacacagccgtctattactgtgcaaccctagggttgggttactattattactttgatgtatggggccagggaacattggtgactgttagctcgggtggcggtggatcgggcggtggtggatctggaggaggtggctcggacatccagatgactcagtcgccctcctcactgtctgcttcagttggagatcgggtgaccattacctgccgcgctagtcaaccgatcagctcctatgtgacgtggtatcagcagaagccaggcaaggcccctaaattgttaatctattcagcgtcccacctcaggagcggcgtccccagcagattttctggaagcgggagtggtaccgatttcactctgacaatatcctctcttcagcccgaggactttgcaacatactactgtcaacagagttacaacgccccacctacattcggtggcgggaccaaagtagaaattaag

SEQ ID NO: 521 - protein, 240 aa, synthetic construct
EVQLLESGGGLVQPGGSLRLSCAASGFPFASHAMTWVRQAPGKGLEWVSTITGSGRSTYYADSVKGRFTISRDNSKNTLYLQMNSLRAEDTAVYYCASASSYYEPGDPWGQGTLVTVSSGGGGSGGGGSGGGGSDIQMTQSPSSLSASVGDRVTITCRASQTISSFVSWYQQKPGKAPKLLIYAATTLQKGVPSRFSGSGSGTDFTLTISSLQPEDFATYYCQQSYHTRSFGGGTKVEIK

SEQ ID NO: 522 - DNA, 720 bp, synthetic construct
  source 1..720: mol_type = other DNA
gaggtgcagctcctcgaaagcggcggcggattagtccagcctgggggatcgctgaggttgtcatgcgccgcctcaggctttcccttcgcatcccacgcaatgacttgggtgagacaggcgccagggaaggggcttgagtgggtgtctacgattaccggcagcgggcggtccacatattatgccgatagtgttaagggccgattcactatctcacgtgataactccaaaaacaccctgtacctgcagatgaatagtctacgcgctgaggacaccgctgtatactattgtgcttctgcctctagctactacgaacccggtgacccgtggggacaaggtacactggtgacagtctccagcggtggcggtggatcgggcggtggtggatctggaggaggtggctcggacattcaaatgacacagagcccatctagtttgagcgcatccgtaggcgatagagtgacgatcacttgtcgggcttcacagaccatatcttccttcgtgtcttggtatcagcagaaaccgggtaaggcgcccaaattactcatctacgccgccacaaccctgcaaaagggcgttccttcacgcttttcagggagcgggtccggaacagacttcacacttaccattagttcgctgcagcccgaggatttcgctacttattactgccagcagtcctaccacactaggagctttggcggagggaccaaagtcgaaatcaag

SEQ ID NO: 523 - protein, 240 aa, synthetic construct
EVQLLESGGGLVQPGGSLRLSCAASGLTFSNFAMTWVRQAPGKGLEWVSSIRGSGATTYYADSVKGRFTISRDNSKNTLYLQMNSLRAEDTAVYYCARSREAYGFDYWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGGGTKVEIK

SEQ ID NO: 524 - DNA, 720 bp, synthetic construct
  source 1..720: mol_type = other DNA
gaagtgcagctcctagagagcgggggcgggctggtgcagcccggagggtcactgaggctgtcttgcgctgcctctggcttaacattctccaatttcgcaatgacatgggtgcgccaggcccctgggaaaggtcttgagtgggtgtcctcgattaggggatctggtgccaccacttactacgctgactccgtcaagggcagatttacgatcagcagagataacagtaagaataccctgtacttgcagatgaacagtcttcgggccgaagatacagctgtctattattgtgcacgaagccgtgaggcgtatggatttgactactggggccaaggcaccctcgtaactgttagctcaggtggcggtggatcgggcggtggtggatctggaggaggtggctcggatatacagatgacacaatcaccctccagcctttcggcctcagtgggtgacagagttaccattacctgccgggcttcacagagcatcagctcctacttgaactggtaccagcaaaagcccggaaaagctccaaagctcttaatctacgccgcatcttctctgcagtctggggtgccttctaggtttagcggaagtggcagcggcaccgactttacactgactattagttccctgcagcctgaagatttcgcgacatattactgtcagcagtcctatagtactccatataccttcggtggcgggacgaaggtcgagatcaaa

SEQ ID NO: 525 - protein, 239 aa, synthetic construct
EVHLLESGGGLVQPGGSLRLSCAASGFTFSHYSMNWVRQAPGKGLEWVSGISGSGSATYYADSVKGRFTISRDNSKNTLYLQMNSLRAEDTAVYYCASDRYLTFDVWGQGTLVTVSSGGGGSGGGGSGGGGSDIQMTQSPSSLSASVGDRVTITCRASQSISTHLNWYQQKPGKAPKLLIYAASSLQSGVPSRFSGSGSGTDFTLTISSLQPEDFATYYCQQSHRTPLIFGGGTKVEIK

SEQ ID NO: 526 - DNA, 717 bp, synthetic construct
  source 1..717: mol_type = other DNA
gaagtccacctgttggagagcggcggtggcctggttcaacctgggggttctctacgtttgtcgtgtgcagcctctgggtttacatttagccattatagcatgaattgggtgagacaggcacccggaaagggacttgagtgggttagcgggatctccgggtcaggctccgcgacttactatgccgattcagtgaaaggccgcttcaccatttctcgggacaacagtaagaatactctgtacctccagatgaacagtctccgagctgaagacaccgccgtgtactactgcgcttccgacaggtatctgaccttcgatgtctggggacagggcacgttagtgacagtatcctcaggtggcggtggatcgggcggtggtggatctggaggaggtggctcggacatccagatgacgcaatccccttctagcctgtcagcgtcagtgggcgatcgggtcacaatcacttgtagagcttctcagagtattagtacccatctgaactggtaccagcagaaaccaggcaaggcacccaaactcctcatatatgccgcttccagcttacagagtggtgtgccatcgaggttcagcggttccggcagcggaactgactttaccttgaccatctcctctctgcagcctgaagattttgccacatattactgccaacagtcacaccgcaccccccttattttcggaggggggacaaaggttgagattaag

SEQ ID NO: 527 - protein, 241 aa, synthetic construct
EVQLLESGGGLVQPGGSLRLSCAASGFDFSKFAMSWVRQAPGKGLEWVSHISGSGGTIYYADSVKGRFTISRDNSKNTLYLQMNSLRAEDTAVYYCASASSYYEPGDPWGQGTLVTVSSGGGGSGGGGSGGGGSDIQMTQSPSSLSASVGDRVTITCRASQAIKNYLNWYQQKPGKAPKLLIYAGSTLQSGVPSRFSGSGSGTDFTLTISSLQPEDFATYYCQQTYSTPLTFGGGTKGEIK

SEQ ID NO: 528 - DNA, 723 bp, synthetic construct
  source 1..723: mol_type = other DNA
gaggtgcagctgctggagtctggcggcggtcttgtccagcccggggggtcactccgactaagctgcgctgcgagtggatttgacttctccaagtttgccatgtcctgggtgagacaggcaccaggaaagggacttgagtgggtgtcacacatatctgggagcggcggaactatttactacgctgatagcgttaaaggccgcttcacaatctcacgggacaacagtaaaaacaccctctatttgcaaatgaatagtctgagggccgaagatacagcagtctattactgtgcctccgcctctagctactatgaacctggcgacccgtgggggcagggtacgctggtgaccgtatcgtccggtggcggtggatcgggcggtggtggatctggaggaggtggctcggatattcagatgacacagagcccctcgtcactgtctgcgtccgtcggtgatcgggttacaatcacctgtagggcttctcaagctatcaagaactacctcaattggtaccagcaaaaacctggcaaggccccaaaactgttaatttatgcagggtctacattgcagagtggcgtgccttcaagattcagcggctccgggtccggaactgacttcacccttacaatcagcagtctgcagcccgaggactttgccacctattattgccagcagacgtacagcaccccactcacttttgggggtggaactaaaggagaaataaag

SEQ ID NO: 529 - protein, 240 aa, synthetic construct
EVQFLESGGGLVQPGGSLRLSCAASGFSFDNYGINWVRQAPGKGLEWVSSISSSGGSAYYADSVKGRFTISRDNSKNTLYLQMNSLRAEDTAVYYCARGVVPGGFDYWGQGTLVTVSSGGGGSGGGGSGGGGSDIQMTQSPSSLSASVGDRVTITCRASQSINNYLNWYQQKPGKAPKLLIYAASSLQSGVPSRFSGSGSGTDFTLTISSLQPEDFATYYCQQSRTTPWTFGGGTKVEIK

SEQ ID NO: 530 - DNA, 720 bp, synthetic construct
  source 1..720: mol_type = other DNA
gaggtgcagttcctcgaatccggtgggggtttagtgcagcccgggggcagccttcggctgtcctgcgccgcctctgggtttagcttcgacaattacggtatcaactgggttcgacaagcaccagggaagggactggaatgggtgtcatcaatttcctcgtctggaggctccgcgtattacgcagattcagtgaaaggccgttttaccataagtagggacaatagcaagaacacactgtacctccagatgaacagtctgcgcgctgaggacaccgccgtctattactgtgctagaggcgtagtccctggaggattcgattattggggccagggcacgttggttactgtgtctagcggtggcggtggatcgggcggtggtggatctggaggaggtggctcggacatccaaatgacacagtccccaagttctctttccgcctctgtgggtgacagagtgacaatcacctgtcgcgcatcccagtcaattaataactatctcaactggtaccagcagaagcctgggaaggcgcccaaactgctgatttatgccgcttccagcttacaaagtggtgtcccatcaaggttttcaggcagcggaagcgggaccgatttcactctgacgataagctcgttgcagcctgaagatttcgctacctactactgccagcagtctcggactaccccctggacttttggcggcggaacaaaggttgagatcaaa

SEQ ID NO: 531 - protein, 245 aa, synthetic construct
EVQLLESGGGLVQPGGSLRLSCAASGFDFSKFAMSWVRQAPGKGLEWVSSITGTSGATYYADSVKGRFTISRDNSKNTLYLQMNSLRAEDTAVYYCARDIRVRRSSWAMDPWGQGTLVTVSSGGGGSGGGGSGGGGSDIQMTQSPSSLSASVGDRVTITCRASQTIGIYLNWYQQKPGKGPKPAIYSASPLQRGVPSRFSGCGSGTDFTLTISSLQPEDFATYYCQQSYSTPHITFGGGTKVEIK

SEQ ID NO: 532 - DNA, 735 bp, synthetic construct
  source 1..735: mol_type = other DNA
gaagtgcagcttcttgagtccggcgggggactagttcagcccggcggctcactgcgactgagttgtgcggcttcagggtttgatttcagcaaattcgcaatgtcttgggtgagacaggccccaggaaaaggtttggagtgggtgagctctattaccgggacctcgggcgccacctactatgctgattccgttaagggtagatttactatctctagggacaatagcaagaacacactctacctccaaatgaacagtctgcgggccgaagacactgccgtatactattgcgctagggatatacgcgtgcggcgtagctcctgggcaatggacccttggggacagggcacgctggtcacagtctcctcaggtggcggtggatcgggcggtggtggatctggaggaggtggctcggacatccagatgacgcaaagtcctagcagcctgtccgcctcagtgggcgaccgggtgactatcacatgtagggcttctcagaccattggcatatatctgaactggtaccagcagaagcccgggaaaggtcccaaaccggcgatctactccgcatccccacttcagcgcggcgtcccttccagattctcggggtgcggctcaggtacagattttactttgacaatttctagtctccagcccgaagatttcgccacttactattgccaacagagctattctaccccacacattacctttggagggggaaccaaggttgagatcaag

SEQ ID NO: 533 - protein, 241 aa, synthetic construct
EVQLLESGGGLVQPGGSLRLSCAASGFDFSKFAMSWVRQAPGKGLEWVSHISGSGGTIYYADSVKGRFTISRDNSKNTLYLQMNSLRAEDTAVYYCASASSYYEPGDPWGQGTLVTVSSGGGGSGGGGSGGGGSDIQMTQSPSSLSASVGDRVTITCRASQAIKNYLNWYQQKPGKAPKLLIYAGSTLQSGVPSRFSGSGSGTDFTLTISSLQPEEFATYYCQQTYSTPLTFGGGTKGENQ

SEQ ID NO: 534 - DNA, 723 bp, synthetic construct
  source 1..723: mol_type = other DNA
gaggtacaactcctggagtcaggcggagggctcgtccagccaggaggttccctaagactttcttgtgccgcctcaggctttgacttcagcaagttcgcaatgagctgggtgaggcaggcgcctgggaaggggttagaatgggtgtctcacattagtggctctggaggaactatctactatgccgattcggtgaaagggcgatttacaatatcccgcgacaattcaaaaaacacactgtacctgcagatgaacagtttgcgggctgaagataccgcagtctactattgcgcttccgcctccagctattacgagcccggtgacccgtggggccagggcaccctggttacggtgagtagcggtggcggtggatcgggcggtggtggatctggaggaggtggctcggacattcaaatgacacagagtcccagtagcctcagcgcttccgttggcgatagagtgacaattacctgtagggcctcccaggcaataaagaactatcttaactggtaccagcagaaaccaggtaaggcccctaaactcttaatctatgctggctcaaccctgcaatctggggtcccctcgcggttctcagggtctggaagcggcacagacttcactttgaccatctctagcctgcagccagaggagtttgcgacttactattgccaacagacatactccacccctctgacgtttggagggggtactaagggagaaaatcag

SEQ ID NO: 535 - protein, 241 aa, synthetic construct
EVQLLESGGGLVQPGGSLRLSCAASGFTFTNYALAWVRQAPGKGLEWVSHISGSGGTIYYADSVKGRFTISRDNSKNTLYLQMNSLRAEDTAVYYCASASSYYEPGDPWGQGTLVTVSSGGGGSGGGGSGGGGSDIQMTQSPSSLSASVGDRVTITCRASQAIKNYLNWYQQKPGKAPKLLIYAGSTLQSGVPSRFSGSGSGTDFTLTISSLQPEDFATYYCQQTYSTPLTFGGGTKVEIK

SEQ ID NO: 536 - DNA, 723 bp, synthetic construct
  source 1..723: mol_type = other DNA
gaagtgcagctgctagaatccggagggggcttagtccagcctggcggctcccttcggctctcttgtgccgcttccggatttacgttcactaactatgcactggcctgggtgcgacaggcgccagggaaggggctggagtgggtaagccacatctctggatcaggaggtacaatttactatgctgacagcgtgaaggggcgcttcaccataagcagagataactctaaaaataccctttatctgcaaatgaatagtctcagggccgaggatactgcagtgtattactgcgcttcggcctcaagctactacgagcccggcgacccgtggggtcagggcacattggtcaccgttagttccggtggcggtggatcgggcggtggtggatctggaggaggtggctcggatatccaaatgacacagtctcccagttcattgtccgcttccgttggagatagagtcaccattacctgcagggccagtcaggcgataaaaaactaccttaattggtatcagcagaagcctgggaaagctcccaagctgctgatctacgccggctcaactctgcagtctggggtgccttctcggttttccggttcgggcagcgggacggactttacactcacaattagcagcttacaaccagaggacttcgcaacttattactgtcagcagacctatagcacaccactcactttcggcggaggtaccaaggtggaaatcaaa

SEQ ID NO: 537 - protein, 241 aa, synthetic construct
EVQLLESGGGLVQPGGSLRLSCAASGFDFSKFAMSWVRQAPGKGLEWVSHISGSGGTIYYADSVKGRFTISRDNSKNTLYLQMNSLRAEDTAVYYCASASSYYEPGDPWGQGTLVTVSSGGGGSGGGGSGGGGSDIQMTQSPSSLSASVGDRVTITCRASQAIKNYLNWYQQKPGKAPKLLIYAGSTLQSGVPSRFSGSGSGTDFTLTISSLQPEDFATYYCQQTYSTPLTFGGGTKVEIK

SEQ ID NO: 538 - DNA, 723 bp, synthetic construct
  source 1..723: mol_type = other DNA
gaggtgcagctgctggagtctgggggcgggctagtccaacccggaggatcgctgaggctttcctgcgccgcgtcagggtttgacttcagcaagtttgccatgtcttgggtgcgacaggcacctggaaaaggattggaatgggtgtcacacataagcggttctggcggcactatctattacgccgactccgtcaaaggccgcttcacaattagccgggataacagcaagaatacgctctacttacagatgaacagtctcagagccgaggataccgctgtttactattgtgcatcagctagtagttactatgaaccaggtgacccgtgggggcagggcaccctggtgacagtatcctccggtggcggtggatcgggcggtggtggatctggaggaggtggctcggatatccagatgacccagtcgccaagctctctctccgcttctgttggagacagggtcacaattacttgccgggcatcccaagcgatcaagaactatttaaattggtaccagcagaagcctgggaaagctcctaaacttctgatatatgccggctctaccctgcagtcaggggtgcccagtagattctcagggagcggcagtggtaccgatttcacactgaccattagcagcttgcaaccagaggactttgccacatactattgtcagcagacgtactccactcccctcacatttggtggcggaactaaagtggaaatcaag

SEQ ID NO: 539 - protein, 241 aa, synthetic construct
EVQLLESGGGLVQPGGSLRLSCAASGIRFSSYAMSWVRQAPGKGLEWVSHISGSGGTIYYADSVKGRFTISRDNSKNTLYLQMNSLRAEDTAVYYCASASSYYEPGDPWGQGTLVTVSSGGGGSGGGGSGGGGSDIQMTQSPSSLSASVGDRVTITCRASQAIKNYLNWYQQKPGKAPKLLIYAGSTLQSGVPSRFSGSGSGTDFTLTISSLQPEDFATYYCQQTYSTPLTFGGGTKVEIK

SEQ ID NO: 540 - DNA, 723 bp, synthetic construct
  source 1..723: mol_type = other DNA
gaggtccagctgctagagtctggaggcgggctggtgcagccgggaggtagtttacgcctgtcctgtgcagcttcgggcattaggttttcaagctacgcaatgtcttgggtgcgacaagcgcctggtaagggactggaatgggtgagccacatcagcggcagcggcggaactatatattatgccgactccgtaaaggggcggttcacgatctcacgtgataactcaaaaaacacattgtatctccagatgaatagtcttagagccgaggataccgccgtttactactgcgcttccgcctccagttactatgaaccaggggacccctggggccaggggacactcgtcaccgtgtcttctggtggcggtggatcgggcggtggtggatctggaggaggtggctcggatattcagatgactcagagcccatctagcctcagcgcttctgtgggggacagggttacgattacatgtagagcaagtcaggccatcaagaactaccttaattggtaccagcaaaagcctggcaaagctcctaaactgctcatctacgcggggtccacattgcaatctggagtcccctcgcggtttagtggaagcggctccggtacagacttcactctgaccatctcatcattacagccagaggatttcgccacatattattgccagcagacctattccactcccctgacctttggcggcgggaccaaagtggaaataaag

SEQ ID NO: 541 - protein, 241 aa, synthetic construct
EVQLLESGGGLVQPGGSLRLSCAVSGFDFSKFVMSGVRQAPGKGLEWVSHISGSGGTIYYADSVKGRFTISRDNSKNTLYLQMNSLRAEDTAVYYCASASSYYEPGDPWGQGTLVTVSSGGGGSGGGGSGGGGSDIQMTQSPSSLSASVGDRVTITCRASQAIKNYLNWYQQKPGKAPKLLIYAGSTLQSGVPSRFSGSGSGTDFTLTISSLQPEDFATYYCQQTYSTPLTFGGGTKVEIK

SEQ ID NO: 542 - DNA, 723 bp, synthetic construct
  source 1..723: mol_type = other DNA
gaagtgcagctcttagagagcgggggcggactggtccagccaggcggtagcctgcgcctaagttgcgccgtttctgggttcgatttttctaagttcgtgatgagtggcgtgcggcaggctcctggcaaaggtctggagtgggtttcacacatatccgggtctgggggaacgatttactacgctgattcagtgaaaggaagatttaccatctccagggacaacagcaagaatactctctatctgcaaatgaacagtcttcgagcggaagacacagccgtctactattgtgcatccgcctcgtcatattacgagccgggtgacccctggggacagggcacattggtgaccgtaagctccggtggcggtggatcgggcggtggtggatctggaggaggtggctcggacatccagatgacccagtctccttcctcccttagtgcttcggtgggtgatagggtcaccattacatgcagagccagtcaggcaataaagaactacttgaattggtaccagcaaaaacccggcaaggcccccaaactgctgatctatgctggttccacgttacagtctggagtgccaagccggtttagcggctcagggtctggaactgatttcacactgacaattagctcactccagccagaagacttcgcgacttattattgtcagcaaacctacagcacccctctcacttttgggggcgggacaaaggttgagatcaaa

SEQ ID NO: 543 - protein, 241 aa, synthetic construct
EVQLLESGGGLVQPGGSLRLSCAASGFDFSKFAMSWVRQAPGKGLEWVSHISGSGGTIYYADSVKGRFTISRDNSKNTLYLQMNSLRAEDTAVYYCASASSYYEPGDPWGQGTLVTVSSGGGGSGGGGSGGGGSDIQMTQSPSSLSASVGDRVTITCRASQAIKNYLNWYQQKPGKAPKLLIYAGSTLQSGVPSRFSGSGSGTDFTLTISSLQPEDFPTYYFQQNYSTPFAFGGGTKVEIK

SEQ ID NO: 544 - DNA, 723 bp, synthetic construct
  source 1..723: mol_type = other DNA
gaagtgcagctcctagagtccggaggtggcctcgtgcagccgggaggctccctgcgcctgtcctgcgcggcctctggctttgactttagtaagttcgctatgagctgggtgagacaggctccaggtaagggcctggagtgggtatctcacatctccgggtcaggagggaccatatattacgccgactctgtcaaaggccggttcactatttcaagggataattcaaaaaacacattataccttcaaatgaacagtctgcgagccgaagacacagcagtgtactattgtgccagcgcaagcagctactatgagcctggagatccctgggggcaggggaccttggtcacggttagttcgggtggcggtggatcgggcggtggtggatctggaggaggtggctcggatatacagatgacccagtcaccctccagcttgtcagctagtgttggggaccgggtcaccatcacatgcagagcctctcaggcgatcaagaattaccttaactggtatcagcagaaaccaggaaaagctccgaagttactgatttacgcaggtagcacactgcagtcgggcgtgccctctaggttcagtgggagcggctccggaacagactttactctgaccatttcttccctccaaccagaagatttccctacttattattttcagcaaaactacagcacgcctttcgcctttggcggtgggaccaaagtggagatcaag

SEQ ID NO: 545 - protein, 241 aa, synthetic construct
EVQLLESGGGLVQPGGSLRLSCAASGFDFSKFAMSWVRPAPCKGLEWVSHISGSGGTIYYADSVKGRFTISRDNSKNTLYLQMNSLRAEDTAVYYCASASSYYEPGDPWGQGTLVTVSSGGGGSGGGGSGGGGSDIQMTQSPSSLSASVGDRVTITCRASQAIKNYLNWYQQKPGKAPKLLIYAGSTLQSGVPSRFSGSGSGTDFTLTISSLQPEDFATYYCQQTYSTPLTFGGGTKVEIK

SEQ ID NO: 546 - DNA, 723 bp, synthetic construct
  source 1..723: mol_type = other DNA
gaggtgcagcttctggagagcggagggggactggtccagcctggcggcagtctaagattgtcctgcgccgcgtcagggttcgatttttccaaatttgccatgtcatgggtgcgcccagctccctgtaagggattagaatgggtgagtcacatctcaggttctgggggcacaatttattacgcagactccgtcaaggggcgattcacgatatcccgggacaactctaaaaacactctgtacctccaaatgaatagtctcagggccgaggacaccgctgtgtactattgcgcctcggcaagcagctattacgaacccggcgatccgtggggccagggtaccctggtaacagttagctctggtggcggtggatcgggcggtggtggatctggaggaggtggctcggacatccagatgacacagagtccatctagcttatccgcgagtgttggggacagggtcaccatcacatgtagagcctctcaggcaattaaaaactacctgaattggtatcaacaaaagcccggaaaggccccaaagctgctcatctacgccggctccaccctgcagagcggagtgcctagccggtttagcggctctggctcggggactgattttacattgactatttcatcacttcagcccgaagatttcgctacatattattgccagcagacctactccactcctctcacgttcggtggtgggaccaaagtggagataaaa

SEQ ID NO: 547 - protein, 241 aa, synthetic construct
EVQLLESGGGLVQPGGSLRLSCAASGFDFSKFAMSWVRQAPGKGLEWVSHISGSGGTIYYADSVKGRFTISRDNSKNTLYLQMNSLRAEDTAVYYCASASSYYEPGDPWGQGTLVTVSSGGGGSGGGGSGGGGSDIQMTQSPSSLSASVGDRVTITCRASQAIKNYLNWYQQKPGKAPKLLIYAGSTLQSGVPSRFSGSGSGTDFTLTISSLQPEDFATYYCQQNYSTPLTFGGGTKVEIK

SEQ ID NO: 548 - DNA, 723 bp, synthetic construct
  source 1..723: mol_type = other DNA
gaggtgcagctgctggaaagcggaggagggttagtgcaaccaggaggatctctccggctgagctgcgctgcctcaggctttgatttcagcaagttcgccatgtcctgggtccgacaggcaccggggaaaggccttgagtgggtgagccacatatccggctcaggtgggaccatttactacgctgattctgtaaaagggagatttacgatctcacgcgacaacagtaagaatacactctacctgcagatgaacagtttgagggccgaagacacagccgtgtattattgtgcgtctgcatcctcgtactatgagcctggcgacccctggggccagggtaccctagtcactgtttccagtggtggcggtggatcgggcggtggtggatctggaggaggtggctcggatatacaaatgacacagagtccctcatccctgtcagcttctgtgggtgatagggtcacaatcacatgcagagccagccaggcgattaaaaattacctcaactggtatcagcagaagcctggcaaggctccaaaattacttatctacgcaggtagcaccctgcagtccggagtgccttctcggttcagcgggtccggctctgggaccgactttaccctcaccatcagtagcctgcagccagaagacttcgccacttattactgtcagcaaaactattcgactcccttgacgtttggaggcgggactaaagttgagattaag

SEQ ID NO: 549 - protein, 240 aa, synthetic construct
EVQFLKSGGGLVNPAGILELSCAASGFPFSSHGMSWVRQAPGKGLEWVSVISGSGATTYYADSVKGRFTISRDNSKNTLYLQMNSLRAEDTAVYYCARGQPYYGFDYWGQGTLVTVSSGGGGSGGGGSGGGGSDIQMTQSPSSLSASVGDRVTITCRASHSIASYLHWYQQKPGKAPKLLIYRASTLHTGVPSRFSGSGSGTDFTLTISSLQPEDFATYYCQQTYSIPWTFGGGTKVEIK

SEQ ID NO: 550 - DNA, 720 bp, synthetic construct
  source 1..720: mol_type = other DNA
gaggtccaatttctgaaaagcggtggcgggctggttaatcccgcgggcatccttgagctctcttgtgcagcctccgggttcccgtttagcagtcacggtatgtcatgggtgaggcaggctccaggaaagggactagaatgggtatctgtgatttcaggctccggagccaccacctattacgcagattccgttaagggccgcttcactatatcgagagacaacagcaaaaatactttatatttgcagatgaacagtctgcgagccgaagacacagctgtctattactgcgcccgggggcagccttactatggcttcgattactggggacaggggacgctcgtgacagtgagctctggtggcggtggatcgggcggtggtggatctggaggaggtggctcggacatacagatgacacagtctcccagctctttatccgcttccgtgggcgatagagtgactattacatgccgcgcaagtcactccatcgccagctatctccactggtaccaacaaaagccaggtaaggcgcccaaactgctgatctacagggccagcacgctgcatacaggagtcccttctcggttttccggaagtggctcagggacagatttcactcttaccatttcatcgttgcagcctgaggactttgctacctattattgtcagcagacctacagcatcccgtggactttcggtggcgggaccaaagttgaaattaag

SEQ ID NO: 551 - protein, 241 aa, synthetic construct
EVQFLESGGGLVQPGGSLRLSCAASGFDFSKFAMSWVRQAPGKGLEWVSHISGSGGTIYYADSVKGRFTISRDNSKNTLYLQMNSLRAEDTAVYYCASASSYYEPGDPWGQGTLVTVSSGGGGSGGGGSGGGGSDIQMTQSPSSLSASVGDRVTITCRASQAIKNYLNWYQQKPGKAPKLLIYGASNLQSGVPSRFSGSGSGTDFTLTISSLQPEDFATYYCQQSESTPLTFGGGTKVEIK

SEQ ID NO: 552 - DNA, 723 bp, synthetic construct
  source 1..723: mol_type = other DNA
gaggtgcaatttctggagtcaggggggggtttggttcagccgggaggcagccttaggttatcctgtgccgcctcagggttcgatttcagcaaatttgcaatgagttgggtccggcaggcacccggcaagggactggaatgggtaagccacatttctggctctggaggcaccatctactacgccgactccgtgaagggacgattcacgatatcgcgcgacaattcaaaaaacaccctgtatctccagatgaacagtctgagagctgaggatactgccgtctattactgcgcttccgcgtctagttactatgaaccaggtgacccttggggccaggggacactcgtgacagtgagctccggtggcggtggatcgggcggtggtggatctggaggaggtggctcggacattcagatgacacagtcaccaagtagtctgtccgcgtctgttggggatcgggtgacgattacatgcagggcttcgcaggccatcaaaaactacttaaactggtatcaacagaagcccggcaaggcccctaagctgcttatatacggggcttccaatctgcagtcaggagtcccttccagattttctggatctggtagcggtactgattttacactcaccatctccagcctccaacccgaggacttcgcaacctattactgtcagcagagcgaaagcactccattgaccttcgggggcggcaccaaagtggagatcaaa

SEQ ID NO: 553 - protein, 241 aa, synthetic construct
EVQLLESGGGLVQPGGSLRLSCAASGFALTDFAMSWVRQAPGKGLEWVSQISVSGGVGYYADSVKGRFTISRDNSKNTLYLQMNSLRAEDTAVYYCARSRSYYAGAFDVWGQGTLVTVSSGGGGSGGGGSGGGGSDIQMTQSPSSLSASVGDRVTITCRASAPVGRYLNWYQQKPGKAPKLLIYAASSLQSGVPSRFSGSGSGTDFTLTISSLQPEDFATYYCQQSYSAVTFGGGTKVEIK

SEQ ID NO: 554 - DNA, 723 bp, synthetic construct
  source 1..723: mol_type = other DNA
gaagtgcagcttttagaatcaggcggtggactcgtacagcctgggggctccctgagactgagctgcgctgcctcaggcttcgccctcacagactttgccatgtcctgggtgcggcaagctcccgggaagggactagagtgggtgtcccagatctcggtgtctggtggcgttgggtactacgccgacagcgtcaaagggaggttcacgatttccagggataacagtaagaataccctgtacctgcaaatgaacagtttgcgcgcggaggatacagcagtttattattgtgcacgaagccgttcttattacgctggagcatttgacgtgtggggccagggaacccttgtcactgtcagctctggtggcggtggatcgggcggtggtggatctggaggaggtggctcggatatccaaatgacccagtctccttcctcgctcagcgcaagtgtgggcgacagggtgaccattacatgtcgcgccagtgctccagtcggaagatatctgaactggtatcagcagaaacctgggaaggcccccaagctgcttatctatgcagcctcatccttacagtctggggtgccctcccggttctcaggtagcggtagcgggactgactttacactgaccatttcatctttgcagccagaggattttgctacgtactactgccagcaaagctactccgcggttacattcggaggcggcactaaagtagaaataaag

SEQ ID NO: 555 - protein, 242 aa, synthetic construct
EVQLLESGGGLVQPGGSLRLSCAASGFTFGSYAMTWVRQAPGKGLEWVSAIGTTDRYTYYADSVKGRFTISRDNSKNTLYLQMNSLRAEDTAVYYCARSRSYYAGAFDVWGQGTLVTVSSGGGGSGGGGSGGGGSDIQMTQSPSSLSASVGDRVTITCRASQTLRNYLNWYQQKPGKAPKLLIYAASNLQTGVPSRFSGSGSGTDFTLTISSLQPEDFATYYCQQRFSPPWTFGGGTKVEIK

SEQ ID NO: 556 - DNA, 726 bp, synthetic construct
  source 1..726: mol_type = other DNA
gaggtccagctactggaaagcgggggcgggttagttcagcccgggggtagcttgcgcctctcatgcgccgcctccggattcaccttcggctcttatgccatgacctgggtgagacaggcacctggcaagggactggaatgggtgtctgctattggcactactgacagatacacgtactacgctgatagcgtgaaaggccgttttaccatctcccgagacaacagtaagaacacactgtatctccagatgaatagtctgagggctgaggatacagccgtttattactgtgcaaggtcgcggtcctactatgcgggtgcatttgacgtctggggacaagggacccttgtaacagtgtcttcaggtggcggtggatcgggcggtggtggatctggaggaggtggctcggatattcagatgacacagagcccctctagtctcagcgcctccgtgggtgacagagttaccattacctgcagggcttcgcagactctgcggaactacttgaactggtaccaacaaaaaccagggaaagcgccgaagctccttatctacgcagcctccaatttacagactggcgtcccctctcgctttagtggctcaggatcaggcacggacttcactctgaccataagctctctgcagccagaggatttcgctacctattattgtcagcagcgattttcccctccttggacattcggaggtgggacaaaggtggaaatcaag

SEQ ID NO: 557 - protein, 242 aa, synthetic construct
EVQLLESGGGLVQPGGSLRLSCAASGYTFSNFAISWVRQAPGKGLEWVSSITGSGVRTFYADSVKGRFTISRDNSKNTLYLQMNSLRAEDTAVYYCARSRSYYAGAFDVWGQGTLVTVSSGGGGSGGGGSGGGGSDIQMTQSPSSLSASVGDRVTITCRASQSIGSSVNWYQQKPGKAPKLLIYAASSLQSGVPSRFSGSGSGTDFTLTISSLQPEDFATYYCQQSDNNPWTFGGGTKVEIK

SEQ ID NO: 558 - DNA, 726 bp, synthetic construct
  source 1..726: mol_type = other DNA
gaggtacagctcctcgaaagtggaggagggctggtccagcccggcggctcactaagactttcatgtgcggcatcggggtatactttcagcaatttcgctatttcctgggtgcggcaagcccctggcaaagggctggaatgggtgagcagcataactggttctggtgtgcgtacattctacgccgattctgttaagggaagatttaccatctccagggacaatagcaagaacacattatatctgcagatgaacagtttgcgcgccgaggataccgctgtctactattgcgcaaggtcacgatcctactacgctggcgcctttgacgtttgggggcagggcaccctggtgacggtgtcttccggtggcggtggatcgggcggtggtggatctggaggaggtggctcggatattcagatgactcaatcccctagcagcctgtctgcgagtgtgggcgacagagttaccatcacttgcagggcttcacaatccatagggtcatccgtcaattggtatcagcagaaacctgggaaagctcccaagttgcttatctatgccgcctcttctctgcagtccggcgtgccatctcggttcagcggcagcggaagtgggacagactttacactcaccatctcgtcattacagccagaagatttcgcaacgtactactgtcagcagagtgacaacaacccctggacctttggaggtggtacaaaggtagagattaag

SEQ ID NO: 559 - protein, 242 aa, synthetic construct
EVQLLKSGSGLVQPGGSLRLSCAASGFTFNNAWMHWVRQAPGKGLEWVSAISSSGGYTYYADSVKGRFTISRDNSKNTLYLQMNSLRAEDTAVYYCARSRSYYAGAFDVWGQGTLVTVSSGGGGSGGGGSGGGGSDIQMTQSPSSLSASVGDRVTITCRASAPVGRYLNWYQQKPGKAPKLLIYAASSLQSGVPSRFSGSGSGTDFTLTISSLQPEDFATYYCQQAYSIPITFGGGTKVEIK

SEQ ID NO: 560 - DNA, 726 bp, synthetic construct
  source 1..726: mol_type = other DNA
gaggtacaactcttaaagtccggatctggattggtccagcctggcggcagcctcagactgtcatgcgcagccagcggatttaccttcaacaatgcctggatgcactgggtgaggcaggcacccggcaaaggtcttgagtgggtgtcggccatctcaagctccgggggctatacatattatgcagattcagttaaggggcggttcaccatttctcgtgacaactccaaaaatactctgtacctgcagatgaacagtcttcgagctgaagatacagctgtgtattactgtgctaggtcccgctcttactacgcgggcgcctttgacgtttgggggcagggtactctggtcacggtgagcagtggtggcggtggatcgggcggtggtggatctggaggaggtggctcggatattcagatgacacagtccccaagctcgctgtctgcttccgttggcgatagagtaactatcacatgccgcgccagcgctccagtggggaggtacctcaactggtaccagcagaaacccgggaaggcacctaaattgctgatctatgccgcctcttcactgcagtcaggtgtcccctctcggtttagtgggagcggtagtggaactgactttacacttaccatttcctccttacagcctgaagacttcgcaacgtattactgtcaacaagcgtatagcatccccataaccttcggaggcggcaccaaggtggagattaag

SEQ ID NO: 561 - protein, 242 aa, synthetic construct
EVQLLESGGGLVQPGGSLRLSCAASGFSVRDFAMNWVRQAPGKGLEWVSTITGTDRTPYYADSVKGRFTISRDNSKNTLYLQMNSLRAEDTAVYYCARSRSYYAGAFDVWGQGTLVTVSSGGGGSGGGGSGGGGSDIQMTQSPSSLSASVGDRVTITCRAPQNIFSYINWYQQKPGKAPKLLIYAASSLQSGVPSRFSGSGSGTDFTLTISSLQPEDFATYYCQQTHSIPQTFGGGTKGEIK

SEQ ID NO: 562 - DNA, 726 bp, synthetic construct
  source 1..726: mol_type = other DNA
gaggttcagctgttagaatcaggaggggggttggtgcagcctggaggctctctccggctgtcctgcgccgcatcgggcttctctgtccgtgacttcgccatgaattgggttcggcaggcccccggtaaggggctggagtgggtgagcaccatcacaggtactgatagaacaccatactacgcagattcagtaaagggaagatttactattagcagggacaattctaaaaacacgctttacctccagatgaacagtctaagggctgaagatacagccgtgtattattgtgctcgcagccgatcctactatgctggcgcgtttgacgtgtggggccaaggcaccctggtcaccgtgagttccggtggcggtggatcgggcggtggtggatctggaggaggtggctcggatatacagatgacccagtctcccagcagcctttctgcctcagtcggggatagagttacgatcacatgtcgggctccacagaatatcttcagctacattaactggtaccagcagaagccgggcaaggcacctaagttgctgatctatgcggctagttccttacaatccggagtgccatcgaggttttccggctctgggtccggtacagactttaccctgactattagctcactccagcctgaggacttcgccacctattactgccagcaaacacacagtatcccccaaactttcggcggtgggaccaaaggagaaattaaa

SEQ ID NO: 563 - protein, 243 aa, synthetic construct
EVQLLESGGGLVQPGGSLRLSCAASGFTFSSHNMAWVRQAPGKGLEWVSSIGGGGRTTNYADSVKGRFTISRDNSKNTLYLQMNSLRAEDTAVYYCARAAPGAYAYALDYWGQGTLVTVSSGGGGSGGGGSGGGGSDIQMTQSPSSLSASVGDRVTITCRASQRISTYINWYQQKPGKAPKLLIYGASYLHSGVPSRFSGSGSGTDFTLTISSLQPEDFATYYCQQTYSTPPTFGGGTKVEIK

SEQ ID NO: 564 - DNA, 729 bp, synthetic construct
  source 1..729: mol_type = other DNA
gaagtgcaattgctcgaaagcggcggaggccttgttcagcctggaggtagcctgaggctgtcttgtgcggcttccgggttcaccttcagctcacacaacatggcttgggtgcggcaggcaccaggaaaggggctagagtgggtgagttccatcggtggcggtggaaggactacgaactatgccgactcagtcaaagggcgttttacaatttcccgcgataattctaagaatactctttacctccagatgaacagtctgcgagccgaggataccgctgtctattactgcgccagagctgcacccggcgcctacgcctatgcattagactactggggccaggggaccctggtgacagtatcgtctggtggcggtggatcgggcggtggtggatctggaggaggtggctcggatattcagatgacccaatcaccaagcagccttagcgcatccgttggtgatcgggtgacaattacctgcagggcctctcagagaatatccacgtacatcaactggtaccagcagaagcctgggaaagcgcctaagctcctgatttatggcgccagttatttgcactctggcgtgccctcgcgctttagcggtagtggatcagggaccgactttactctgactatctcttccttacagcccgaagacttcgctacatattactgtcaacagacatactccacacccccaactttcgggggcggaaccaaagtcgagatcaag

SEQ ID NO: 565 - protein, 243 aa, synthetic construct
EVQLLESGGGLVQPGGSLRLSCAASGFSVRDFAMTWVRQAPGKGLEWVSAIRISGGGTFYADSVKGRFTISRDNSKNTLYLQMNSLRAEDTAVYYCARAAPGAYAYALDYWGQGTLVTVSSGGGGSGGGGSGGGGSDIQMTQSPSSLSASVGDRVTITCRASQRISTYINWYQQKPGKAPKLLIYSASVLENGVPSRFSGSGFGTDFTLTISSLQPEDFATYYCQQNYRTPPTFGSGTKGENK

SEQ ID NO: 566 - DNA, 729 bp, synthetic construct
  source 1..729: mol_type = other DNA
gaagtacagcttctggagtctggcggaggactagttcagcccggcgggtcactccggctgagctgtgctgcatcagggtttagcgtccgtgatttcgccatgacatgggtcagacaggctcctggtaagggccttgaatgggtgtccgccatcagaatatcgggggggggaaccttttatgccgacagcgtgaaaggccgattcactatttcccgcgacaattctaagaacactctgtatttgcaaatgaacagtctgagggccgaggatacagccgtctactactgcgcaagggcggcaccaggtgcttacgcttatgccctcgactactggggacagggcacgttagtgaccgtgagttccggtggcggtggatcgggcggtggtggatctggaggaggtggctcggacattcagatgacccagtcaccgtcttcactctccgccagtgtgggtgacagagtgactatcacctgtcgagccagccaaaggatatctacttatatcaactggtatcagcagaaaccagggaaggcacctaagttattgatctactcggcgtccgttctggaaaatggagtcccctctcggtttagtgggagcggattcggcacggattttacacttaccattagctccctgcagccagaggatttcgctacctactactgccaacagaattatcgcactcctcccacattcggtagcggcacaaaaggcgagaacaag

SEQ ID NO: 567 - protein, 244 aa, synthetic construct
EVQLLESGGGLVQPGGSLRLSCAASGFTFSNYAMTWVRQAPGKGLEWVSTITSSASKTNYADSVKGRFTISRDNSKNTLYLQMNSLRAEDTAVYYCARTRYLERFAGGLDIWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSFTAPLTFGGGTKVEIK

SEQ ID NO: 568 - DNA, 732 bp, synthetic construct
  source 1..732: mol_type = other DNA
gaggtccagctgctggaatcaggcggagggctggttcagcccggcggctccctcaggttaagctgcgcggcaagcgggtttaccttctctaattacgccatgacatgggtgagacaagcccctggaaaaggtttggagtgggtgtcgaccatcacgagctctgcttccaagactaactatgcagattctgtgaaaggccggttcacaatttccagagataactcaaagaatactctttacctacagatgaacagtctgcgagccgaagacactgctgtctattactgtgcccgtaccaggtatcttgagcgctttgctggcggtctggacatatggggacaggggacactcgtgaccgtaagcagtggtggcggtggatcgggcggtggtggatctggaggaggtggctcggacatccagatgacccagtctcctagtagcctttctgcaagcgttggagatcgggtgaccatcacatgcagagccagccaaagtataagctcatacttgaactggtaccagcagaagccaggtaaggctccaaaactgttaatttacgctgcctcttccctgcagtcgggcgtgcctagtaggttttcagggtcagggtccggaactgacttcaccctcacaatctcctccctgcagcccgaggatttcgccacctattattgtcaacagtcttttacggcgcccctcactttcggcggtggcacaaaggtcgaaattaaa

SEQ ID NO: 569 - protein, 242 aa, synthetic construct
EVQLLESGGGLVQPGGSLRLSCAASGLTFSNYTMTWVRQAPGKGLEWVSGISGTGGSTWYADSVKGRFTISRDNSKNTLYLQMNSLRAEDTAVYYCARSRSYYAGAFDVWGQGTLVTVSSGGGGSGGGGSGGGGSDIQMTQSPSSLSASVGDRVTITCRASVSISTFLNWYQQKPGKAPKLLIYAASTLQSGVPSRFSGSGSGTDFTLTISSLQPEDFATYYCQQSLRTPITFGGGTKVEIK

SEQ ID NO: 570 - DNA, 726 bp, synthetic construct
  source 1..726: mol_type = other DNA
gaggtgcaactgctggaatctggtggaggtctggtccagcccggaggcagtcttaggctcagctgtgccgcatccggcctaacttttagcaattacaccatgacatgggttcgtcaggcccctgggaaagggttggaatgggtttctggcatctcaggaaccgggggttcgacatggtacgctgattcagtgaagggcagattcaccatttcccgagacaacagcaagaatacgctgtatcttcagatgaacagtttaagggcggaggacactgcagtctattattgcgcccgctcccggtcctactacgctggggccttcgatgtgtggggccagggaaccctcgtaacagtgtctagcggtggcggtggatcgggcggtggtggatctggaggaggtggctcggatattcagatgactcaatcaccctcatcgctgtctgcgagtgtaggggaccgcgtcacaatcacctgtagagcctccgtgagcatcagtacgtttttaaactggtatcaacagaaacccgggaaggctcctaagctgctcatatacgcagcttcaacacttcagagcggtgttccaagcaggttttctgggtccggatctggaactgacttcacattgactatcagctccctccagccagaagatttcgccacctattactgccagcagtccctgcggacccctattaccttcggcggcggcacaaaggtggagattaaa

SEQ ID NO: 571 - protein, 243 aa, synthetic construct
EVQLLESGGGLVQPGGSLRLSCAASGFPFSSFVMSWVRQAPGKGLEWVSSLSGSGDITYYADSVKGRFTISRDNSKNTLYLQMNSLRAEDTAVYYCARAAPGAYAYALDYWGQGTLVTVSSGGGGSGGGGSGGGGSDIQMTQSPSSLSASVGDRVTITCRTSQSVSTYFNWYQQKPGKAPKLLIYAASSLQSGVPSRFSGSGSGTDFTLTISSLQPEDFATYYCQQSYRTPPTFGGGTKVEIK

SEQ ID NO: 572 - DNA, 729 bp, synthetic construct
  source 1..729: mol_type = other DNA
gaagtccagctcttggaatccggaggcggcctggtgcagcccggggggtcattgcgactgagttgcgccgcatctggtttccctttttctagcttcgtgatgtcctgggttagacaggccccggggaagggtttagagtgggtgagctcgctgtccggaagcggggacattacttattatgcagattctgttaagggccgctttaccatcagccgggacaactccaaaaatacactctaccttcaaatgaacagtctacgtgccgaggacaccgccgtgtactactgtgctagggctgccccaggcgcgtatgcatatgctctggattactggggacagggcacgctggtcacagtatcatcaggtggcggtggatcgggcggtggtggatctggaggaggtggctcggatatccaaatgacacagtcgccttcttcccttagcgcatcagtcggggaccgcgtgacaattacctgtaggacctcccagagcgtgagtacttactttaactggtaccaacagaagcccggcaaagcccccaagctgctgatctatgccgcgtctagtttgcagagtggtgttccttcacggttctcagggtccggctctggcacagatttcaccttaactatttccagcctccagccagaggactttgctacgtattactgccagcagagctatagaactccgccaacattcggaggaggtaccaaggtagaaataaaa

SEQ ID NO: 573 - protein, 242 aa, synthetic construct
EVQLLESGGGLVQPGGSLRLSCAASGLTFNAYAMSWVRQAPGKGLEWVSSLTGSGASTFYADSVKGRFTISRDNSKNTLYLQMNSLRAEDTAVYYCARSRSYYAGAFDVWGQGTLVTVSSGGGGSGGGGSGGGGSDIQMTQSPSSLSASVGDRVTITCRASRTVTSYLNWYQQKPGKAPKLLIYGASYLHSGVPSRFSGSGSGTDFTLTISSLQPEDFATYYCQQSYRTPPTFGGGTKVEIK

SEQ ID NO: 574 - DNA, 726 bp, synthetic construct
  source 1..726: mol_type = other DNA
gaggtgcagctactggagtcaggaggcgggctggttcagcctggggggtcattgcgactgagctgtgccgcatctggcctcacctttaacgcctacgccatgagctgggtccgtcaggcgcccggcaaaggcttggaatgggtgtccagcttaactgggtctggagcctccaccttctacgctgactcggtcaagggtagattcacaatctctcgggacaattcaaagaatacactttatctgcagatgaacagtctgagggctgaagatacggctgtgtactattgcgcacgctccaggtcctactatgccggtgcatttgatgtttggggccaaggaaccctcgtaactgtgagcagtggtggcggtggatcgggcggtggtggatctggaggaggtggctcggatatccagatgacacagtcgcccagctccctgagcgcttcagtgggggaccgcgtaactattacatgtagagccagtcgaaccgtcacctcctatctcaactggtaccaacagaagcctggcaaggcgccaaaattactgatatacggagcatcttatttgcactctggtgtgccgtctaggttctccggcagtgggagcggaaccgacttcactcttactatctccagcctgcagccagaagattttgccacatattactgccaacagtcataccggacccctcccacgtttgggggtggcacaaaggttgagattaaa

SEQ ID NO: 575 - protein, 241 aa, synthetic construct
EVQLLESGGGLVQPGGSLRLSCAASGFSVRDFAMNWVRQAPGKGLEWVSSIHRTGGGTYYADSVKGRFTISRDNSKNTLYLQMNSLRAEDTAVYYCARSRSYYAGAFDVWGQGTLVTVSSGGGGSGGGGSGGGGSDIQMTQSPSSLSASVGDRVTITCRANQNIGNYLNWYQQKPGKAPKLLIYAASSLQSGVPSRFSGSGSGTDFTLTISSLQPEDFATYYCQQSYSTSTFGGGTKVEIK

SEQ ID NO: 576 - DNA, 723 bp, synthetic construct
  source 1..723: mol_type = other DNA
gaggtccagctcctggaatcaggtgggggcctggtgcagcccggcgggagcttgcggctttcttgtgctgcaagcggattttcagtgagagacttcgccatgaattgggtccgtcaagcccctggaaaggggttagaatgggtgtctagcattcacaggacaggcgggggcacatactacgcagatagtgttaaaggcaggttcacgatctccagagacaattccaagaacaccctctacctacagatgaacagtctgcgagcggaggacaccgccgtttattactgcgcccggagccgctcctattatgctggagcttttgatgtgtggggtcagggaactctggtgactgtatcttcgggtggcggtggatcgggcggtggtggatctggaggaggtggctcggatattcagatgacgcagtcccccagtagcttgagtgccagcgtgggtgaccgggttactatcacatgtagagcgaaccagaatatcggcaactaccttaattggtaccaacagaagcctgggaaggctcccaaactcctgatctatgcagcctcatccctgcagtcaggtgtgccgtccaggttttcggggtccgggagcggaaccgatttcaccctgacaatttcttctttacaaccagaagactttgctacctactattgccagcagtcatatagcacctctactttcggcggcggaacaaaggtcgagataaaa

SEQ ID NO: 577 - protein, 242 aa, synthetic construct
EVQLLESGGGLVQPGGSLRLSCAASGFDFRSYPMAWVRQAPGKGLEWVSVISGGGGSTNYADSVKGRFTISRDNSKNTLYLQMNSLRAEDTAVYYCARSRSYYAGAFDVWGQGTLVTVSSGGGGSGGGGSGGGGSDIQMTQSPSSLSASVGDRVTITCRASQKIARYVNWYQQKPGKAPKLLIYAASDLQSGVPSRFSGSGSGTDFTLTISSLQPEDFATYYCQQAYSIPITFGGGTKVEIK

SEQ ID NO: 578 - DNA, 726 bp, synthetic construct
  source 1..726: mol_type = other DNA
gaggtccagctgttggaaagcggggggggcttagtacagcctggaggttccctccgtctaagctgcgccgcatcgggattcgactttcgctcctacccaatggcttgggtgcggcaggcccccggcaagggcctggaatgggttagtgttatttctggtggaggcggttcaaccaactacgcggacagcgtcaaagggagatttactatctctagggacaattccaagaacacgctgtacctgcagatgaatagtctccgagcagaggatacagccgtgtattattgtgccaggtcaagatcctactatgctggagctttcgatgtgtggggccaagggacacttgtgaccgtgagctctggtggcggtggatcgggcggtggtggatctggaggaggtggctcggacatacagatgacacagtctccatctagccttagtgcctccgttggagatcgggtgaccatcacatgccgcgcctcacagaagattgcaagatatgtcaactggtaccagcagaaacctggtaaggcaccaaagctcttaatctatgctgcgagcgacttgcaaagcggcgtgccctctaggttttccggcagcggttcaggcaccgatttcaccctgacgatctcctcgctgcagcctgaggactttgctacttattactgtcagcaagcctacagtattcccattacattcgggggagggactaaagtagaaatcaaa

SEQ ID NO: 579 - protein, 240 aa, synthetic construct
EVQLLESGGGLVQPGGSLRLSCAASGFRFSNYAMTWVRQAPGKGLEWVSGISGGGGRTFYADSVKGRFTISRDNSKNTLYLQMNSLRAEDTAVYYCARGYGAEYFDPWGQGTLVTVSSGGGGSGGGGSGGGGSDIQMTQSPSSLSASVGDRVTITCRASQPISRYLNWYQQKPGKAPKLLIYDASRLQVGVPSRFSGSGSGTDFTLTISSLQPEDFATYYCQQSHSIPWTFGGGTKVEIK

SEQ ID NO: 580 - DNA, 720 bp, synthetic construct
  source 1..720: mol_type = other DNA
gaggtgcaactccttgagtcaggtggcggactcgtgcagcctggcggatcgctgagactgtcatgtgcggcttctgggtttcgtttctccaactacgcaatgacatgggtgaggcaggcccccgggaaaggcctggagtgggtatccggaatcagcggcggcgggggcaggaccttctatgcagatagtgtcaagggtagatttaccattagccgggacaattccaagaatacattatacctacagatgaacagtctgcgcgccgaagatacggctgtctattactgcgcccgaggttacggggccgaatatttcgacccgtgggggcagggaactttggttactgtgagctctggtggcggtggatcgggcggtggtggatctggaggaggtggctcggacatccagatgacccagtcaccctcttccctgtccgcaagtgtgggcgaccgggtgactattacttgtcgcgccagccagcccatcagtagatacttaaactggtatcagcagaagccgggcaaagcgccaaagctgttgatatatgacgcttctcgacttcaagtcggcgtacctagcaggttctcagggagcggttctggaacggatttcacactgaccatctcgtccctccaaccagaggattttgccacatactactgccagcagagccactccattccttggacatttgggggtggaaccaaggttgaaattaaa

SEQ ID NO: 581 - protein, 240 aa, synthetic construct
EVQLLESGGGLVQPGGSLRLSCAASGFRFSNYAMTWVRQAPGKGLEWVSGISGGGGRTFYADSVKGRFTISRDNSKNTLYLQMNSLRAEDTAVYYCARGYGAEYFDPWGQGTLVTVSSGGGGSGGGGSGGGGSDIQMTQSPSSLSASVGDRVTITCRASQPISRHLNWYQQKPGKAPKLLIYSASSLQSGVPSRFSGSGSGTDFTLTISSLQPEDFATYYCQQSYDRTWTFGGGTKGESK

SEQ ID NO: 582 - DNA, 720 bp, synthetic construct
  source 1..720: mol_type = other DNA
gaggtgcagctgctcgaatcgggcgggggcctggtccagcccggggggtctttgaggctctcctgtgctgcctcagggtttaggttcagtaattacgcgatgacttgggtgcgacaggccccaggaaagggcctagagtgggtgagcggcatttcaggtggaggaggcagaacattttatgcagatagcgttaaaggtagattcacgatctctcgggacaactccaagaataccctttacttacaaatgaacagtctgcgcgcagaggataccgccgtatactattgcgctcgtgggtatggagccgaatacttcgacccttggggccagggtaccctggtcacagtgagctccggtggcggtggatcgggcggtggtggatctggaggaggtggctcggacatccaaatgacccagagcccatcatccctgagcgcatcagtgggagatagagtcactatcacctgtcgcgcttctcaaccaatatcccgacacctcaactggtaccagcagaaacccggaaaggcccctaagctgttgatttatagtgccagttccctgcagtcaggtgttcctagcaggttttctggctctgggtccggcacagacttcacgcttactatttctagcttacagcccgaagattttgcgacatactactgccagcagagttatgaccggacatggaccttcggtgggggcaccaagggggagtcgaaa

SEQ ID NO: 583 - protein, 240 aa, synthetic construct
EVQLLESGGGLVQPGGSLRLSCAASGFRFSNYAMTWVRQAPGKGLEWVSGISGGGGRTFYADSVKGRFTISRDNSKNTLYLQMNSLRAEDTAVYYCARGYGAEYFDPWGQGTLVTVSSGGGGSGGGGSGGGGSDIQMTQSPSSLSASVGDRVTITCRASQRIATYLNWYQQKPGKAPKLLIYAASHLHGGVPSRFSGSGSGTDFTLTISSLQPEDFATYYCQQSYSIPLTFGGGTKVEIK

SEQ ID NO: 584 - DNA, 720 bp, synthetic construct
  source 1..720: mol_type = other DNA
gaagtccagctcttggaatccgggggggggttagtgcagcctggaggctctctcagactgagttgcgccgccagcggatttaggttctccaattatgccatgacgtgggtgagacaggcccccgggaaggggctggagtgggtgtctggcatttccggcggaggcggacggactttctatgcagactcagtaaagggtaggttcacaatcagccgagacaactcgaaaaataccctttacctgcaaatgaacagtctgcgcgcggaggataccgctgtgtactactgtgcacgtggttacggcgctgagtattttgatccgtggggccagggtacactagtcactgttagctcaggtggcggtggatcgggcggtggtggatctggaggaggtggctcggacattcagatgacccagtcacccagtagcttgtctgcctctgtgggagatcgcgtcacaataacttgcagggcttcccaacggatcgccacctacctgaactggtaccagcaaaaacctggtaaggcaccaaagctgttaatttacgcggcctctcaccttcatggaggcgtgccatccagattctccggcagcgggtcaggcacagattttaccctgactatctcctcgctccagcccgaagacttcgctacgtattattgtcagcagagttatagcatccctctcacatttggtggggggaccaaggttgagattaaa

SEQ ID NO: 585 - protein, 246 aa, synthetic construct
GGAALKSGGGLVQPGGSLRLSCAASGFTVSRDYMSWVRQAPGKGLEWVSVISTGGGSTYYADSVKGRFTISRDNSKNTLYLQMNSLRAEDTAVYYCAKPRPYSIAWFADPFDYWGQGTLVTVSSGGGGSGGGGSGGGGSDIQMTQSPSSLSASVGDRVTITCRTSQTISIYLNWYQQKPGKAPKLLIYAASILHGGVPSRFSGSGSGTDFTLTISSLQPEDFATYYCQQNYSVPPTFGGGTKVEIK

SEQ ID NO: 586 - DNA, 738 bp, synthetic construct
  source 1..738: mol_type = other DNA
ggaggcgcggccttaaagtccggaggtggcctggtccagcccggagggtccctcagattgagctgtgctgcctctggctttacagtgtcacgggattatatgtcgtgggtccgccaagctccgggcaagggactggaatgggtgagtgtgattagcaccgggggtgggtccacgtactatgccgactctgttaaggggaggttcacaatctccagggataattcaaaaaacaccctgtaccttcagatgaacagtctccgtgctgaggacaccgcagtttattattgcgcaaaaccccgaccatacagcatagcatggttcgccgacccttttgattactggggccagggtactctggtgactgtaagctctggtggcggtggatcgggcggtggtggatctggaggaggtggctcggatatccaaatgactcagagcccttccagcttgagtgcgtccgtgggggatagggtaactatcacatgtagaacaagccagaccatatctatttacctcaactggtatcagcagaagcccggaaaggccccaaaattactgatttatgcagcctcaatcctgcacggaggtgttccgtcacggttctccgggtcgggctctggtaccgactttacccttacgattagtagcctgcagcctgaggacttcgctacctactactgccaacagaattattctgtcccacccacttttgggggcggcacaaaagtggaaatcaag

SEQ ID NO: 587 - protein, 243 aa, synthetic construct
ELQLLESGGGLVQPGGSLRLSCAASGFPFSNNAMSWVRQAPGKGLEWVSVISGSYGTTYYADSVKGRFTISRDNSKNTLYLQMNSLRAEDTAVYYCARVADGAAAYAMDYWGQGTLVTVSSGGGGSGGGGSGGGGSDIQMTQSPSSLSASVGDRVTITCRASQPISRYLNWYQQKPGKAPKLLIYDASRLQVGVPSRFSGSGSGTDFTLTISSLQPEDFATYYCQQSHSIPWTFGGGTKVEIK

SEQ ID NO: 588 - DNA, 729 bp, synthetic construct
  source 1..729: mol_type = other DNA
gagctgcaactcttagaatctggcggaggcctggttcagcccggaggcagtctgcgtctatcctgtgccgcatctgggttccctttctccaacaacgccatgtcatgggtccgccaggccccaggtaaaggtcttgaatgggtttctgtgatcagcggcagctacgggactacatactatgccgattccgtgaagggcagatttaccattagccgggataattcgaagaacacgctttatctgcagatgaatagtttgagggctgaggacaccgcagtgtattactgcgcccgagtggctgacggggcagctgcttacgcgatggactattgggggcagggaactctcgtcacagtaagctcaggtggcggtggatcgggcggtggtggatctggaggaggtggctcggacattcagatgacccagtcaccatccagtctgtcagcttcggtgggcgatagggtaacaatcacttgcagagcgtcccagcccatctctcgctatcttaactggtaccagcaaaagccggggaaagcaccaaagctgttaatttacgacgcctctcggctgcaggtcggcgttcctagccgattcagcggttctgggtccggaacagattttaccctcacaattagctccttgcagcccgaagactttgccacctattactgtcagcaaagtcacagcatcccttggacgttcggtggaggcactaaggtggagataaaa

SEQ ID NO: 589 - protein, 240 aa, synthetic construct
EVQLLESGGGLVQPGGSLRLSCAASGFRFSNYAMTWVRQAPGKGLEWVSGISGGGGRTFYADSVKGRFTISRDNSKNTLYLQMNSLRAEDTAVYYCARGYGAEYFDPWGQGTLVTVSSGGGGSGGGGSGGGGSDIQMTQSPSSLSASVGDRVTITCRASQPISRYLNWYQQKPGKAPKLLIYAASNLKKGVPSRFSGSGFGTDFTLTISSLQPEDFATYYCQQSYNPPLTFGGGTKVEIK

SEQ ID NO: 590 - DNA, 720 bp, synthetic construct
  source 1..720: mol_type = other DNA
gaggtgcaactgcttgagagtggcggaggcctggtgcagcctgggggcagcctcagattatcttgcgcggcctcaggttttcgtttctctaactatgctatgacatgggtccgacaggctccagggaaagggctagaatgggtatcgggaatttccggtggcgggggccggactttctacgccgatagcgtcaagggaaggttcacaatcagccgcgacaattcaaagaataccctctacctgcagatgaacagtttgagggccgaggacactgcagtgtattactgtgccagaggctacggagcagaatattttgatccctgggggcagggtaccctggtgacggtttcctccggtggcggtggatcgggcggtggtggatctggaggaggtggctcggacatccagatgacccaaagtccctcttctcttagcgcttcggtgggcgatcgggtgaccattacttgtagagcgtcacagccaataagcaggtatctcaattggtatcagcagaagcccggaaaagcacccaagttgctgatctacgccgcctccaacttaaagaagggggttcctagccgctttagtgggtccggcttcggaaccgatttcacactcacaatctcatccctgcagccggaggactttgctacgtactattgccaacagtcttacaaccctccactgactttcggtggcgggacaaaagtcgaaattaaa

SEQ ID NO: 591 - protein, 243 aa, synthetic construct
EVQLLESGGGLVQPGGSLRLSCAASGFTFPHSAMSWVRQAPGKGLEWVSSIAGRGGSPNYADSVKGRFTISRDNSKNTLYLQMNSLRAEDTAVYYCARVADGGAAYAFDYWGQGTLVTVSSGGGGSGGGGSGGGGSDIQMTQSPSSLSASVGDRVTITCRSSQNIITYLNWYQQKPGKAPKLAIYGASRVQSGVPSRFSGSGSGADFTLTISSLQPEDFSTYYCQQSFSTPLTFGGGTKVEIK

SEQ ID NO: 592 - DNA, 729 bp, synthetic construct
  source 1..729: mol_type = other DNA
gaggtgcagctgctggagagcggaggcggactagtgcagccaggtgggtccctgcgacttagctgcgccgcttcggggtttacgtttccgcactctgcaatgtcttgggtgcgtcaggcacctggaaagggcttagaatgggtcagcagcatcgctggcagaggtggctctcccaattacgccgactccgtgaagggtaggttcactatttcccgcgataactcaaaaaatacactctatctgcagatgaacagtttgagggccgaagacaccgcggtatattattgtgcacgggttgccgatggcggggctgcctacgctttcgactactgggggcaaggaaccctcgttacagtcagttcaggtggcggtggatcgggcggtggtggatctggaggaggtggctcggacattcaaatgacacagagtccttcttccttgtctgcatccgtcggtgatcgcgttaccatcacttgccggtcgagccagaacataatcacttatctcaattggtaccagcagaaacccggaaaagccccaaagttagcgatttacggtgcctccagggtgcagagcggcgtgcccagcagattcagcgggagtgggtcaggggctgatttcacactgaccatcagttctcttcagcctgaggacttttcaacctattactgtcaacagtccttctcaacgccactgacctttggcggaggcacaaaggtagaaattaag

SEQ ID NO: 593 - protein, 240 aa, synthetic construct
EVQLLESGGGLVQPGGSLRLSCAASGFRFSNYAMTWVRQAPGKGLEWVSGISGGGGRTFYADSVKGRFTISRDNSKNTLYLQMNSLRAEDTAVYYCARGYGAEYFDPWGQGTLVTVSSGGGGSGGGGSGGGGSDIQMTQSPSSLSASVGDRVTITCRASQPISRYLNWYQQKPGKAPKLLIYDASRLQVGVPSRFSGSGSGTDFTLTISSPQPEDFATYYCQQSHSIPWTFGGGTKGEIK

SEQ ID NO: 594 - DNA, 720 bp, synthetic construct
  source 1..720: mol_type = other DNA
gaggtgcagctcctggaaagcggagggggcttggtgcagcccggagggtccctccgattatcctgtgctgcctcagggtttcgtttttcaaattacgcaatgacatgggtccggcaggcccctggcaaggggctggagtgggtgagcgggatttccggaggtggtggcagaacgttctacgcagattcggtgaagggtagattcaccatcagtagggacaattctaaaaacactctatatctgcagatgaacagtctgagggctgaagacactgccgtttattattgcgcgcgcggatacggcgccgagtacttcgatccgtggggccaaggcacccttgtcacagtaagctctggtggcggtggatcgggcggtggtggatctggaggaggtggctcggacatccagatgacacagtcaccctccagcctgtctgcctctgtgggggacagggtcaccattacttgtcgggcatcgcaaccaattagcagatacttgaactggtatcagcagaagccaggaaaggcccccaagttactcatttacgacgctagtcgacttcaagtgggtgttccttctcgcttctccgggtccggaagcggcactgattttaccctgaccatctcaagtccccagcctgaagattttgcgacatattactgccagcagtcccacagcataccgtggacgttcggtgggggcacaaaaggcgagatcaaa

SEQ ID NO: 595 - protein, 243 aa, synthetic construct
EVQLLESGGGLVQPGGSLRLSCAASGFTFPHSAMSWVRQAPGKGLEWVSTVTGSGSPTYYADSVKGRFTISRDNSKNTLYLQMNSLRAEDTAVYYCARVAGGAYGYAMDYWGQGTLVTVSSGGGGSGGGGSGGGGSDIQMTQSPSSLSASVGDRVTITCRASQSISSYLNWYQQKPGKAPKLLIYDASNLQSGVPSRFSGSGSGTDFTLTISSLQPEDFATYYCQQSFSNLYTFGGGTKVEIK

SEQ ID NO: 596 - DNA, 729 bp, synthetic construct
  source 1..729: mol_type = other DNA
gaagtgcagctcctggagtcaggcgggggactggtccagcccggaggttcactgcgtctaagctgcgctgcatctggcttcacattcccacactccgccatgtcttgggttcgccaggcccctgggaagggattggaatgggtgtccaccgttacaggttccggcagcccgacatattatgccgatagcgtgaaagggcgatttaccatcagcagggacaacagtaagaatacgctgtatcttcagatgaacagtctccgggctgaggatactgcggtctactactgtgcaagagtggctggaggggcctacggttatgcaatggactactggggccaaggcactttagtgaccgtatcgtctggtggcggtggatcgggcggtggtggatctggaggaggtggctcggacatacagatgacccagtccccgtcaagtctcagcgcctcggtcggggacagagttacaatcacgtgtcgggcatctcaatccatttccagctatctgaattggtaccagcagaaacctgggaaggctccaaagctgcttatctacgatgccagcaacctgcagagcggagtgccctcaaggttctccggcagtggctctgggactgattttaccctcacaatttcttctttacagcccgaagactttgcgacttactattgccagcaatcattcagtaacttgtataccttcggtggaggcacaaaggtggagatcaaa

SEQ ID NO: 597 - protein, 243 aa, synthetic construct
EVQLLESGGGLVQPGGSLRLSCAASGFPFSVYAMTWVRQAPGKGLEWVSSFGGSGHSPYYADSVKGRFTISRDNSKNTLYLQMNSLRAEDTAVYYCARVAAGSYAYAMDYWGQGTLVTVSSGGGGSGGGGSGGGGSDIQMTQSPSSLSASVGDRVTITCRSSQNIITYLNWYQQKPGKAPKLLIYGASRLQSGVPSRFSGSGSGTDFTLTISSLQPEDFATYYCQQSFSTPLTFGGGTKEVDK

SEQ ID NO: 598 - DNA, 729 bp, synthetic construct
  source 1..729: mol_type = other DNA
gaggtgcagcttctggaatcagggggtggactggttcagcctgggggttccctccgcctgtcgtgtgccgcttctggctttccattctccgtatatgcaatgacatgggtgaggcaggccccgggcaagggattggagtgggtgagttcttttggcggcagcgggcactccccctattacgcagattccgtgaaggggcgtttcacaatctcaagagataattctaaaaacacgctgtatctacagatgaacagtctccgggctgaagacaccgccgtttactactgcgcgcgagtggccgctggcagctacgcctatgcaatggactactggggacaaggaaccttagtcactgtcagcagcggtggcggtggatcgggcggtggtggatctggaggaggtggctcggatatccagatgactcagagcccatcatcgctcagcgcatctgtcggggacagggttaccataacctgcagaagttcacagaacatcatcacctaccttaattggtaccagcaaaagcctggcaaagcccctaaactgctcatttatggagcctcccggctgcagagcggcgtgcccagtcgcttctccggttctggatcaggtaccgatttcacgctgacaattagctccttacaacccgaagactttgctacttactattgtcagcagtccttctctactccattgacatttggcggggggacaaaggaggtggacaag

SEQ ID NO: 599 - protein, 240 aa, synthetic construct
EVQLLESGGGLVQPGGSLRLSCAASGFRFSNYAMTWVRQAPGKGLEWVSGISGGGGRTFYADSVKGRFTISRDNSKNTLYLQMNSLRAEDTAVYYCARGYGAEYFDPWGQGTLVTVSSGGGGSGGGGSGGGGSDIQMTQSPSSLSASVGDRVTITCRASQPISRYLNWYQQKPGKAPKLLIYDASRLQVGVPSRFSGSGSGTDFTLTISSLQPEDFATYYCQQSFSTPLTFGGGTKVEIK

SEQ ID NO: 600 - DNA, 843 bp, synthetic construct
  source 1..843: mol_type = other DNA
gaagtccagcttctggagagcggaggcggactggtgcagccaggcgggagcttgcgtctgtcctgtgcggcaagcgggttccgcttctctaactatgccatgacctgggtaaggcaagcacctggcaaaggtcttgagtgggtgtccggaatttccggcgggggcggacggacgttctacgccgattcagtgaagggtcgatttaccatctctagggataatagtaagaacactctctacctccagatgaatagtttgagagctgaagacacagccgtgtactattgcgctagagggtacggtgccgagtattttgacccctgggggcagggcactctggttacagtctcatcgggtggcggtggatcgggcggtggtggatctggaggaggtggctcggatattcagatgacacagagcccatcttcactgtccgcttcggtaggggatagagtgacaataacatgccgcgcaagccagcctatctcacggtacctcaactggtaccaacagaaacccgggaaagccccgaagctgttaatctatgacgcgagtaggctgcaagtgggtgtccccagccgattcagcggttccggatctggcaccgacttcaccctcactatctcttccttgcagccagaagactttgccacgtattactgtcagcagtcctttagtacccctcttaccttcggcggaggcactaaggttgagattaagtttggggagctggacccttcgactattgggggcagggcacattagtgacagtgtcgagcaatagtctccgcgccgaggatacggctgtctactactgcgctaaaccgcgtccaggttcaattt

SEQ ID NO: 601 - protein, 446 aa, synthetic construct
EVQLLESGGGLVQPGGSLRLSCAASGFDFSKFAMSWVRQAPGKGLEWVSSITRGSETTYYADSVKGRFTISRDNSKNTLYLQMNSLRAEDTAVYYCATLGLGYYYYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 602 - protein, 214 aa, synthetic construct
DIQMTQSPSSLSASVGDRVTITCRASQPISSYVTWYQQKPGKAPKLLIYSASHLRSGVPSRFSGSGSGTDFTLTISSLQPEDFATYYCQQSYNAPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC